(19) United States
(12) Patent Application Publication
(10) Pub. No.: US 2024/0100180 A1
(43) Pub. Date: Mar. 28, 2024

(54) TUMOR-SPECIFIC CLAUDIN 18.2 ANTIBODY-DRUG CONJUGATES

(71) Applicant: SOTIO Biotech a.s., Prague 7 (CZ)

(72) Inventors: Lukas Bammert, Basel (CH); Lenka Kyrych Sadilkova, Horomerice (CZ); Simona Hoskova, Prague 9 (CZ); Iva Valentova, Ceske Budejovice (CZ); Lorenz Waldmeier, Nidau (CH); Roger Beerli, Adlikon bei Regensdorf (CH); Ulrich Moebius, Gauting (DE)

(21) Appl. No.: 18/269,240

(22) PCT Filed: Dec. 23, 2021
(86) PCT No.: PCT/EP2021/087495

§ 371 Date: Jun. 22, 2023

(30) Foreign Application Priority Data
Dec. 23, 2020 (EP) 20 216 800.1

(51) Int. Cl. A61K 47/68; A61K 31/704; A61P 35/00; C07K 16/28

(52) U.S. Cl. CPC A61K 47/6849; A61K 31/704; A61K 47/6809; A61K 47/6889; A61P 35/00; C07K 16/28

(57) ABSTRACT

The invention provides an ADC based on an antibody binding to CLDN18.2, wherein the antibody or fragment thereof exhibits increased binding to tumor tissue expressing CLDN18.2 over healthy tissue expressing CLDN18.2.

### BACKGROUND

Tight junctions are multiprotein complexes connecting adjacent epithelial or endothelial cells to form a barrier, preventing molecules from passing in between the cells, and helping to maintain the cell and tissue polarity. Tight junctions consist of three main groups of transmembrane proteins: claudins and occludin, cytoplasmic plaque proteins, and cingulin. They also contain cytoskeletal and signaling proteins, e.g. actin, myosin II, and PKC. These proteins interact to maintain the tight junction structure (Yu and Turner 2008).

Claudins form a family of 23 proteins (Hewitt, Agarwal, and Morin 2006). Claudin 18 is a human protein encoded by the CLDN18 gene which forms tight junction strands in epithelial cells. The human CLDN18 can be alternatively spliced with two alternative first exons, resulting in two protein isoforms, CLDN18.1 (or Claudin 18.1) and CLDN18.2 (or Claudin 18.2). CLDN18.2 was first disclosed as Zsig28 protein in WO2000/015659. The two isoforms differ in the N-terminal 69 amino acids encompassing the first extracellular loop. The first extracellular domain spans from amino acid 28 to amino acid 80. Within this stretch there are 8 amino acid differences between CLDN18.1 and CLDN18.2. The two different isoforms are expressed in different tissues, with CLDN18.1 being predominantly expressed in lung tissue whereas CLDN18.2 displays stomach specificity (Niimi et al. 2001). CLDN18.2 expression in normal stomach is restricted to the differentiated short-lived cells of stomach epithelium. CLDN18.2 expression has further been identified in various tumor tissues. For example, CLDN18.2 has been found to be expressed in pancreatic, esophageal, ovarian, and lung tumors, correlating with distinct histologic subtypes (Sahin et al. 2008). The amino acid sequence of human CLDN18.2 protein has the NCBI reference sequence: NP 001002026.1 The sequence can also be derived from SEQ ID NO: 135.

In view of its restricted expression pattern in normal tissues, and of its ectopic expression in human cancers, CLDN18.2 is an attractive cancer target for antibody therapy of epithelial tumors. A number of studies have been made towards such an antibody therapy. WO2004/047863 identified the splice variants of CLDN18 and screened antibodies against different peptides derived from CLDN18.2: peptide DQWSTQDLYN (SEQ ID NO: 57), N-terminal extracellular of CLDN18.2, independent of glycosylation; peptide NNPVTAVFNYQ (SEQ ID NO: 58), N-terminal extracellular of CLDN18.2, mainly unglycosylated; and peptide STQDLYNNPVTAVF (SEQ ID NO: 59), N-terminal extracellular domain of CLDN18.2, unglycosylated. It also disclosed polyclonal rabbit antibodies screened with a pan-CLDN18 peptide TNFWMSTANMYTG (SEQ ID NO: 60) in the C-terminal extracellular domain common to both CLDN18.1 and CLDN18.2 isoforms. WO2005/113587 discloses antibodies against specific epitopes of CLDN18.2 defined by the peptide sequences: ALMIVGIVLGAIGLLV (SEQ ID NO: 61) and RIGSMEDSAKANMTLTSGIMFIVS (SEQ ID NO: 62). WO2007/059997 discloses CLDN18.2 specific monoclonal antibodies obtained by immunization with the peptide METDTLLLWVLLLWVPGSTGDAAQPARRARRTKLGTELGSTPVWWNSADGRMDQ WSTQDLYNNPVTAVFNYQGLWRSCVRESSGFTECRGYFTLLGLPAMLQAVRAAIQH SGGRSRRARTKTHLRRGSE (SEQ ID NO: 63), including the first extracellular domain of CLDN18.2 with N- and C-terminal extensions. Antibodies obtained by this immunization mediate cell killing by complement dependent cytotoxicity (CDC) and antibody-dependent cell-mediated cytotoxicity (ADCC). Antibody IMAB362, also known as Claudiximab or Zolbetuximab, is disclosed in WO2007/059997 and WO2016/165762. IMAB362 is an IgG1 antibody derived from a murine monoclonal antibody and has been chimerized to display the human IgG1 constant region for clinical use. WO2008/145338 also discloses antibodies binding to overlapping peptides within the first extracellular domain (MDQWSTQDLYNNPVT (SEQ ID NO: 64), LYNNPVTAVFNYQGL (SEQ ID NO: 65), VFNYQGLWRSCVRES (SEQ ID NO: 66), QGLWRSCVRESSGFT (SEQ ID NO: 67), and RSCVRESSGFTECRG (SEQ ID NO: 68)). In an effort to produce antibodies targeting the C-terminal portion of CLDN18.2 for diagnostic purposes to detect CLDN18.2 expression in cells of cancer tissue sections, WO2013/167259 discloses antibodies binding to C-terminal epitopes of CLDN18.2. The sequences of the two epitopes are TEDEVQSYPSKHDYV (SEQ ID NO: 69) and EVQSYPSKHDYV (SEQ ID NO: 70). WO2013/174509 presents combinations of anti-CLDN18.2 antibodies with agents stabilizing γδ T cells or with agents stabilizing or increasing the expression of CLDN18.2. Antibodies may be conjugated to a therapeutic moiety such as a cytotoxin, a drug (e.g. an immunosuppressant) or a radioisotope. WO2014/075788 discloses a method of treatment a cancer disease using a bispecific antibody binding CLDN18.2 and CD3. WO2014/127906 discloses combination agents stabilizing or increasing the expression of CLDN18.2. WO2016/166122 discloses anti-CLDN18.2 monoclonal antibodies that can be highly efficiently internalized upon CLDN18.2 binding and therefore are suitable for antibody-drug conjugate (ADC) development. Furthermore, the conjugation of such antibodies to the drugs DM4 and MMAE using cleavable SPDB or Valine-Citrulline linkers, respectively, is disclosed. However, despite all the antibodies disclosed in the patent applications, only the chimeric IMAB362, disclosed in WO2007/059997 and WO2016/165762, is currently tested in clinical trial. In addition to these antibodies and ADCs, WO2018/006882 discloses chimeric antigen receptors (CAR) based on anti-CLDN18.2 monoclonal antibodies. Antibodies of WO2018/006882 have been humanized and their sequence is disclosed in the Supplementary Materials section associated with Jiang et al 2018 (Jiang et al. 2018). CAR T-cells based on the humanized antibody are currently tested in a phase I clinical trial (ClinicalTrials.gov Identifier: NCT03159819) in patients with advanced gastric adenocarcinoma and pancreatic adenocarcinoma. CN109762067 discloses other anti-CLDN18.2 monoclonal antibodies mediating cell killing by CDC and ADCC. WO2019/173420 discloses anti-CLDN18.2 humanized monoclonal antibodies with ADCC activity. WO2019/175617 discloses anti-CLDN18.2 monoclonal antibodies binding to a different epitope than IMAB362. WO2019/219089 discloses monoclonal antibodies binding to a mutant of CLDN18.2. Other antibodies binding to CLDN18.2 have been disclosed in WO2019/242505, WO2020/038404, WO2020/043044, WO2020/063988, WO2020/082209, WO2020/018852, WO2020/023679, WO2020/135674, WO2020/135201, WO2020/139956, WO2020/025792, WO2020160560, CN111808194 and WO2020200196.

CLDN18.2 has been described to exist in different conformations and contains a potential extracellular N-glycosylation site (see WO2007/059997 page 3, first para.), which may lead to potentially different topologies/differential glycosylation between normal and tumor cells (see WO2007/059997 page 4, second para.). However, none of the reported antibodies is preferentially targeting CLDN18.2 expressed on tumor cells. Since CLDN18.2 is expressed not only in tumors, but also in healthy tissue, namely in stomach tissue (Sahin et al. 2008), it clearly would be beneficial to have antibodies targeting only CLDN18.2 expressed in tumor in order to avoid safety issues and side effect very often associated with the on-target effect of therapeutic antibodies to healthy organs/tissues (Hansel et al. 2010), in particular as reported for IMAB362 (Sahin et al. 2018; Tureci et al. 2019).

In addition to binding to targets with high affinity, therapeutic antibodies should maintain their desired properties during development, production, storage and clinical application (in vivo). Antibody stability may be compromised by post-translational modifications (PTM) (Lu et al. 2019; Gervais 2016). Since uncontrolled PTM may lead to antibodies with less than desired efficacy, activity, potency or stability, it is therefore very important while developing therapeutic antibodies to design them with the minimal possible PTMs. PTMs can also have a profound effect on regulatory acceptance, technology transfer or processes and development of biosimilars. The predominant modifications are oxidation, deamidation and isomerization. Further, IMAB362 is a chimeric antibody still having extended mouse sequence, which could lead to antidrug antibodies in some patients, which, e.g. upon repeated application, may lead to decreased efficacy of the treatment.

As already mentioned above, IMAB362 has also been developed as an antibody-drug conjugate (ADC) (disclosed in WO2016/165762), where the antibody has been conjugated to the MIME or DM4 drugs. The DM4 drug was coupled to IMAB362 via SPBD (N-succinimidyl-3-(2 pyridyldithio)butyrate), an amino and sulfhydryl reactive heterobifunctional protein crosslinker which reacts via an N-hydroxysuccinimide (NETS) ester with primary amines (as found in lysine side chains or the N-terminus of proteins) of the antibody. The valine-citrulline-MMAE drug was coupled to thiolated IMAB362. In that case, IMAB362 was initially thiolated with the heterobifunctional linker 2-IT (2-iminothilane) which reacts with the free amines of lysine residues. The valine-citrulline is a linker cleavable by cathepsins. All the caveats listed above related to IMAB362 also apply to an ADC based on the same antibody.

Therefore, there is a need for improved antibodies and ADCs specific to CLDN18.2 for use in the treatment of tumor patients.

### Definitions

“Antibodies” or “antibody”, also called “immunoglobulins” (Ig), generally comprise four polypeptide chains, two heavy (H) chains and two light (L) chains, and are therefore multimeric proteins, or comprise an equivalent Ig homologue thereof (e.g., a camelid antibody comprising only a heavy chain, single-domain antibodies (sdAb) or nanobody which can be either be derived from a heavy or light chain). The term “antibodies” includes antibody-based binding protein, modified antibody format retaining target binding capacity. The term “antibodies” also includes full length functional mutants, variants, or derivatives thereof (including, but not limited to, murine, chimeric, humanized and fully human antibodies) which retain the essential epitope binding features of an Ig molecule, and includes dual specific, bispecific, multispecific, and dual variable domain Igs. Ig molecules can be of any class (e.g., IgG, IgE, IgM, IgD, IgA, and IgY), or subclass (e.g., IgG1, IgG2, IgG3, IgG4, IgA1, and IgA2) and allotype. Ig molecules may also be mutated e.g. to enhance or reduce affinity for Fcγ receptors or the neonatal Fc receptor (FcRn).

An “antibody fragment”, as used herein, relates to a molecule comprising at least one polypeptide chain derived from an antibody that is not full length and exhibits target binding. Antibody fragments are capable of binding to the same epitope or target as their corresponding full-length antibody. Antibody fragments include, but are not limited to (i) a Fab fragment, which is a monovalent fragment consisting of the variable light (VL), variable heavy (VH), constant light (CL) and constant heavy 1 (CH1) domains; (ii) a F(ab′)2 fragment, which is a bivalent fragment comprising two Fab fragments linked by a disulfide bridge at the hinge region (reduction of a F(ab′)2 fragment result in two Fab′ fragment with a free sulfhydryl group); (iii) a heavy chain portion of a Fab (Fa) fragment, which consists of the VH and CH1 domains; (iv) a variable fragment (Fv) fragment, which consists of the VL and VH domains of a single arm of an antibody; (v) a domain antibody (dAb) fragment, which comprises a single variable domain; (vi) an isolated complementarity determining region (CDR); (vii) a single chain Fv fragment (scFv); (viii) a diabody, which is a bivalent, bispecific antibody in which VH and VL domains are expressed on a single polypeptide chain, but using a linker that is too short to allow for pairing between the two domains on the same chain, thereby forcing the domains to pair with the complementarity domains of another chain and creating two antigen binding sites; (ix) a linear antibody, which comprises a pair of tandem Fv segments (VH-CH1-VH-CH1) which, together with complementarity light chain polypeptides, form a pair of antigen binding regions; (x) Dual-Variable Domain Immunoglobulin; (xi) other non-full length portions of immunoglobulin heavy and/or light chains, or mutants, variants, or derivatives thereof, alone or in any combination.

An “antibody-based binding protein”, as used herein, may represent any protein that contains at least one antibody-derived VH, VL, or CH immunoglobulin domain in the context of other non-immunoglobulin, or non-antibody derived components. Such antibody-based proteins include, but are not limited to (i) Fc-fusion proteins of binding proteins, including receptors or receptor components with all or parts of the immunoglobulin CH domains, (ii) binding proteins, in which VH and or VL domains are coupled to alternative molecular scaffolds, or (iii) molecules, in which immunoglobulin VH, and/or VL, and/or CH domains are combined and/or assembled in a fashion not normally found in naturally occurring antibodies or antibody fragments.

The term “modified antibody format”, as used herein, encompasses antibody-drug-conjugates (ADCs), polyalkylene oxide-modified scFv, monobodies, diabodies, camelid antibodies, domain antibodies, bi- or trispecific antibodies, IgA, or two IgG structures joined by a J chain and a secretory component, shark antibodies, new world primate framework and non-new world primate CDR, IgG4 antibodies with hinge region removed, IgG with two additional binding sites engineered into the CH3 domains, antibodies with altered Fc region to enhance or reduce affinity for Fc gamma receptors, dimerized constructs comprising CH3, VL, and VH, and the like.

The Kabat numbering scheme (Martin and Allemn 2014) has been applied to the disclosed antibodies.

The term “Antibody-Drug conjugate” or “ADC” refers to an antibody or antibody fragment to which toxins (or drugs) have been linked. In an ADC, toxins are conjugated to the antibody or antibody fragment by cleavable or non-cleavable linkers. Cleavable linker may be designed to be cleaved extracellularly in the tumor environment or intracellularly within the cytosol. Cleavable linkers exploit differential conditions of reducing power or enzymatic degradation that can be present either outside or inside the target cell. Non-cleavable linkers require the ADC to be internalized, the antibody-linker component needs to be degraded by lysosomal proteases for the toxins to be released. Conjugation of the linker to the antibody may also vary. Conjugation may rely on the presence of lysine and cysteine residues within the polypeptide structure of the antibody as the point of conjugation. Reactive groups on the linker can e.g. be conjugated to the side chain of lysine residues through amide or amidine bond formation. Conjugation via cysteine residues requires a partial reduction of the antibody. Alternatively, site-specific enzymatic conjugation can be used. This requires enzymes that react with the antibody and can induce site- or amino acid sequence-specific modifications. Peptide sequences recognized by these enzymes may have to be inserted into the genetically engineered antibodies or fragments to be conjugated. Enzymes which have been used for such purpose are sortase, transglutaminase, galactosyltransferase, sialyltransferase and tubulin-tyrosine ligase. An overview of ADC linker conjugation and toxins can be found in Ponziani et al, 2020 (Ponziani et al. 2020). An overview of conjugation of toxins to antibody fragments can be found in Aguiar et al, 2018 (Aguiar et al. 2018). The type of linker and the method of conjugation used to conjugate the toxin to the antibody or antibody fragment may determine the drug-to-antibody ratio (DAR).

The term “toxin” refers to a cytotoxic and/or cytostatic agent that can be based on a synthetic, plant, fungal, or bacterial molecule. Cytotoxic or cytostatic means that they inhibit the growth of and/or inhibit the replication of and/or kill cells, particularly malignant cells typically due to their increased turnover. In a preferred embodiment, the toxin is selected from the group consisting of anthracyclines and derivatives thereof. Anthracyclines are antibiotic compounds that exhibit cytotoxic activity, and may kill cells by different mechanisms, including intercalation of the drug molecules into the DNA of the cell or DNA severing activity thereby inhibiting DNA-dependent nucleic acid synthesis, generation of free radicals by the drug which react with cellular macromolecules to cause damage to the cells, DNA alkylation and/or interactions of the drug molecules with the cell membrane. Anthracyclines include doxorubicin, epirubicin, idarubicin, daunomycin, nemorubicin, and derivatives thereof. A well-known and preferred anthracycline derivative is PNU-159682, or in short PNU, CAS No. 202350-68-3. It is a highly potent metabolite of nemorubicin having outstanding cytotoxicity. Anthracycline derivatives are understood as including also the toxin as a result of conjugation to specific ligands, where due to the conjugation chemistry used, some atoms of the original toxin may be missing (Broggini 2008; Quintieri et al. 2005). In some instances, the term anthracycline derivatives may be understood as a result of lysosomal degradation, where fragment of the linker may remain attached to the anthracycline molecule. The term “anthracyclines” as used herein refers to anthracyclines and anthracycline derivatives.

The term “selectively binds to CLDN18.2” or “selective binding to CLDN18.2” as referred to herein refers to an antibody exhibiting binding to CLDN18.2, while exhibiting no (specific) binding to CLDN18.1. Hence, the antibodies selectively binding to CLDN18.2 do not exhibit cross-reactivity to CLDN18.1.

Where the term “comprising” is used in the present description and claims, it does not exclude other elements. For the purposes of the present invention, the term “consisting of” is considered to be a preferred embodiment of the term “comprising of”. If hereinafter a group is defined to comprise at least a certain number of embodiments, this is also to be understood to disclose a group, which preferably consists only of these embodiments.

Where an indefinite or definite article is used when referring to a singular noun, e.g. “a”, “an” or “the”, this includes a plural of that noun unless something else is specifically stated.

Technical terms are used by their common sense. If a specific meaning is conveyed to certain terms, definitions of terms will be given in the following in the context of which the terms are used.

### DESCRIPTION OF THE INVENTION

The inventors have surprisingly identified novel antibody-drug conjugates (ADCs) involving anti-CLDN18.2 antibodies and a toxin as further described herein, which exhibit increased binding to tumor cells expressing CLDN18.2 compared to healthy stomach cells expressing CLDN18.2 and/or have improved stability and/or are humanized while retaining their improved properties.

The invention provides an ADC based on an antibody binding to CLDN18.2, wherein the antibody or fragment thereof exhibits increased binding to tumor tissue expressing CLDN18.2 over healthy tissue expressing CLDN18.2. In one embodiment, the healthy cells or tissue used for the comparison are healthy stomach cells or healthy stomach tissue.

Increased binding to tumor tissue by the antibody or fragment thereof provided herein may be shown by bioanalytical methods such as flow cytometry (FC) or immunohistochemistry (IHC), as shown in Examples 4 and 5, respectively. A tumor expressing CLDN18.2 may be generated by subcutaneously injecting CLDN18.2-expressing A549 cells into a Balb/c mouse. The CLDN18.2-expressing A549 cells may be generated as shown in Example 4 and are available under the accession number DSM ACC3360 deposited on 6 Dec. 2019 at the DSMZ-Deutsche Sammlung von Mikroorganismen and Zellkulturen GmbH Inhoffenstr. 7B 38124 Braunschweig DE. The healthy tissue (e.g. healthy stomach tissue) may also originate from the mouse bearing the tumor. Increased binding to tumor tissue over healthy tissue may thus be shown on the tumor tissue and healthy tissue obtained from the same animal.

Increased binding to CLDN18.2 expressed in tumor tissue may be due to posttranslational modification such as differential glycosylation of CLDN18.2, or misfolding of CLDN18.2, when compared to CLDN18.2 expressed in healthy tissue.

Flow cytometry (FC) may be used as a bioanalytical method to test antibody binding. The percentage of CLDN18.2-positive cells can for example be measured by FC for a specific anti-CLDN18.2 antibody. Another possible binding read-out may for example be the ratio of the percentage of CLDN18.2-positive cells in a tumor cell sample versus the percentage of CLDN18.2-positive cells in a cell sample obtained from healthy tissue, such as healthy stomach tissue. Increased binding of an antibody to tumor cells expressing CLDN18.2 generated from CLDN18.2-expressing A549 cells compared to healthy cells, such as healthy stomach cells, may be shown by a ratio of >2, >5, ≥10, preferably ≥15, and more preferably ≥20.

Increased binding of an antibody to tumor cells expressing CLDN18.2 generated from CLDN18.2-expressing A549 cells compared to healthy cells, such as heathy stomach cells, may also be described by showing that the antibody binds at least 2 times more, at least 5 times more, at least 10 times more, preferably at least 15 times more, preferably at least 20 times more tumor cells than healthy cells, such as healthy stomach cells.

Immunohistochemistry (IHC) may be used as a bioanalytical method to test antibody binding. The tissue sample used for IHC should preferably be snap frozen after resection and, once thawed, fixed in acetone as, e.g., shown in Example 5. Since CLDN18.2 is a tight-junction protein in healthy tissue, positive CLDN18.2 staining should result in visualization of a predominantly membranous staining at the cell-cell interface in healthy tissue and/or tumor tissue. Negative CLDN18.2 staining or weak staining should therefore result in absence of membranous staining.

In another embodiment, the antibody or fragment thereof binds to CLDN18.2 with a half maximal effective concentration (EC50) value of above 0.4 μg/ml, above 0.5 μg/ml, preferably above 0.6 μg/ml, but not above 1 μg/ml when measured by flow cytometry (FC) titration on HEK293T cells overexpressing CLDN18.2. HEK293T cells overexpressing CLDN18.2 may be generated as described in Example 3. The EC50 value of the antibody may be, when measured by flow cytometry (FC) titration on HEK293T cells overexpressing CLDN18.2, between 0.4 and 1 μg/ml, between 0.5 and 1 μg/ml or preferably between 0.6 and 1 μg/ml.

Alternatively, the EC50 value of the antibody may be compared to the EC50 value of IMAB362 when measured by flow cytometry on HEK293T cells overexpressing CLDN18.2, wherein the EC50 value of the antibody is at least 1.1 times higher, at least 1.2 times higher, preferably at least 1.5 times higher, more preferably at least 2 times higher, even more preferably at least 2.5 times higher than the EC50 value of IMAB362 but not more than 5 times higher than the EC50 value of IMAB362. The EC50 value of the antibody may be between 1.1 times higher and 2.5 times higher, between 1.2 times higher and 2.5 times higher, preferably between 1.5 times higher and 2.5 times higher, or more preferably between 2 times higher and 2.5 times higher than the EC50 value of IMAB362 when measured by flow cytometry on HEK293T cells overexpressing CLDN18.2.

In another embodiment, the antibody or fragment thereof binds to CLDN18.2 with an EC50 value of above 0.6 μg/ml, above 1 μg/ml, preferably above 1.5 μg/ml, more preferably above 2 μg/ml, but not above 3 μg/ml when measured by flow cytometry titration on PA-TU-8988S-High cells. PA-TU-8988S-High cells may be generated as described in Example 2. The EC50 value of the antibody, when measured by flow cytometry titration on PA-TU-8988S-High cells, may be between 0.6 and 3 μg/ml, between 1 and 3 μg/ml, preferably between 1.5 and 3 μg/ml, or more preferably between 2 and 3 μg/ml.

Alternatively, the EC50 value of the antibody may be compared to the EC50 value of IMAB362 when measured by flow cytometry on PA-TU-8988S-High cells, wherein the EC50 value of the antibody is at least 1.5 times higher, at least 2 times higher, preferably at least 3 times higher, more preferably at least 4 times higher, but not more than 5 times higher than the EC50 value of IMAB362. The EC50 value of the antibody, when measured by flow cytometry on PA-TU-8988S-High cells, may be between 1.5 times higher and 5 times higher, between 2 times higher and 5 times higher, between 3 times higher and 5 times higher or between 4 times higher and 5 times higher than the EC50 value of IMAB362.

In another embodiment, the antibody or fragment thereof binds to CLDN18.2 with a maxMFI values within +/−40% of the maxMFI value of IMAB362 when measured by flow cytometry on HEK293T cells overexpressing CLDN18.2. The antibody or fragment thereof may also bind to CLDN18.2 with maxMFI values equal or up to 2 times higher than the maxMFI value of IMAB362 when measured by flow cytometry on PA-TU-8988S-High cells.

An antibody or functional fragment thereof with increased binding to tumor tissue expressing CLDN18.2 compared to healthy tissue expressing CLDN18.2 may have therapeutic advantages over antibodies unable to discriminate healthy tissue expressing CLDN18.2 from tumor tissue expressing CLDN18.2. Tumor-specific antibodies may not lead to safety issues and side effects, which are very often associated with the on-target effect of therapeutic antibodies in healthy organs/tissues (Hansel et al. 2010). Such undesirable effects have been reported for, e.g., IMAB362 (Sahin et al. 2018; Tureci et al. 2019).

The invention also provides an ADC comprising an antibody or fragment thereof binding to CLDN18.2 comprising the heavy chain complementarity determining region (HCDR) HCDR1, HCDR2 and HCDR3 sequences of SEQ ID NO: 21, SEQ ID NO: 22, and SEQ ID NO: 23, respectively and the light chain CDR LCDR1, LCDR2 and LCDR3 sequences of SEQ ID NO: 24, SEQ ID NO: 25, and SEQ ID NO: 26, respectively, and a toxin. In one embodiment, the toxin is an anthracycline.

In another embodiment, the inventors have engineered novel ADCs based on the novel anti CLDN18.2 antibodies from above, which surprisingly exhibit better cytotoxic activity on tumor cells compared to a similar ADC based on IMAB362.

The ADC of the invention has the general formula A-(L-T)ₙ, wherein
a. A is an antibody or fragment thereof binding to CLDN18.2 comprising the heavy chain complementarity-determining regions (CDRs) HCDR1, HCDR2 and HCDR3 sequences of SEQ ID NO: 21, SEQ ID NO: 22, and SEQ ID NO: 23, respectively and the light chain CDRs LCDR1, LCDR2 and LCDR3 sequences of SEQ ID NO: 24, SEQ ID NO: 25, and SEQ ID NO: 26, respectively,
b. L is a linker, and
c. T is a toxin,
   wherein the toxin is an anthracycline.

In one embodiment, n is an integer ≥1 and ≤10. The invention also relates to a pharmaceutical acceptable salt or ester of the ADC.

The invention also provides an ADC comprising an antibody binding to CLDN18.2 comprising the heavy chain HCDR3 sequence of SEQ ID NO: 23 and the light chain LCDR3 sequence of SEQ ID NO: 26.

The respective consensus sequences can be found in Table 1. It is understood that any ADC comprising an antibody or fragment thereof based on any combination of CDRs derived from the consensus sequences and binding to CLDN18.2 is part of the invention.

TABLE 1
isolated antibody CDR consensus sequences

| CDRs | Sequence | SEQ ID |
|---|---|---|
| HCDR1 | DYAMX | SEQ ID NO: 21 |
| | X in 5^{th} position is H or Y | |
| HCDR2 | WINXYTGKPTYXXXFXG | SEQ ID NO: 22 |
| | X in 4^{th} position is T or A; | |
| | X in 12^{th} position is A or S; | |
| | X in 13^{th} position is D or Q; | |
| | X in 14^{th} position is D or K; | |
| | X in 16^{th} position is K or Q | |
| HCDR3 | AVXYGYTMDA | SEQ ID NO: 23 |
| | X in 3^{rd} position is F or Y | |
| LCDR1 | RXSEDIYSNXA | SEQ ID NO: 24 |
| | X in 2^{nd} position is A or T; | |
| | X in 10^{th} position is L or F | |
| LCDR2 | XXXRLQD | SEQ ID NO: 25 |
| | X in 1^{st} position is S or A; | |
| | X in 2^{nd} position is V or I; | |
| | X in 3^{rd} position is K or N | |
| LCDR3 | LQGSXFPLT | SEQ ID NO: 26 |
| | X in 5^{th} position is K or N | |

In one embodiment, the linker L of the ADC of the invention comprises at least one non-cleavable linker element. A non-cleavable linker element may be defined as a linker element that is only subjected to lysosomal degradation, that is not the substrate of specific enzymes and that is stable in plasma and cytosol.

The non-cleavable linker element may be selected from the group consisting of:
a. ethylenediamine (EDA),
b. N-formyl-N,N′-dimethylethylenediamine,
c. diethylamine (DEA),
d. a piperazine-derived compound of the following formula:

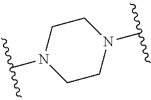 [SMILES: *N1CCN(*)CC1]

wherein the wavy lines indicate attachments to the toxin and another linker element,
e. the compound of the following formula:

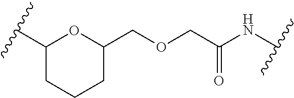 [SMILES: *NC(=O)COCC1CCCC(*)O1]

wherein the wavy lines indicate attachments to the toxin and another linker element,
f. the compound of the following formula:

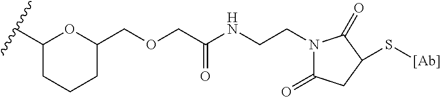

wherein the wavy line indicates attachment to the toxin and [Ab] indicates the antibody or fragment thereof,
g. a maleimidocaproyl compound of the following formula:

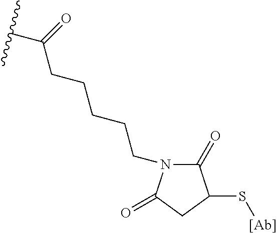

wherein the wavy line indicates attachment to another linker element and [Ab] indicates the antibody or fragment thereof,
h. the compound of the following formula:

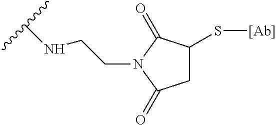

wherein the wavy line indicates attachment to a toxin and [Ab] indicates the antibody or fragment thereof,
and wherein the non-cleavable linker element is conjugated to the toxin by means of an amide bond or an ether bond.

The non-cleavable linker element may be directly covalently attached to the antibody (and thereby form the linker) or it may be attached via other linker elements such as oligopeptide linker elements. Alternatively, or additionally, cleavable linker elements may be present in the linker.

The non-cleavable linker element may be linked to the antibody via amino-acids of the antibody sequence that have side-chains with available nucleophilic groups such as ε-N₂ of lysine and the sulfhydryl SH group of cysteine. Maleimide chemistry allows linkage to the cysteine side-chain while acylation chemistry is usually used for linkage to the lysine side-chain. Ample information on such linkages can be found in Jain et al, 2015 (Jain et al. 2015). Linkage of a non-cleavable linker element to an oligopeptide linker element may be carried out by carbodiimide crosslinking chemistry. Guidance for such crosslinking chemistry may be found in the Thermo Scientific Crosslinking Technical Handbook (2012) (“Crosslinking Technical Handbook” 2012).

The non-cleavable linker element may also be directly attached to the anthracycline. In one embodiment, the non-cleavable linker element is attached to the anthracycline of formula I by means of an amide bond to C₁₃ or an ether bond to C₁₄, wherein R₁ is hydrogen atom, hydroxy or methoxy group and R₂ is a C₁-C₅ alkoxy group.

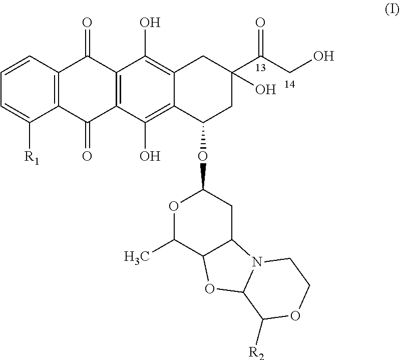

It is understood that a combination of one or more linker elements may be used to form the linker in order to link the antibody to the toxin, including enzyme-cleavable linker elements.

In another element, the linker further comprises an oligopeptide linker element and/or enzyme-cleavable linker element and/or a spacer element.

The oligopeptide linker element is understood as being an oligopeptide that is present in addition to the peptidic chain forming the antibodies or fragment thereof. The oligopeptide linker element may be directly attached to the C-termini of the heavy and/or light chains forming the antibody, or the fragments thereof. In one embodiment, the DNA coding sequence of the oligopeptide linker element may be part of the DNA coding for the respective heavy and/or light chain forming the antibody or fragment thereof.

In another embodiment, the oligopeptide linker element may be the result of peptide ligation used to link two or more oligopeptide linker elements. Ligation may be catalyzed by peptide ligases such as sortases (i.e. Sortase A), asparaginyl endoproteases (i.e. Butelase 1), trypsin related enzymes (i.e. Trypsiligase) or subtilisin-derived variants (i.e. Peptiligase) (Nuijens et al. 2019). The oligopeptide linker elements may thus include peptide ligase recognition motifs.

The term spacer element, in the context of the invention, is to be understood as spacers added to the linker to avoid steric hindrance and to allow proper conjugation of the toxin to the antibody or fragment thereof.

In one embodiment, the oligopeptide linker element comprises a sortase recognition motif oligopeptide selected from: -LPXTGₘ-, -LPXAGₘ-, -LPXSGₘ-, -LAXTGₘ-, -LPXTGₘ-, -LPXTAₘ-, -NPQTGₘ- or -NPQTNₘ-, with Gₘ being an oligoglycine with m being an integer between ≥1 and ≤21, Am being an oligoalanine with m being an integer between ≥1 and ≤21, Nm being an oligoasparagine with m being an integer between ≥1 and ≤21 and X being any conceivable amino acid. Preferably, m is 2 or 3, especially 2. In a preferred embodiment, the sortase recognition motif oligopeptide is -LPQTGG- or -LPETGG-. The sortase recognition motif oligopeptide may be present at the C-termini of the heavy and/or light chains, of the antibody or of fragments thereof, preferably at the C-termini of the light chains.

In a further preferred embodiment, the oligopeptide linker element of the ADC comprises the sequence SEQ ID NO: 131. In one embodiment, the sequence SEQ ID NO: 131 is at the C-terminus of the antibody heavy chain and in another preferred embodiment at the C-terminus of the antibody light chain.

In another embodiment, an enzyme-cleavable linker element is present in the linker. The enzyme-cleavable linker element may comprise a val-cit-PAB linker according to the compound of the following formula:

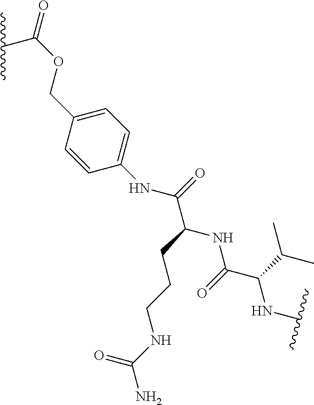 [SMILES: *N[C@H](C(=O)N[C@@H](CCCNC(N)=O)C(=O)Nc1ccc(COC(*)=O)cc1)C(C)C]

wherein the wavy lines indicate attachments to other linker elements or the antibody or the toxin. The enzyme-cleavable linker element may be attached to another linker element or the antibody or the toxin by know crosslinker chemistry as described above for the non-cleavable linker elements.

In yet another embodiment, the linker further comprises a spacer element. In one embodiment, the spacer element comprises a peptidic flexible oligopeptide. Flexible linker elements can be applied when the linked components require a certain degree of movement or interaction. Flexible oligopeptides are generally composed of small, non-polar (e.g. G) or polar (e.g. S or T) amino acids. The small size of these amino acids provides flexibility and allows for mobility of the connected functional components. The incorporation of S or T can maintain stability of the linker in aqueous solutions by forming hydrogen bonds with water molecules, and therefore reduces the unfavorable interaction between the linker and protein moieties. Further guidance on peptidic flexible oligopeptides may be found in Chen et al, 2013 (Chen, Zaro, and Shen 2013).

Preferably the spacer element comprises a peptidic flexible oligopeptide consisting of G and S, more preferably the peptidic flexible oligopeptide is (GGGGS)ₒ with o being 1, 2, 3, 4 or 5.

The invention also provides ADCs of the following structures:
a. A-([oligopeptide linker element-non-cleavable linker element]-T)ₙ and preferably wherein the linker is selected from:

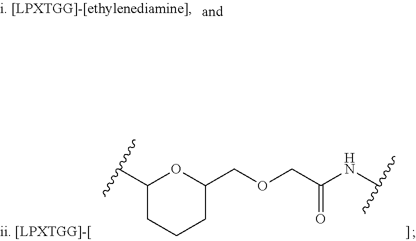 [SMILES: *NC(=O)COCC1CCCC(*)O1]

b. A-([oligopeptide linker element-enzyme cleavable linker element-non-cleavable linker element]-T)ₙ and preferably wherein the linker is selected from:

i.
[LPXTGG]-[vc-PAB]-[N-formyl-N,N′-
dimethylethylenediamine],
and
ii.
[LPXTGG]-[vc-PAB]-[piperazine];

c. A-([spacer element-oligopeptide linker element-non-cleavable linker element]-T)ₙ and preferably wherein the linker is selected from:

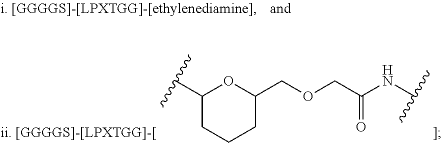 [SMILES: *NC(=O)COCC1CCCC(*)O1]

or
d. A-([spacer element-oligopeptide linker element-enzyme cleavable linker element-non-cleavable linker element]-T)ₙ and preferably wherein the linker is selected from:

i.
[GGGGS]-[LPXTGG]-[vc-PAB]-[N-formyl-N,N′-
dimethylethylenediamine],
and
ii.
[GGGGS]-[LPXTGG]-[vc-PAB]-[piperazine].

where A is an antibody or fragment thereof binding to CLDN18.2 comprising the HCDR1, HCDR2 and HCDR3 sequences of SEQ ID NO: 21, SEQ ID NO: 22 and SEQ ID NO: 23 respectively, and the LCDR1, LCDR2 and LCDR3 sequences of SEQ ID NO: 24, SEQ ID NO: 25 and SEQ ID NO: 26 respectively, and T is a anthracycline.

In one embodiment, n is an integer ≥1 and ≤10. The invention also relates to a pharmaceutical acceptable salt or ester of the ADC.

It is understood that the toxin may be conjugated via the linker to the C-termini of the antibody heavy and/or light chains, or at the C-termini of the antibody fragments.

25 In a preferred embodiment, the non-cleavable linker element is ethylenediamine and the oligopeptide linker element is LPXTGG wherein X is Q or E, preferably wherein X is Q.

In one embodiment,
a. (L-T) is covalently linked to both light chains of the antibody,
b. (L-T) is covalently linked to both heavy chains of the antibody, or
c. (L-T) is covalently linked to both light chains and both heavy chains of the antibody.

In one embodiment, (L-T)
a. is linked to the C-terminus of the antibody light chain or antibody heavy chain, or
b. is linked to an amino acid side chain of the antibody light chain or antibody heavy chain.

In case the toxin is linked to the C-terminus of the antibody light chain or antibody heavy chain, an oligopeptide linker element and an optional spacer element may be part of the antibody amino-acid sequence when the antibody is recombinantly expressed with such C-terminal tag. In case the toxin is linked to an amino-acid side chain of the antibody amino acid sequence, the linker element may be linked by maleimide chemistry or acylation chemistry, depending on the amino acid side chain of choice.

Surprisingly, the ADCs of the invention, with the toxin either conjugated via an oligopeptide peptide linker element—non-cleavable enzyme linker element at the HC only, via a spacer element—oligopeptide peptide linker element—non-cleavable enzyme linker element at the LC only, or such linker-toxin combinations at the HC and LC have a higher cytotoxic activity on cells expressing CLDN18.2 than a similar ADC based on IMAB362 (see FIGS. 11 to 19 and Example 8) showing the superiority of the newly identified antibodies over the prior art antibody also in the ADC context. The ADCs of the invention have also a higher cytotoxic activity than an ADC based on IMAB362 and conjugated to MMAE via an MC-vc-PAB linker as previously disclosed in WO2016/165762 (see FIG. 11).

In one embodiment, the anthracycline has the following formula (I):

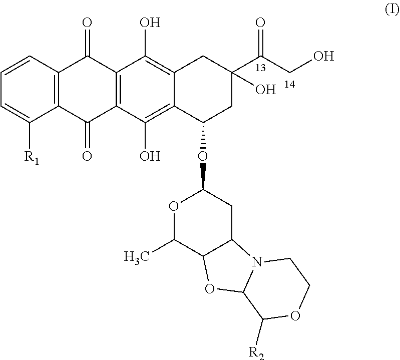

wherein R₁ is a hydrogen atom, a hydroxy or methoxy group, and wherein R₂ is a C₁-C₅ alkoxy group. In one embodiment, the anthracycline is attached to the linker via C₁₃ resulting in the loss of C₁₄ and the hydroxyl group or via C₁₄ resulting in loss of the hydroxyl group.

It is understood that linking the toxin (via C₁₃ or C₁₄) to an antibody will not affect the cytotoxic activity of the toxin.

Further information on the synthesis of PNU-159682 and its use as toxin in ADCs may be found in Holte D et al 2020 (Holte et al. 2020).

PNU-159682 may be linked to the antibody by non-cleavable or enzyme-cleavable linkers as shown below.

The linker may be a maleimide acetal linker:

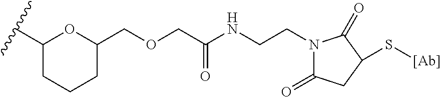

Such a linker was used in an PNU-159682-maleimide acetal-Ab ADC shown below:

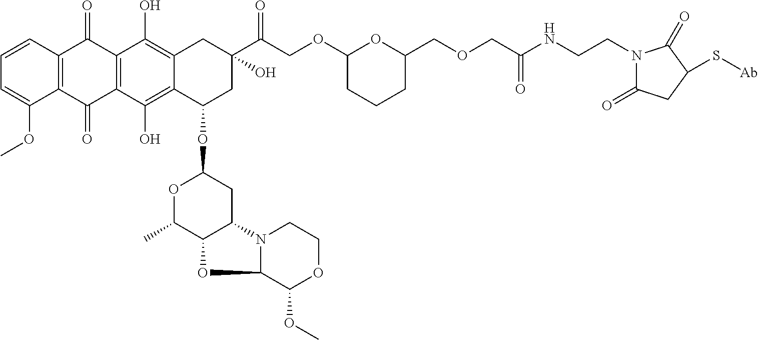

Such a PNU-159682 maleimide acetal-Ab ADC has been disclosed in U.S. Pat. No. 10,435,471, column 90. The PNU-159682 maleimide acetal compound has been disclosed as compound 51 in WO2010/009124 and may be prepared as disclosed in Example 3d (paragraphs [0576] to [0578]), based on the compound prepared in Example 2 (paragraphs to [0550]) of the same application.

PNU-159682 may also be linked to the antibody by a val-cit-PAB enzyme-cleavable linkers to form a PNU-159682-val-cit-PAB-Ab ADC as shown below:

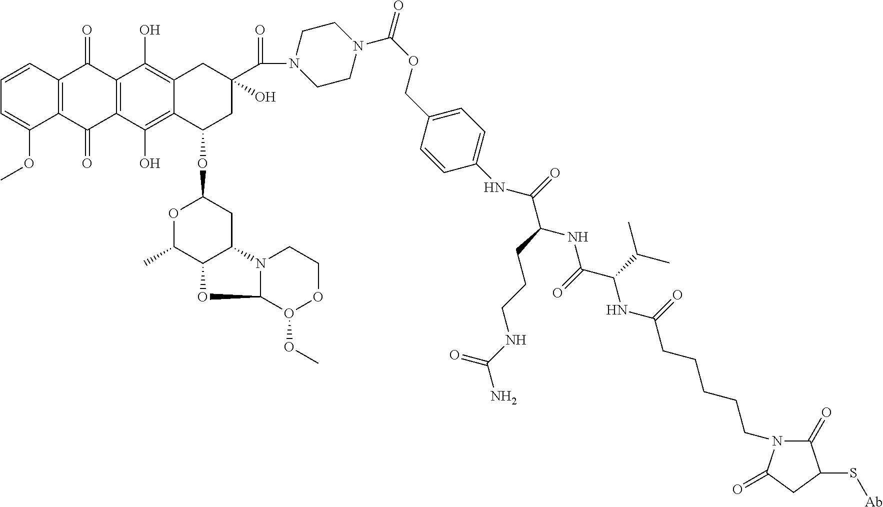

Such an ADC has been disclosed in U.S. Pat. No. 10,435,471, column 91-92. The PNU-159682-val-cit-PAB compound is disclosed as compound 55 in WO2010/009124 and may be prepared as shown in Example 3b (paragraph [0567]-[0573] and FIG. 7d) of the same application.

PNU-159682 may also be linked to the antibody via an enzyme cleavable linker val-cit-PAB and an additional non-cleavable linker element as shown below:

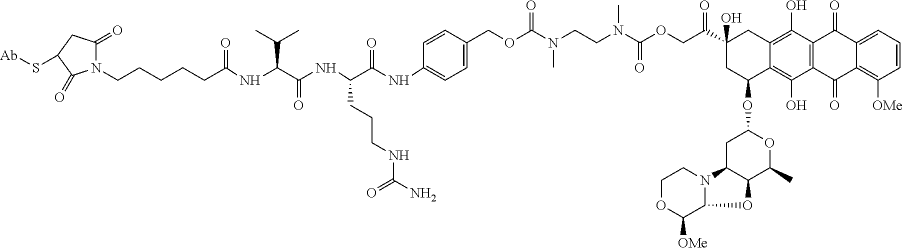

Such an ADC has been disclosed in U.S. Pat. No. 10,435,471, column 91-92 and Yu S F et Clin Cancer Research 2015 (Yu et al. 2015). The PNU-159682-val-cit-PAB+non-cleavable linker compound may be prepared as follows:

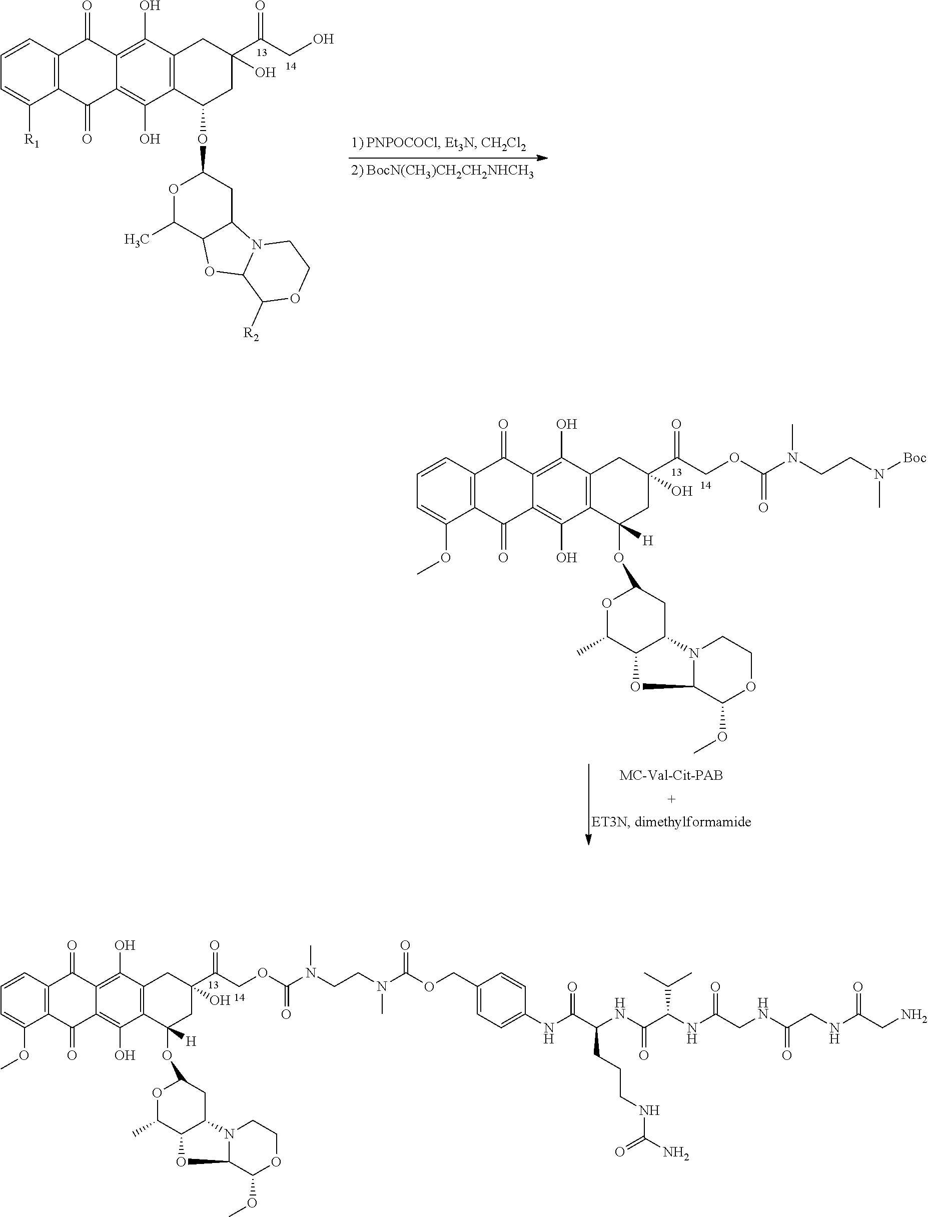

wherein MC-val-cit-PAB is commercially available (MedChemExpress Cat. No.: HY-78738) and Boc is a tert-butyloxycarbonyl protecting group.

PNU-159682 may also be linked to the antibody via a non-cleavable maleimide linker as shown below:

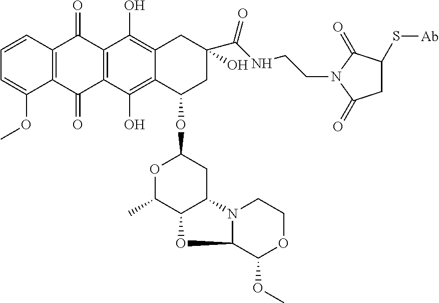

Such ADC has been disclosed in U.S. Pat. No. 10,435,471, column 93. The PNU-159682-maleimid compound is disclosed as compound 55 in WO2010/009124 and its preparation in Example 3a (paragraphs to of the same application).

A combination of non-cleavable, enzyme-cleavable and oligopeptide linker elements has also been used to link PNU-159682 to an antibody. Such ADC is shown below:

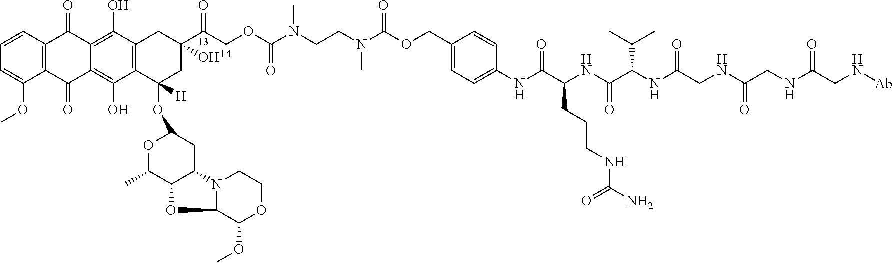

Such a compound is disclosed in Stefan et al. (Stefan et al. 2017). Such an ADC may be synthesized as disclosed above for the PNU-159682-val-cit-PAB+non-cleavable linker ADC, 10 substituting MC-Val-Cit-PAB by Fmoc-Gly3-Val-Cit-PAB (commercially available from MedChemExpress Cat No.: HY-136106), and the resulting linker-toxin compound may be conjugated to an antibody as disclosed in WO2016/102679, page 34, 2^{nd} paragraph.

PNU-159682 may also be linked to an antibody via a non-cleavable EDA linker element combined with an oligopeptide linker element (-GGGGG-) as shown below:

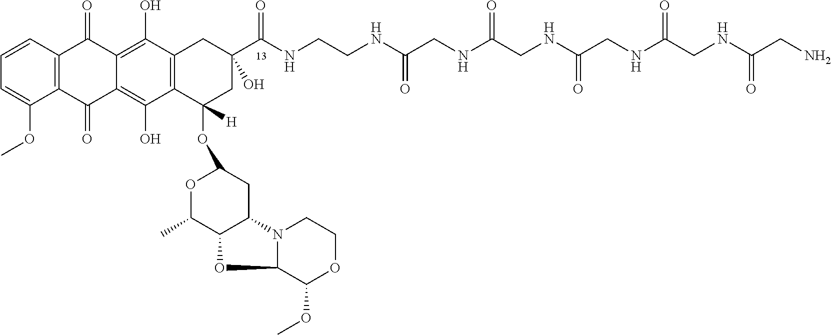

Such a compound is disclosed in WO2016/102679, FIG. 3A. It may be prepared as disclosed in the scheme on FIG. 3B and page 33, last paragraph to page 34, 1^{st} paragraph of WO2016/102679 and the resulting linker-toxin compound may be conjugated to an antibody as disclosed in WO2016/102679, page 34, 2^{nd} paragraph. The oligo peptide linker element used above may also be (-GGG-) or preferably (-GG-).

Antibody binding or binding affinity is generally expressed in terms of equilibrium association or dissociation constants (Kₐ or K_{d}, respectively), which are in turn reciprocal ratios of dissociation and association rate constants (k_{off} and kₒₙ, respectively). Thus, equivalent affinities may correspond to different rate constants, so long as the ratio of the rate constants remains the same. Binding affinities and/or rate constants can be determined using techniques well known in the art or described herein, such as ELISA, flow cytometry titration, isothermal titration calorimetry (ITC), Biacore (SPR), biolayer inferometry or fluorescent polarization. In some cases, due to the nature of the antigen, the Kₐ or K_{d} of antibodies may be difficult to measure. This is especially true for integral membrane proteins such as Claudins (Hashimoto et al. 2018). In such cases, the integral membrane protein may be expressed as proteoliposomes or lipoparticles. Such lipoparticles may be immobilized on plastic and used in ELISA assay to determine the binding affinity of antibodies to the immobilized antigen. Instead of Kₐ or K_{d} values, half maximal effective concentration (EC50) values may thus be calculated for each tested antibody or functional fragment thereof, reflecting its binding affinity (or strength of binding) to the antigen. Example 2 and FIG. 1 below exemplify ELISA assay binding affinity curves of antibodies with CDRs comprised in the consensus sequences of Table 1. The EC50 value and the maximal binding value can be used for quantification of the binding of the antibodies to CLDN18.2. Example 3 below relates to the calculation of EC50 values by flow cytometry on cells expressing CLDN18.2 of antibodies with CDRs comprised in the consensus sequences of Table 1.

The cytotoxic activity of ADCs can be characterized by EC50 values retrieved from an ADC cytotoxic assay. Example 8 and Table 9 below relates to the calculation of EC50 values of the ADCs of the invention using cytotoxic assays with cells expressing CLDN18.2.

Although the antibody binding EC50 (μg/ml) values of all the hCl antibodies measured on the HEK293T cells lines overexpressing CLDN18.2 and on the PA-TU-8988S-High cell lines is higher than the antibody binding EC50 value of the reference antibody IMAB362 on the same cell lines (see Table 4 and Example 2), i.e. the hCl antibodies provided herein bind CLDN18.2 with lower affinity compared to IMAB362, the inventors have now surprisingly shown that the ADC cytotoxicity EC50 (ng/ml) value of the ADCs of the invention measured on the HEK293T and A549 cells lines overexpressing CLDN18.2 and on the PA-TU-8988S-High cell lines were lower than the cytotoxicity EC50 value of an ADC based on IMAB362 on the same cell lines (see Table 9 and Example 8). This shows that the ADCs of the invention have a higher cytotoxic activity than an ADC based on IMAB362, despite the antibodies having a lower binding affinity to the target than IMAB362.

Likewise, the ADCs of the invention showed higher in-vivo efficacy in patient-derived tumor xenograft models than an ADC based on IMAB362 (see Example 9).

In one embodiment, the antibody or fragment thereof binds to CLDN18.2 and comprises the heavy chain CDRs HCDR1, HCDR2 and HCR3 sequences of SEQ ID NO: 21, SEQ ID NO: 126, and SEQ ID NO: 23, respectively and the light chain CDRs LCDR1, LCDR2 and LCDR3 sequences of SEQ ID NO: 24, SEQ ID NO: 25, and SEQ ID NO: 26, respectively.

In one embodiment, the antibody or fragment thereof binds to CLDN18.2 and comprises:
a. the HCDR1, HCDR2 and HCDR3 sequences of SEQ ID NO: 1, SEQ ID NO: 15 and SEQ ID NO: 3, respectively, and the LCDR1, LCDR2 and LCDR3 sequences of SEQ ID NO: 4, SEQ ID NO: 5 and SEQ ID NO: 6, respectively;
b. the HCDR1, HCDR2 and HCDR3 sequences of SEQ ID NO: 1, SEQ ID NO: 16 and SEQ ID NO: 3, respectively, and the LCDR1, LCDR2 and LCDR3 sequences of SEQ ID NO: 4, SEQ ID NO: 5 and SEQ ID NO: 6, respectively;
c. the HCDR1, HCDR2 and HCDR3 sequences of SEQ ID NO: 1, SEQ ID NO: 16 and SEQ ID NO: 3, respectively, and the LCDR1, LCDR2 and LCDR3 sequences of SEQ ID NO: 17, SEQ ID NO: 14 and SEQ ID NO: 11, respectively;
d. the HCDR1, HCDR2 and HCDR3 sequences of SEQ ID NO: 1, SEQ ID NO: 16 and SEQ ID NO: 3, respectively, and the LCDR1, LCDR2 and LCDR3 sequences of SEQ ID NO: 18, SEQ ID NO: 19 and SEQ ID NO: 11, respectively;
e. the HCDR1, HCDR2 and HCDR3 sequences of SEQ ID NO: 12, SEQ ID NO: 15 and SEQ ID NO: 3, respectively, and the LCDR1, LCDR2 and LCDR3 sequences of SEQ ID NO: 4, SEQ ID NO: 5 and SEQ ID NO: 6, respectively;
f. the HCDR1, HCDR2 and HCDR3 sequences of SEQ ID NO: 1, SEQ ID NO: 20, and SEQ ID NO: 3, respectively, and the LCDR1, LCDR2 and LCDR3 sequences of SEQ ID NO: 4, SEQ ID NO: 5 and SEQ ID NO: 6, respectively;
10 g. the HCDR1, HCDR2 and HCDR3 sequences of SEQ ID NO: 1, SEQ ID NO: 20 and SEQ ID NO: 3, respectively, and the LCDR1, LCDR2 and LCDR3 sequences of SEQ ID NO: 18, SEQ ID NO: 19 and SEQ ID NO: 11, respectively;
h. the HCDR1, HCDR2 and HCDR3 sequences of SEQ ID NO: 12, SEQ ID NO: 20 and SEQ ID NO: 8, respectively, and the LCDR1, LCDR2 and LCDR3 sequences 15 of SEQ ID NO: 4, SEQ ID NO: 5 and SEQ ID NO: 6, respectively; or
i. the HCDR1, HCDR2 and HCDR3 sequences of SEQ ID NO: 12, SEQ ID NO: 20 and SEQ ID NO: 8, respectively, and the LCDR1, LCDR2 and LCDR3 sequences of SEQ ID NO: 17, SEQ ID NO: 14 and SEQ ID NO: 11, respectively.

In another preferred embodiment, the ADCs based on the antibodies have a higher cytotoxic activity on CLDN18.2-expressing cells than the corresponding ADC based on IMAB362 as for example shown by the EC50 values for cytotoxic activity.

In yet another embodiment, the antibody or fragment thereof binds to CLDN18.2 and comprises:
a. the HCDR1, HCDR2 and HCDR3 sequences of SEQ ID NO: 1, SEQ ID NO: 2 and SEQ ID NO: 3, respectively, and the LCDR1, LCDR2 and LCDR3 sequences of SEQ ID NO: 4, SEQ ID NO: 5 and SEQ ID NO: 6, respectively;
b. the HCDR1, HCDR2 and HCDR3 sequences of SEQ ID NO: 1, SEQ ID NO: 7 and SEQ ID NO: 8, respectively, and the LCDR1, LCDR2 and LCDR3 sequences of SEQ ID NO: 9, SEQ ID NO: 10 and SEQ ID NO: 11, respectively; or
c. the HCDR1, HCDR2 and HCDR3 sequences of SEQ ID NO: 12, SEQ ID NO: 2 and SEQ ID NO: 3, respectively, and the LCDR1, LCDR2 and LCDR3 sequences of SEQ ID NO: 13, SEQ ID NO: 14 and SEQ ID NO: 11, respectively.

In yet another embodiment, the antibody or fragment thereof binds to CLDN18.2 and comprises:
a. a VH sequence of SEQ ID NO: 27 and a VL sequence of SEQ ID NO: 28;
b. a VH sequence of SEQ ID NO: 29 and a VL sequence of SEQ ID NO: 30;
c. a VH sequence of SEQ ID NO: 31 and a VL sequence of SEQ ID NO: 32.

In another embodiment, the antibody or fragment thereof binds to CLDN18.2 and comprises:
a. a VH sequence of: SEQ ID NO: 33;
b. a VH sequence of SEQ ID NO: 34;
c. a VH sequence of SEQ ID NO: 35;
d. a VH sequence of SEQ ID NO: 36; or
e. a VH sequence of SEQ ID NO: 37;
   and
f. a VL sequence of SEQ ID NO: 38;
g. a VL sequence of SEQ ID NO: 39;
h. a VL sequence of SEQ ID NO: 40; or
i. a VL sequence of SEQ ID NO: 41.

In a further embodiment, the antibody or fragment thereof binds to CLDN18.2 and comprises:
a. a VH sequence of SEQ ID NO: 33 and a VL sequence of SEQ ID NO: 38;
b. a VH sequence of SEQ ID NO: 34 and a VL sequence of SEQ ID NO: 38;
c. a VH sequence of SEQ ID NO: 34 and a VL sequence of SEQ ID NO: 39;
d. a VH sequence of SEQ ID NO: 34 and a VL sequence of SEQ ID NO: 40;
e. a VH sequence of SEQ ID NO: 35 and a VL sequence of SEQ ID NO: 38;
f. a VH sequence of SEQ ID NO: 36 and a VL sequence of SEQ ID NO: 41;
g. a VH sequence of SEQ ID NO: 36 and a VL sequence of SEQ ID NO: 40;
h. a VH sequence of SEQ ID NO: 37 and a VL sequence of SEQ ID NO: 41;
i. a VH sequence of SEQ ID NO: 37 and a VL sequence of SEQ ID NO: 38; or
j. a VH sequence of SEQ ID NO: 37 and a VL sequence of SEQ ID NO: 39.

In another embodiment, the antibody binds to CLDN18.2 and comprises:
a. the heavy chain sequence of SEQ ID NO: 46 and light chain sequence of SEQ ID NO: 51;
b. the heavy chain sequence of SEQ ID NO: 47 and light chain sequence of SEQ ID NO: 51;
c. the heavy chain sequence of SEQ ID NO: 47 and light chain sequence of SEQ ID NO: 52;
d. the heavy chain sequence of SEQ ID NO: 47 and light chain sequence of SEQ ID NO: 53;
e. the heavy chain sequence of SEQ ID NO: 48 and light chain sequence of SEQ ID NO: 51;
f. the heavy chain sequence of SEQ ID NO: 47 and light chain sequence of SEQ ID NO: 54;
g. the heavy chain sequence of SEQ ID NO: 49 and light chain sequence of SEQ ID NO: 53;
h. the heavy chain sequence of SEQ ID NO: 50 and light chain sequence of SEQ ID NO: 54;
i. the heavy chain sequence of SEQ ID NO: 50 and light chain sequence of SEQ ID NO: 51; or
j. the heavy chain sequence of SEQ ID NO: 50 and light chain sequence of SEQ ID NO: 52.

The constant light chain region CL and the constant heavy chain region CH1 and Fc region of the disclosed antibodies may have the amino acid sequence of SEQ ID NO: 127 and SEQ ID NO: 128, respectively.

The ADCs of the present invention, with an anthracycline conjugated to the light chain only, have a higher cytotoxic activity on cells expressing CLDN18.2 than IMAB362 with an anthracycline derivative conjugated to the light chain only (see FIGS. 11 to 19). The ADCs of the invention with an anthracycline conjugated either to the heavy and light chain or only to the heavy chain or only to the light chain have also a higher cytotoxic activity than IMAB362-MC-vc-PAB-MMAE previously disclosed in WO2016/165762 (see FIG. 11).

The inventors have also shown that the ADCs of the present have a higher in-vivo cytotoxic activity on patient-derived gastric tumor xenograft models, colon tumor xenograft models, pancreatic tumor xenograft models and lung tumor xenograft models than an identical ADC based on IMAB362 (see FIGS. 21 to 24, respectively and Example 9). In a preferred embodiment, the antibody binds to CLDN18.2 and comprises the heavy chain sequence of SEQ ID NO: 46 and light chain sequence of SEQ ID NO: 51.

In a further preferred embodiment, the antibody binds to CLDN18.2 and consists of the heavy chain sequence of SEQ ID NO: 46 and light chain sequence of SEQ ID NO: 51.

The antibody may have an amino acid sequence with at least 80% identity, at least 85%, at least 90%, at least 95% or at least 98% identity to the amino acid sequence of the antibody of the invention, exhibiting increased binding to tumor cells expressing CLDN18.2 compared to healthy stomach cells expressing CLDN18.2.

In one embodiment, the antibody binds to CLDN18.2 and has an amino acid sequence with at least 80% identity, at least 85%, at least 90%, at least 95% or at least 98% identity to an antibody comprising:
a. a VH sequence of SEQ ID NO: 27 and a VL sequence of SEQ ID NO: 28;
b. a VH sequence of SEQ ID NO: 29 and a VL sequence of SEQ ID NO: 30;
c. a VH sequence of SEQ ID NO: 31 and a VL sequence of SEQ ID NO: 32.

In a further embodiment, the antibody binds to CLDN18.2 and has an amino acid sequence with at least 80% identity, at least 85%, at least 90%, at least 95% or at least 98% identity to an antibody comprising:
a. a VH sequence of SEQ ID NO: 33 and a VL sequence of SEQ ID NO: 38;
b. a VH sequence of SEQ ID NO: 34 and a VL sequence of SEQ ID NO: 38;
c. a VH sequence of SEQ ID NO: 34 and a VL sequence of SEQ ID NO: 39;
d. a VH sequence of SEQ ID NO: 34 and a VL sequence of SEQ ID NO: 40;
e. a VH sequence of SEQ ID NO: 35 and a VL sequence of SEQ ID NO: 38;
f a VH sequence of SEQ ID NO: 36 and a VL sequence of SEQ ID NO: 41;
g. a VH sequence of SEQ ID NO: 36 and a VL sequence of SEQ ID NO: 40;
h. a VH sequence of SEQ ID NO: 37 and a VL sequence of SEQ ID NO: 41;
i. a VH sequence of SEQ ID NO: 37 and a VL sequence of SEQ ID NO: 38; or
j. a VH sequence of SEQ ID NO: 37 and a VL sequence of SEQ ID NO: 39.

In yet a further embodiment, the antibody binds to CLDN18.2 and has an amino acid sequence with at least 80% identity, at least 85%, at least 90%, at least 95% or at least 98% identity to an antibody consisting of the heavy chain sequence of SEQ ID NO: 46 and light chain sequence of SEQ ID NO: 51.

In another embodiment, the Fc domain of the antibody (or antibody fragment when present) may comprise modifications or mutations, such as the modifications or mutations listed in Table 2 below. Such a modification or mutation may be introduced to modulate the effector activity of the Fc domain of the antibody. Modification of antibodies may also include peptide tags added to the C-terminal end of the antibody HC and/or LC chain. Such tags may be used e.g. for protein purification or protein conjugation. In another embodiment, the antibody or fragment thereof that binds to CLDN18.2 is an IgA1, IgA2, IgD, IgE, IgG1, IgG2, IgG3, IgG4, synthetic IgG, IgM, F(ab)₂, Fv, scFv, IgGACH2, F(ab′)2, scFvCH3, Fab, VL, VH, scFv4, scFv3, scFv2, dsFv, Fv, scFv-Fc, (scFv)₂, a non-depleting IgG, a diabody, a bivalent antibody or Fc-engineered versions thereof. In a preferred embodiment, the antibody is an IgG1 type of antibody. The Fc region of immunoglobulins interacts with multiple Fcγ receptors (FcγR) and complement proteins (e.g. C1q), and mediates immune effector functions, such as elimination of targeted cells via antibody-dependent cellular cytotoxicity (ADCC), antibody-dependent cellular phagocytosis (ADCP) or complement-dependent cytotoxicity (CDC). For therapeutic approaches, it may be beneficial to enhance or silence Fc related effector functions. The type of immunoglobulin (IgA, IgD, IgE, IgG, IgM) may be selected according to the desired effector function of the antibody related to the Fc domain. One may also employ a synthetic immunoglobulin, such as an immunoglobulin with the IgG2 amino acids 118 to 260 and the IgG4 amino acids 261 to 447 or an IgG2 variant with point mutations from IgG4 (e.g. H268Q/V309L/A30S/P331S). Such synthetic immunoglobulins reduce effector functions of the antibody. Fc-engineered immunoglobulins may also be employed to modulate antibody effector function. Table 2 shows example of such Fc engineering. Expression in production cell lines with altered fucosylation may also impact FcγR binding.

TABLE 2
Examples of modifications to modulate antibody effector function. Unless otherwise
noted, the mutations are on the IgG1 subclass (Wang, Mathieu, and Brezski 2018).

| Engineering and intended | | |
|---|---|---|
| function | Mutation | Reference |
| Enhance ADCC | | |
| Increased FcγRIIIa binding | F243L/R292P/Y300L/V305I/P396L | (Stavenhagen et al. 2007) |
| | S239D/I332E | (Lazar et al. 2006) |
| | S298A/E333A/K334A | (Shields et al. 2001) |
| | in one heavy chain: | (Mimoto et al. 2013) |
| | L234Y/L235Q/G236W/S239M/H268D/ | |
| | D270E/S298A, in the opposing | |
| | heavy chain: | |
| | D270E/K326D/A330M/K334E | |
| Increased FcγRIIIa binding, | S239D/I332E/A330L | (Lazar et al. 2006) |
| decreased FcγRIIb binding | | |
| Enhance ADCP | | |
| Increased FcγRIIa binding, | G236A/S239D/I332E | (Richards et al. 2008) |
| Increased FcγRIIIa binding | | |
| Enhance CDC | | |
| Increased C1q binding | K326W/E333S | (Idusogie et al. 2001) |
| | S267E/H268F/S324T | (Moore et al. 2010) |
| | IgG1/IgG3 cross subclass | (Natsume et al. 2008) |
| Hexamerization | E345R/E430G/S440Y | (Diebolder et al. 2014) |
| Reduce effector function | | |
| Aglycosylated | N297A or N297Q or N297G | (Bolt et al. 1993; |
| | | Leabman et al. 2013; |
| | | Tao and Morrison |
| | | 1989; Walker et al. |
| | | 1989) |
| Reduced FcγR and C1q | L235E | (Alegre et al. 1992) |
| binding | IgG1: L234A/L235A or | (Xu et al. 2000; |
| | L234A/L235A/P329G | Lo et al. 2017) |
| | IgG4: F234A/L235A | (Xu et al. 2000) |
| | IgG2/IgG4 cross isotype | (Rother et al. 2007) |
| | IgG2: H268Q/V309L/A330S/P331S | (An et al. 2009) |
| | IgG2: | (Vafa et al. 2014) |
| | V234A/G237A/P238S/H268A/V309L/ | |
| | A330S/P331S | |
| Increase half-life | | |
| Increased FcRn | M252Y/S254T/T256E | (Dall'Acqua et al. 2002) |
| Binding at pH 6.0 | M428L/N434S | (Zalevsky et al. 2010) |
| Increased coengagement | | |
| Increased FcγRIIb binding | S267E/L328F | (Chu et al. 2008) |
| Increased FcγRIIa binding, | N325S/L328F | (Shang et al. 2014) |
| decreased FcγRIIIa binding | | |

In vivo half-life of antibodies may also be modulated. The Fc domain plays a central role in the stability and serum half-life of antibodies. For therapeutic approaches, antibody half-life may be reduced by using an antibody fragment missing the Fc domain or with a truncated Fc domain, such as F(ab)2, Fv, scFv, IgGACH2, F(ab′)2, scFvCH3, Fab, VL, VH, scFv4, scFv3, scFv2, dsFv, Fv, scFv-Fc or (scFv)2. The antibodies may also be in the form of diabodies or bivalent antibodies. Diabodies or bivalent antibodies may be used to increase the affinity to the target allowing lower dosage. Functional fragments missing the Fc domain or with truncated Fc domains may also be used in the development of other therapeutic approaches such as chimeric antigen receptor T cell (CAR T cells) or bispecific T cell engagers (BiTEs). In CAR constructs, one VH and one VL domain are typically connected by a short peptide linker to form a single-chain variable fragment (scFv), and the scFv fragment is further linked to a transmembrane domain and an intracytoplasmic T cell immunoreceptor tyrosine-based activation motif (from e.g. CD3ζ) and further domains of co-stimulatory molecules (from e.g. CD28, 4-1BB (CD127), or OX40) (Chang and Chen 2017). The VH and VL domains used in the scFv fragment may be the ones of the antibodies listed in Table 3. BiTEs typically consist of the fusion of two scFv of two different antibodies. One scFv domain may be of the isolated antibodies binding CLDN18.2 listed in Table 3, while the other scFv domain is from an antibody that binds e.g. to CD3, CD16, NKG2D, NKp46, CD2, CD28 or CD25. Ample guidance on BiTEs antibody formats and other bispecific antibody formats used for T-cell redirecting may be found in the review by Diego Ellerman (2019).

In another embodiment, the antibody or fragment thereof binds to CLDN18.2, the antibody having the constant light chain region (CL) of SEQ ID NO: 127 and preferably the constant heavy chain region CH1 and Fc region of SEQ ID NO: 129 with reduced FcγR binding having the L234A/L235A mutations in the constant heavy chain region CH2. More preferably, the antibody has the constant heavy chain region CH1 and Fc region of SEQ ID NO: 130 having a L234A/L235A/P329G mutation in the constant heavy chain region CH1 and Fc region with even further reduced FcγR binding.

The inventors have now surprisingly shown that ADCs of the present invention based on antibodies having the L234A/L235A mutations in the constant heavy chain region CH2 have a higher in-vivo cytotoxic activity on patient-derived tumor xenograft models than an identical ADC based on IMAB362 (see FIGS. 21C and 23B and Example 9).

In a another preferred embodiment, the antibody or fragment thereof binds to CLDN18.2 and comprises the VH sequence of SEQ ID NO: 33, the VL sequence of SEQ ID NO: 38, the constant light chain region (CL) of SEQ ID NO: 127 and the constant heavy chain region CH1 and Fc region of SEQ ID NO: 129 with L234A/L235A.

In a another preferred embodiment, the antibody or fragment thereof binds to CLDN18.2 and consists of the VH sequence of SEQ ID NO: 33, the VL sequence of SEQ ID NO: 38, the constant light chain region (CL) of SEQ ID NO: 127 and the constant heavy chain region CH1 and Fc region of SEQ ID NO: 129 with L234A/L235A.

In another embodiment, the antibody or fragment thereof binds to CLDN18.2, wherein the antibody or fragment thereof is humanized. Humanization of monoclonal antibodies is well-established. The Handbook of Therapeutic Antibodies, Second Edition, gives ample information on humanization of monoclonal antibodies (Saldanha 2014), bioinformatics tools for analysis of such antibodies (Martin and Allemn 2014) and development and manufacture of therapeutic antibodies (Jacobi et al. 2014).

In another embodiment, the antibody or fragment thereof is an isolated antibody or isolated fragment binding to CLDN18.2.

In a further embodiment, the antibody or fragment thereof binds to CLDN18.2, wherein the antibody or fragment thereof does not bind to CLDN18.1. Hence, the antibody does not exhibit cross-reactivity or cross-binding to CLDN18.1. Binding of an antibody to a target protein can be tested by flow cytometry on cells expressing the target protein. Specific binding of a tested antibody to its target protein can be visualized on a histogram plot. Such plot results in a peak with high fluorescent signal when the antibody specifically binds to the expressed target protein, and in a peak with low fluorescent signal when the antibody does not, or only very weakly bind to the expressed target protein. The degree of binding can also be expressed in a bar graph showing the maximal mean fluorescent intensity (maxMFI) measured by flow cytometry, with high maxMFI reflecting strong binding and low/no maxMFI reflecting no binding or very weak binding. Comparing maxMFI values for different antibodies in a same experimental set up may also be indicative of the affinity of the antibodies to the target, with a higher maxMFI indicating a lower off rate and higher affinity. Examples of such binding assays can be found in Example 3 and FIGS. 4 and 5.

In another embodiment, the ADC is bound to another moiety. The binding of the antibody or fragment thereof to another moiety may be covalent or no-covalent. The moiety may include radioisotopes, fluorescent tags, histological markers, cytotoxins or cytokines. Covalent binding of the moiety to the antibody may be facilitated by linkers known in the art.

In yet another embodiment, the -specific antibody or fragment thereof binds to CLDN18.2, wherein the antibody is less susceptible to posttranslational deamidation than IMAB362. In a further embodiment, the tumor-specific antibody or fragment thereof binds to CLDN18.2, wherein the antibody does not undergo posttranslational deamidation. Posttranslational modifications (PTM) are an important concern in both antibody development and antibody production and storage. Uncontrolled PTM may lead to antibodies with less efficacy, activity, potency or stability. PTMs may be N-glycosylation, lysine glycation and cysteines capped with other cysteines, glutathione, or other sulfhydryl-containing compounds from cell culture media during bioprocessing, or formation of dimers and higher oligomers due to cysteines linked by covalent disulfide bridges. Among PTMs, deamidation of asparagine (Asn, N) residues, isomerization of aspartate (aspartic acid, Asp, D) residues, and formation of succinimide intermediates are the most frequent modification reactions for therapeutic antibodies during production, storage or in vivo after administration. Deamidation of Asn and isomerization of Asp depend on sequence liabilities, the structural environment and on the storage conditions, particularly the solution pH and storage temperature. These modifications may lead to decreased or even loss of function or biological activity, especially if the affected residues are involved in target binding. Asn and Asp residues are at risk for modifications particularly when they are located in structurally flexible regions such as CDR loops, and when certain other structural prerequisites are met, whereas framework regions have been observed to be comparatively resistant to modifications. In addition to the structural location of Asn and Asp residues, canonic motifs of Asn deamidation and of Asp isomerization have also been identified. These canonical motifs are NG, NS, NN, NT, NH, and DG, DS, DD, DT and DH, respectively (Lu et al. 2019). Upon in-silico analysis, the disclosed antibodies present a DG Asp-isomerization motif in the last amino acid of CDR2 of the VL domain and in the CH2 and CH3 regions of the HC (VL-CDR2 (at position 62), CH2 (at position 282), CH3 (at position 403)).

Isomerization of Asp can be tested by subjecting the antibodies to low pH (i.e. pH 5.5) and heat (i.e. 40° C.) for two weeks, while Asn deamidation of antibodies can be tested by subjecting the antibodies to high pH (i.e. pH 8.0) and heat (i.e. 40° C.) for one week, mimicking production and storage conditions.

The inventors have now shown that the disclosed antibodies, under these harsh conditions, albeit containing Asn and Asp in their CDRs, and bearing an Asp-Gly (DG) Asp-isomerization motif, surprisingly were free of Asn deamidation (see Table 6) and Asp isomerization (see Table 7) and that their binding affinity to CLDN18.2 was not affected. IMAB362 on the other hand showed Asn deamidation under such conditions, inducing a loss of binding affinity (as seen in Table 6 and FIG. 10). The invention thus provides isolated antibodies or fragments thereof that bind to CLDN18.2 and which are less prone than IMAB362 to PTMs during production, storage and clinical application (in vivo) and that warrants for maintained binding affinity to CLDN18.2 during production, storage and clinical application (in vivo).

In one embodiment, the antibody binds to the same epitope as an antibody comprising a heavy chain sequence of SEQ ID NO: 46 and a light chain sequence of SEQ ID NO: 51.

The invention further provides an antibody competing for binding with an antibody described herein. In one embodiment, the antibody competes for binding with an antibody comprising a heavy chain sequence of SEQ ID NO: 46 and a light chain sequence of SEQ ID NO: 51.

The invention further provides an antibody that competitively inhibits binding of an antibody described herein to Claudin 18.2. In one embodiment, the antibody competitively inhibits binding of an antibody comprising a heavy chain sequence of SEQ ID NO: 46 and a light chain sequence of SEQ ID NO: 51 to Claudin 18.2.

Suitable methods to detect binding of antibodies to the same antigen include approaches to map the antigen-antibody interactions. Such approaches have been described in Abbott 2014 (Abbott, Damschroder, and Lowe 2014). Suitable methods to detect competition include competitive assays by epitope binning, as described in Abdiche 2009 (Abdiche et al. 2009). Suitable method for detecting competitive inhibition include ELISA assays.

In another embodiment, the invention relates to a method of producing an ADC of the invention.

In one embodiment, the method comprises the following steps:
a. providing A, an antibody or fragment thereof with one or more linker elements,
b. providing one or more toxins T with one or more linker elements, and
c. conjugating the antibody and the toxin resulting in the antibody-drug conjugate.

In one embodiment, the method comprises the following steps:
d. providing A, an antibody or fragment thereof with an oligopeptide linker element preferably at its C-terminus, optionally preceded by a spacer element at the antibody light and/or heavy chains,
e. providing one or more toxins T with a non-cleavable linker element optionally followed by an oligopeptide linker element, and
f. conjugating the antibody and the toxin resulting in the antibody-drug conjugate.

It is understood that any antibody A herein disclosed may be provided with any oligopeptide linker element and optional spacer element herein disclosed. Likewise, any anthracycline toxin T may be linked with any non-cleavable linker element herein disclosed. The type of conjugation may depend on the linker element and/or on the method for preparing the ADC. A representation of an ADC produced by this method can be found in FIG. 25.

In a preferred embodiment, the ADC of the invention consists of:
the antibody consisting of two heavy chains of the amino acid sequence according to SEQ ID NO: 46, and two light chains of the amino acid sequence according to SEQ ID NO: 51, wherein the antibody binds to CLDN18.2,
the linker [GGGGS]-[LPQTGG]-[ethylenediamine] at the C-terminus of the light chains, and
the anthracycline-based small molecule toxin 3′-deamino-3″,4′-anhydro-[2″(S)-methoxy-3″(R)-oxy-4″-morpholinyl]doxorubicin (PNU-159682), linked covalently to the ethylenediamine of the linker at C₁₃, resulting in the loss of C₁₄ and of the hydroxyl group.

In another preferred embodiment, the ADC of the invention consists of:
the antibody consisting of two heavy chains of the amino acid sequence according to SEQ ID NO: 133, and two light chains of the amino acid sequence according to SEQ ID NO: 51, wherein the antibody binds to CLDN18.2,
the linker [GGGGS]-[LPQTGG]-[ethylenediamine] at the C-terminus of the light chains, and
the anthracycline-based small molecule toxin 3′-deamino-3″,4′-anhydro-[2″ (S)-methoxy-3″ (R)-oxy-4″-morpholinyl]doxorubicin (PNU-159682), linked covalently to the ethylenediamine of the linker at C₁₃, resulting in the loss of C₁₄ and of the hydroxyl group.

In yet another preferred embodiment, the ADC of the invention consists of:
the antibody consisting of two heavy chains of the amino acid sequence according to SEQ ID NO: 134 and two light chains of the amino acid sequence according to SEQ ID NO: 51, wherein the antibody binds to CLDN18.2,
the linker [GGGGS]-[LPQTGG]-[ethylenediamine] at the C-terminus of the light chains, and
the anthracycline-based small molecule toxin 3′-deamino-3″,4′-anhydro-[2″ (S)-methoxy-3″ (R)-oxy-4″-morpholinyl]doxorubicin (PNU-159682), linked covalently to the ethylenediamine of the linker at C₁₃, resulting in the loss of C₁₄ and of the hydroxyl group.

The invention also relates to a pharmaceutical composition comprising the disclosed ADCs and an excipient.

Also provided are nucleic acid sequences encoding the isolated tumor-specific antibodies or functional fragments thereof that bind CLDN18.2 for their use in the manufacture of an ADC. The nucleic acid sequences may encode for the CDRs alone, for the VH and VL regions, or for the entire heavy and light chains of the antibodies. These nucleic acid sequences may be found in Table 3. The nucleic acid sequence may also encode for F(ab)₂, Fv, scFv, IgGACH2, F(ab′)2, scFvCH3, Fab, VL, VH, scFv4, scFv3, scFv2, dsFv, Fv, scFv-Fc, (scFv)₂, a non-depleting IgG, a diabody, a bivalent antibody or Fc-engineered versions thereof. The encoded immunoglobin may be an IgA1, IgA2, IgD, IgE, IgG1, IdG2, IgG3, IgG4, synthetic IgG, IgM or mutated and Fc-engineered versions thereof. The nucleic acids may additionally comprise coding sequences for oligopeptide linker elements that are directly fused to the C-termini of the antibody heavy chains and or the antibody light chains.

Also provided are expression vectors comprising a nucleic acid of the invention or a degenerate nucleic acid as a result of codon degeneracy. The expression vector may be an expression vector for protein expression in mammalian cells, bacteria, fungal or insect cells, and chosen for the type of host cell bearing the expression vector comprising the nucleic acid encoding the antibodies or functional fragments thereof. Ample guidance for the construction of such vectors may be found in Green and Sambrook (Green and Sambrook 2012). Preferred are expression vectors for mammalian cells, especially CHO cells.

Also provided are host cells comprising a nucleic acid or an expression vector of the present invention. The host cell may be a mammalian cell or cell line, a bacterial cell, a fungal cell or an insect cell. Preferred are mammalian cells, especially CHO cells.

In another embodiment, the invention relates to an ADC of the invention binding to CLDN18.2 for use in treatment.

In another embodiment of the invention relates to an ADC of the invention for use in the treatment of a subject that is suffering from a cancer/neoplastic disease.

In another embodiment, the invention relates to an ADC for use in the treatment of a subject that is at risk of developing a neoplastic disease, and/or for use in the treatment of a subject being diagnosed for a neoplastic disease.

The disclosed ADCs may be used as monotherapy. In a preferred embodiment, the disclosed ADCs are used in combination with the established standard of care of the neoplastic disease.

The neoplastic disease may be at least one disease selected from the group consisting of pancreatic, gastric, esophageal, ovarian and lung cancer. It is understood that the neoplastic disease to be treated expresses CLDN18.2.

In one embodiment, the subject is a mammal. In a preferred embodiment, the subject is a human.

Another embodiment of the invention provides a method for treating a neoplastic disease, including pancreatic, gastric, esophageal, ovarian or lung cancer, with an ADC as provided herein, wherein the method comprises administering a pharmaceutically effective amount of the ADC to a subject in need thereof. The method of treatment may be a monotherapy or preferably a combination therapy with the established standard of care of the neoplastic disease.

The amino acid sequence of human CLDN18.2 protein has the NCBI reference sequence: NP_001002026.1. The sequence can also be derived from SEQ ID NO: 135.

### DESCRIPTION OF DRAWINGS

FIG. 1: Evaluation by ELISA of the binding to lipoparticles containing CLDN18.2 or null-lipoparticles of selected chimeric and humanized anti-CLDN18.2 antibodies as indicated. A. Chimeric antibodies cCl1-1, cCl1-2, cCl1-3, IMAB362 and only secondary antibody; B. Humanized antibodies hCl1a to hCl1j, chimeric cCl1-1, IMAB362 and only secondary antibody. All newly generated antibodies bind to liposomal CLDN18.2.

FIG. 2: Sorting of PA-TU-8988S cells for expression levels of CLDN18.2. A. FC profile of PA-TU-9888S stained with IMAB362. B. FC profile of PA-TU-8988S cells sorted by FACS for high expression of CLDN18.2.

FIG. 3: Generation of HEK293T cells overexpressing huCLDN18.2. HEK293T cells, not expressing endogenously CLDN18.2, were transfected with a plasmid coding for huCLDN18.2 to stably express CLDN18.2 or coding for huCLDN18.1 to stably express CLDN18.1. The expression was analyzed by FC after staining with IMAB362, and a panCLDN18.1 antibody or an anti-human IgG secondary antibody only. A. FC profile of un-transfected HEK293T cells. B. FC profile of transfected HEK293T cells stably expressing CLDN18.1. C. FC profile of transfected HEK293T cells stably expressing CLDN18.2.

FIG. 4: Flow cytometry binding assay of chimeric cCl1-1, cCl1-2 and cCl1-3 antibodies to pre-B cell L11 cells overexpressing CLDN18.1 or CLDN18.2. The chimeric antibodies bind to CLDN18.2 and not to CLDN18.1. IMAB362 was used as positive binding control.

FIG. 5: Flow cytometry binding assay of humanized hCl1a to hCl1j antibodies to HEK293T cells overexpressing CLDN18.1 or CLDN18.2. The humanized antibodies bind to CLDN18.2 and not to CLDN18.1. IMAB362 and cCL1-1 were used as positive binding control.

FIG. 6: FACS expression profiles of A549 cells overexpressing CLDN18.2. A549 cells, not expressing endogenously CLDN18.2, were stably transfected with a plasmid coding for CLDN18.2 and the expression of CLDN18.2 was analyzed by FACS using IMAB362.

FIG. 7: Flow cytometry live-cell staining. Graph representing the percentage of isolated single cells bound by CLDN18.2 antibodies (cCl1-1, hCl1a, hCl1b, hCl1c, hCl1f and IMAB362). Single cells were isolated either from a mouse tumor expressing CLDN18.2 arising from injected A549 cells overexpressing CLDN18.2 (solid bars) or from a mouse healthy stomach expressing CLDN18.2 (open bars).

FIG. 8: Staining of frozen stomach tissue. Frozen tissue slides of mouse healthy stomach tissue expressing CLDN18.2 have been stained with hCl1a (A), hCl1b (B), hCl1c (C), hCl1f (D) or IMAB362 (E) antibodies. Pictures are representative IHC images.

FIG. 9: Staining of frozen tumor tissue arising from injected A549 cells overexpressing CLDN18.2. Frozen tissue slides of mouse tumor expressing CLDN18.2 have been stained with hCl1a (A), hCl1f (B), IMAB362 (C) or the Abcam 34H14L15 pan-CLDN18 antibodies. Pictures are representative IHC images.

FIG. 10: Effect of deamidation on the binding activity of IMAB362. The affinity of IMAB362 to CLDN18.2 decreases after deamidation.

FIG. 11: In-vitro cytotoxic assay on HEK-293T-CLDN18.2 cells of the ADCs were PNU is conjugated either to the HC or LC or HC and LC of the chimeric antibodies cCl1-1 (A), cCl1-2 (B) or cCl1-3 (C). The cytotoxic activity of the ADCs is compared to the cytotoxic activity of and ADC based on IMAB362 or the isotype control Ac10 conjugated to PNU is the same manner, or, when shown, to an ADC based on IMAB362 were the toxin MMAE is conjugated to the antibody via a MC-vc-PAB enzyme-cleavable linker. Figure legend: the ADCs are labeled HC-LC-PNU when PNU is conjugated to the heavy and light chains of the antibody, labeled HC-PNU when PNU is conjugated to the heavy chains only and labeled LC-PNU when PNU is conjugated to the light chains only. All ADCs conjugated with PNU have a -LPQTGG-oligopeptide linker and an ethylenediamine linker. A flexible oligopeptide -GGGGS- is also present when PNU is conjugated to the light chains. When present in the label, G2 stands for the two glycine in the oligopeptide linker.

FIG. 12: In-vitro cytotoxic assay on HEK-293T-CLDN18.1 cells of the ADCs were PNU is conjugated either to the HC or LC or HC and LC of the chimeric antibodies cCl1-1 (A), cCl1-2 (B) or cCl1-3 (C). The cytotoxic activity of the ADCs is compared to the cytotoxic activity of IMAB362 or the isotype control Ac10 conjugated to PNU is the same manner. Figure legend: the ADCs are labeled HC-LC-PNU when PNU is conjugated to the heavy and light chains of the antibody, labeled HC-PNU when PNU is conjugated to the heavy chains only and labeled LC-PNU when PNU is conjugated to the light chains only. All ADCs conjugated with PNU have a -LPQTGG- oligopeptide linker and an ethylenediamine linker. A flexible oligopeptide -GGGGS- is also present when PNU is conjugated to the light chains. When present in the label, G2 stands for the two glycine in the oligopeptide linker.

FIG. 13: In-vitro cytotoxic assay on BxPC-3-CLDN18.2 cells of the ADCs were PNU is conjugated either to the HC or LC or HC and LC of the chimeric antibodies cCl1-1 (A), cCl1-2 (B) or cCl1-3 (C). The cytotoxic activity of the ADCs is compared to the cytotoxic activity of IMAB362 or the isotype control Ac10 conjugated to PNU is the same manner. Figure legend: the ADCs are labeled HC-LC-PNU when PNU is conjugated to the heavy and light chains of the antibody, labeled HC-PNU when PNU is conjugated to the heavy chains only and labeled LC-PNU when PNU is conjugated to the light chains only. All ADCs conjugated with PNU have a -LPQTGG- oligopeptide linker and an ethylenediamine linker. A flexible oligopeptide -GGGGS- is also present when PNU is conjugated to the light chains. When present in the label, G2 stands for the two glycine in the oligopeptide linker.

FIG. 14: In-vitro cytotoxic assay on A549-CLDN18.2 cells of the ADCs were PNU is conjugated either to the HC or LC or HC and LC of the chimeric antibodies cCl1-1 (A), cCl1-2 (B) or cCl1-3 (C). The cytotoxic activity of the ADCs is compared to the cytotoxic activity of IMAB362 or the isotype control Ac10 conjugated to PNU is the same manner, or to an ADC based on IMAB362 were the toxin MMAE is conjugated to the antibody via a MC-vc-PAB enzyme-cleavable linker. Figure legend: the ADCs are labeled HC-LC-PNU when PNU is conjugated to the heavy and light chains of the antibody, labeled HC-PNU when PNU is conjugated to the heavy chains only and labeled LC-PNU when PNU is conjugated to the light chains only. All ADCs conjugated with PNU have a -LPQTGG- oligopeptide linker and an ethylenediamine linker. A flexible oligopeptide -GGGGS- is also present when conjugated PNU is to the light chains. When present in the label, G2 stands for the two glycine in the oligopeptide linker.

FIG. 15: In-vitro cytotoxic assay on A549-CLDN18.1 cells of the ADCs were PNU is conjugated either to the HC or LC or HC and LC of the chimeric antibodies cCl1-1 (A), cCl1-2 (B) or cCl1-3 (C). The cytotoxic activity of the ADCs is compared to the cytotoxic activity of IMAB362 or the isotype control Ac10 conjugated to PNU is the same manner, or to an ADC based on IMAB362 were the toxin MMAE is conjugated to the antibody via a MC-vc-PAB enzyme-cleavable linker. Figure legend: the ADCs are labeled HC-LC-PNU when PNU is conjugated to the heavy and light chains of the antibody, labeled HC-PNU when PNU is conjugated to the heavy chains only and labeled LC-PNU when PNU is conjugated to the light chains only. All ADCs conjugated with PNU have a -LPQTGG- oligopeptide linker and an ethylenediamine linker. A flexible oligopeptide -GGGGS- is also present when PNU is conjugated to the light chains. When present in the label, G2 stands for the two glycine in the oligopeptide linker.

FIG. 16: In-vitro cytotoxic assay on PATU-8988-S-High cells of the ADCs were PNU is conjugated either to the HC or LC or HC and LC of the chimeric antibodies cCl1-1 (A), cCl1-2 (B) or cCl1-3 (C). The cytotoxic activity of the ADCs is compared to the cytotoxic activity of IMAB362 or the isotype control Ac10 conjugated to PNU is the same manner. Figure legend: the ADCs are labeled HC-LC-PNU when PNU is conjugated to the heavy and light chains of the antibody, labeled HC-PNU when PNU is conjugated to the heavy chains only and labeled LC-PNU when PNU is conjugated to the light chains only. All ADCs conjugated with PNU have a -LPQTGG- oligopeptide linker and an ethylenediamine linker. A flexible oligopeptide -GGGGS- is also present when PNU is conjugated to the light chains. When present in the label, G2 stands for the two glycine in the oligopeptide linker.

FIG. 17: In-vitro cytotoxic assay on A549-CLDN18.2 cells of the ADCs were PNU is conjugated to the LC of the humanized antibodies hCl1a to hCl1c (A), hCl1d to hCl1f (B), hCl1g to hCl11 (C) and hCl1j (D). The cytotoxic activity of the ADCs is compared to the cytotoxic activity of ADCs were PNU is conjugated to the LC of the chimeric cCl1-1 antibody or IMAB362. Figure legend: the ADCs, labeled LC-PNU, have PNU conjugated to the light chains only via a [GGGGS]-[LPQTGG]-[ethylenediamine] linker. When present in the label, G2 stands for the two glycine in the oligopeptide linker.

FIG. 18: In-vitro cytotoxic assay on HEK-293T-CLDN18.2 cells of the ADCs were PNU is conjugated to the LC of the humanized antibodies hCl1a to hCl1c (A), hCl1d to hCl1f (B), hCl1g to hCl11 (C) and hCl1j (D). The cytotoxic activity of the ADCs is compared to the cytotoxic activity of ADCs were PNU is conjugated to the LC of the chimeric cCl1-1 antibody or IMAB362. Figure legend: the ADCs, labeled LC-PNU, have PNU conjugated to the light chains only via a [GGGGS]-[LPQTGG]-[ethylenediamine] linker. When present in the label, G2 stands for the two glycine in the oligopeptide linker.

FIG. 19: In-vitro cytotoxic assay on HEK-293T-CLDN18.1 cells of the ADCs were PNU is conjugated to the LC of the humanized antibodies hCl1a to hCl1c (A), hCl1d to hCl1f (B), hCl1g to hCl11 (C) and hCl1j (D). The cytotoxic activity of the ADCs is compared to the cytotoxic activity of ADCs were PNU is conjugated to the LC of the chimeric cCl1-1 antibody or IMAB362. Figure legend: the ADCs, labeled LC-PNU, have PNU conjugated to the light chains only via a [GGGGS]-[LPQTGG]-[ethylenediamine] linker. When present in the label, G2 stands for the two glycine in the oligopeptide linker.

FIG. 20: In-vitro cytotoxic assay on PATU-8988-S-High cells of the ADCs were PNU is conjugated to the LC of the humanized antibodies hCl1a to hCl1c (A), hCl1d to hCl1f (B), hCl1g to hCl11 (C) and hCl1j (D). The cytotoxic activity of the ADCs is compared to the cytotoxic activity of ADCs were PNU is conjugated to the LC of the chimeric cCl1-1 antibody or IMAB362. Figure legend: the ADCs, labeled LC-PNU, have PNU conjugated to the light chains only via a [GGGGS]-[LPQTGG]-[ethylenediamine] linker. When present in the label, G2 stands for the two glycine in the oligopeptide linker.

FIG. 21: In-vivo efficacy of ADC hCl1a-LC-G2-PNU (A), hCl1f-LC-G2-PNU (B) and hCl1a(LALA)-LC-G2-PNU (C) in the gastric patient-derived tumor xenograft model GXA 3037, compared to the ADC IMAB362-LC-G2-PNU. Each ADC is tested either at 0.2 mg/kg/day, 0.6 mg/kg/day or 2 mg/kg/day. Figure legend: all ADCs have PNU conjugated to the light chains only via a [GGGGS]-[LPQTGG]-[ethylenediamine] linker.

FIG. 22: In-vivo efficacy of ADC hCl1a-LC-G2-PNU in the colon cancer patient-derived tumor xenograft model CXF 742, compared to the isotype control ADC Ac10-LC-G2-PNU. Each ADC is tested at 2 mg/kg/day. Figure legend: all ADCs have PNU conjugated to the light chains only via a [GGGGS]-[LPQTGG]-[ethylenediamine] linker.

FIG. 23: In-vivo efficacy of ADC hCl1a-LC-G2-PNU (A) and hCl1a(LALA)-LC-G2-PNU (B) in the pancreatic cancer patient-derived tumor xenograft model PAXF 2175, compared to the ADC IMAB362-LC-G2-PNU. Each ADC is tested either at 0.2 mg/kg/day or 0.6 mg/kg/day. Figure legend: all ADCs have PNU conjugated to the light chains only via a [GGGGS]-[LPQTGG]-[ethylenediamine] linker.

FIG. 24: In-vivo efficacy of ADC hCl1a-LC-G2-PNU in the lung cancer patient-derived tumor xenograft model LIXFC 2050, compared to the isotype control ADC Ac10-LC-G2-PNU. Each ADC is tested at 2 mg/kg/day. Figure legend: all ADCs have PNU conjugated to the light chains only via a [GGGGS]-[LPQTGGHethylenediamine] linker.

FIG. 25: Graphical representation of and ADC were PNU has been conjugated to the antibody LC via a spacer element -GGGGS-, an oligopeptide linker element -LPQTGG- and a non-cleavable linker element EDA, linked to the C₁₃ of PNU. Figure legend: all ADCs have PNU conjugated to the light chains only via a [GGGGS]-[LPQTGG]-[ethylenediamine] linker.

### EXAMPLES

### Example 1: Generation of Chimeric and Humanized Antibodies

Techniques to generate monoclonal antibodies have been well-established. The Handbook of Therapeutic Antibodies, Second Edition (2014), gives ample information on these techniques, such as the production of monoclonal antibodies by immunization of mice or rats (Moldenhauer 2014), humanization of monoclonal antibodies (Saldanha 2014), bioinformatics tools for analysis of antibodies (Martin and Allemn 2014) or development and manufacture of therapeutic antibodies (Jacobi et al. 2014). In brief, monoclonal antibodies against CLDN18.2 were generated by DNA immunization of rats with a plasmid coding for the human CLDN18.2 cDNA (huCLDN18.2) (NCBI Reference Sequence: NM 001002026.3). The specific reactivity of rat immune sera against huCLDN18.2 was analyzed by flow cytometry (FC analysis) and ELISA. Hybridoma clones were subsequently generated from lymphocytes isolated from the 5 immunized rats to obtain chimeric antibodies. Three clones were identified as being CLDN18.2-specific, resulting in the chimeric antibodies named cCl1-1, cCl1-2 and cCl1-3 with similar CDRs (see Table 3). Subsequently, cCl1-1 cCl1-2 and cCl1-3 were humanized, resulting in 10 humanized clones named hCl1a, hCl1b, hCl1c, hCl1d, hCl1e, hCl1f, hCl1g, hCl1h, hCl1i and hCl1j antibodies (see Table 3). These antibodies were also used to generate ADCs.

As a control, the IMAB362 antibody was synthesized using the sequences of the heavy (SEQ ID NO: 55) and light chain (SEQ ID NO: 56) as published in WO2013/174509 and designated as monoclonal antibody 182-D1106-362, accession no. DSM ACC2810, deposited on 26 Oct. 2006 at the DSMZ-Deutsche Sammlung von Mikroorganismen and Zellkulturen GmbH Inhoffenstr. 7 B 38124 Braunschweig DE.

TABLE 3
antibody nucleic acid and amino acid sequences

| | | |
|---|---|---|
| NAME | SEQUENCE | SEQ ID NO |

cCl1-1

| | | |
|---|---|---|
| HCDR1 | DYAMH | SEQ ID NO: 1 |
| HCDR2 | WINTYTGKPTYADDFKG | SEQ ID NO: 2 |
| HCDR3 | AVFYGYTMDA | SEQ ID NO: 3 |
| VH | QIQLVQSGPELKKPGESVKISCKASGYTFTDYAMHWVKQAPGK | SEQ ID NO: 27 |
| | GLKWMGWINTYTGKPTYADDFKGRFVFSLEASASTANLQISNL | |
| | KNEDTATYFCARAVFYGYTMDAWGQGTSVTVSS | |
| HCDR1 | gactacgcgatgcac | SEQ ID NO: 71 |
| HCDR2 | tggatcaacacgtacacggggaagccgacatacgcggacgact | SEQ ID NO: 72 |
| | tcaagggg | |
| HCDR3 | gccgtcttctacggatatacgatggacgcg | SEQ ID NO: 73 |
| VH | cagatccagctcgtccagagcgggccggagctgaagaagccgg | SEQ ID NO: 74 |
| | gggagagcgtgaagatctcgtgcaaggcgagcggatatacgtt | |
| | cacggactacgcgatgcactgggtcaagcaagcgccggggaaa | |
| | gggctgaagtggatggggtggatcaacacgtacacggggaagc | |
| | cgacatacgcggacgacttcaaggggcgattcgtgttctcgct | |
| | ggaggcgagcgcgagcacggcgaacctgcaaatctcgaacctg | |
| | aagaacgaggacacggcgacgtacttctgcgcgcgggccgtct | |
| | tctacggatatacgatggacgcgtgggggcagggtaccagcgt | |
| | gacggtctcgagc | |
| LCDR1 | RASEDIYSNLA | SEQ ID NO: 4 |
| LCDR2 | SVKRLQD | SEQ ID NO: 5 |
| LCDR3 | LQGSNFPLT | SEQ ID NO: 6 |
| VL | DIQMTQSPASLSASLGETISIACRASEDIYSNLAWYQQKSGKS | SEQ ID NO: 28 |
| | PQLLIFSVKRLQDGVPSRFSGSGSGTQYSLKISGMQPEDEGDY | |
| | FCLQGSNFPLTFGSGTKLEIK | |
| LCDR1 | cgggcgagcgaggacatctactcgaacctggcg | SEQ ID NO: 75 |
| LCDR2 | tccgtcaagcggctgcaagac | SEQ ID NO: 76 |
| LCDR3 | ctgcaagggagcaacttcccgctgacg | SEQ ID NO: 77 |
| VL | gacatccagatgacgcagagcccggcgtcgctgagcgcgagcc | SEQ ID NO: 78 |
| | tgggggagacgatctcgatcgcgtgccgggcgagcgaggacat | |
| | ctactcgaacctggcgtggtatcaacagaagagcgggaagagc | |
| | ccgcagctgctgatcttctccgtcaagcggctgcaagacggcg | |
| | tcccgagccgattctcggggagcgggagcgggacgcagtactc | |
| | gctgaagatctcggggatgcagccggaggacgagggggactac | |
| | ttctgcctgcaagggagcaacttcccgctgacgttcgggtcgg | |
| | gtaccaaactcgagatcaaa | |

cCl1-2

| | | |
|---|---|---|
| HCDR1 | DYAMH | SEQ ID NO: 1 |
| HCDR2 | WINAYTGKPTYADDFKG | SEQ ID NO: 7 |
| HCDR3 | AVYYGYTMDA | SEQ ID NO: 8 |
| VH | QIQLVQSGPELKKPGESVKISCKTSGYTFTDYAMHWVKQGPGK | SEQ ID NO: 29 |
| | GMKWMGWINAYTGKPTYADDFKGRFVLSLEASASTANLQISNL | |
| | KNEDTATYFCARAVYYGYTMDAWGQGTSVIVSS | |
| HCDR1 | gactacgcgatgcac | SEQ ID NO: 71 |
| HCDR2 | tggatcaacgcgtacacggggaagccgacctacgcggacgact | SEQ ID NO: 79 |
| | tcaagggg | |
| HCDR3 | gccgtctactacggatatacgatggac | SEQ ID NO: 80 |
| VH | cagatccagctcgtccagagcgggccggagctgaagaagccgg | SEQ ID NO: 81 |
| | gggagagcgtgaagatctcgtgcaagacgagcggatatacgtt | |
| | cacggactacgcgatgcactgggtcaagcaggggccagggaaa | |
| | gggatgaagtggatggggtggatcaacgcgtacacggggaagc | |
| | cgacctacgcggacgacttcaaggggcgattcgtgctgagcct | |
| | ggaggcgagcgcctcgacggcgaacctgcaaatctcgaacctg | |
| | aagaacgaggacacggcgacgtacttctgcgcgcgggccgtct | |
| | actacggatatacgatggacgcgtgggggcagggtaccagcgt | |
| | gatcgtctcgagc | |
| LCDR1 | RTSEDIYSNFA | SEQ ID NO: 9 |
| LCDR2 | SVNRLQD | SEQ ID NO: 10 |
| LCDR3 | LQGSKFPLT | SEQ ID NO: 11 |
| VL | DIQMTQSPASLSASLGETISIECRTSEDIYSNFAWFQQKSGKS | SEQ ID NO: 30 |
| | PQLLIYSVNRLQDGVPSRFSGSGSGTQYSLKISGMQPEDEGDY | |
| | FCLQGSKFPLTFGSGTKLEIK | |
| LCDR1 | cggacgagcgaggacatctactcgaacttcgcg | SEQ ID NO: 82 |
| LCDR2 | tcagtcaaccggctgcaagac | SEQ ID NO: 83 |
| LCDR3 | ctgcaagggagcaagttcccgctgacg | SEQ ID NO: 84 |
| VL | gacatccagatgacgcagagcccggcgagcctgagcgcgagcc | SEQ ID NO: 85 |
| | tgggggagacgatctcgatcgagtgccggacgagcgaggacat | |
| | ctactcgaacttcgcgtggttccagcagaagagcgggaagagc | |
| | ccgcagctgctgatctactcagtcaaccggctgcaagacggcg | |
| | tcccgagccgattctcggggagcgggagcgggacgcagtactc | |
| | gctgaagatctcggggatgcagccggaggacgagggggactac | |
| | ttctgcctgcaagggagcaagttcccgctgacgttcgggagcg | |
| | gtaccaaactcgagatcaaa | |

cCl1-3

| | | |
|---|---|---|
| HCDR1 | DYAMY | SEQ ID NO: 12 |
| HCDR2 | WINTYTGKPTYADDFKG | SEQ ID NO: 2 |
| HCDR3 | AVFYGYTMDA | SEQ ID NO: 3 |
| VH | QIQLVQSGPELKKPGESVKISCKASGYTFTDYAMYWVKQVPGK | SEQ ID NO: 31 |
| | GLRWMGWINTYTGKPTYADDFKGRFVFSLEASASTANLQISNL | |
| | KNEDTATYFCARAVFYGYTMDAWGQGTSVTVSS | |
| HCDR1 | gactacgcgatgtac | SEQ ID NO: 86 |
| HCDR2 | tggatcaacacgtacacggggaagccgacctacgcggacgact | SEQ ID NO: 87 |
| | tcaagggg | |
| HCDR3 | gccgtcttctacggatatacgatggacgcg | SEQ ID NO: 73 |
| VH | cagatccagctcgtccagagcgggccggagctgaagaagccgg | SEQ ID NO: 88 |
| | gggagagcgtgaagatctcgtgcaaggcgagcggatatacgtt | |
| | cacggactacgcgatgtactgggtcaagcaagtgccggggaaa | |
| | gggctgcgatggatggggtggatcaacacgtacacggggaagc | |
| | cgacctacgcggacgacttcaaggggcgattcgtgttctcgct | |
| | ggaggcgagcgcgagcacggcgaacctgcaaatctcgaacctg | |
| | aagaacgaggacacggcgacgtacttctgcgcgcgggccgtct | |
| | tctacggatatacgatggacgcgtgggggcagggtaccagcgt | |
| | gacggtctcgagc | |
| LCDR1 | RTSEDIYSNLA | SEQ ID NO: 13 |
| LCDR2 | AIKRLQD | SEQ ID NO: 14 |
| LCDR3 | LQGSKFPLT | SEQ ID NO: 11 |
| VL | DIQMTQSPASLSASLGETISIACRTSEDIYSNLAWYQQKSGKS | SEQ ID NO: 32 |
| | PQLLIFAIKRLQDGVPSRFSGSGSGTQYSLKISGMQPEDEGDY | |
| | FCLQGSKFPLTFGSGTKLEIK | |
| LCDR1 | cggacgagcgaggacatctactcgaacctggcg | SEQ ID NO: 89 |
| LCDR2 | gcgatcaagcggctgcaagac | SEQ ID NO: 90 |
| LCDR3 | ctgcaagggagcaagttcccgctgacg | SEQ ID NO: 84 |
| VL | gacatccagatgacgcagagcccggcgagcctgagcgcgagcc | SEQ ID NO: 91 |
| | tgggggagacgatctcgatcgcgtgccggacgagcgaggacat | |
| | ctactcgaacctggcgtggtatcaacagaagagcgggaagagc | |
| | ccgcagctgctgatcttcgcgatcaagcggctgcaagacggcg | |
| | tcccgagccgattctcggggagcgggagcgggacgcagtactc | |
| | gctgaagatctcggggatgcagccggaggacgagggggactac | |
| | ttctgcctgcaagggagcaagttcccgctgacgttcgggtcgg | |
| | gtaccaaactcgagatcaaa | |

hCl1a

| | | |
|---|---|---|
| HCDR1 | DYAMH | SEQ ID NO: 1 |
| HCDR2 | WINTYTGKPTYAQKFQG | SEQ ID NO: 15 |
| HCDR3 | AVFYGYTMDA | SEQ ID NO: 3 |
| VH | QVQLVQSGAEVKKPGASVKVSCKASGYTFTDYAMHWVRQAPGQ | SEQ ID NO: 33 |
| | RLEWMGWINTYTGKPTYAQKFQGRVTITRDTSASTAYMELSSL | |
| | RSEDTAVYYCARAVFYGYTMDAWGQGTLVTVSS | |
| Heavy | QVQLVQSGAEVKKPGASVKVSCKASGYTFTDYAMHWVRQAPGQ | SEQ ID NO: 46 |
| chain | RLEWMGWINTYTGKPTYAQKFQGRVTITRDTSASTAYMELSSL | |
| | RSEDTAVYYCARAVFYGYTMDAWGQGTLVTVSSASTKGPSVFP | |
| | LAPSSKSTSGGTAALGCLVKDYFPEPVTVSWNSGALTSGVHTF | |
| | PAVLQSSGLYSLSSVVTVPSSSLGTQTYICNVNHKPSNTKVDK | |
| | KVEPKSCDKTHTCPPCPAPELLGGPSVFLFPPKPKDTLMISRT | |
| | PEVTCVVVDVSHEDPEVKFNWYVDGVEVHNAKTKPREEQYNST | |
| | YRVVSVLTVLHQDWLNGKEYKCKVSNKALPAPIEKTISKAKGQ | |
| | PREPQVYTLPPSRDELTKNQVSLTCLVKGFYPSDIAVEWESNG | |
| | QPENNYKTTPPVLDSDGSFFLYSKLTVDKSRWQQGNVFSCSVM | |
| | HEALHNHYTQKSLSLSPGX X is R or K | |
| HCDR1 | gactacgcgatgcac | SEQ ID NO: 71 |
| HCDR2 | tggatcaatacatacacggggaagccgacttatgcgcaaaaat | SEQ ID NO: 92 |
| | tccaagga | |
| HCDR3 | gcggtcttctacggatatacgatggatgcc | SEQ ID NO: 93 |
| VH | caggtccaactagtccaaagcggggcggaagtcaagaagcccg | SEQ ID NO: 94 |
| | gagcatccgtcaaagtcagctgcaaggcgagcggatatacatt | |
| | tacggactacgcgatgcactgggtcaggcaagcccctgggcaa | |
| | aggctcgaatggatgggatggatcaatacatacacggggaagc | |
| | cgacttatgcgcaaaaattccaaggaagagtcacaattacgcg | |
| | ggatacatccgcatctaccgcctacatggagctaagctcgctg | |
| | cggagcgaggatacggcggtctactattgcgcccgagcggtct | |
| | tctacggatatacgatggatgcctgggggcagggtaccctggt | |
| | cacggtctcgagc | |
| LCDR1 | RASEDIYSNLA | SEQ ID NO: 4 |
| LCDR2 | SVKRLQD | SEQ ID NO: 5 |
| LCDR3 | LQGSNFPLT | SEQ ID NO: 6 |
| VL | DIQMTQSPSSLSASVGDRVTITCRASEDIYSNLAWYQQKPGKA | SEQ ID NO: 38 |
| | PKLLIFSVKRLQDGVPSRFSGSGSGTDFTLTISSLQPEDFATY | |
| | YCLQGSNFPLTFGQGTKVEIK | |
| Light | DIQMTQSPSSLSASVGDRVTITCRASEDIYSNLAWYQQKPGKA | SEQ ID NO: 51 |
| chain | PKLLIFSVKRLQDGVPSRFSGSGSGTDFTLTISSLQPEDFATY | |
| | YCLQGSNFPLTFGQGTKVEIKRTVAAPSVFIFPPSDEQLKSGT | |
| | ASVVCLLNNFYPREAKVQWKVDNALQSGNSQESVTEQDSKDST | |
| | YSLSSTLTLSKADYEKHKVYACEVTHQGLSSPVTKSFNRGEC | |
| LCDR1 | agggcctccgaagacatctactccaacctggca | SEQ ID NO: 95 |
| LCDR2 | agcgtcaaaagactacaagat | SEQ ID NO: 96 |
| LCDR3 | ttgcaaggaagcaatttccccttgact | SEQ ID NO: 97 |
| VL | gacattcaaatgacgcaaagcccatcatcgctgagcgcatcgg | SEQ ID NO: 98 |
| | tcggggatagagtcaccataacatgcagggcctccgaagacat | |
| | ctactccaacctggcatggtatcaacaaaaaccggggaaggct | |
| | ccgaagctgctgatatttagcgtcaaaagactacaagatggag | |
| | taccgagccgattttcgggaagcgggagcgggacggatttcac | |
| | gctgaccatatcaagtttgcaaccggaggattttgcgacatac | |
| | tattgcttgcaaggaagcaatttccccttgactttcgggcaag | |
| | gtaccaaggtcgagatcaaa | |

hCl1b

| | | |
|---|---|---|
| HCDR1 | DYAMH | SEQ ID NO: 1 |
| HCDR2 | WINTYTGKPTYSQKFQG | SEQ ID NO: 16 |
| HCDR3 | AVFYGYTMDA | SEQ ID NO: 3 |
| VH | QVQLVQSGAEVKKPGASVKVSCKASGYTFTDYAMHWVRQAPGQ | SEQ ID NO: 34 |
| | RLEWMGWINTYTGKPTYSQKFQGRVTITRDTSASTAYMELSSL | |
| | RSEDTAVYYCARAVFYGYTMDAWGQGTLVTVSS | |
| Heavy | QVQLVQSGAEVKKPGASVKVSCKASGYTFTDYAMHWVRQAPGQ | SEQ ID NO: 47 |
| chain | RLEWMGWINTYTGKPTYSQKFQGRVTITRDTSASTAYMELSSL | |
| | RSEDTAVYYCARAVFYGYTMDAWGQGTLVTVSSASTKGPSVFP | |
| | LAPSSKSTSGGTAALGCLVKDYFPEPVTVSWNSGALTSGVHTF | |
| | PAVLQSSGLYSLSSVVTVPSSSLGTQTYICNVNHKPSNTKVDK | |
| | KVEPKSCDKTHTCPPCPAPELLGGPSVFLFPPKPKDTLMISRT | |
| | PEVTCVVVDVSHEDPEVKFNWYVDGVEVHNAKTKPREEQYNST | |
| | YRVVSVLTVLHQDWLNGKEYKCKVSNKALPAPIEKTISKAKGQ | |
| | PREPQVYTLPPSRDELTKNQVSLTCLVKGFYPSDIAVEWESNG | |
| | QPENNYKTTPPVLDSDGSFFLYSKLTVDKSRWQQGNVFSCSVM | |
| | HEALHNHYTQKSLSLSPGX X is R or K | |
| HCDR1 | gattatgcaatgcac | SEQ ID NO: 99 |
| HCDR2 | tggattaacacctacacgggcaagcccacatactcccaaaaat | SEQ ID NO: 100 |
| | tccaagga | |
| HCDR3 | gctgtattctatggatatacaatggatgcc | SEQ ID NO: 101 |
| VH | caggtccaattagtccaaagcggggcggaagtcaagaagccgg | SEQ ID NO: 102 |
| | gggcgagcgtcaaagtctcatgcaaagcgagcggatacacatt | |
| | tacggattatgcaatgcactgggtcaggcaagcacccggacaa | |
| | aggctggaatggatgggatggattaacacctacacgggcaagc | |
| | ccacatactcccaaaaattccaaggaagggtcacgataacgag | |
| | agacacgagcgcgagcaccggaatggatgggatggattaacac | |
| | ctacacgggcaagcccacatactcccaaaaattccaaggaagg | |
| | gtcacgataacgagagacacgagcgcgagcaccgtaccctggt | |
| | caccgtctcgagc | |
| LCDR1 | RASEDIYSNLA | SEQ ID NO: 4 |
| LCDR2 | SVKRLQD | SEQ ID NO: 5 |
| LCDR3 | LQGSNFPLT | SEQ ID NO: 6 |
| VL | DIQMTQSPSSLSASVGDRVTITCRASEDIYSNLAWYQQKPGKA | SEQ ID NO: 38 |
| | PKLLIFSVKRLQDGVPSRFSGSGSGTDFTLTISSLQPEDFATY | |
| | YCLQGSNFPLTFGQGTKVEIK | |
| Light | DIQMTQSPSSLSASVGDRVTITCRASEDIYSNLAWYQQKPGKA | SEQ ID NO: 51 |
| chain | PKLLIFSVKRLQDGVPSRFSGSGSGTDFTLTISSLQPEDFATY | |
| | YCLQGSNFPLTFGQGTKVEIKRTVAAPSVFIFPPSDEQLKSGT | |
| | ASVVCLLNNFYPREAKVQWKVDNALQSGNSQESVTEQDSKDST | |
| | YSLSSTLTLSKADYEKHKVYACEVTHQGLSSPVTKSFNRGEC | |
| LCDR1 | agggcctccgaagacatctactccaacctggca | SEQ ID NO: 95 |
| LCDR2 | agcgtcaaaagactacaagat | SEQ ID NO: 96 |
| LCDR3 | ttgcaaggaagcaatttccccttgact | SEQ ID NO: 97 |
| VL | gacattcaaatgacgcaaagcccatcatcgctgagcgcatcgg | SEQ ID NO: 98 |
| | tcggggatagagtcaccataacatgcagggcctccgaagacat | |
| | ctactccaacctggcatggtatcaacaaaaaccggggaaggct | |
| | ccgaagctgctgatatttagcgtcaaaagactacaagatggag | |
| | taccgagccgattttcgggaagcgggagcgggacggatttcac | |
| | gctgaccatatcaagtttgcaaccggaggattttgcgacatac | |
| | tattgcttgcaaggaagcaatttccccttgactttcgggcaag | |
| | gtaccaaggtcgagatcaaa | |

hCl1c

| | | |
|---|---|---|
| HCDR1 | DYAMH | SEQ ID NO: 1 |
| HCDR2 | WINTYTGKPTYSQKFQG | SEQ ID NO: 16 |
| HCDR3 | AVFYGYTMDA | SEQ ID NO: 3 |
| VH | QVQLVQSGAEVKKPGASVKVSCKASGYTFTDYAMHWVRQAPGQ | SEQ ID NO: 34 |
| | RLEWMGWINTYTGKPTYSQKFQGRVTITRDTSASTAYMELSSL | |
| | RSEDTAVYYCARAVFYGYTMDAWGQGTLVTVSS | |
| Heavy | QVQLVQSGAEVKKPGASVKVSCKASGYTFTDYAMHWVRQAPGQ | SEQ ID NO: 47 |
| chain | RLEWMGWINTYTGKPTYSQKFQGRVTITRDTSASTAYMELSSL | |
| | RSEDTAVYYCARAVFYGYTMDAWGQGTLVTVSSASTKGPSVFP | |
| | LAPSSKSTSGGTAALGCLVKDYFPEPVTVSWNSGALTSGVHTF | |
| | PAVLQSSGLYSLSSVVTVPSSSLGTQTYICNVNHKPSNTKVDK | |
| | KVEPKSCDKTHTCPPCPAPELLGGPSVFLFPPKPKDTLMISRT | |
| | PEVTCVVVDVSHEDPEVKFNWYVDGVEVHNAKTKPREEQYNST | |
| | YRVVSVLTVLHQDWLNGKEYKCKVSNKALPAPIEKTISKAKGQ | |
| | PREPQVYTLPPSRDELTKNQVSLTCLVKGFYPSDIAVEWESNG | |
| | QPENNYKTTPPVLDSDGSFFLYSKLTVDKSRWQQGNVFSCSVM | |
| | HEALHNHYTQKSLSLSPGX X is R or K | |
| HCDR1 | gattatgcaatgcac | SEQ ID NO: 99 |
| HCDR2 | tggattaacacctacacgggcaagcccacatactcccaaaaat | SEQ ID NO: 100 |
| | tccaagga | |
| HCDR3 | gctgtattctatggatatacaatggatgcc | SEQ ID NO: 101 |
| VH | caggtccaattagtccaaagcggggcggaagtcaagaagccgg | SEQ ID NO: 102 |
| | gggcgagcgtcaaagtctcatgcaaagcgagcggatacacatt | |
| | tacggattatgcaatgcactgggtcaggcaagcacccggacaa | |
| | aggctggaatggatgggatggattaacacctacacgggcaagc | |
| | ccacatactcccaaaaattccaaggaagggtcacgataacgag | |
| | agacacgagcgcgagcaccggaatggatgggatggattaacac | |
| | ctacacgggcaagcccacatactcccaaaaattccaaggaagg | |
| | gtcacgataacgagagacacgagcgcgagcaccgtaccctggt | |
| | caccgtctcgagc | |
| LCDR1 | RTSEDIYSNLA | SEQ ID NO: 17 |
| LCDR2 | AIKRLQD | SEQ ID NO: 14 |
| LCDR3 | LQGSKFPLT | SEQ ID NO: 11 |
| VL | DIQMTQSPSSLSASVGDRVTITCRTSEDIYSNLAWYQQKPGKA | SEQ ID NO: 39 |
| | PKLLIFAIKRLQDGVPSRFSGSGSGTDFTLTISSLQPEDFATY | |
| | YCLQGSKFPLTFGQGTKVEIK | |
| Light | DIQMTQSPSSLSASVGDRVTITCRTSEDIYSNLAWYQQKPGKA | SEQ ID NO: 52 |
| chain | PKLLIFAIKRLQDGVPSRFSGSGSGTDFTLTISSLQPEDFATY | |
| | YCLQGSKFPLTFGQGTKVEIKRTVAAPSVFIFPPSDEQLKSGT | |
| | ASVVCLLNNFYPREAKVQWKVDNALQSGNSQESVTEQDSKDST | |
| | YSLSSTLTLSKADYEKHKVYACEVTHQGLSSPVTKSFNRGEC | |
| LCDR1 | cgaacgagcgaggacatatactcaaaccttgca | SEQ ID NO: 103 |
| LCDR2 | gcgataaagaggctgcaagac | SEQ ID NO: 104 |
| LCDR3 | ttgcaaggctccaaatttcccctgaca | SEQ ID NO: 105 |
| VL | gacatccaaatgactcaaagcccatcatcgctatcggcatcgg | SEQ ID NO: 106 |
| | tcggggatagagtcacgataacatgccgaacgagcgaggacat | |
| | atactcaaaccttgcatggtatcaacaaaagccggggaaggcc | |
| | ccgaagctactgatattcgcgataaagaggctgcaagacggag | |
| | ttccatcacgattttcgggatctggctcggggaccgattttac | |
| | gctgactatatcatcgctgcaaccggaagattttgcaacatac | |
| | tactgcttgcaaggctccaaatttcccctgacattcggacaag | |
| | gtaccaaggtcgagatcaaa | |

hCl1d

| | | |
|---|---|---|
| HCDR1 | DYAMH | SEQ ID NO: 1 |
| HCDR2 | WINTYTGKPTYSQKFQG | SEQ ID NO: 16 |
| HCDR3 | AVFYGYTMDA | SEQ ID NO: 3 |
| VH | QVQLVQSGAEVKKPGASVKVSCKASGYTFTDYAMHWVRQAPGQ | SEQ ID NO: 34 |
| | RLEWMGWINTYTGKPTYSQKFQGRVTITRDTSASTAYMELSSL | |
| | RSEDTAVYYCARAVFYGYTMDAWGQGTLVTVSS | |
| Heavy | QVQLVQSGAEVKKPGASVKVSCKASGYTFTDYAMHWVRQAPGQ | SEQ ID NO: 47 |
| chain | RLEWMGWINTYTGKPTYSQKFQGRVTITRDTSASTAYMELSSL | |
| | RSEDTAVYYCARAVFYGYTMDAWGQGTLVTVSSASTKGPSVFP | |
| | LAPSSKSTSGGTAALGCLVKDYFPEPVTVSWNSGALTSGVHTF | |
| | PAVLQSSGLYSLSSVVTVPSSSLGTQTYICNVNHKPSNTKVDK | |
| | KVEPKSCDKTHTCPPCPAPELLGGPSVFLFPPKPKDTLMISRT | |
| | PEVTCVVVDVSHEDPEVKFNWYVDGVEVHNAKTKPREEQYNST | |
| | YRVVSVLTVLHQDWLNGKEYKCKVSNKALPAPIEKTISKAKGQ | |
| | PREPQVYTLPPSRDELTKNQVSLTCLVKGFYPSDIAVEWESNG | |
| | QPENNYKTTPPVLDSDGSFFLYSKLTVDKSRWQQGNVFSCSVM | |
| | HEALHNHYTQKSLSLSPGX X is R or K | |
| HCDR1 | gattatgcaatgcac | SEQ ID NO: 99 |
| HCDR2 | tggattaacacctacacgggcaagcccacatactcccaaaaat | SEQ ID NO: 100 |
| | tccaagga | |
| HCDR3 | gctgtattctatggatatacaatggatgcc | SEQ ID NO: 101 |
| VH | caggtccaattagtccaaagcggggcggaagtcaagaagccgg | SEQ ID NO: 102 |
| | gggcgagcgtcaaagtctcatgcaaagcgagcggatacacatt | |
| | tacggattatgcaatgcactgggtcaggcaagcacccggacaa | |
| | aggctggaatggatgggatggattaacacctacacgggcaagc | |
| | ccacatactcccaaaaattccaaggaagggtcacgataacgag | |
| | agacacgagcgcgagcaccggaatggatgggatggattaacac | |
| | ctacacgggcaagcccacatactcccaaaaattccaaggaagg | |
| | gtcacgataacgagagacacgagcgcgagcaccgtaccctggt | |
| | caccgtctcgagc | |
| LCDR1 | RTSEDIYSNFA | SEQ ID NO: 18 |
| LCDR2 | SVNRLQD | SEQ ID NO: 19 |
| LCDR3 | LQGSKFPLT | SEQ ID NO: 11 |
| VL | DIQMTQSPSSLSASVGDRVTITCRTSEDIYSNFAWYQQKPGKA | SEQ ID NO: 40 |
| | PKLLIYSVNRLQDGVPSRFSGSGSGTDFTLTISSLQPEDFATY | |
| | YCLQGSKFPLTFGQGTKVEIK | |
| Light | DIQMTQSPSSLSASVGDRVTITCRTSEDIYSNFAWYQQKPGKA | SEQ ID NO: 53 |
| chain | PKLLIYSVNRLQDGVPSRFSGSGSGTDFTLTISSLQPEDFATY | |
| | YCLQGSKFPLTFGQGTKVEIKRTVAAPSVFIFPPSDEQLKSGT | |
| | ASVVCLLNNFYPREAKVQWKVDNALQSGNSQESVTEQDSKDST | |
| | YSLSSTLTLSKADYEKHKVYACEVTHQGLSSPVTKSFNRGEC | |
| LCDR1 | cggacgagcgaggatatttattcgaactttgca | SEQ ID NO: 107 |
| LCDR2 | cagtcaatcggctacaagat | SEQ ID NO: 108 |
| LCDR3 | ctacaagggagcaaattcccgctgaca | SEQ ID NO: 84 |
| VL | gacatccaaatgacgcaatcaccgagctcgctgagcgcatctg | SEQ ID NO: 109 |
| | tcggggaccgtgtcacaatcacatgccggacgagcgaggatat | |
| | ttattcgaactttgcatggtatcaacaaaaaccgggcaaggct | |
| | ccgaaacttttgatttattcagtcaatcggctacaagatggcg | |
| | tcccgagccgatttagcgggagcggatcgggaaccgactttac | |
| | gctgacgatatcatcgctacaaccggaggacttcgcgacttat | |
| | tactgcctacaagggagcaaattcccgctgacattcggacaag | |
| | gtaccaaggtcgagatcaaa | |

hCl1e

| | | |
|---|---|---|
| HCDR1 | DYAMY | SEQ ID NO: 12 |
| HCDR2 | WINTYTGKPTYAQKFQG | SEQ ID NO: 15 |
| HCDR3 | AVFYGYTMDA | SEQ ID NO: 3 |
| VH | QVQLVQSGAEVKKPGASVKVSCKASGYTFTDYAMYWVRQAPGQ | SEQ ID NO: 35 |
| | RLEWMGWINTYTGKPTYAQKFQGRVTITRDTSASTAYMELSSL | |
| | RSEDTAVYYCARAVFYGYTMDAWGQGTLVTVSS | |
| Heavy | QVQLVQSGAEVKKPGASVKVSCKASGYTFTDYAMYWVRQAPGQ | SEQ ID NO: 48 |
| chain | RLEWMGWINTYTGKPTYAQKFQGRVTITRDTSASTAYMELSSL | |
| | RSEDTAVYYCARAVFYGYTMDAWGQGTLVTVSSASTKGPSVFP | |
| | LAPSSKSTSGGTAALGCLVKDYFPEPVTVSWNSGALTSGVHTF | |
| | PAVLQSSGLYSLSSVVTVPSSSLGTQTYICNVNHKPSNTKVDK | |
| | KVEPKSCDKTHTCPPCPAPELLGGPSVFLFPPKPKDTLMISRT | |
| | PEVTCVVVDVSHEDPEVKFNWYVDGVEVHNAKTKPREEQYNST | |
| | YRVVSVLTVLHQDWLNGKEYKCKVSNKALPAPIEKTISKAKGQ | |
| | PREPQVYTLPPSRDELTKNQVSLTCLVKGFYPSDIAVEWESNG | |
| | QPENNYKTTPPVLDSDGSFFLYSKLTVDKSRWQQGNVFSCSVM | |
| | HEALHNHYTQKSLSLSPGX X is R or K | |
| HCDR1 | gattacgcaatgtac | SEQ ID NO: 110 |
| HCDR2 | tggataaatacctatacgggaaagccaacatacgcccaaaaat | SEQ ID NO: 111 |
| | tccaaggc | |
| HCDR3 | gccgtcttttatggatatacgatggacgca | SEQ ID NO: 112 |
| VH | caggtccaactggtccaatcgggggctgaagtcaaaaagccgg | SEQ ID NO: 113 |
| | gggcgagcgtcaaagtcagctgcaaagcatcgggatacacatt | |
| | tacggattacgcaatgtactgggtcaggcaagcacccggccaa | |
| | cgactggaatggatgggctggataaatacctatacgggaaagc | |
| | caacatacgcccaaaaattccaaggccgcgtcacaataacgcg | |
| | ggacacgagcgcatcgacggcttatatggaactatcatcgctg | |
| | cgatcggaagacacggcggtctattattgcgcacgcgccgtct | |
| | tttatggatatacgatggacgcatgggggcagggtaccctggt | |
| | cacggtctcgagc | |
| LCDR1 | RASEDIYSNLA | SEQ ID NO: 4 |
| LCDR2 | SVKRLQD | SEQ ID NO: 5 |
| LCDR3 | LQGSNFPLT | SEQ ID NO: 6 |
| VL | DIQMTQSPSSLSASVGDRVTITCRASEDIYSNLAWYQQKPGKA | SEQ ID NO: 38 |
| | PKLLIFSVKRLQDGVPSRFSGSGSGTDFTLTISSLQPEDFATY | |
| | YCLQGSNFPLTFGQGTKVEIK | |
| Light | DIQMTQSPSSLSASVGDRVTITCRASEDIYSNLAWYQQKPGKA | SEQ ID NO: 51 |
| chain | PKLLIFSVKRLQDGVPSRFSGSGSGTDFTLTISSLQPEDFATY | |
| | YCLQGSNFPLTFGQGTKVEIKRTVAAPSVFIFPPSDEQLKSGT | |
| | ASVVCLLNNFYPREAKVQWKVDNALQSGNSQESVTEQDSKDST | |
| | YSLSSTLTLSKADYEKHKVYACEVTHQGLSSPVTKSFNRGEC | |
| LCDR1 | agggcctccgaagacatctactccaacctggca | SEQ ID NO: 95 |
| LCDR2 | agcgtcaaaagactacaagat | SEQ ID NO: 96 |
| LCDR3 | ttgcaaggaagcaatttccccttgact | SEQ ID NO: 97 |
| VL | gacattcaaatgacgcaaagcccatcatcgctgagcgcatcgg | SEQ ID NO: 98 |
| | tcggggatagagtcaccataacatgcagggcctccgaagacat | |
| | ctactccaacctggcatggtatcaacaaaaaccggggaaggct | |
| | ccgaagctgctgatatttagcgtcaaaagactacaagatggag | |
| | taccgagccgattttcgggaagcgggagcgggacggatttcac | |
| | gctgaccatatcaagtttgcaaccggaggattttgcgacatac | |
| | tattgcttgcaaggaagcaatttccccttgactttcgggcaag | |
| | gtaccaaggtcgagatcaaa | |

hCl1f

| | | |
|---|---|---|
| HCDR1 | DYAMH | SEQ ID NO: 1 |
| HCDR2 | WINAYTGKPTYAQKFQG | SEQ ID NO: 20 |
| HCDR3 | AVFYGYTMDA | SEQ ID NO: 3 |
| VH | QVQLVQSGAEVKKPGASVKVSCKASGYTFTDYAMHWVRQAPGQ | SEQ ID NO: 36 |
| | RLEWMGWINAYTGKPTYAQKFQGRVTITRDTSASTAYMELSSL | |
| | RSEDTAVYYCARAVFYGYTMDAWGQGTLVTVSS | |
| Heavy | QVQLVQSGAEVKKPGASVKVSCKASGYTFTDYAMHWVRQAPGQ | SEQ ID NO: 49 |
| chain | RLEWMGWINAYTGKPTYAQKFQGRVTITRDTSASTAYMELSSL | |
| | RSEDTAVYYCARAVFYGYTMDAWGQGTLVTVSSASTKGPSVFP | |
| | LAPSSKSTSGGTAALGCLVKDYFPEPVTVSWNSGALTSGVHTF | |
| | PAVLQSSGLYSLSSVVTVPSSSLGTQTYICNVNHKPSNTKVDK | |
| | KVEPKSCDKTHTCPPCPAPELLGGPSVFLFPPKPKDTLMISRT | |
| | PEVTCVVVDVSHEDPEVKFNWYVDGVEVHNAKTKPREEQYNST | |
| | YRVVSVLTVLHQDWLNGKEYKCKVSNKALPAPIEKTISKAKGQ | |
| | PREPQVYTLPPSRDELTKNQVSLTCLVKGFYPSDIAVEWESNG | |
| | QPENNYKTTPPVLDSDGSFFLYSKLTVDKSRWQQGNVFSCSVM | |
| | HEALHNHYTQKSLSLSPGX X is R or K | |
| HCDR1 | gactacgcaatgcac | SEQ ID NO: 114 |
| HCDR2 | tggattaatgcctacacggggaagccgacctacgcacaaaaat | SEQ ID NO: 115 |
| | tccaagga | |
| HCDR3 | gccgtcttctatggatatacgatggatgct | SEQ ID NO: 116 |
| VH | caggtccaattggtccaaagcggggcggaggtcaagaagccgg | SEQ ID NO: 117 |
| | gggcgagcgtcaaagtctcatgcaaggcaagcggatatacatt | |
| | tacggactacgcaatgcactgggtccggcaagcccctgggcaa | |
| | cggctggaatggatgggatggattaatgcctacacggggaagc | |
| | cgacctacgcacaaaaattccaaggacgagtcacgattacgcg | |
| | ggatactagcgcgagcaccgcatatatggagctaagctcgctg | |
| | cgatctgaggataccgctgtatactactgcgcgagagccgtct | |
| | tctatggatatacgatggatgcttgggggcagggtaccctggt | |
| | cacggtctcgagc | |
| LCDR1 | RASEDIYSNLA | SEQ ID NO: 4 |
| LCDR2 | SVKRLQD | SEQ ID NO: 5 |
| LCDR3 | LQGSNFPLT | SEQ ID NO: 6 |
| VL | DIQMTQSPSSLSASVGDRVTITCRASEDIYSNLAWYQQKPGKA | SEQ ID NO: 41 |
| | PKLLIYSVKRLQDGVPSRFSGSGSGTDFTLTISSLQPEDFATY | |
| | YCLQGSNFPLTFGQGTKVEIK | |
| Light | DIQMTQSPSSLSASVGDRVTITCRASEDIYSNLAWYQQKPGKA | SEQ ID NO: 54 |
| chain | PKLLIYSVKRLQDGVPSRFSGSGSGTDFTLTISSLQPEDFATY | |
| | YCLQGSNFPLTFGQGTKVEIKRTVAAPSVFIFPPSDEQLKSGT | |
| | ASVVCLLNNFYPREAKVQWKVDNALQSGNSQESVTEQDSKDST | |
| | YSLSSTLTLSKADYEKHKVYACEVTHQGLSSPVTKSFNRGEC | |
| LCDR1 | cgagcttcggaggacatctatagcaacttggct | SEQ ID NO: 118 |
| LCDR2 | agcgtcaaaaggctccaagac | SEQ ID NO: 119 |
| LCDR3 | ctacaaggctctaacttcccattgaca | SEQ ID NO: 120 |
| VL | gatatccaaatgacgcaatcaccatctagcctatcggcctctg | SEQ ID NO: 121 |
| | tgggggaccgagtcaccatcacatgccgagcttcggaggacat | |
| | ctatagcaacttggcttggtatcaacaaaagccggggaaagca | |
| | ccaaagctgctgatatatagcgtcaaaaggctccaagacggag | |
| | tcccaagccgattctcgggctccggctccgggacggattttac | |
| | gctgacaatttcgagcctgcaaccggaggactttgcaacctac | |
| | tattgcctacaaggctctaacttcccattgacatttgggcaag | |
| | gtaccaaggtcgagatcaaa | |

hCl1g

| | | |
|---|---|---|
| HCDR1 | DYAMH | SEQ ID NO: 1 |
| HCDR2 | WINAYTGKPTYAQKFQG | SEQ ID NO: 20 |
| HCDR3 | AVFYGYTMDA | SEQ ID NO: 3 |
| VH | QVQLVQSGAEVKKPGASVKVSCKASGYTFTDYAMHWVRQAPGQ | SEQ ID NO: 36 |
| | RLEWMGWINAYTGKPTYAQKFQGRVTITRDTSASTAYMELSSL | |
| | RSEDTAVYYCARAVFYGYTMDAWGQGTLVTVSS | |
| Heavy | QVQLVQSGAEVKKPGASVKVSCKASGYTFTDYAMHWVRQAPGQ | SEQ ID NO: 49 |
| chain | RLEWMGWINAYTGKPTYAQKFQGRVTITRDTSASTAYMELSSL | |
| | RSEDTAVYYCARAVFYGYTMDAWGQGTLVTVSSASTKGPSVFP | |
| | LAPSSKSTSGGTAALGCLVKDYFPEPVTVSWNSGALTSGVHTF | |
| | PAVLQSSGLYSLSSVVTVPSSSLGTQTYICNVNHKPSNTKVDK | |
| | KVEPKSCDKTHTCPPCPAPELLGGPSVFLFPPKPKDTLMISRT | |
| | PEVTCVVVDVSHEDPEVKFNWYVDGVEVHNAKTKPREEQYNST | |
| | YRVVSVLTVLHQDWLNGKEYKCKVSNKALPAPIEKTISKAKGQ | |
| | PREPQVYTLPPSRDELTKNQVSLTCLVKGFYPSDIAVEWESNG | |
| | QPENNYKTTPPVLDSDGSFFLYSKLTVDKSRWQQGNVFSCSVM | |
| | HEALHNHYTQKSLSLSPGX X is R or K | |
| HCDR1 | gactacgcaatgcac | SEQ ID NO: 114 |
| HCDR2 | tggattaatgcctacacggggaagccgacctacgcacaaaaat | SEQ ID NO: 115 |
| | tccaagga | |
| HCDR3 | gccgtcttctatggatatacgatggatgct | SEQ ID NO: 116 |
| VH | caggtccaattggtccaaagcggggcggaggtcaagaagccgg | SEQ ID NO: 117 |
| | gggcgagcgtcaaagtctcatgcaaggcaagcggatatacatt | |
| | tacggactacgcaatgcactgggtccggcaagcccctgggcaa | |
| | cggctggaatggatgggatggattaatgcctacacggggaagc | |
| | cgacctacgcacaaaaattccaaggacgagtcacgattacgcg | |
| | ggatactagcgcgagcaccgcatatatggagctaagctcgctg | |
| | cgatctgaggataccgctgtatactactgcgcgagagccgtct | |
| | tctatggatatacgatggatgcttgggggcagggtaccctggt | |
| | cacggtctcgagc | |
| LCDR1 | RTSEDIYSNFA | SEQ ID NO: 18 |
| LCDR2 | SVNRLQD | SEQ ID NO: 19 |
| LCDR3 | LQGSKFPLT | SEQ ID NO: 11 |
| VL | DIQMTQSPSSLSASVGDRVTITCRTSEDIYSNFAWYQQKPGKA | SEQ ID NO: 40 |
| | PKLLIYSVNRLQDGVPSRFSGSGSGTDFTLTISSLQPEDFATY | |
| | YCLQGSKFPLTFGQGTKVEIK | |
| Light | DIQMTQSPSSLSASVGDRVTITCRTSEDIYSNFAWYQQKPGKA | SEQ ID NO: 53 |
| chain | PKLLIYSVNRLQDGVPSRFSGSGSGTDFTLTISSLQPEDFATY | |
| | YCLQGSKFPLTFGQGTKVEIKRTVAAPSVFIFPPSDEQLKSGT | |
| | ASVVCLLNNFYPREAKVQWKVDNALQSGNSQESVTEQDSKDST | |
| | YSLSSTLTLSKADYEKHKVYACEVTHQGLSSPVTKSFNRGEC | |
| LCDR1 | cggacgagcgaggatatttattcgaactttgca | SEQ ID NO: 107 |
| LCDR2 | cagtcaatcggctacaagat | SEQ ID NO: 108 |
| LCDR3 | ctacaagggagcaaattcccgctgaca | SEQ ID NO: 84 |
| VL | gacatccaaatgacgcaatcaccgagctcgctgagcgcatctg | SEQ ID NO: 109 |
| | tcggggaccgtgtcacaatcacatgccggacgagcgaggatat | |
| | ttattcgaactttgcatggtatcaacaaaaaccgggcaaggct | |
| | ccgaaacttttgatttattcagtcaatcggctacaagatggcg | |
| | tcccgagccgatttagcgggagcggatcgggaaccgactttac | |
| | gctgacgatatcatcgctacaaccggaggacttcgcgacttat | |
| | tactgcctacaagggagcaaattcccgctgacattcggacaag | |
| | gtaccaaggtcgagatcaaa | |

hCl1h

| | | |
|---|---|---|
| HCDR1 | DYAMY | SEQ ID NO: 12 |
| HCDR2 | WINAYTGKPTYAQKFQG | SEQ ID NO: 20 |
| HCDR3 | AVYYGYTMDA | SEQ ID NO: 8 |
| VH | QVQLVQSGAEVKKPGASVKVSCKASGYTFTDYAMYWVRQAPGQ | SEQ ID NO: 37 |
| | RLEWMGWINAYTGKPTYAQKFQGRVTITRDTSASTAYMELSSL | |
| | RSEDTAVYYCARAVYYGYTMDAWGQGTLVTVSS | |
| Heavy | QVQLVQSGAEVKKPGASVKVSCKASGYTFTDYAMYWVRQAPGQ | SEQ ID NO: 50 |
| chain | RLEWMGWINAYTGKPTYAQKFQGRVTITRDTSASTAYMELSSL | |
| | RSEDTAVYYCARAVYYGYTMDAWGQGTLVTVSSASTKGPSVFP | |
| | LAPSSKSTSGGTAALGCLVKDYFPEPVTVSWNSGALTSGVHTF | |
| | PAVLQSSGLYSLSSVVTVPSSSLGTQTYICNVNHKPSNTKVDK | |
| | KVEPKSCDKTHTCPPCPAPELLGGPSVFLFPPKPKDTLMISRT | |
| | PEVTCVVVDVSHEDPEVKFNWYVDGVEVHNAKTKPREEQYNST | |
| | YRVVSVLTVLHQDWLNGKEYKCKVSNKALPAPIEKTISKAKGQ | |
| | PREPQVYTLPPSRDELTKNQVSLTCLVKGFYPSDIAVEWESNG | |
| | QPENNYKTTPPVLDSDGSFFLYSKLTVDKSRWQQGNVFSCSVM | |
| | HEALHNHYTQKSLSLSPGX X is R or K | |
| HCDR1 | gactacgctatgtat | SEQ ID NO: 122 |
| HCDR2 | tggattaatgcctacaccgggaagccgacttatgcgcaaaaat | SEQ ID NO: 123 |
| | ttcaagga | |
| HCDR3 | gcggtctactatggatatacgatggacgca | SEQ ID NO: 124 |
| VH | caggtccaactggttcaatctggagcggaagtcaagaagcccg | SEQ ID NO: 125 |
| | gagcatccgtcaaagtctcgtgcaaggcatctggatacacatt | |
| | caccgactacgctatgtattgggtccggcaagcccccggacaa | |
| | cggctggaatggatgggatggattaatgcctacaccgggaagc | |
| | cgacttatgcgcaaaaatttcaaggaagggtcacgattacgcg | |
| | ggacacgagcgcctcaaccgcatacatggagctatcgagcctg | |
| | cgaagcgaggacaccgcggtctactactgcgcgcgggcggtct | |
| | actatggatatacgatggacgcatgggggcagggtaccctggt | |
| | cacggtctcgagc | |
| LCDR1 | RASEDIYSNLA | SEQ ID NO: 4 |
| LCDR2 | SVKRLQD | SEQ ID NO: 5 |
| LCDR3 | LQGSNFPLT | SEQ ID NO: 6 |
| VL | DIQMTQSPSSLSASVGDRVTITCRASEDIYSNLAWYQQKPGKA | SEQ ID NO: 41 |
| | PKLLIYSVKRLQDGVPSRFSGSGSGTDFTLTISSLQPEDFATY | |
| | YCLQGSNFPLTFGQGTKVEIK | |
| Light | DIQMTQSPSSLSASVGDRVTITCRASEDIYSNLAWYQQKPGKA | SEQ ID NO: 54 |
| chain | PKLLIYSVKRLQDGVPSRFSGSGSGTDFTLTISSLQPEDFATY | |
| | YCLQGSNFPLTFGQGTKVEIKRTVAAPSVFIFPPSDEQLKSGT | |
| | ASVVCLLNNFYPREAKVQWKVDNALQSGNSQESVTEQDSKDST | |
| | YSLSSTLTLSKADYEKHKVYACEVTHQGLSSPVTKSFNRGEC | |
| LCDR1 | cgagcttcggaggacatctatagcaacttggct | SEQ ID NO: 118 |
| LCDR2 | agcgtcaaaaggctccaagac | SEQ ID NO: 119 |
| LCDR3 | ctacaaggctctaacttcccattgaca | SEQ ID NO: 120 |
| VL | gatatccaaatgacgcaatcaccatctagcctatcggcctctg | SEQ ID NO: 121 |
| | tgggggaccgagtcaccatcacatgccgagcttcggaggacat | |
| | ctatagcaacttggcttggtatcaacaaaagccggggaaagca | |
| | ccaaagctgctgatatatagcgtcaaaaggctccaagacggag | |
| | tcccaagccgattctcgggctccggctccgggacggattttac | |
| | gctgacaatttcgagcctgcaaccggaggactttgcaacctac | |
| | tattgcctacaaggctctaacttcccattgacatttgggcaag | |
| | gtaccaaggtcgagatcaaa | |

hCl1i

| | | |
|---|---|---|
| HCDR1 | DYAMY | SEQ ID NO: 12 |
| HCDR2 | WINAYTGKPTYAQKFQG | SEQ ID NO: 20 |
| HCDR3 | AVYYGYTMDA | SEQ ID NO: 8 |
| VH | QVQLVQSGAEVKKPGASVKVSCKASGYTFTDYAMYWVRQAPGQ | SEQ ID NO: 37 |
| | RLEWMGWINAYTGKPTYAQKFQGRVTITRDTSASTAYMELSSL | |
| | RSEDTAVYYCARAVYYGYTMDAWGQGTLVTVSS | |
| Heavy | QVQLVQSGAEVKKPGASVKVSCKASGYTFTDYAMYWVRQAPGQ | SEQ ID NO: 50 |
| chain | RLEWMGWINAYTGKPTYAQKFQGRVTITRDTSASTAYMELSSL | |
| | RSEDTAVYYCARAVYYGYTMDAWGQGTLVTVSSASTKGPSVFP | |
| | LAPSSKSTSGGTAALGCLVKDYFPEPVTVSWNSGALTSGVHTF | |
| | PAVLQSSGLYSLSSVVTVPSSSLGTQTYICNVNHKPSNTKVDK | |
| | KVEPKSCDKTHTCPPCPAPELLGGPSVFLFPPKPKDTLMISRT | |
| | PEVTCVVVDVSHEDPEVKFNWYVDGVEVHNAKTKPREEQYNST | |
| | YRVVSVLTVLHQDWLNGKEYKCKVSNKALPAPIEKTISKAKGQ | |
| | PREPQVYTLPPSRDELTKNQVSLTCLVKGFYPSDIAVEWESNG | |
| | QPENNYKTTPPVLDSDGSFFLYSKLTVDKSRWQQGNVFSCSVM | |
| | HEALHNHYTQKSLSLSPGX X is R or K | |
| HCDR1 | gactacgctatgtat | SEQ ID NO: 122 |
| HCDR2 | tggattaatgcctacaccgggaagccgacttatgcgcaaaaat | SEQ ID NO: 123 |
| | ttcaagga | |
| HCDR3 | gcggtctactatggatatacgatggacgca | SEQ ID NO: 124 |
| VH | caggtccaactggttcaatctggagcggaagtcaagaagcccg | SEQ ID NO: 125 |
| | gagcatccgtcaaagtctcgtgcaaggcatctggatacacatt | |
| | caccgactacgctatgtattgggtccggcaagcccccggacaa | |
| | cggctggaatggatgggatggattaatgcctacaccgggaagc | |
| | cgacttatgcgcaaaaatttcaaggaagggtcacgattacgcg | |
| | ggacacgagcgcctcaaccgcatacatggagctatcgagcctg | |
| | cgaagcgaggacaccgcggtctactactgcgcgcgggcggtct | |
| | actatggatatacgatggacgcatgggggcagggtaccctggt | |
| | cacggtctcgagc | |
| LCDR1 | RASEDIYSNLA | SEQ ID NO: 4 |
| LCDR2 | SVKRLQD | SEQ ID NO: 5 |
| LCDR3 | LQGSNFPLT | SEQ ID NO: 6 |
| VL | DIQMTQSPSSLSASVGDRVTITCRASEDIYSNLAWYQQKPGKA | SEQ ID NO: 38 |
| | PKLLIFSVKRLQDGVPSRFSGSGSGTDFTLTISSLQPEDFATY | |
| | YCLQGSNFPLTFGQGTKVEIK | |
| Light | DIQMTQSPSSLSASVGDRVTITCRASEDIYSNLAWYQQKPGKA | SEQ ID NO: 51 |
| chain | PKLLIFSVKRLQDGVPSRFSGSGSGTDFTLTISSLQPEDFATY | |
| | YCLQGSNFPLTFGQGTKVEIKRTVAAPSVFIFPPSDEQLKSGT | |
| | ASVVCLLNNFYPREAKVQWKVDNALQSGNSQESVTEQDSKDST | |
| | YSLSSTLTLSKADYEKHKVYACEVTHQGLSSPVTKSFNRGEC | |
| LCDR1 | agggcctccgaagacatctactccaacctggca | SEQ ID NO: 95 |
| LCDR2 | agcgtcaaaagactacaagat | SEQ ID NO: 96 |
| LCDR3 | ttgcaaggaagcaatttccccttgact | SEQ ID NO: 97 |
| VL | gacattcaaatgacgcaaagcccatcatcgctgagcgcatcgg | SEQ ID NO: 98 |
| | tcggggatagagtcaccataacatgcagggcctccgaagacat | |
| | ctactccaacctggcatggtatcaacaaaaaccggggaaggct | |
| | ccgaagctgctgatatttagcgtcaaaagactacaagatggag | |
| | taccgagccgattttcgggaagcgggagcgggacggatttcac | |
| | gctgaccatatcaagtttgcaaccggaggattttgcgacatac | |
| | tattgcttgcaaggaagcaatttccccttgactttcgggcaag | |
| | gtaccaaggtcgagatcaaa | |

hCl1j

| | | |
|---|---|---|
| HCDR1 | DYAMY | SEQ ID NO: 12 |
| HCDR2 | WINAYTGKPTYAQKFQG | SEQ ID NO: 20 |
| HCDR3 | AVYYGYTMDA | SEQ ID NO: 8 |
| VH | QVQLVQSGAEVKKPGASVKVSCKASGYTFTDYAMYWVRQAPGQ | SEQ ID NO: 37 |
| | RLEWMGWINAYTGKPTYAQKFQGRVTITRDTSASTAYMELSSL | |
| | RSEDTAVYYCARAVYYGYTMDAWGQGTLVTVSS | |
| Heavy | QVQLVQSGAEVKKPGASVKVSCKASGYTFTDYAMYWVRQAPGQ | SEQ ID NO: 50 |
| chain | RLEWMGWINAYTGKPTYAQKFQGRVTITRDTSASTAYMELSSL | |
| | RSEDTAVYYCARAVYYGYTMDAWGQGTLVTVSSASTKGPSVFP | |
| | LAPSSKSTSGGTAALGCLVKDYFPEPVTVSWNSGALTSGVHTF | |
| | PAVLQSSGLYSLSSVVTVPSSSLGTQTYICNVNHKPSNTKVDK | |
| | KVEPKSCDKTHTCPPCPAPELLGGPSVFLFPPKPKDTLMISRT | |
| | PEVTCVVVDVSHEDPEVKFNWYVDGVEVHNAKTKPREEQYNST | |
| | YRVVSVLTVLHQDWLNGKEYKCKVSNKALPAPIEKTISKAKGQ | |
| | PREPQVYTLPPSRDELTKNQVSLTCLVKGFYPSDIAVEWESNG | |
| | QPENNYKTTPPVLDSDGSFFLYSKLTVDKSRWQQGNVFSCSVM | |
| | HEALHNHYTQKSLSLSPGX X is R or K | |
| HCDR1 | gactacgctatgtat | SEQ ID NO: 122 |
| HCDR2 | tggattaatgcctacaccgggaagccgacttatgcgcaaaaat | SEQ ID NO: 123 |
| | ttcaagga | |
| HCDR3 | gcggtctactatggatatacgatggacgca | SEQ ID NO: 124 |
| VH | caggtccaactggttcaatctggagcggaagtcaagaagcccg | SEQ ID NO: 125 |
| | gagcatccgtcaaagtctcgtgcaaggcatctggatacacatt | |
| | caccgactacgctatgtattgggtccggcaagcccccggacaa | |
| | cggctggaatggatgggatggattaatgcctacaccgggaagc | |
| | cgacttatgcgcaaaaatttcaaggaagggtcacgattacgcg | |
| | ggacacgagcgcctcaaccgcatacatggagctatcgagcctg | |
| | cgaagcgaggacaccgcggtctactactgcgcgcgggcggtct | |
| | actatggatatacgatggacgcatgggggcagggtaccctggt | |
| | cacggtctcgagc | |
| LCDR1 | RTSEDIYSNLA | SEQ ID NO: 17 |
| LCDR2 | AIKRLQD | SEQ ID NO: 14 |
| LCDR3 | LQGSKFPLT | SEQ ID NO: 11 |
| VL | DIQMTQSPSSLSASVGDRVTITCRTSEDIYSNLAWYQQKPGKA | SEQ ID NO: 39 |
| | PKLLIFAIKRLQDGVPSRFSGSGSGTDFTLTISSLQPEDFATY | |
| | YCLQGSKFPLTFGQGTKVEIK | |
| Light | DIQMTQSPSSLSASVGDRVTITCRTSEDIYSNLAWYQQKPGKA | SEQ ID NO: 52 |
| chain | PKLLIFAIKRLQDGVPSRFSGSGSGTDFTLTISSLQPEDFATY | |
| | YCLQGSKFPLTFGQGTKVEIKRTVAAPSVFIFPPSDEQLKSGT | |
| | ASVVCLLNNFYPREAKVQWKVDNALQSGNSQESVTEQDSKDST | |
| | YSLSSTLTLSKADYEKHKVYACEVTHQGLSSPVTKSFNRGEC | |
| LCDR1 | cgaacgagcgaggacatatactcaaaccttgca | SEQ ID NO: 103 |
| LCDR2 | gcgataaagaggctgcaagac | SEQ ID NO: 104 |
| LCDR3 | ttgcaaggctccaaatttcccctgaca | SEQ ID NO: 105 |
| VL | gacatccaaatgactcaaagcccatcatcgctatcggcatcgg | SEQ ID NO: 106 |
| | tcggggatagagtcacgataacatgccgaacgagcgaggacat | |
| | atactcaaaccttgcatggtatcaacaaaagccggggaaggcc | |
| | ccgaagctactgatattcgcgataaagaggctgcaagacggag | |
| | ttccatcacgattttcgggatctggctcggggaccgattttac | |
| | gctgactatatcatcgctgcaaccggaagattttgcaacatac | |
| | tactgcttgcaaggctccaaatttcccctgacattcggacaag | |
| | gtaccaaggtcgagatcaaa | |

The antibodies described in further Examples 2 to 5 were modified to contain a LPQTGG tag (SEQ ID NO: 131) at the C-terminal end of the HC and/or a GGGGSLPQTGG tag (SEQ ID NO: 132) at the C-terminal end of the LC. The C-terminal lysine (K) on the HC was in this case replaced by the Arg (R) of the tag. The addition of the tags did not change the affinity to and specificity for CLDN18.2 of the antibodies.

### Example 2: ELISA Assay and FC Titration to Confirm the Binding to CLDN18.2 of Chimeric and Humanized Antibody Variants

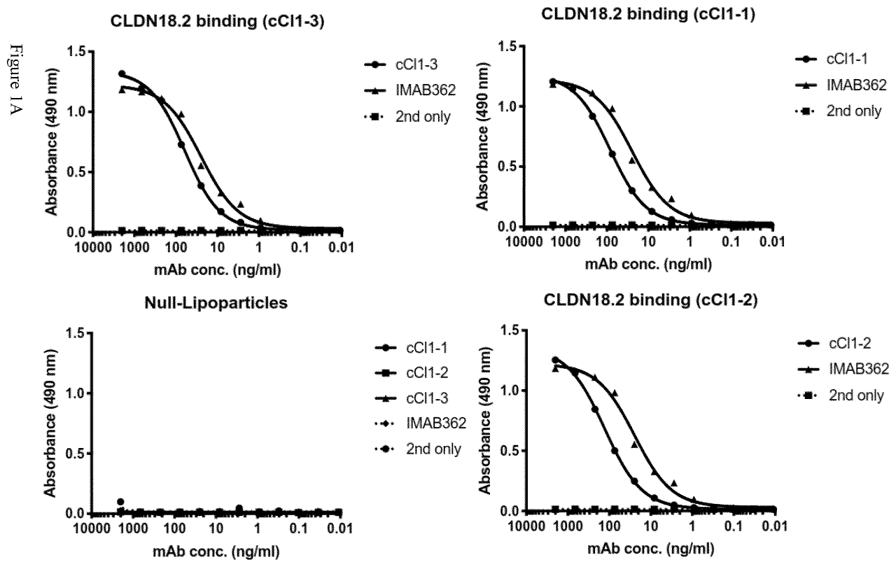

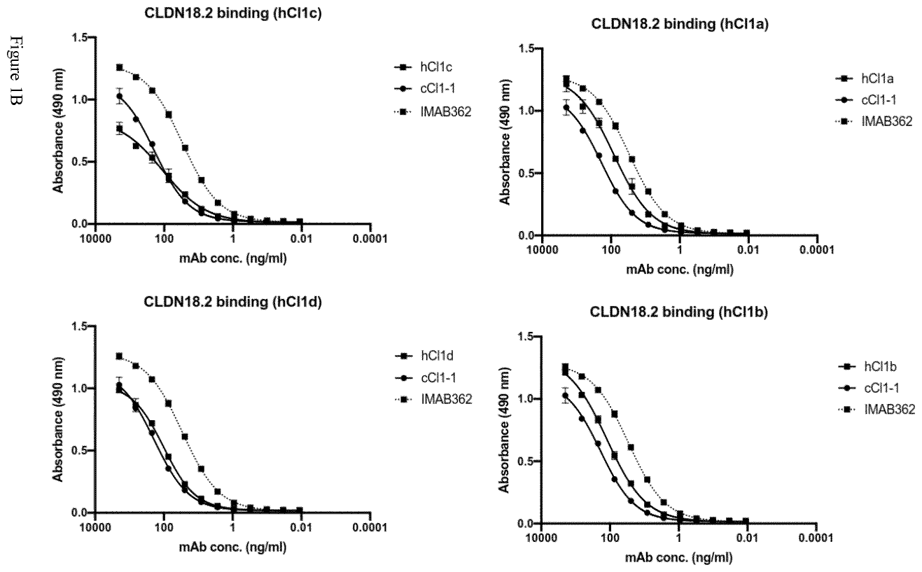

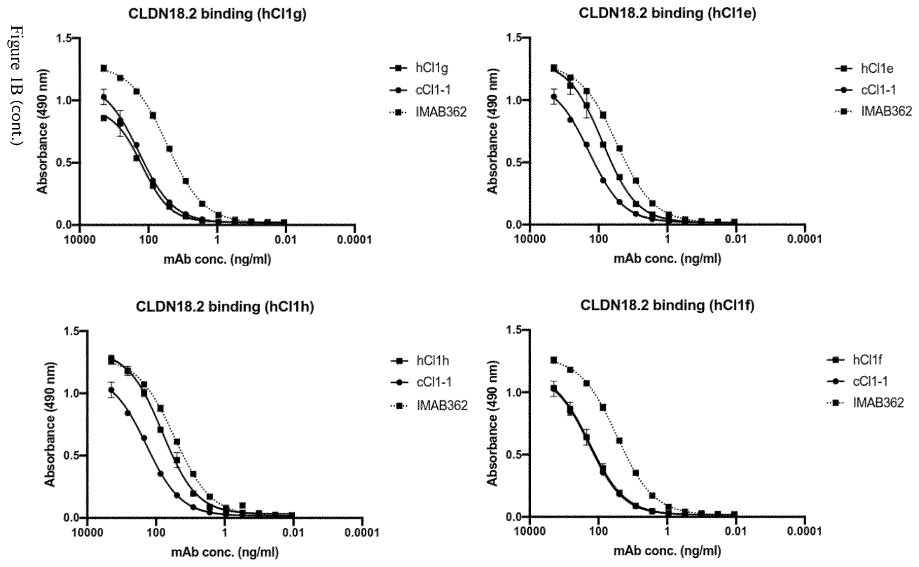

The binding affinity to CLDN18.2 of the chimeric and humanized antibodies (hCl) was tested in an ELISA assay with lipoparticles bearing CLDN18.2 as source of antigen. CLDN18.2-lipoparticles and Null-lipoparticles (without bound antigens as a negative control) were used to coat 96-well plates at a final concentration of 10 U/ml. Upon washing with PBS/0.05% Tween-20 (PBS-T) and blocking with PBS-T/3% BSA for at least 1 h at 37° C., 1:3 serial dilutions of the tested antibodies with a starting concentration of 2 μg/ml were added to the coated wells and incubated for at least 1 h at 37° C. The presence of bound antibodies was revealed through binding of an HRP-goat anti-human secondary antibody, development with SIGMAFAST™ OPD as peroxidase substrate and the reaction was stopped by adding 2M H₂SO₄, followed by reading the OD at 490 nm on an ELISA plate reader. Representative binding curves are shown in FIG. 1. All tested antibodies of the invention bind specifically to CLDN18.2 containing lipoparticles. Interestingly, humanization of the chimeric antibody did not result in decreased affinity as could be expected and even increased its affinity for 6 out of 10 antibodies, compared to the parental chimeric cCl1-1 antibody.

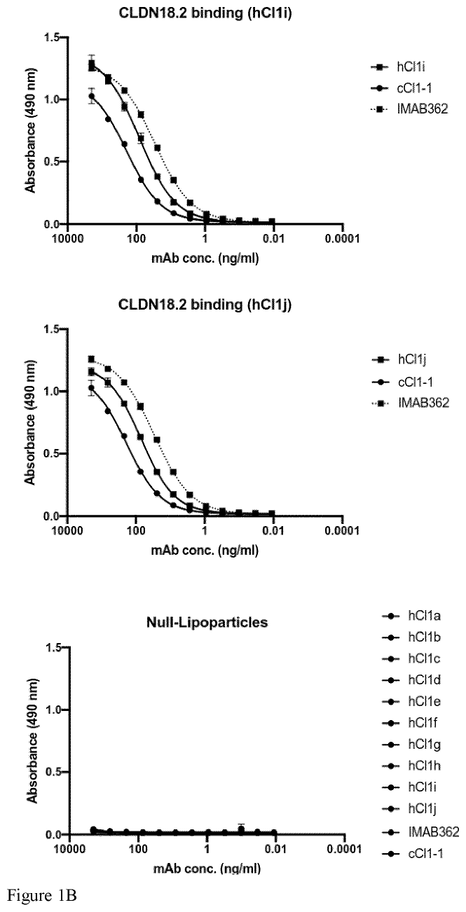

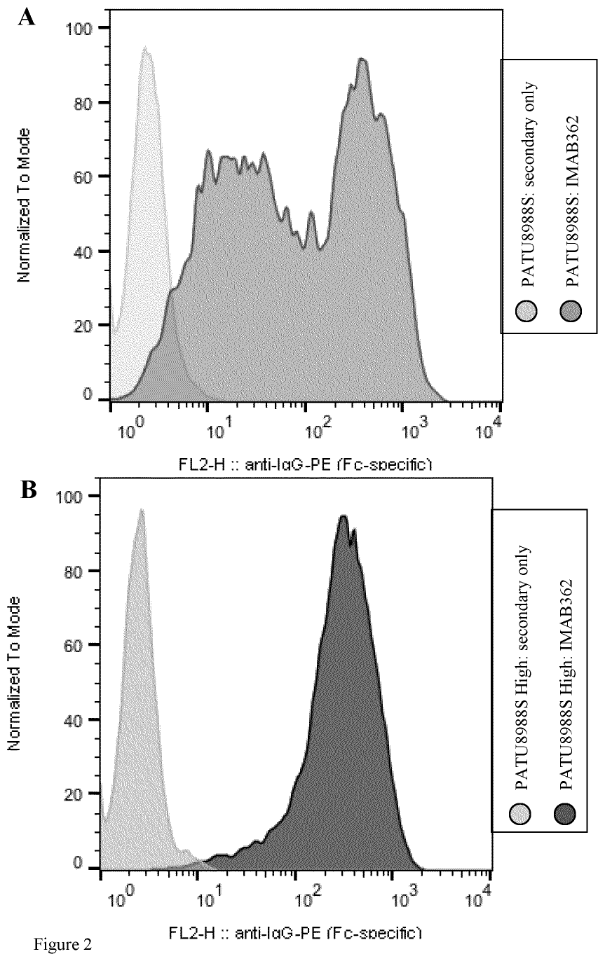

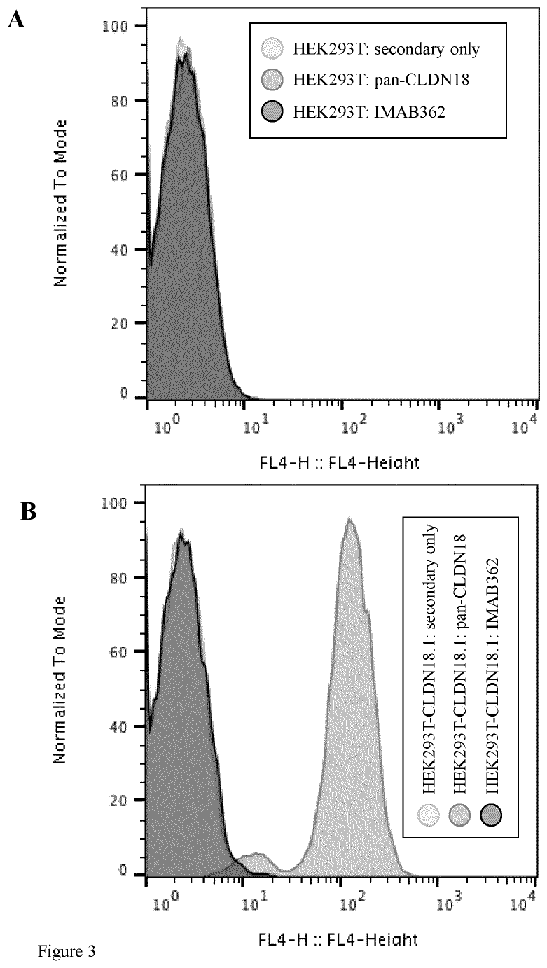

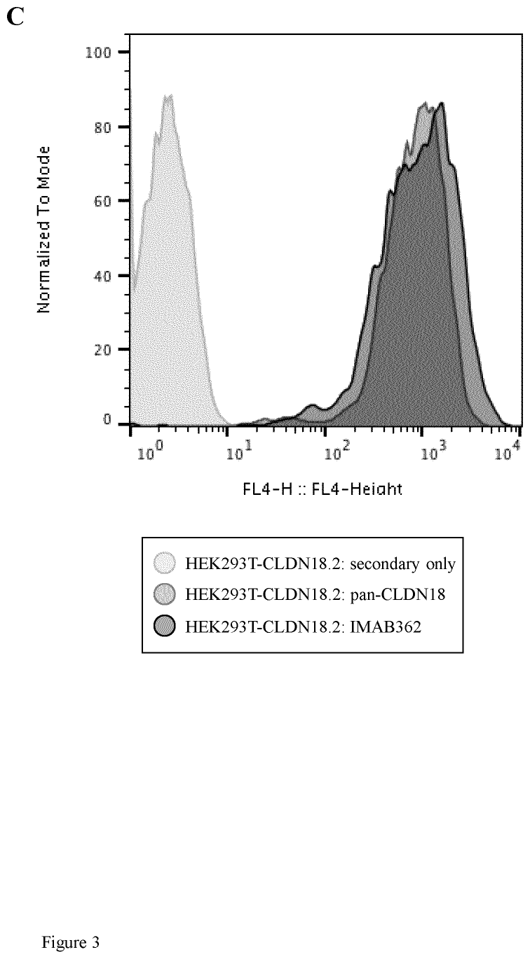

The binding of the chimeric and humanized antibodies to CLDN18.2 was also tested by FC titration with PA-TU-8988S cells (Creative Bioarray, catalog number CSC-00326) and HEK293T (ATCC, CRL-3216™) cells overexpressing CLDN18.2. FC titration allow to measure the half maximal effective concentration (EC50) of tested antibodies. PA-TU-8988S cells expressing high levels of CLDN18.2 were selected by FACS. Herein, these cells are designated as PA-TU-8988S-High cells. Based on FACS staining with IMAB362, the PA-TU-8988S cell population expresses different levels of CLDN18.2, with a high and a medium level of expression (see FIG. 2A). In order to have a more homogenous cell population, the cells were sorted by FACS to select only cells with a the higher CLDN18.2 expression. In brief, PA-TU-8988S cells suspended in FACS buffer (PBS, 2% FCS) were incubated on ice for 30 min with IMAB362 at 2 μg/ml. After wash in FACS buffer, the cells were incubated with the PE-labeled Fcγ specific IgG goat anti-human secondary antibody (eBioscience) on ice for 30 min. After wash, the stained cells were resuspended in FACS buffer, analyzed and sorted by a FACSAria™ instrument, separating medium expressing cells from high expressing cells (FIG. 2B). After sorting the collected PA-TU-8988S-High cells were resuspended in growth media, expanded and frozen aliquots were preserved in liquid N₂. HEK293T cells overexpressing CLDN18.2 or CLDN18.1 were generated as described in Example 3 and the expression of CLDN18.2 was analyzed by flow cytometry (FIG. 3).

In order to quantify the binding of the antibodies to CLDN18.2, 250×10³ cells/well of HEK293T cells overexpressing CLDN18.2 or PA-TU-8988-High cells were seeded in FC buffer (PBS/2% FBS) into 96-well plates and allowed to settle by centrifugation. IMAB362 and the hCl antibodies to be tested were diluted at 20 μg/ml, followed by 1:4 serial dilutions and incubated with the platted cells for 30 min at 4° C. A PE-coupled secondary anti-human IgG antibody was added to the cells for additional 30 min at 4° C. after washes with the FC buffer, followed by further washes with FC buffer. The cells were then resuspended in 100 μl FC buffer and measured with a FACSCalibur™ cell analyzer (BD Biosciences, USA). The FC analysis (see FIG. 5 and Table 4) shows that the hCl antibodies have a higher EC50 value than IMAB362, although having a maxMFI value in the same range as IMAB362. The similar maxMFI values may be indicative of a similar on/off rate for IMAB362 and the hCl antibodies.

TABLE 4
Maximum MFI and EC50 (μg/ml) measured on all the hCl
and IMAB362 antibodies on the HEK293T cells lines overexpressing
CLDN18.2 and on the PA-TU-8988S-High cell lines.

| | HEK293T-CLDN18.2 | | PA-TU-8988S-High | |
|---|---|---|---|---|
| Antibody | Max MFI | EC50 (μg/ml) | Max MFI | EC50 (μg/ml) |
| IMAB362 | 1968 | 0.3878 | 1046 | 0.5082 |
| hCl1a | 1879 | 0.5976 | 1649 | 2.431 |
| hCl1b | 1859 | 0.5715 | 1724 | 1.984 |
| hCl1c | 1233 | 0.7531 | 1048 | 1.472 |
| hCl1d | 1642 | 0.5411 | 1530 | 1.933 |
| hCl1e | 1935 | 0.5583 | 1862 | 2.241 |
| hCl1f | 1721 | 0.7948 | 1602 | 2.144 |
| hCl1g | 1438 | 0.6779 | 1254 | 1.77 |
| hCl1h | 2076 | 0.4325 | 1949 | 1.75 |
| hCl1i | 2175 | 0.4437 | 2087 | 1.231 |
| hCl1j | 1848 | 0.4081 | 1705 | 1.157 |

### Example 3: Generation of Pre-B Cell L11 Cells, BxPC-3 and HEK293 T Cells Stably Expressing hCLDN18.1 and hCLDN18.2, Test of Binding Specificity of the Chimeric and Humanized Antibodies

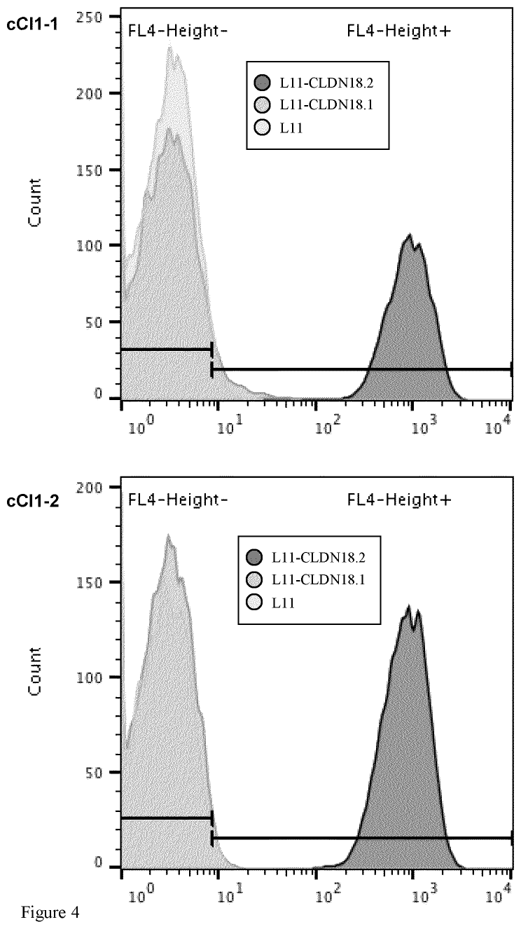

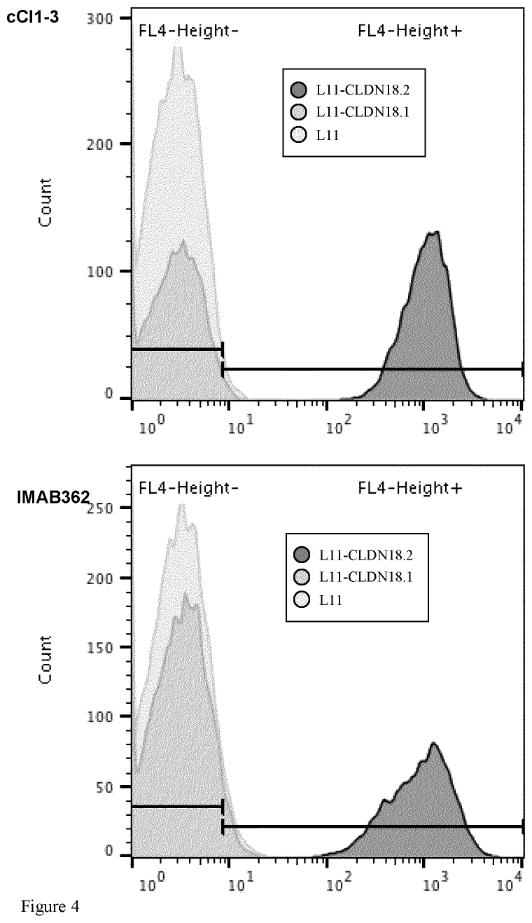

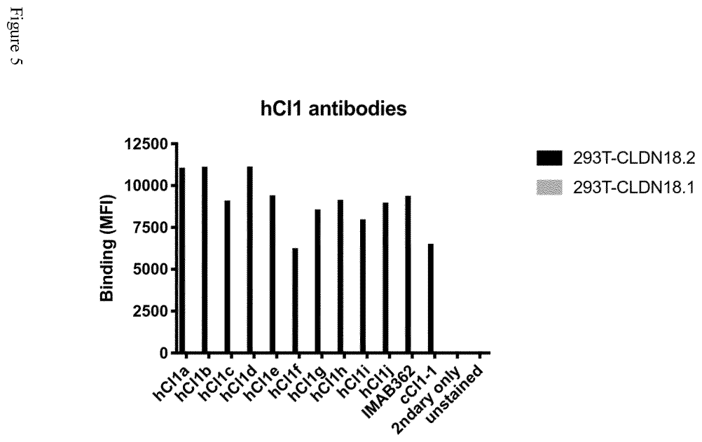

The pre-B cell L11 cell line (Waldmeier et al. 2016), BxPC-3 (ATCC CRL-1687 ™) cell line and HEK293T (ATCC CRL-32i6 ™) and A549 (ATCC CCL-185 ™) cell line do not endogenously express CLDN18.1 or CLDN18.2. Therefore, in order to test antibody binding, CLDN18.1 or CLDN18.2 were recombinantly overexpressed in the HEK293T and A549 cell lines. Cells were co-transected by electroporation with a transposase expression construct (pcDNA3.1-by-mPB), a construct bearing transposable full-length huCLDN18.1 (pPB-Puro-huCLDN18.1) or huCLDN18.2 (pPB-Puro-huCLDN18.2) along with a puromycin resistance cassette and a construct carrying EGFP as transfection control (pEGFP-N3) (Waldmeier et al. 2016). Upon electroporation, cells were allowed to recover for two days in growth media at 37° C. in a humidified incubator in a 7.5% CO₂ atmosphere for L11 cells and 5% CO₂ atmosphere for HEK293T cells and A549 cells. Transfection was verified by FC analysis of the EGFP expression. Cells expressing CLDN18.1 or CLDN18.2 were then selected by the addition of puromycin into culture at 1 μg/ml, and further expanded to allow the generation of frozen stocks in FCS with 10% DMSO. The expression of CLDN18.1 and CLDN18.2 in the transfected cells was analyzed by FC. (see FIG. 3). In brief, trypsinized HEK293T and A549 cells, and Lllcells grown in suspension were collected, by centrifugation, resuspended in PBS/2% FCS and stained for CLDN18.2 using IMAB362 as primary antibody at 2 μg/ml on ice for 30 min and, upon washing in PBS/2% FCS, stained with anti-human IgG (Fc gamma-specific) PE goat antibody (eBioscience) as secondary antibody for 30 min on ice. Upon further wash, resuspended stained cells in ice-cold FC buffer were analyzed using a FACSCalibur™ instrument (see FIG. 4 and FIG. 5). Un-transfected parental cells, not expressing CLDN18.2, were used as negative control. The expression of CLDN18.1 was analyzed in a similar fashion, using a proprietary pan-CLDN18 antibody recognizing CLDN18.1 and CLDN18.2 (see FIG. 3). Any pan-CLDN18 antibody usable for flow cytometry measurement would also be adequate such as antibody anti-Claudin-18/CLDN18 (C-term) provided by OriGene Technologies (catalog number AP50944PU-N), CLDN18 (C-Term) Rabbit pAb from MyBioSource (catalog number MBS8555451) or the CLDN18 Antibody from ProSci (catalog number 63-847).

The L11 and HEK293T cells stably expressing huCLDN18.1 and huCLDN18.2 were consequently used to test the binding specificity of the chimeric antibodies cCl1-1, cCl1-2, cCl1-3 and the humanized antibodies to CLDN18.2 and not to CLDN18.1. The cells were stained on ice for 30 min using the antibodies at 2 μg/ml and, upon washing in PBS/2% FCS, stained with anti-human IgG (Fc gamma-specific) PE goat antibody (eBioscience) as secondary antibody for 30 min on ice. All three chimeric antibodies (FIG. 4) and humanized antibodies (FIG. 5) bind to huCLDN18.2 expressed by L11 or HEK293T cells, and not to huCLDN18.1. Furthermore, the humanized antibodies bind to huCLDN18.2 with a similar affinity as IMAB362 and with an at least as good affinity as cCl1-1 (FIG. 5).

### Example 4: Testing of Humanized CLDN18.2 Antibodies Binding Activity by Flow Cytometry on Live Tumor Tissue and Live Stomach Tissue

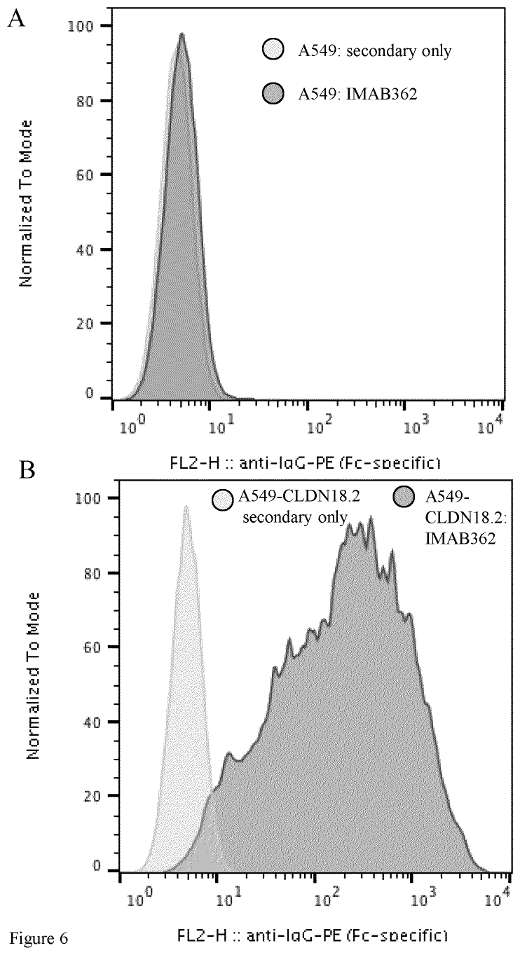

The A549 (ATCC CCLi85™) cell line does not endogenously express CLDN18.1 or CLDN18.2. In order to test antibody binding to CLDN18.2, CLDN18.2 was expressed in A549 cells. A549 cells were co-transfected by electroporation with a transposase expression construct (pcDNA3.1-by-mPB) (Klose et al. 2017), a construct bearing transposable full-length huCldn18.2 (pPB-Puro-huCldn18.1) along with puromycin expression cassette and a construct carrying EGFP as transfection control (pEGFP-N3) (Waldmeier et al. 2016). Upon electroporation, cells were allowed to recover for two days in growth media at 37° C. in a humidified incubator in a 5% CO₂ atmosphere. Transfection was verified by FC analysis of the EGFP expression. Cells expressing CLDN18.1 or CLDN18.2 were then selected by the addition of puromycin into culture at 1 μg/ml, and further expanded to allow the generation of frozen stocks in FCS with 10% DMSO. The expression of CLDN18.2 in the transfected cells was analyzed by FC. In brief, trypsinized A549 cells were collected by centrifugation, resuspended in PBS/2% FCS and stained for CLDN18.2 using IMAB362 as primary antibody at 2 μg/ml on ice for 30 min and, upon washing in PBS/2% FCS, stained with anti-human IgG (Fc gamma-specific) PE goat antibody at 2.5 μg/ml (eBioscience) as secondary antibody for 30 min on ice. Upon further wash, resuspended stained cells in ice-cold FC buffer were analyzed using a FACSCalibur™ instrument (see FIG. 6). Un-transfected parental cells, not expressing CLDN18.2, were used as negative control. The cells were deposited on 6 Dec. 2019 at the DSMZ-Deutsche Sammlung von Mikroorganismen and Zellkulturen GmbH Inhoffenstr. 7B 38124 Braunschweig DE and are available under the accession number DSM ACC3360.

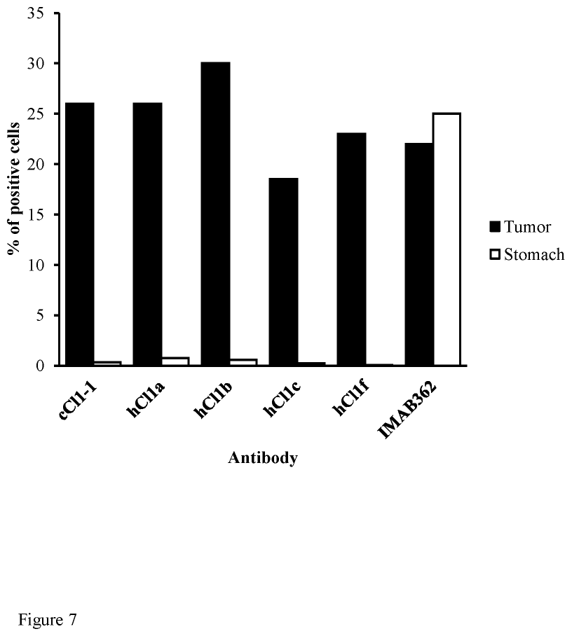

Two Balb/c mice were implanted subcutaneously with 1×10⁶ A549 cells expressing CLDN18.2 in 100 μl of 50% Matrigel and tumors growth was monitored over a few weeks until the tumor reached the desired size between 150-450 mm³. Healthy stomach tissue and tumor tissue was collected for FC analysis. The collected tissues were cut into small pieces and digested with the Miltenyi tumor dissociation kit (MACS Miltenyi Biotec, Germany). Tissue pieces were incubated with dissociation buffer (prepared according to the manufacturer instruction) in 6 well plates for 30 min in 37° C. under permanent gentle rocking motion. Samples were resuspended and strained through a 70 μm cell strainer (Corning, USA) followed by a wash with 20 ml FC buffer (PBS+2% FBS). Cell suspensions were centrifuged (5 min at 400 g for 4° C.) and the supernatants were discarded. If needed, cell suspensions were passed through a strainer and centrifuged repeatedly and pellets resuspended in 5 ml of red blood cell lysis buffer (Biolegend, USA), incubated on ice for 4 min. After incubation, 25 ml of PBS was added, and the suspensions were centrifuged again (5 min at 400 g for 4° C.). Pellets were resuspended in FC buffer (0.5-3 ml based on pellets). Equal number of cells were transferred into 96 well plates and further processed for FC analysis. The cells in the plates were washed with PBS and centrifuged (400 g for 2 min at 4° C.). Pellets were resuspended in 50 μl/well of staining mix consisting of the antibody of choice (cCl1-1, hCl1a, hCl1b, hCl1c and hCl1f at 4 μg/ml; IMAB364 at 2 μg/ml) and the AF488-labelled AE1/AE3 pan-cytokeratin antibody (Thermo Fisher Scientific, USA) diluted in PBS and incubated for 25 min on ice. After incubation, cells were washed twice in PBS and centrifuged (400 g for 2 min at 4° C.). Pellets were resuspended in 50 μl/well of secondary staining mix (PBS+PE-labelled anti-human antibody) (Thermo Fisher Scientific, USA), and incubated 25 min on ice. After incubation cells were washed again twice in PBS. Pellets were resuspended in 10011.1 of PBS containing DAPI. Plates were kept on ice until FC analysis. For FC analysis, live cells were separated from dead cells by forward scatter and DAPI stain. Live cells were then gated for the presence of cytokeratin (AF888 positive) and bound CLDN18.2 antibodies (PE positive cells). Results of the FC analysis can be seen in FIG. 7 and Table 5. The results are the average of data obtained from two mice.

All the tested antibodies (cCl1-1, hCl1a, hCl1b, hCl1c, hCl1f and IMAB364) bound to a similar percentage of tumor cells bearing CLDN18.2, approximately between 20% and 30%. However, surprisingly, only IMAB362 bound to healthy stomach cells bearing CLDN18.2 while binding of cCl1-1, hCl1a, hCl1b, hCl1c and hCl1f was barely detectable, binding less than 1% of healthy stomach cells. The difference in the binding capacity between CLDN18.2 expressed in tumor cells originating for injected A549 cells expressing CLDN18.2 and healthy stomach cells was also expressed as a ratio of the % of positive tumor cells divided by the % of positive 5 stomach cells (see last column in Table 5). This ratio was below 5 and on average close to 1 for IMAB362, and above 15, on average above 30, for the tested humanized clones of cCl1-1 (hCl1a, hCl1b, hCl1c and hCl1f).

TABLE 5
FC binding data and binding ratio of selected antibodies
to healthy stomach cells and tumor cells.

| | % of positive | | | % of positive healthy | | | Ratio | | |
|---|---|---|---|---|---|---|---|---|---|
| | tumor cells | | | stomach cells | | | tumor/stomach | | |
| | Mouse | Mouse | | Mouse | Mouse | | Ratio | Ratio | Ratio |
| | 1 | 2 | Average | 1 | 2 | Average | Mouse 1 | Mouse 2 | Average |
| cCl1-1 | 37 | 15 | 26 | 0.4 | 0.3 | 0.35 | 92.5 | 50 | 74.3 |
| hCl1a | 34 | 18 | 26 | 1.2 | 0.3 | 0.75 | 28.3 | 60 | 34.7 |
| hCl1b | 43 | 17 | 30 | 1 | 0.13 | 0.565 | 43 | 130.7 | 53.1 |
| hCl1c | 29 | 8 | 18.5 | 0.1 | 0.4 | 0.25 | 290 | 20 | 74 |
| hCl1f | 32 | 14 | 23 | 0.04 | 0.1 | 0.07 | 800 | 140 | 328.6 |
| IMAB362 | 33 | 11 | 22 | 13 | 37 | 25 | 2.53 | 0.29 | 0.88 |

Therefore, cCl1-1 and the tested humanized clones of cCl1-1 (hCl1a, hCl1b, hCl1c and hCl1f) show increased binding to tumor cells vs. healthy stomach cells and are therefore tumor-specific CLDN18.2 antibodies. In contrast IMAB362 does not allow to discriminate tumor cells bearing CLDN18.2 form healthy stomach cells bearing CLDN18.2.

### Example 5: Testing of Humanized CLDN18.2 Antibodies by Immunohistochemistry (IHC) on Frozen Tissue Samples

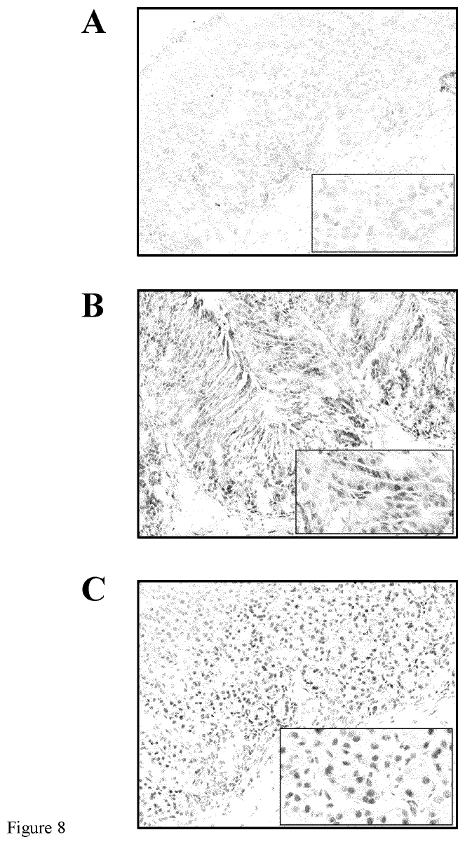

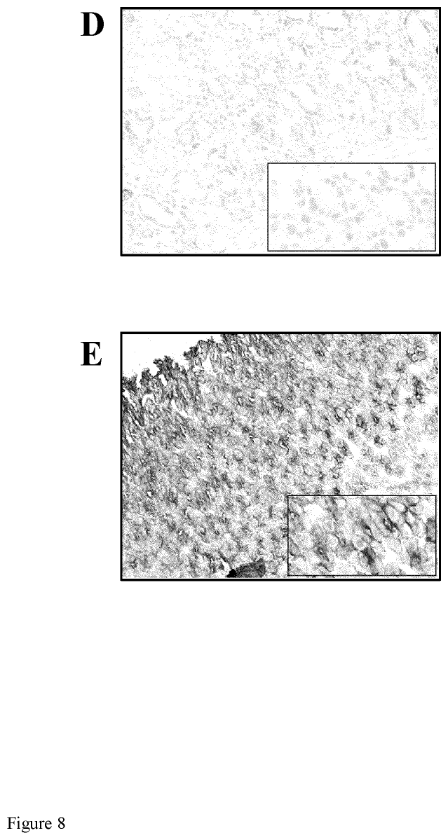

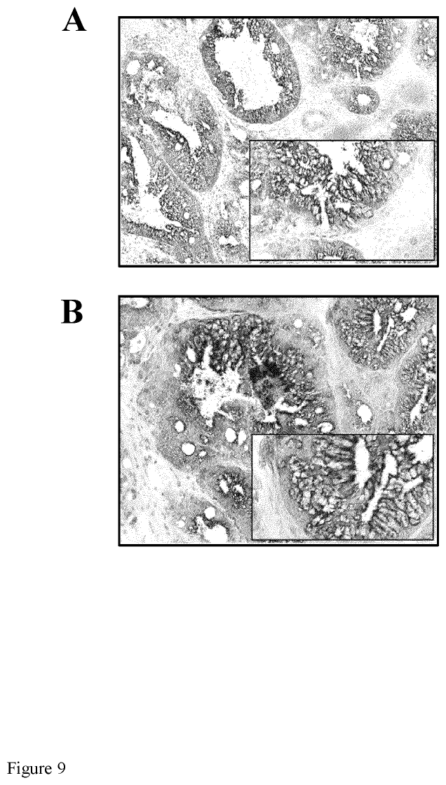

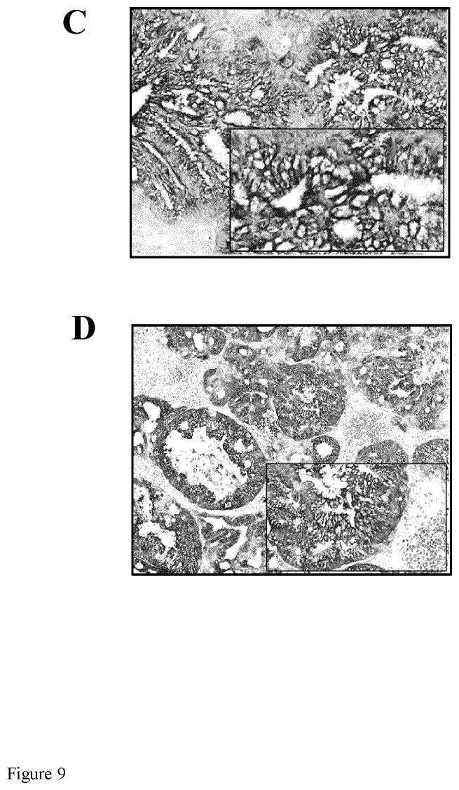

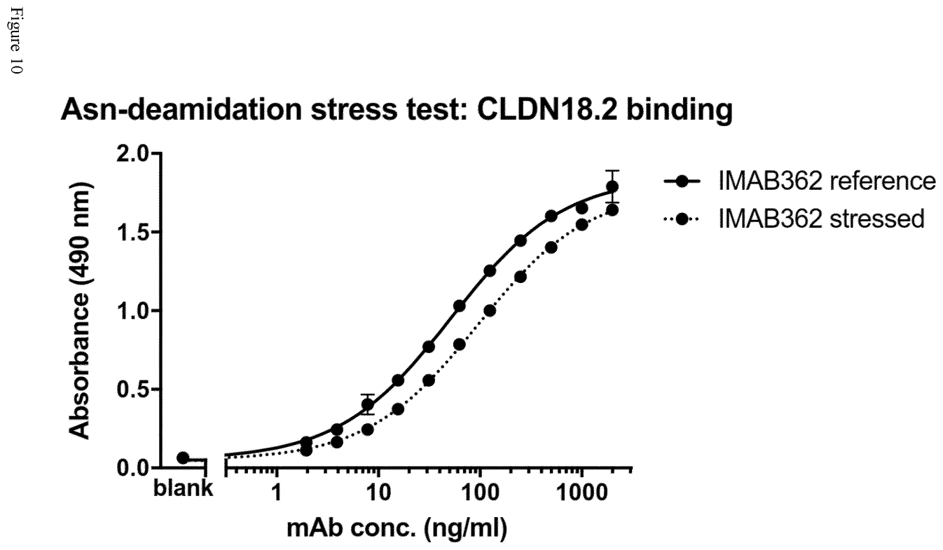

Fresh stomach and tumor tissue samples expressing CLDN18.2 obtained from Balb/c mice subcutaneously implanted with 1×10⁶ A549 cells expressing CLDN18.2 were snap-frozen in OCT in a suitable tissue mold. 5-15 μm thick tissue sections were cut with a cryostat at −20° C., 20 transferred to microscope slide at room temperature (RT) and subsequently kept frozen until IHC staining. Before staining, slides were brought back to RT and fixed in pre-cooled acetone (−20° C.) for 10 min. After evaporation of the acetone at RT, the slides were rinsed in TBS and processed to block non-specific staining sites: slides were incubated in 0.3% H₂O₂ for 15 min at RT, followed by TBS washes and incubation in a peroxidase-blocking solution (Agilent, USA) for 60 min at RT. After blocking, the slides were processed for antibody staining: the slides were incubated with the primary antibodies (hCL1a, hCl1b, hCl1c, hCl1f, IMAB362 and the 34H14L15 pan-CLDN18 antibody (Abcam, USA)) for 120 min at RT, washed in TBS, followed by incubation with an HRP-conjugated anti-human antibody (or anti-rabbit antibody for the pan-CLDN18 antibody) for 30 min at RT. Antibody binding to CLDN18.2 or pan-CLDN18 on the tissue sections was revealed by treating the slides with the DAB+substrate Chromogen system (Agilent, USA) according the manufacturer's instructions. After subsequent TBS washes, the slides were counterstained in hematoxylin, rinsed in dH2O for 15 min, dehydrated in sequential 95% and 100% ethanol washes, further followed by cleaning of the slides in xylene. Finally, the slides were mounted with a coverslip in a glycerol mounting medium (Agilent, USA). Representative microscopy images of the staining of healthy mouse stomach tissue and mouse tumor tissue can be found in FIG. 8 and FIG. 9, respectively.

FIG. 8 shows representative staining of healthy stomach tissue. Only hematoxylin stain of the nuclei is visible in tissue co-stained with hCL1a, hCl1b, hCl1c and hCl1f (respectively panels A, B, C and D), while tissue stained co-stained with IMAB362 (panel E) shows membranous CLDN18.2 DAB stain. Therefore, the tested humanized clones of cCl1-1 (hCL1a, hCl1b, hCl1c and hCl1f) do not bind healthy stomach tissue expressing CLDN18.2 in contrast to IMAB362, which binds healthy stomach tissue expressing CLDN18.2. Furthermore, FIG. 9 shows representative staining of tumor tissue, panel A, B, C and D are representative image of tumor tissue stained with hCl1a, hCl1f, IMAB362 and the Abcam 34H14L15 pan-CLDN18 antibody, respectively. All the tumor stained with the tested antibodies show strong membranous CLDN18.2 DAB stain. The tested humanized clones of cCl1-1 (hCL1a and hCl1f) bound to mouse tumor tissue expressing CLDN18.2 in similarly to IMAB362 or the pan-CLDN18 antibody. Therefore, the humanized clones of cCl1-1 exhibit increased binding to tumor tissue expressing CLDN18.2 compared to heathy stomach tissue expressing CLDN18.2.

### Example 6: Asn-Deamidation and Asp-Isomerization Liability Analysis of Humanized Antibody (hCl) Variants and IMAB362

Deamidation of Asn (N) residues and isomerization of Asp (D) residues may occur during biopharmaceutical manufacturing, storage or clinical application (in vivo). Deamidation and isomerization may lead to potential changes in protein structure, function, activity, stability and immunogenicity. Therefore, it must be minimized and controlled, particularly in a regulatory context. The presence of Asn deamidation and Asp isomerization motifs can be analyzed in-silico. The most common Asn deamidation motif is the NG motif and the most common Asp-isomerization motif in the DG motif.

Such in-silico analysis revealed that all hCl antibodies had a potential DG Asp-isomerization motif in the 2n d CDR of the VL, and that none of the hCl antibodies or IMAB362 had potential NG deamidation motifs in their CDRs. To verify the in-silico predictions, hCl antibodies and IMAB362 were stressed under high pH or low pH and heat to accelerate the modification that may to occur during manufacturing processes and long-term storage. In brief, antibody samples were buffer exchanged with Amicon centrifugal filters to 20 mM sodium phosphate buffer, pH 8.0 for the Asn-deamidation stress test or 20 mM citrate buffer, pH 5.5 for the Asp-isomerization stress test, and the samples were diluted to a final concentration of 3.0 mg/ml. 30 μl of sample was incubated for 1 week (Asn-deamidation) or 2 weeks (Asp-isomerization) at 40° C. in a thermoblock with a heated anti-condensation lid. The stressed and non-stressed sample was stored at −80° C. Asn-deamidation and Asp-isomerization of the samples was analyzed by strong cation exchange (SCX) chromatography. Deamidation of Asn leads in a SCX chromatogram to an increase of the peak area before the main peak (bM), while Asp-isomerization leads in a SCX chromatograph to an increase of the peak area after the main peak (aM) (Du et al. 2012). SCX chromatography was performed on a MAbPac SCX-10 Column (ThermoFisher Scientific, Basel, CH), with buffer A at pH 4.0 and buffer B at pH 11.0. The flow rate was of 0.5 ml/min with a pH gradient of 30-80% buffer B. 10 μg of the sample in 20 μl of buffer A was injected into the column. Sample detection was performed by protein absorbance at 280 nm. The hCl antibodies showed only an increase of bM of about 27.9-32.2% (see Table 6), which was not rated as critical. However, IMAB362 showed a pronounced increase in bM of 40.9% (see Table 6), even though this antibody does not have a NG motif in the variable domains. In contrast to the anti-CLDN18.2 monoclonal antibodies of the invention, IMAB362 has two NS motifs at positions HC CDR3 (aa 103-104) (SEQ ID NO: 55) and LC CDR 1 (aa 31-32) (SEQ ID NO: 56). NS motifs are the second most liable motifs for deamidation.

TABLE 6
Deamidation stress test of mAB, strong
cation exchange (SCX) chromatography

| | stressed | Proportion | Increase proportion of bM |
|---|---|---|---|
| mAb | yes (+)/no (−) | of bM (%) | after stress test (%) |
| hCl1a | − | 20.9 | 27.9 |
| | + | 48.8 | |
| hCl1b | − | 19.7 | 29.1 |
| | + | 48.8 | |
| hCl1c | − | 19.4 | 31.2 |
| | + | 50.6 | |
| hCl1d | − | 18.2 | 32.2 |
| | + | 50.4 | |
| hCl1e | − | 21.4 | 28.1 |
| | + | 49.5 | |
| hCl1f | − | 18.7 | 28.9 |
| | + | 47.6 | |
| hCl1g | − | 18.8 | 28.6 |
| | + | 47.4 | |
| hCl1h | − | 17.5 | 31.6 |
| | + | 49.1 | |
| hCl1i | − | 20.5 | 30.0 |
| | + | 50.5 | |
| hCl1j | − | 20.2 | 30.0 |
| | + | 50.2 | |
| IMAB362 | − | 26.0 | 40.9 |
| | + | 66.9 | |

The impact of the Asn-deamidation stress test on binding affinity to CLDN18.2 of hCl1a, hCl1i and IMAB362 was tested in an ELISA assay with lipoparticles bearing CLDN18.2 as source of antigen. CLDN18.2-lipoparticles and Null-lipoparticles (without antigens) were used to coat 96-well plates at a final concentration of 10 U/ml in 100 mM sodium carbonate, pH 9.6. Upon washing with PBS/0.05% Tween-20 (PBS-T) and blocking with PBS-T/3% BSA for at least 1 h 10 at 37° C., 1:3 serial dilutions of hCl antibodies with a starting concentration of 2 μg/ml were added and incubated for at least 1 h at 37° C. The presence of bound antibodies was revealed through binding an HRP-goat anti-human secondary antibody, developed with Sigma-Fast OPD as peroxidase substrate, the reaction was stopped by adding 2 M H₂SO₄ and reading was performed at OD-490 on an ELISA plate reader. The IMAB362 EC50 value was 1.8 times higher after the deamidation stress test (non-stressed reference: EC50 of 51.5 ng/ml, stressed: EC50 of 95.09 ng/ml) (see FIG. 10). This might be related to the increase of bM of 40.9% in SCX after deamidation stress test (see Table 6). Confirming the SCX Asn-deamidation results, no significant difference in antigen binding was observed after deamidation stress test for hCl1a and hCl1i (see Table 6). The deamidation stress test thus shows that the hCl antibodies are less prone to deamidation and potential decreased target binging than IMAB362 and predictably are more stable during manufacturing, storage and clinical application (in vivo) resulting in a more uniform and active antibody/product.

Although all hCl antibodies had a potential DG Asp-isomerization motif in the 2n d CDR of the VL and in the CH2 and CH3 domain of the HC (VL-CDR2 (at position 62), CH2 (at position 282), CH3 (at position 403)), the Asp-isomerization stress test did not reveal Asp-isomerization (see Table 7) contrary to what could have been predicted from Du et al (Du et al. 2012). The aM values of the non-stressed samples (except for IMAB362) were already noticeably high. This may be due to lysine clipping variants of the heavy chain. IMAB362 was the only antibody without a high aM in the non-stressed sample. IMAB362 is the only tested anti-CLDN18.2 antibody without C-terminal Lys, implying that for the hCl antibodies the C-terminal Lys clipping is the most probable reason for increased aM in non-stressed and stressed samples.

TABLE 7
Asp-isomerization stress test of mAbs, strong
cation exchange (SCX) chromatography

| | stressed | Proportion | Increase proportion of aM |
|---|---|---|---|
| mAb | yes (+)/no (−) | of aM (%) | after stress test (%) |
| hCl1a | − | 45.1 | −6.5 |
| | + | 38.6 | |
| hCl1b | − | 45.2 | −5.7 |
| | + | 39.5 | |
| hCl1c | − | 40.3 | −2.3 |
| | + | 38.1 | |
| hCl1d | − | 41.3 | −4.6 |
| | + | 36.7 | |
| hCl1e | − | 44.4 | −4.2 |
| | + | 40.2 | |
| hCl1f | − | 43.5 | −1.8 |
| | + | 41.7 | |
| hCl1g | − | 44.5 | −6.4 |
| | + | 38.0 | |
| hCl1h | − | 43.2 | −4.7 |
| | + | 38.5 | |
| hCl1i | − | 44.1 | −4.6 |
| | + | 39.5 | |
| hCl1j | − | 43.7 | −7.7 |
| | + | 36.0 | |
| IMAB362 | − | 1.5 | 4.1 |
| | + | 5.6 | |

### Example 7: Conjugation of mAbs with Glycine-Modified Toxin to Form ADCs Using Sortase-Mediated Conjugation

Sortase A enzyme: Recombinant and affinity purified Sortase A enzyme from Staphylococcus aureus was produced in E. coli as disclosed in WO2014140317A1.

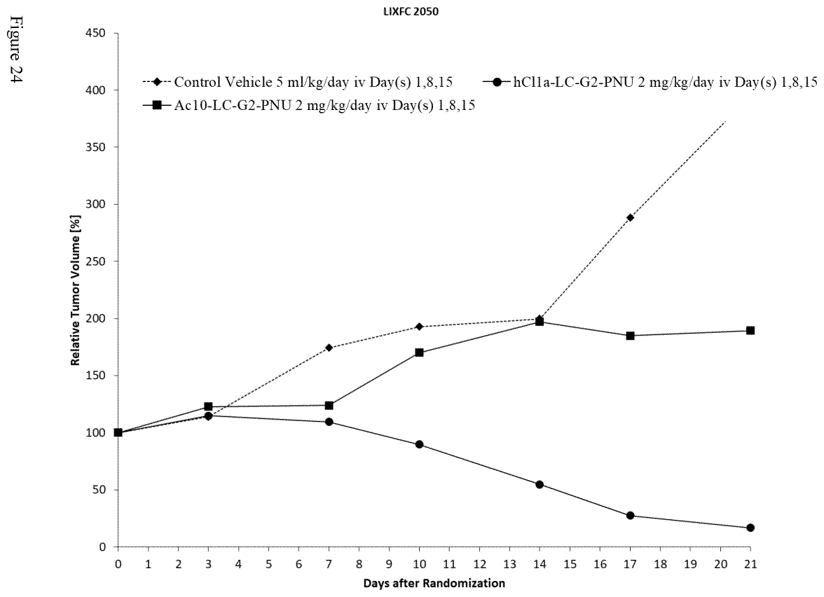

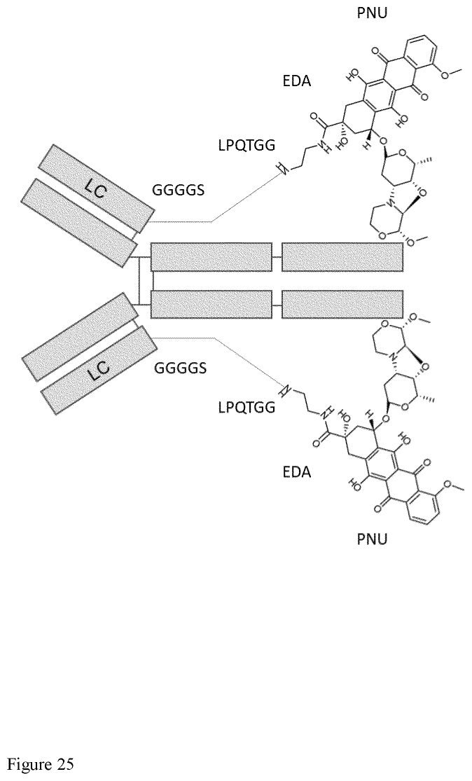

Generation of glycine-modified toxins: the biglycine-modified EDA-anthracycline derivative GG-EDA-PNU-159682 (see also FIG. 25) was manufactured by Levena Biopharma, San Diego, USA. Here the toxin PNU-159682 was synthesized to already include the non-cleavable linker EDA and an oligopeptide linker GG. The identity and the purity of the glycine-modified toxin was confirmed by mass-spectrometry and HPLC. The glycine-modified toxins exhibited >95% purity, as determined by HPLC chromatography.

Sortase-mediated antibody conjugation: the above-mentioned toxins were conjugated to the heavy chain and light chain or only light chain LPQTG-tagged anti-CLDN18.2 antibodies as per Table 3 and comparative antibodies (IMAB362, the CD30-specific antibody AC10). Alternatively, toxins were conjugated only to the light chain of the antibodies. The antibodies were conjugated to the toxins by incubating heavy and light chains or light chain-only LPQTG-tagged mAbs at 20 μM with glycine-modified toxin at 100 μM and Sortase A at 4 μM in the conjugation buffer (50 mM HEPES pH 7.5, 150 mM NaCl, 1 mM CaCl₂, 10% glycerol) for 3.5 h at 25° C. or overnight at 4° C. The reaction was stopped by passage through a rProtein A GraviTrap column (GE Healthcare). The column was washed with 36 column volumes of wash buffer (25 mM HEPES pH 7.5, 150 mM NaCl, 10% (v/v) Glycerol). Bound conjugate was eluted with 5 column volumes of elution buffer (0.1 M glycine pH 2.7, 50 mM NaCl, 10% (v/v) Glycerol), with 0.5 column volume fractions collected into tubes containing 1M HEPES pH 8 to neutralize the acid. Protein containing fractions were pooled and formulated in Histidine buffer (15 mM Histidine, pH 6.5, 175 mM Sucrose, 0.02% Tween 20) using a Zeba Spin (Thermo Fisher) desalting column. Endotoxins were removed using Pierce High Capacity Endotoxin Removal Resin (Thermo Fisher) and sterile filtered through a 0.22 μm filter. The final concentration of the ADCs was measured by UV-visible spectroscopy.

The ADC IMAB362-MC-vc-PAB-MMAE was generated as disclosed in WO2016/166122 (Example 1, section 3, page 75-76).

ADC analytics: DAR was assessed by Reverse Phase Chromatography performed on a PLRP-S, 300 Å, 2.1×150 mm, 3 μm column (Agilent) run at 0.7 ml/min at 60° C. with a 9-minute linear gradient (25-40%) followed by a 4-minute linear gradient (40-75%) between 0.1% TFA/3% CH₃CN/H₂O and 0.1% TFA/CH₃CN. Samples were first reduced by incubation with 10% v/v 0.5 M DTT, pH 8.0 at 37° C. for 15 minutes. All generated ADCs had a DAR LC=2 or a DAR HC-LC=4.

### Example 8: In-Vitro Cytotoxic Assays of Anti-CLDN18.2 Antibody-Based ADCs on CLDN18.2-Expressing Cells [Data from NBET′2483]

In Example 8 and following Example 9, an ADC of the formula [antibody]-HC-LC-PNU is an ADC where the antibody is conjugated at the heavy and light chain with the toxin PNU-159682 and has a DAR=4; an ADC of the formula [antibody]-HC-PNU or [antibody]-LC-PNU is conjugated at the heavy or light chain, respectively, with the toxin PNU-159682 and has a DAR=2. All these ADCs also have an -LPQTGG- oligopeptide linker and ethylenediamine non-cleavable linker. The structure of an ADC of the formula [antibody]-LC-PNU can be seen in FIG. 25.

Cytotoxicity of anti-CLDN18.2 ADCs was investigated using A549 cells or HEK293T cells or BxPC-3 engineered to overexpress hCLDN18.2 (see Example 3 and 4) or PA-TU-8988S-high cells (see Example 2) endogenously expressing hCLDN18.2 and compared to IMAB362-HC-G2-PNU, IMAB362-LC-G2-PNU, IMAB362-HC-LC-G2-PNU or IIVIAB362-MC-vc-PAB-MMAE. HEK293T and A549 cells engineered to overexpress hCLDN18.1 (see Example 3) were used to show specificity to CLDN18.2 and not to CLDN18.1.

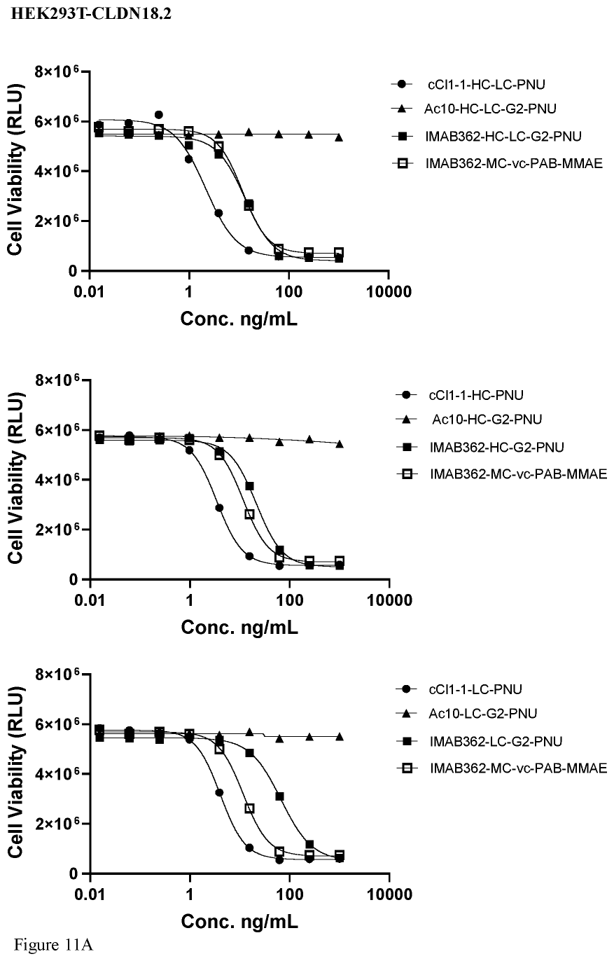

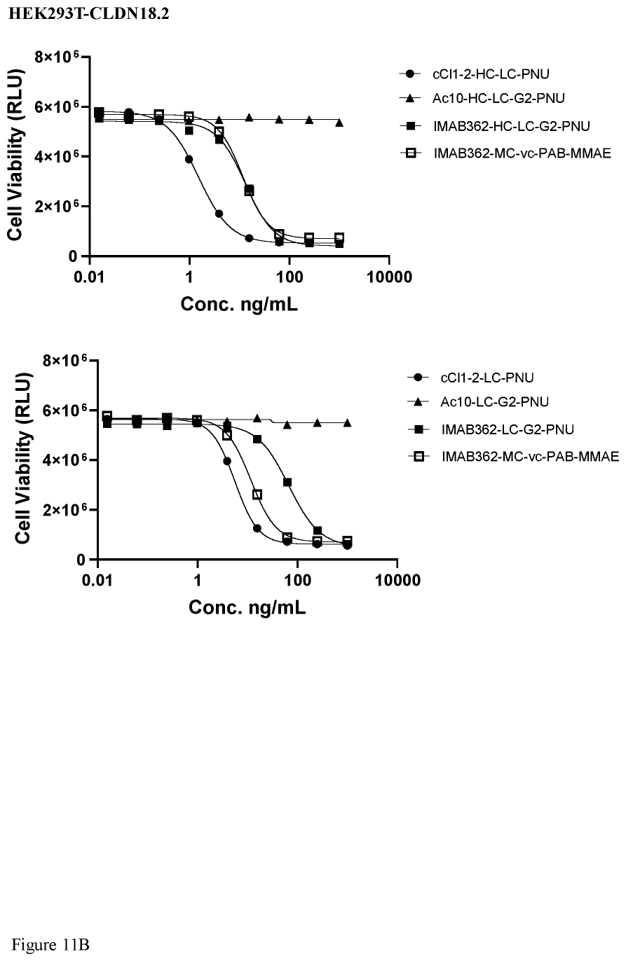

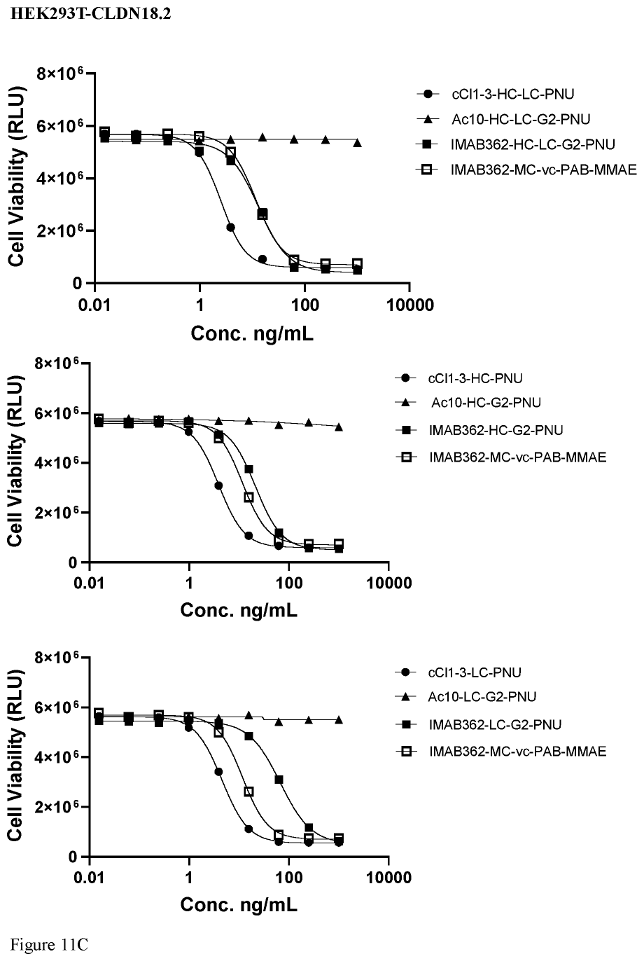

In brief, 1000 cells/well of A549 cells or HEK293T cells, 5000 cells/well of BxPC-3 cells or 10000 cells/well of PA-TU-8988S-high cells were platted in white clear bottom 96-well plates (Greiner) (excluding edge wells, which contained water) in 75 μl DMEM high glucose, 10% FCS, 100 IU/ml Pen/Step/Fungizone, 2 mM L-Glutamine and were grown at 37° C. in a humidified incubator at 7.5% CO₂ atmosphere. After one day of incubation, each ADC was added to respective wells in an amount of 25 μl of 4-fold serial dilution in complete growth medium resulting in concentration of ADCs from 5000 to 0.076 ng/ml for A549 cells, from 1000 to 0.015 ng/ml for HEK293-T cells, from 20000 to 0.25 ng/ml for BxPC-3 cells and from 20000 to 0.31 ng/ml for PA-TU-8988S cells. After 4 additional days, plates were removed from the incubator and equilibrated to room temperature. After approximately 30 min, 50 μl of CellTiter-Glo® 2.0 Luminescent Solution (Promega) was added to each well. After shaking the plates at 450 rpm for 5 min followed by 10 min incubation without shaking, luminescence was measured on a Tecan Spark 10M plate reader with an integration time of 250 ms per well. Curves of luminescence versus ADC concentration (ng/ml) were fitted with the Graphpad Prism Software (see FIGS. 11 to 19).

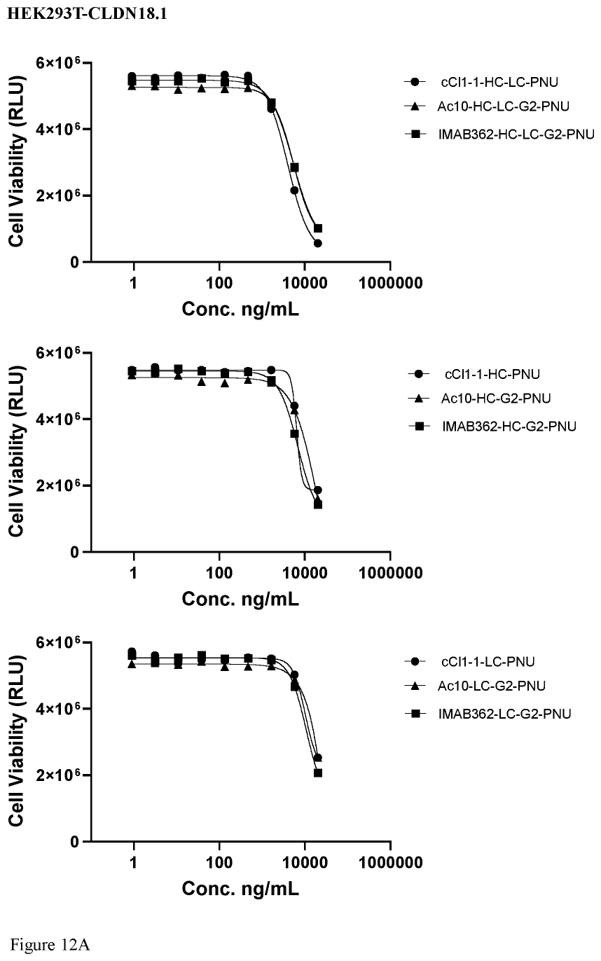

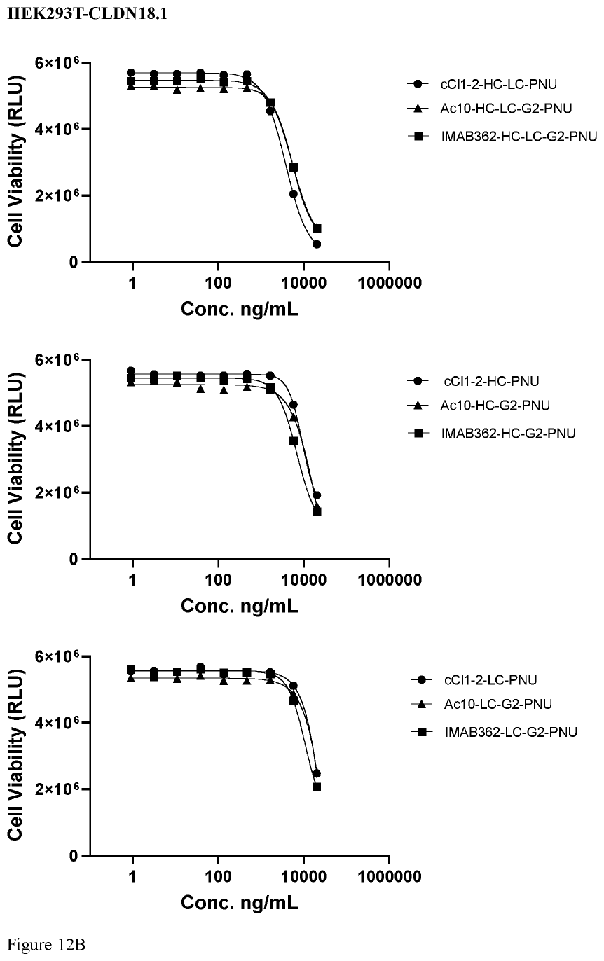

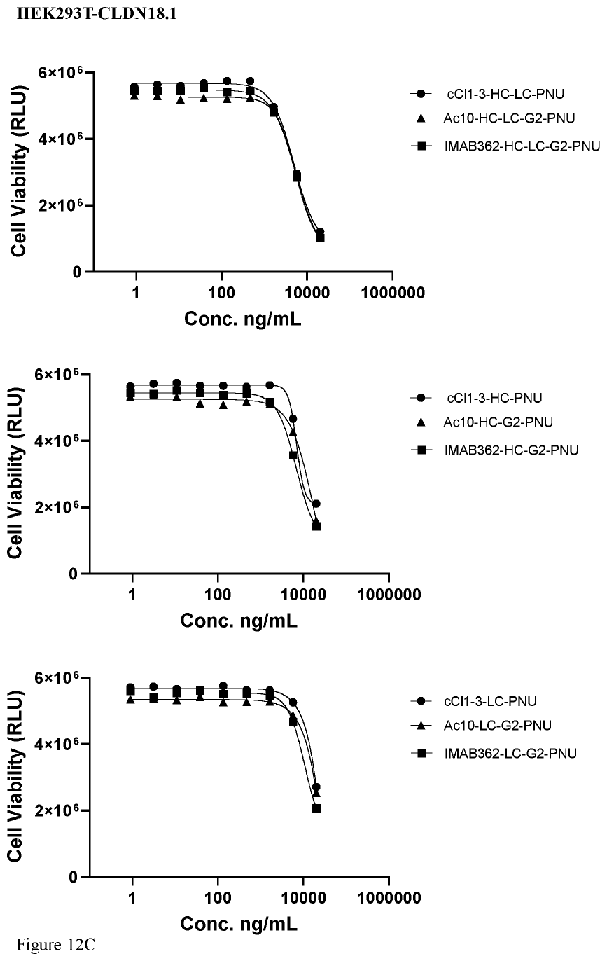

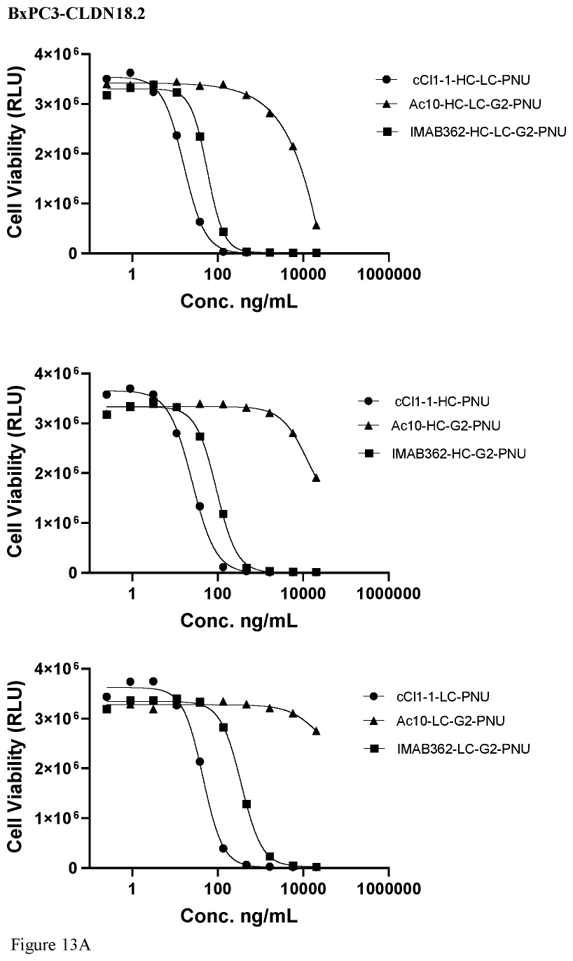

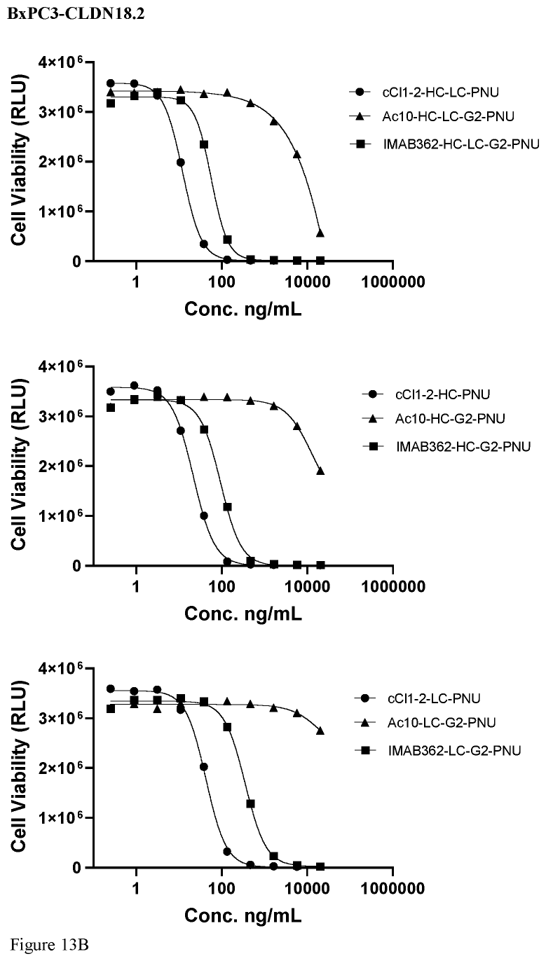

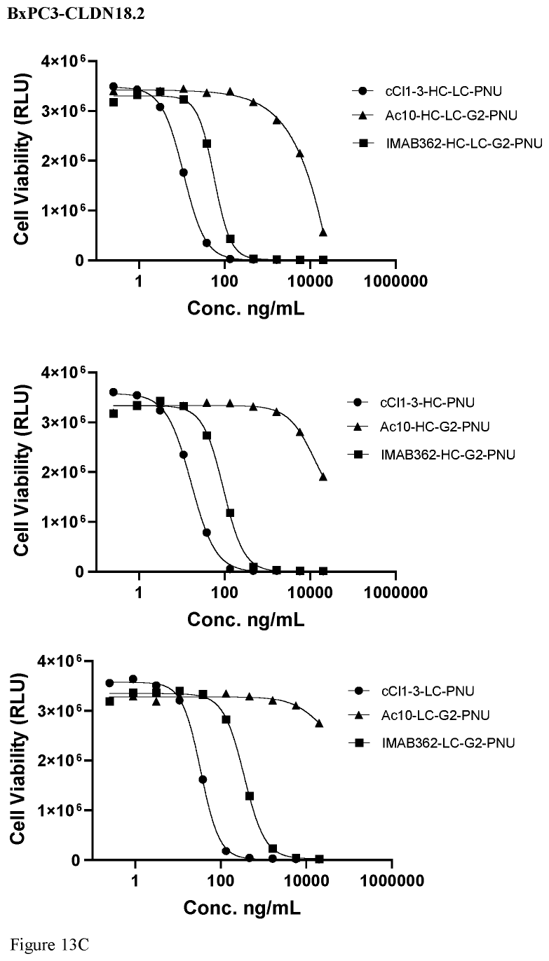

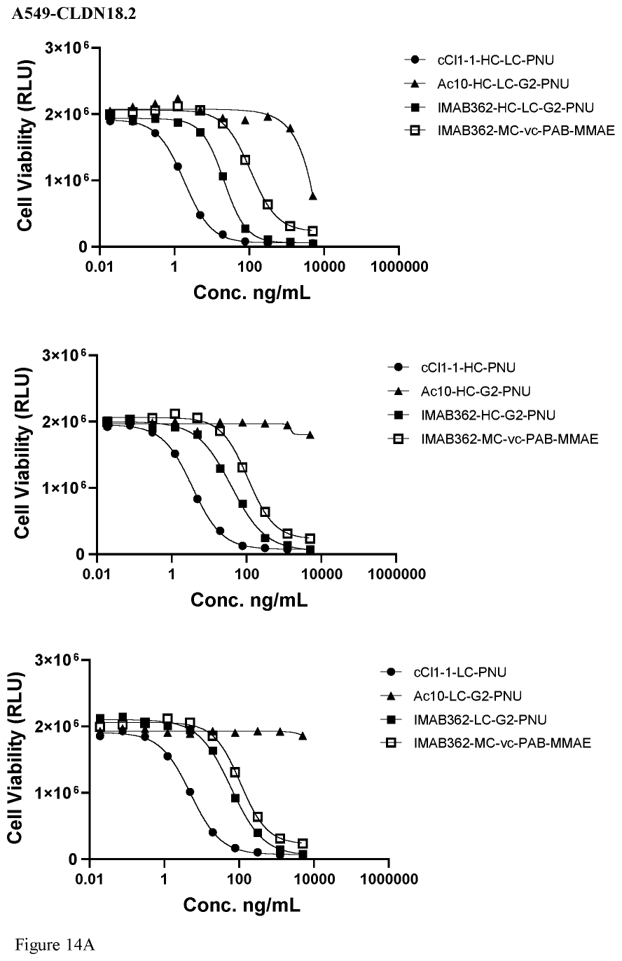

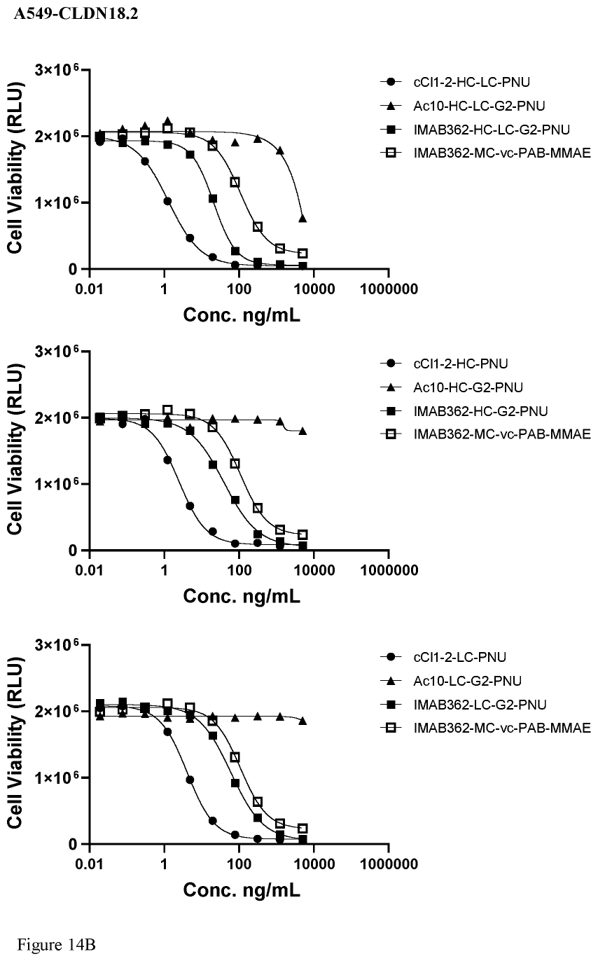

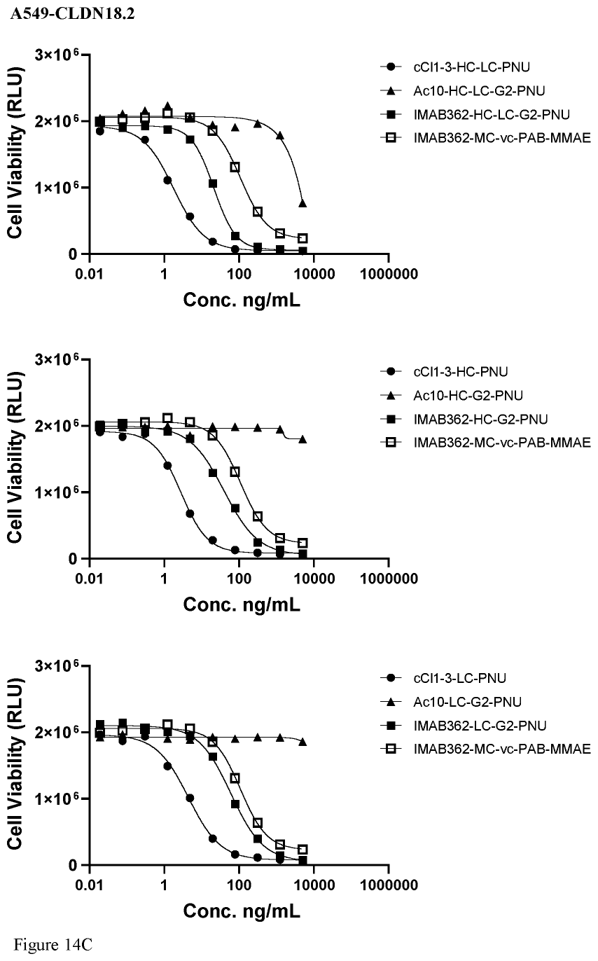

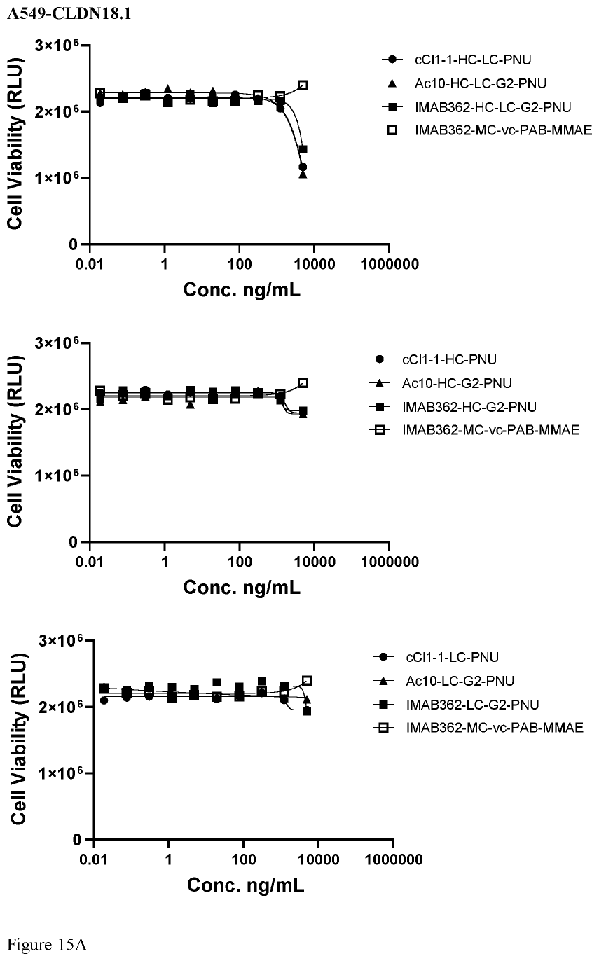

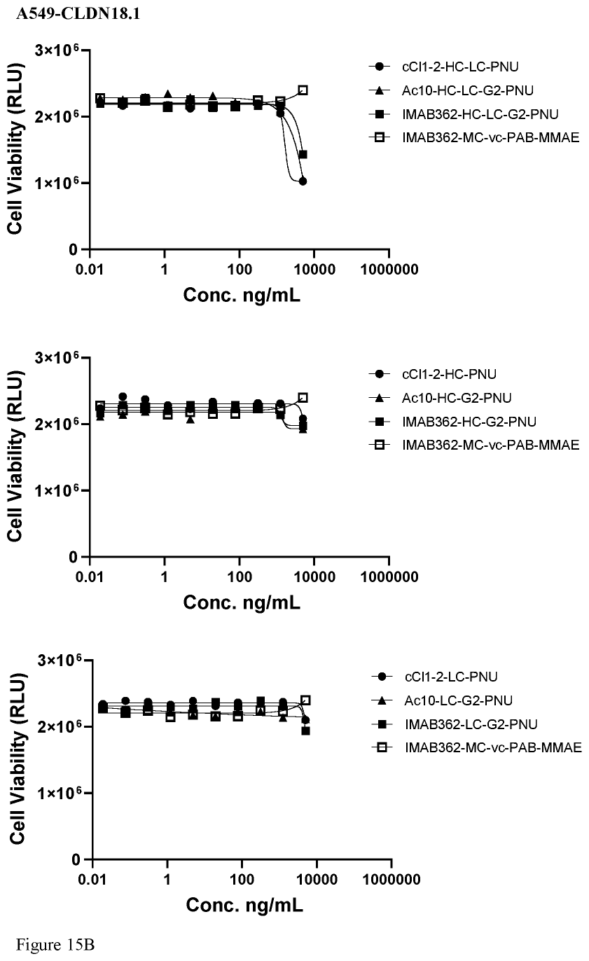

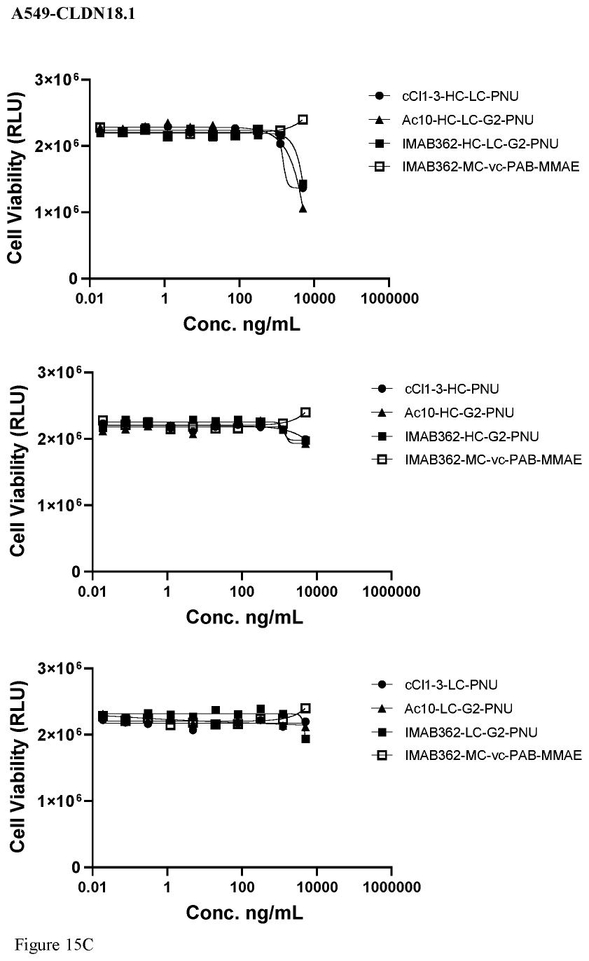

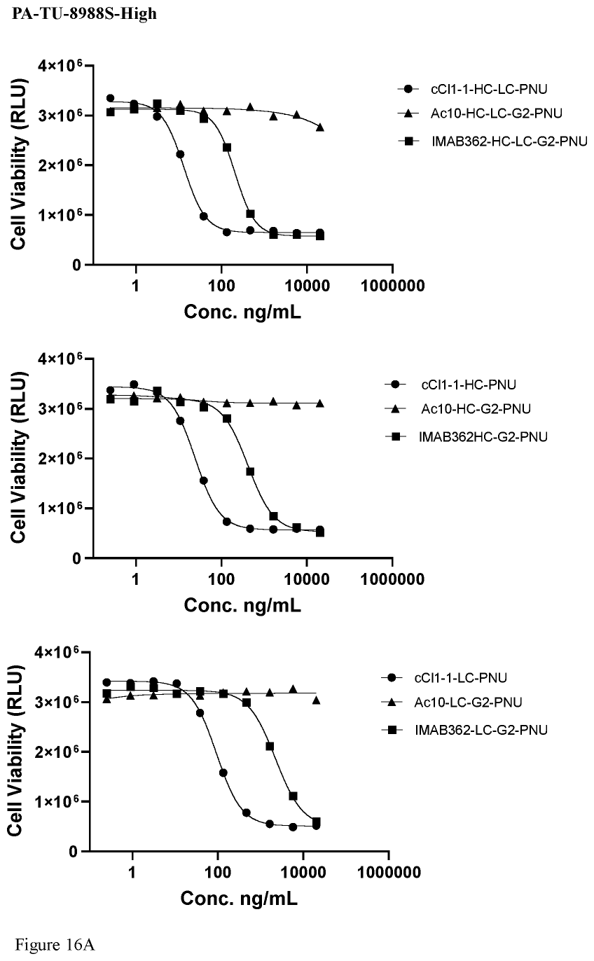

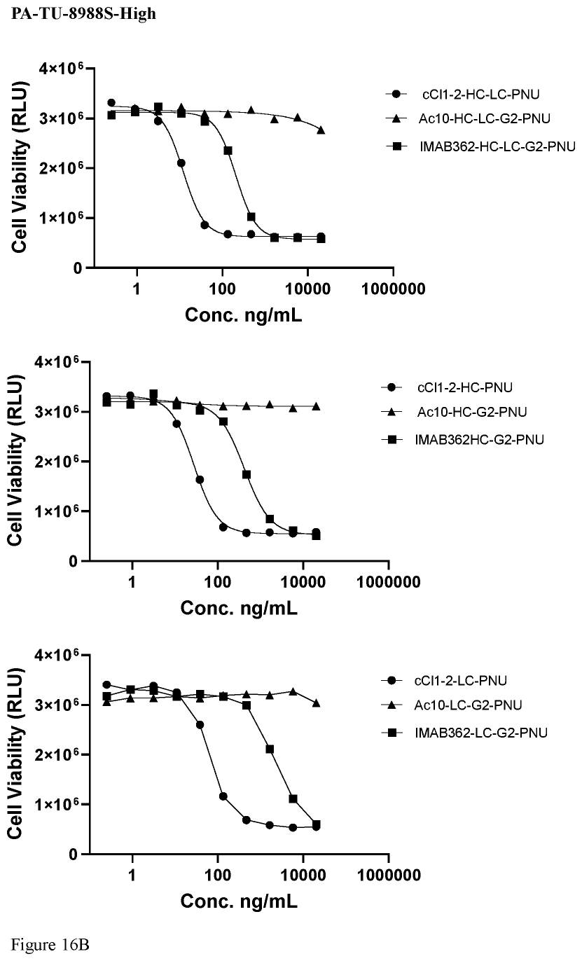

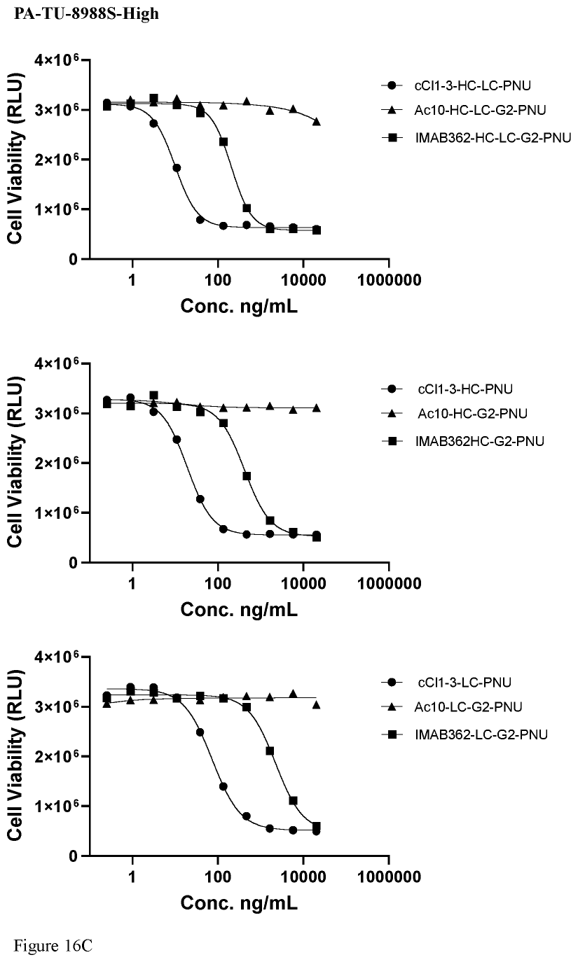

The in-vitro cytotoxicity assays show that cCl1-1, cCl1-2 and cCl1-3, either conjugated at the HC only, at the LC only or at both HC and LC showed a better cytotoxic activity than IMAB362 comparably conjugated and IMAB362-MC-vc-PAB-MMAE on HEK293T cells overexpressing CLDN18.2 (see FIG. 11), on BxPC-3 cells overexpressing CLDN18.2 (see FIG. 13), on A549 cells overexpressing CLDN18.2 (see FIG. 14) or on PA-TU-8988S-High cells (see FIG. 16), while the cytotoxic activity was only observed on very high concentrations of ADCs in HEK293T cells overexpressing CLDN18.1 (see FIG. 12) or A549 cells overexpressing CLDN18.1 (see FIG. 15). Any cytotoxic activity on cells overexpressing CLDN18.1 was attributed to the at least 1000×higher concentration of toxins and was only observed for a DAR4 conjugation (toxins conjugated at the antibody heavy ad light chains). Likewise, the control ADC based on the Ac10 antibody not targeting CLDN18.2 had only cytotoxic activity at very high concentration of ADC (see FIG. 14, 15).

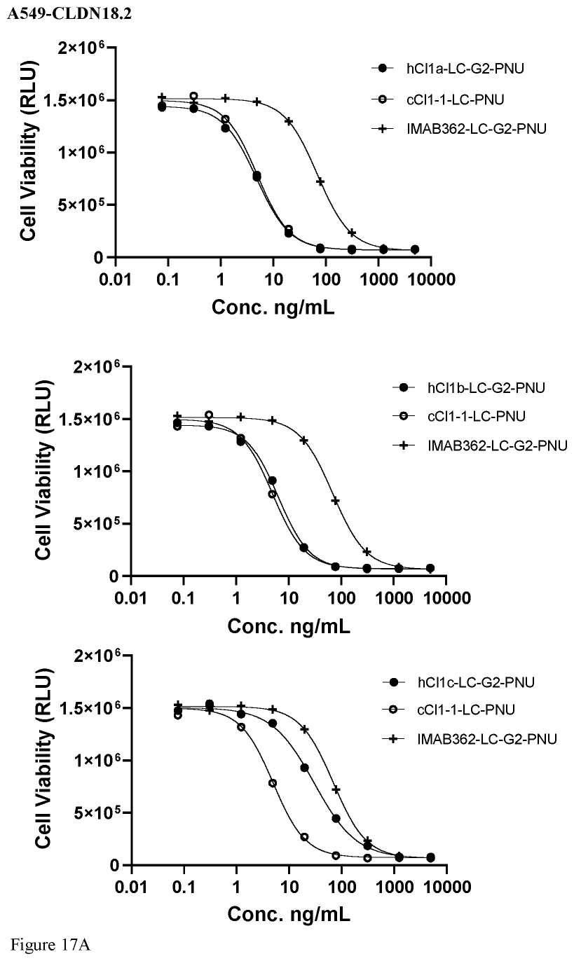

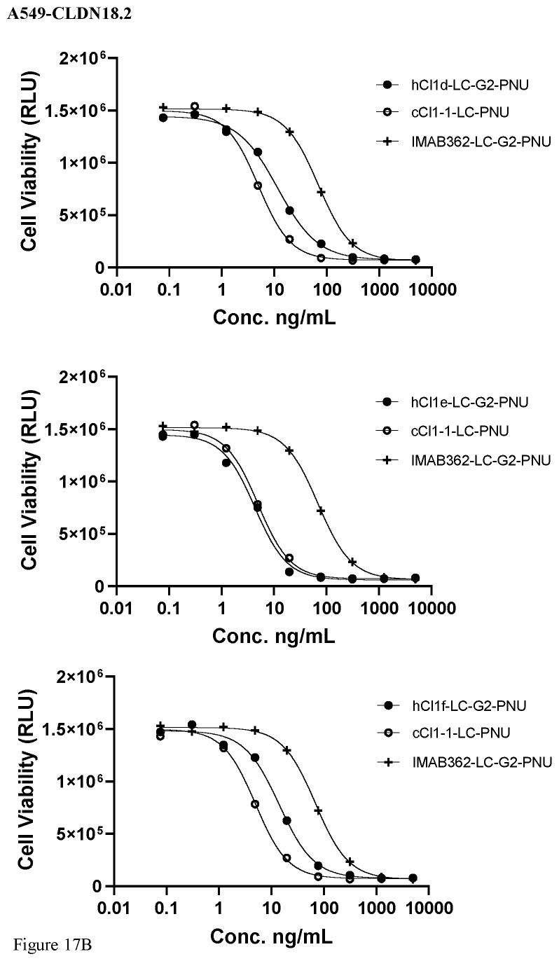

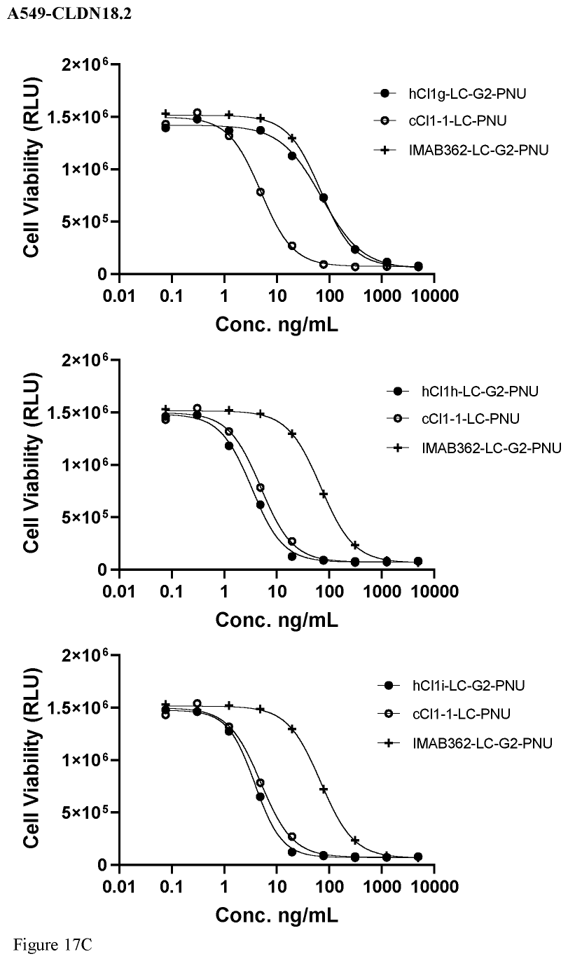

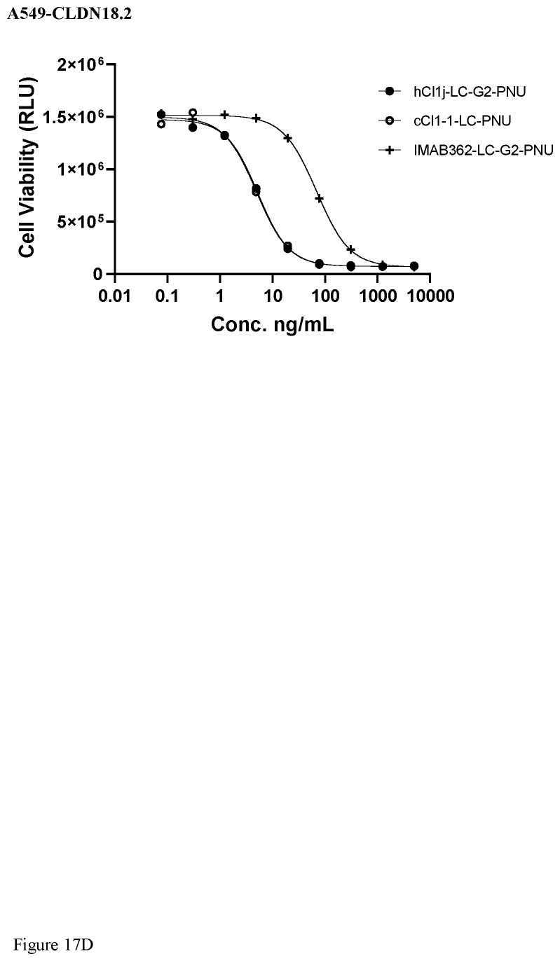

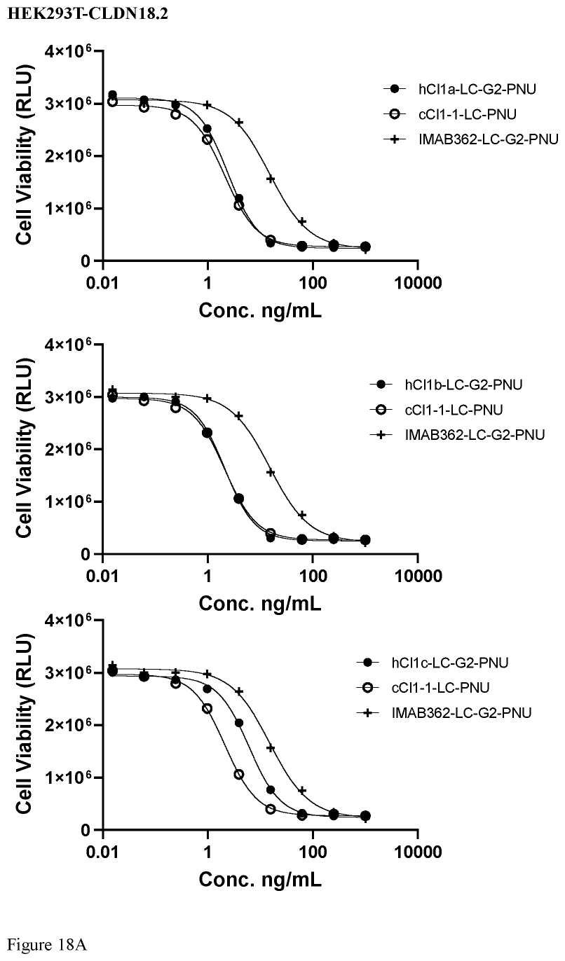

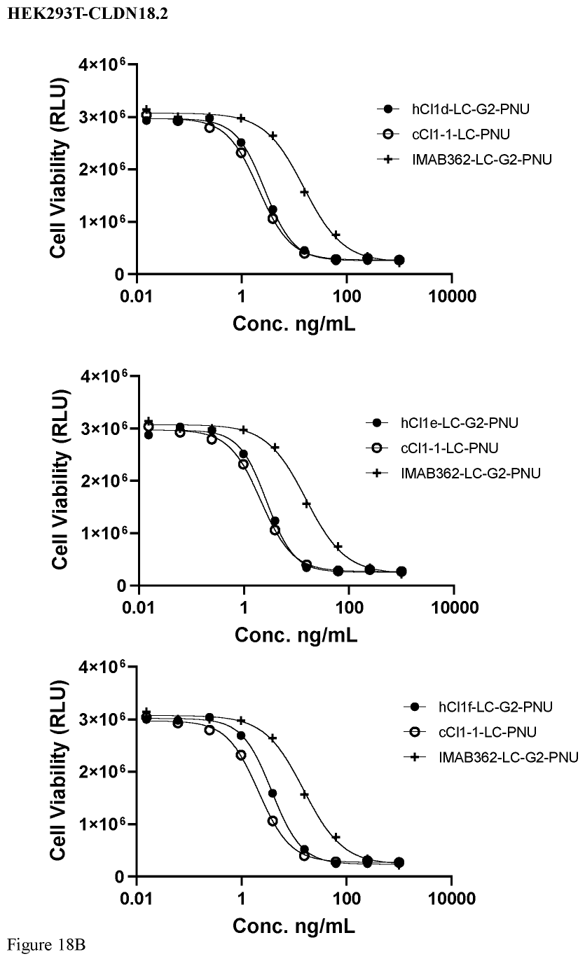

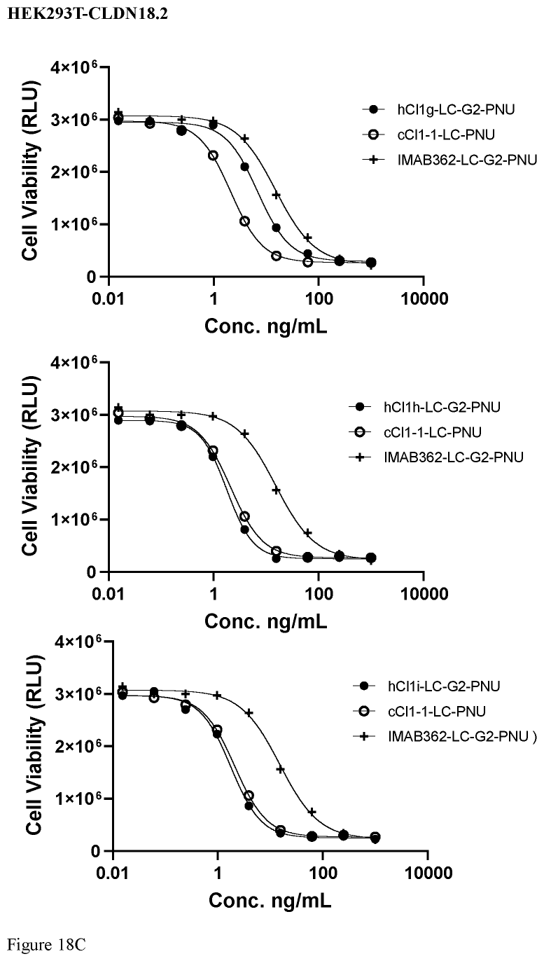

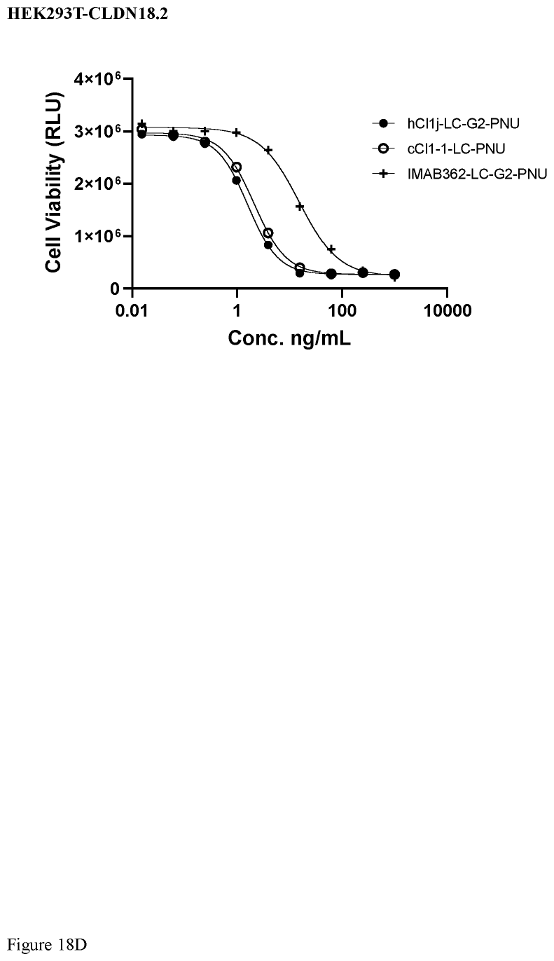

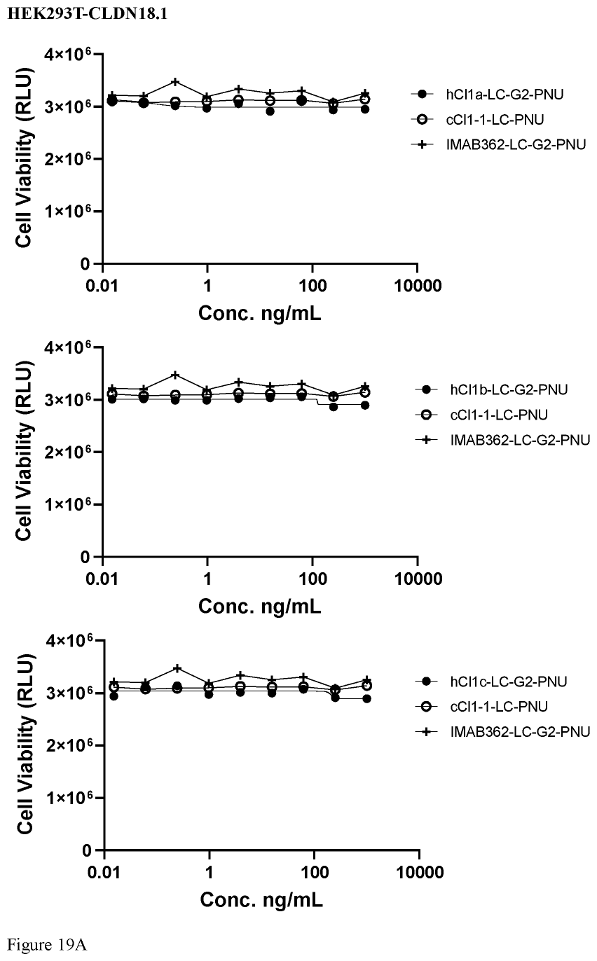

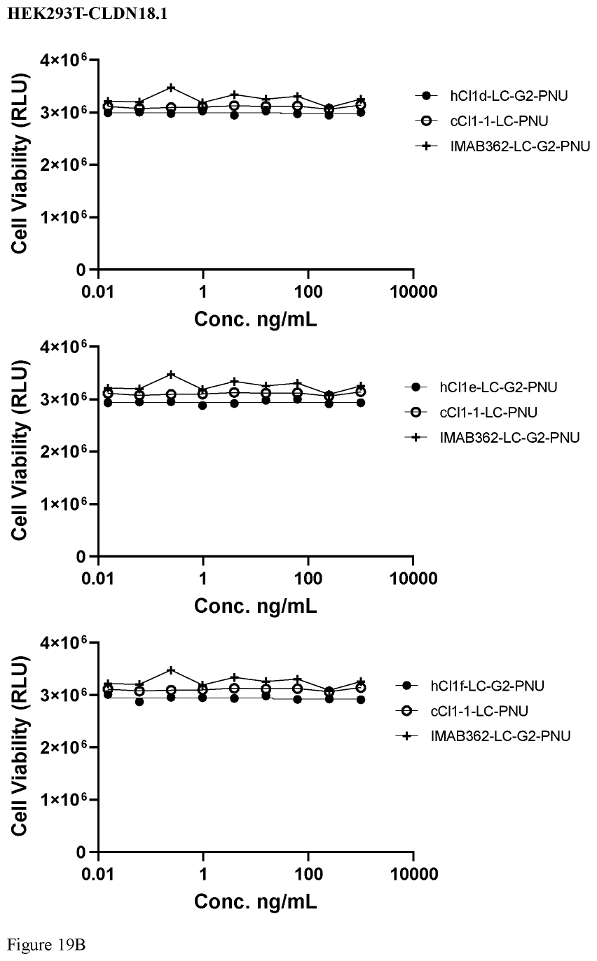

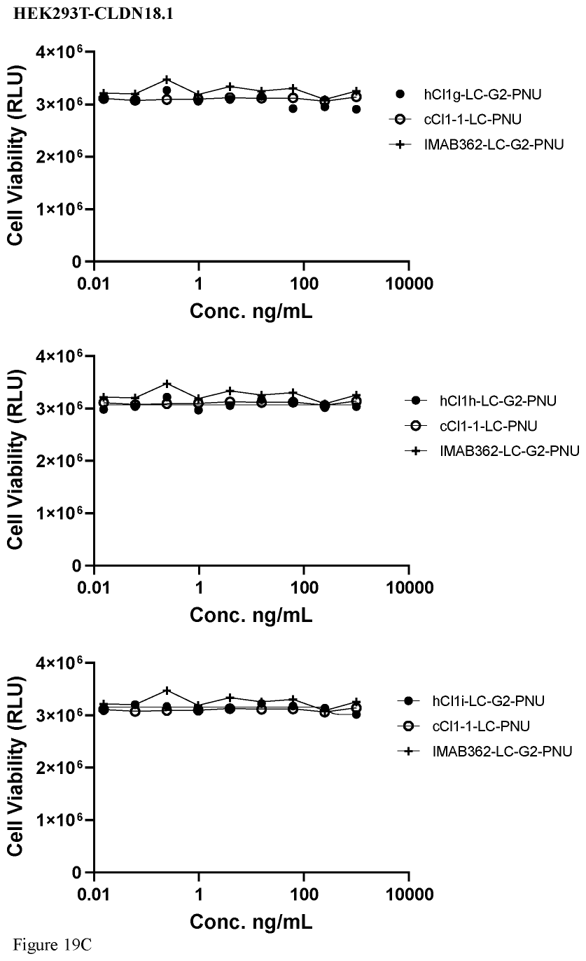

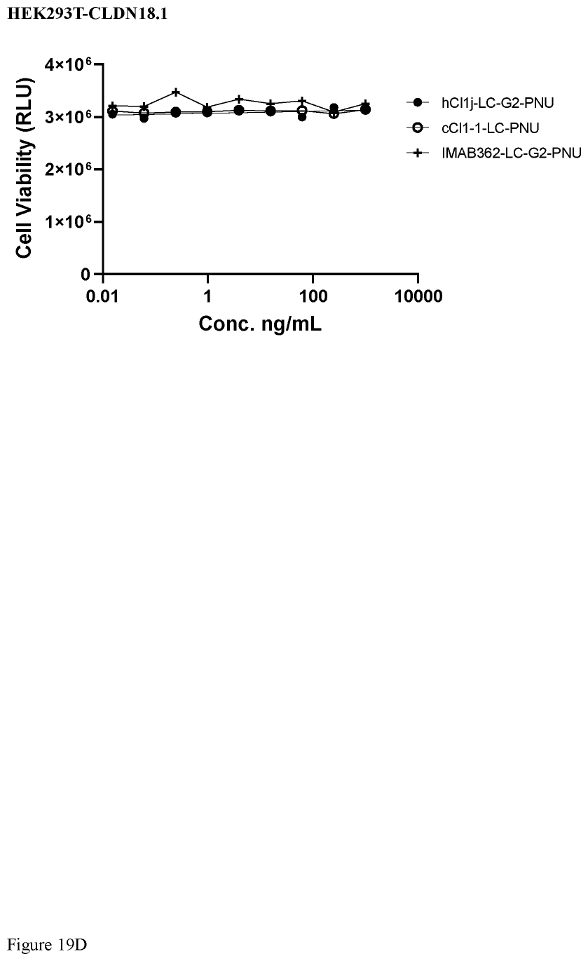

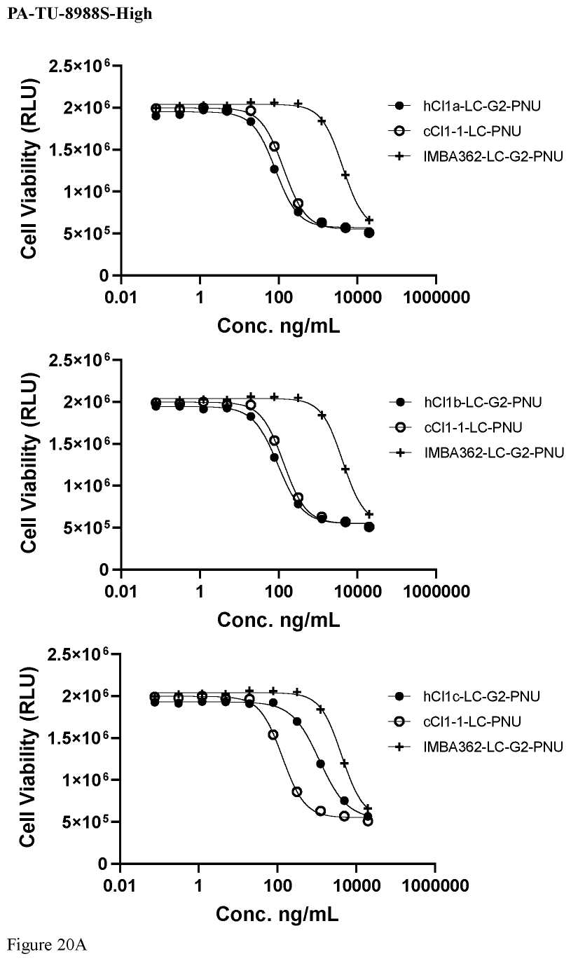

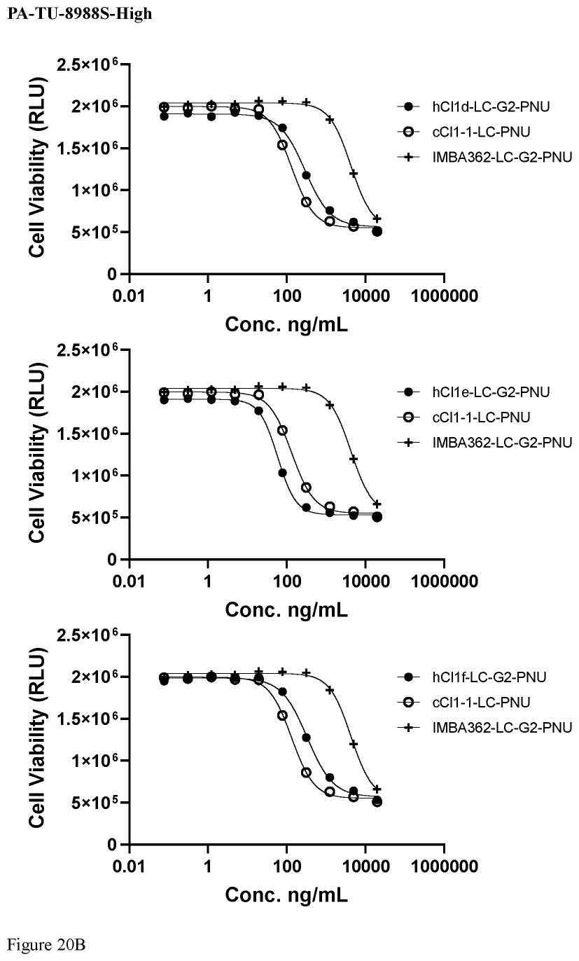

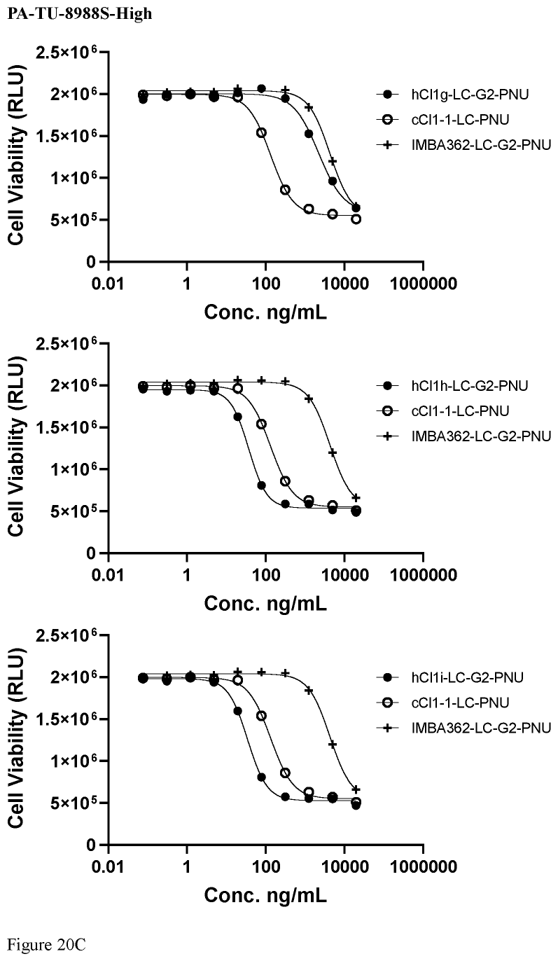

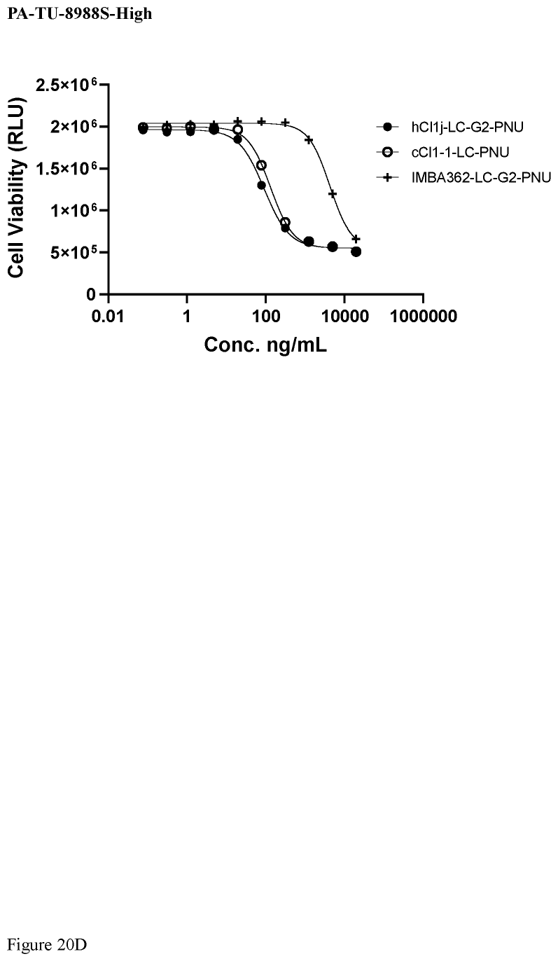

The in-vitro cytotoxicity assays also show that ADC based on the antibodies hCl1a to hCl1j, with the toxin conjugated to the LC only (resulting in a DAR 2), had a superior cytotoxic activity on A549 cells overexpressing CLDN18.2 (see FIG. 17), HEK293T cells overexpressing CLDN18.2 (see FIG. 18) or PA-TU-8988S cells (see FIG. 20) than the ADC based on IMAB362 likewise with the toxin conjugated at the LC. The cytotoxic activity of the ADC based on the antibodies hCl1a to hCl1j was selective for cell overexpressing CLDN18.2, they had no cytotoxic activity on HEK293T cells overexpressing CLDN18.1 (see FIG. 19). EC₅₀ values for humanized antibodies conjugated at their LC, determined using built-in “log(inhibitor) vs. response—variable slope (four parameters)” EC₅₀ determination function of the Prism Software, are reported in Table 9.

TABLE 9
EC50 (ng/ml) values for tested ADCs
based on in-vitro cytotoxicity assays

| | Cell line | | | | |
|---|---|---|---|---|---|
| | A549- | | HEK293T- | PATU8988S- | |
| ADC | CLDN18.2 | | CLDN18.2 | high | |
| hCL1a-LC-G2-PNU | 4.8 | 4.4 | 2.4 | 56.5 | 83.0 |
| hCL1b-LC-G2-PNU | 6.4 | 4.4 | 2.1 | 64.3 | 96.8 |
| hCL1c-LC-G2-PNU | 30.0 | 30.6 | 6.1 | 727 | 1165 |
| hCL1d-LC-G2-PNU | 11.6 | 11.3 | 2.7 | 234 | 293 |
| hCL1e-LC-G2-PNU | 4.3 | 4.0 | 2.7 | 42.7 | 59.1 |
| hCL1f-LC-G2-PNU | 14.0 | 11.1 | 3.8 | 234.2 | 331.1 |
| hCL1g-LC-G2-PNU | 70.7 | 57.3 | 6.9 | 1753 | 2213 |
| hCL1h-LC-G2-PNU | 3.3 | 2.9 | 1.8 | 37.9 | 37.2 |
| hCL1i-LC-G2-PNU | 3.8 | 2.4 | 1.8 | 35.1 | 34.8 |
| hCL1j-LC-G2-PNU | 5.3 | 4.0 | 1.6 | 64.2 | 91.8 |
| IMAB362-LC-G2-PNU | 70.25 | 61.41 | 15.23 | 3071 | 4264 |

Overall, all the of the invention have a high in vitro cytotoxic potential, with a higher cytotoxic activity than IMAB362-LC-G2-PNU.

### Example 9: Analysis of In-Vivo Efficacy of ADC hCl1a-LC-G2-PNU and hCl1f-LC-G2-PNU in Patient-Derived Tumor Xenograft Models

The following studies were performed at Charles River GmbH (Freiburg, Germany).

TABLE 10
Patient-derived tumor xenograft models used for evaluation
of anti-CLDN18.2 ADC hCl1a-LC-G2-PNU and hCl1f-LC-G2-PNU

| Model | Mouse Strain and Sex | Tumor Establishment |
|---|---|---|
| GXA 3037 (Gastric | Female NMR1 nude | Tumor implantation |
| adenocarcinoma) | mice, implantation | unilateral subcutaneous. |
| CXF 742 (Colon | at 5-7 weeks of age | Tumor volume at |
| adenocarcinoma) | | randomization |
| PAXF 2175 | | 50 to 250 mm³ |
| (Pancreatic ductal | | (preferably 80-200 |
| carcinoma) | | mm³) |
| LIXFC 2050 (Lung | | |
| adenocarcinoma) | | |

The anti-CLDN18.2 ADCs hCl1a-LC-G2-PNU, hCl1a(LALA)-LC-G2 and hCl1f-LC-G2-PNU were investigated in the patient-derived tumor xenograph (PDX) models according to the following study protocol:

TABLE 11
Protocols used for evaluation of anti-CLDN18.2
hCl1a-LC-G2-PNU, hCl1a(LALA)-LC-G2 and
hCl1f-LC-G2-PNU ADCs in PDX models.

| PDX | | No. of | Total Daily | | Dosing | |
|---|---|---|---|---|---|---|
| model | Group | Mice | Dose | | days | Route |
| GXA | Control | 3 | 5 ml/kg of | | 1, 8, 15 | intravenous |
| 3037 | vehicle | | saline | | | |
| | hCl1a-LC- | 3 | 0.6 | mg/kg | 1, 8, 15 | intravenous |
| | G2-PNU | | | | | |
| | hCl1a-LC- | 3 | 2 | mg/kg | 1, 8, 15 | intravenous |
| | G2-PNU | | | | | |
| | hCl1a(LALA)- | 3 | 0.2 | mg/kg | 1, 8, 15 | intravenous |
| | LC-G2-PNU | | | | | |
| | hCl1f-LC- | 3 | 0.6 | mg/kg | 1, 8, 15 | intravenous |
| | G2-PNU | | | | | |
| | hCl1f-LC- | 3 | 2 | mg/kg | 1, 8, 15 | intravenous |
| | G2-PNU | | | | | |
| | IMAB362- | 3 | 0.2 | mg/kg | 1, 8, 15 | intravenous |
| | LC-G2-PNU | | | | | |
| | IMAB362- | 3 | 0.6 | mg/kg | 1, 8, 15 | intravenous |
| | LC-G2-PNU | | | | | |
| | IMAB362- | 3 | 2 | mg/kg | 1, 8, 15 | intravenous |
| | LC-G2-PNU | | | | | |
| CXF | Control | 3 | 5 ml/kg of | | 1, 8, 15 | intravenous |
| 742 | vehicle | | saline | | | |
| | hCl1a-LC- | 3 | 2 | mg/kg | 1, 8, 15 | intravenous |
| | G2-PNU | | | | | |
| | AC10-LC-G2- | 3 | 2 | mg/kg | 1, 8, 15 | intravenous |
| | PNU (isotype | | | | | |
| | control) | | | | | |
| PAXF | Control | 3 | 5 ml/kg of | | 1, 8, 15 | intravenous |
| 2175 | vehicle | | saline | | | |
| | hCl1a-LC- | 3 | 0.2 | mg/kg | 1, 8, 15 | intravenous |
| | G2-PNU | | | | | |
| | hCl1a-LC- | 3 | 0.6 | mg/kg | 1, 8, 15 | intravenous |
| | G2-PNU | | | | | |
| | hCl1a(LALA)- | 3 | 0.2 | mg/kg | 1, 8, 15 | intravenous |
| | LC-G2-PNU | | | | | |
| | IMAB362- | 3 | 0.2 | mg/kg | 1, 8, 15 | intravenous |
| | LC-G2-PNU | | | | | |
| LIXFC | Control | 3 | 5 ml/kg of | | 1, 8, 15 | intravenous |
| 2050 | vehicle | | saline | | | |
| | hCl1a-LC- | 3 | 2 | mg/kg | 1, 8, 15 | intravenous |
| | G2-PNU | | | | | |
| | AC10-LC-G2- | 3 | 2 | mg/kg | 1, 8, 15 | intravenous |
| | PNU (isotype | | | | | |
| | control) | | | | | |

Mice were subcutaneously implanted unilaterally with PDX material. Mice allocated into groups when tumors reached randomization criteria and were treated with ADCs as indicated in Table 11 or vehicle for a total of 3 times. Tumor volumes were determined by caliper 5 measurements and body weight was recorded twice weekly. Mice were euthanized on reaching a tumor burden of 2000 mm³, or on significant body weight loss (overall more than 30%, or more than 20% in two days).

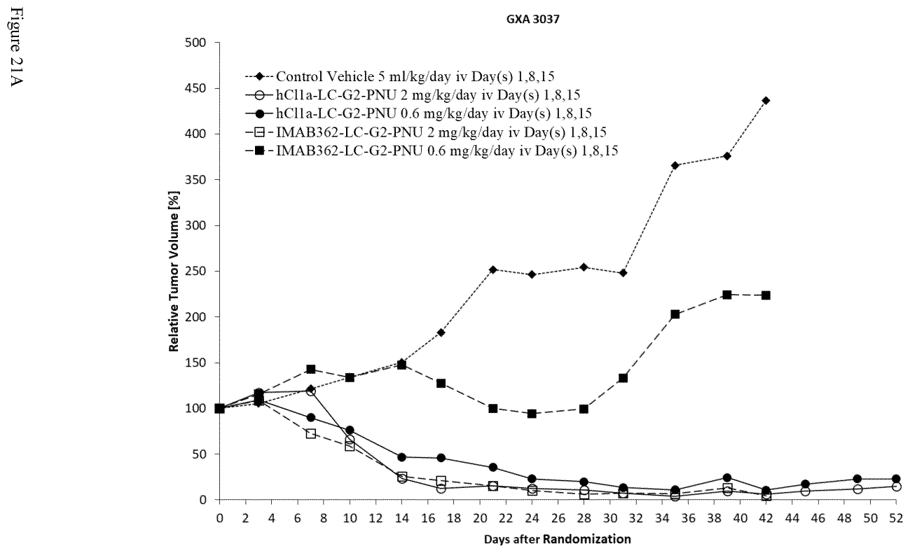

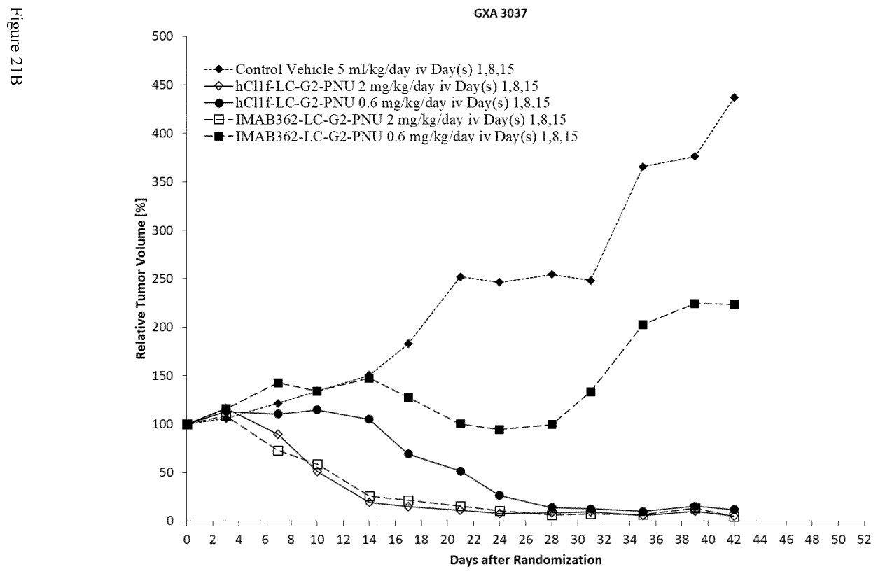

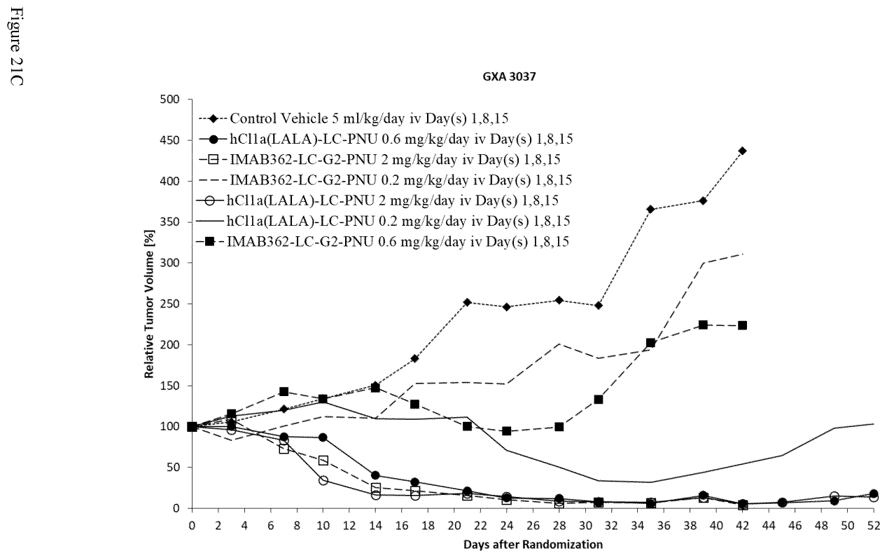

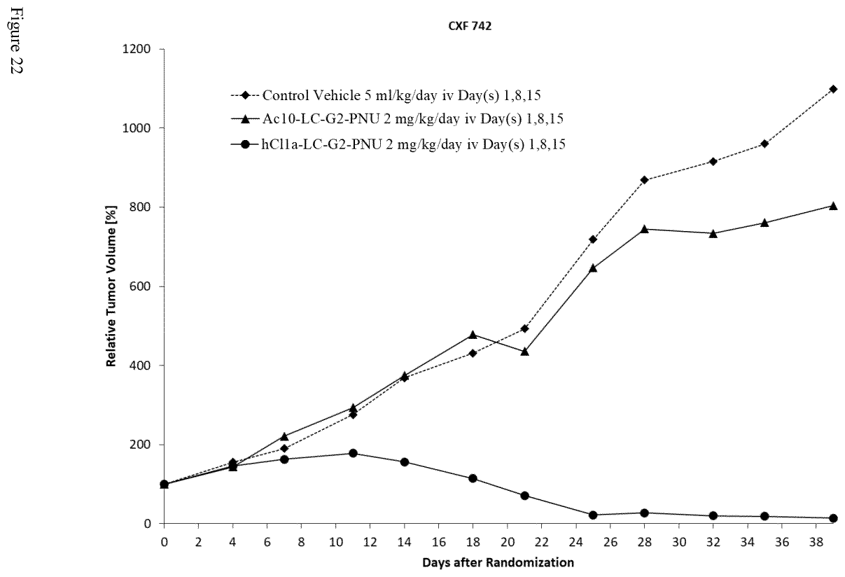

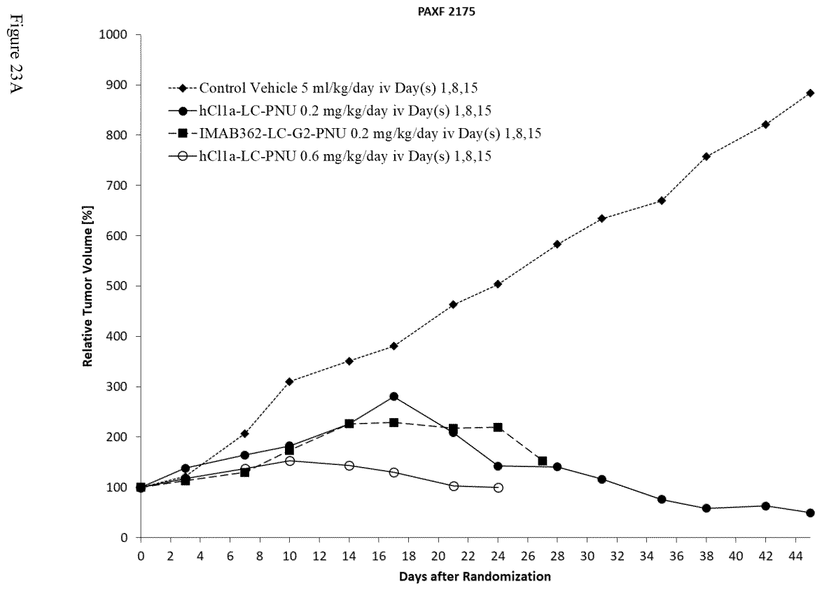

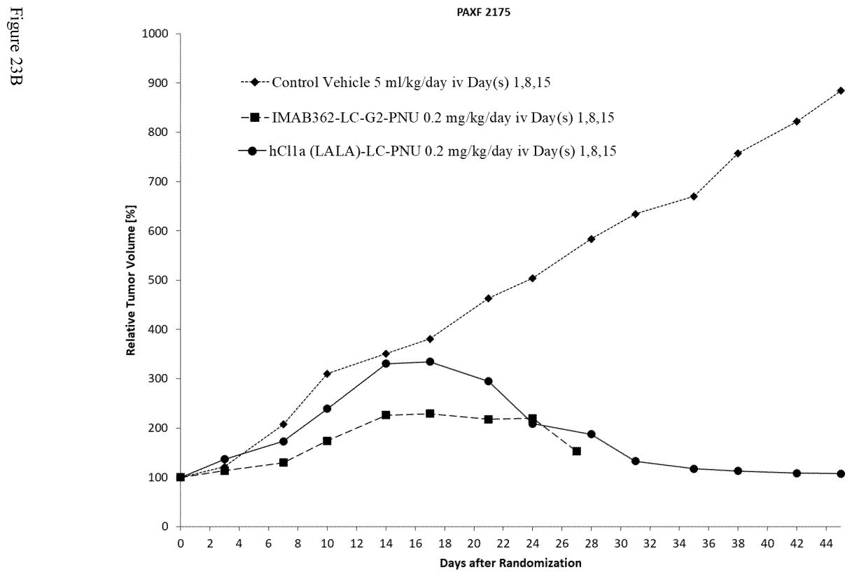

FIGS. 20 to 23 show the relative tumor volume evolution over the studies in the different PDX models. Tumor xenografts established with patient-derived tumor material having CLDN18.2 expression responded significantly to treatment with the ADCs of the invention. The response (delayed tumor growth or tumor shrinkage) with the ADCs of the invention when administered at lower doses (0.2 mg/kg or 0.6 mg/kg) was better than the similar ADC based on the anti-CLDN18.2 antibody IMAB362 administered at the same doses and comparably good when administered at the higher dose of 2 mg/kg.

### EMBODIMENTS

1. An antibody-drug conjugate having the general formula A-(L-T)ₙ, wherein
a. A is an antibody or fragment thereof binding to CLDN18.2 comprising the HCDR1, HCDR2 and HCDR3 sequences of SEQ ID NO: 21, SEQ ID NO: 22 and SEQ ID NO: 23 respectively, and the LCDR1, LCDR2 and LCDR3 sequences of SEQ ID NO: 24, SEQ ID NO: 25 and SEQ ID NO: 26 respectively,
b. L is a linker, and
c. Tisa toxin,
   wherein the toxin is an anthracycline,
   wherein n is an integer between ≥1 and ≤10;
   or a pharmaceutically acceptable salt or ester thereof.

2. The antibody-drug conjugate of embodiment 1, wherein the linker L comprises at least one a non-cleavable linker element.

3. The antibody-drug conjugate of embodiment 2, wherein the non-cleavable linker element is selected from the group consisting of
i. ethylenediamine (EDA),
j. N-formyl-N,N′-dimethylethylenediamine,
k. diethylamine (DEA),
l. a piperazine-derived compound of the following formula:

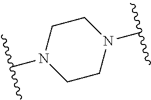 [SMILES: *N1CCN(*)CC1]

wherein the wavy lines indicate attachments to the toxin and another linker element,
m. the compound of the following formula:

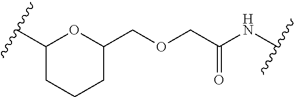 [SMILES: *NC(=O)COCC1CCCC(*)O1]

wherein the wavy lines indicate attachments to the toxin and another linker element,
n. the compound of the following formula:

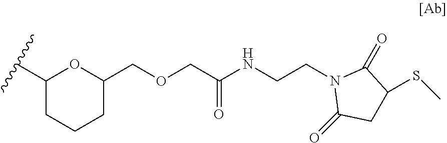 [SMILES: *C1CCCC(COCC(=O)NCCN2C(=O)CC(SC)C2=O)O1]

wherein the wavy lines indicate attachments to the toxin and [Ab] indicates the antibody or fragment thereof,
o. a maleimidocaproyl compound of the following formula:

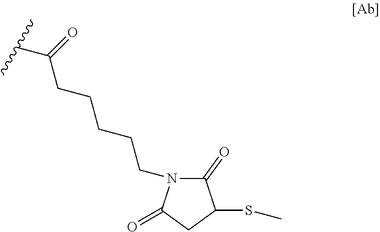 [SMILES: *C(=O)CCCCCN1C(=O)CC(SC)C1=O]

wherein the wavy lines indicate attachments to another linker element and [Ab] indicates the antibody or fragment thereof,
p. the compound of the following formula:

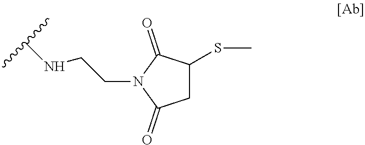 [SMILES: *NCCN1C(=O)CC(SC)C1=O]

wherein the wavy lines indicate attachments to a toxin and [Ab] indicates the antibody or fragment thereof,
and wherein the non-cleavable linker element is conjugated to the toxin by means of an amide bond or an ether bond.

4. The antibody-drug conjugate of embodiment 2 or embodiment 3, wherein the linker further comprises an oligopeptide linker element and/or enzyme-cleavable linker element and/or a spacer element.

5. The antibody-drug conjugate of embodiment 4, wherein one oligopeptide linker element comprises a sortase recognition motif oligopeptide selected from: -LPXTGₘ-, -LPXAGₘ-, -LPXSGₘ-, -LAXTGₘ-, -LPXTGₘ-, -LPXTAₘ-, -NPQTGₘ- or -NPQTNₘ-, with Gₘ being an oligoglycine with m being an integer between ≥1 and ≤21, A m being an oligoalanine with m being an integer between ≥1 and ≤21, Nm being an oligoasparagine with m being an integer between ≥1 and ≤21 and X being any conceivable amino acid, preferably the sortase recognition motif oligopeptide being -LPQTGG- or -LPETGG-.

6. The antibody-drug conjugate of embodiment 5, wherein the oligopeptide linker element comprises:
a. the sequence SEQ ID NO: 131, or
b. the sequence SEQ ID NO: 132.

7. The antibody-drug conjugate of any of embodiments 4 to 6, wherein the enzyme-cleavable linker element comprises a val-cit-PAB linker according to the compound of the following formula:

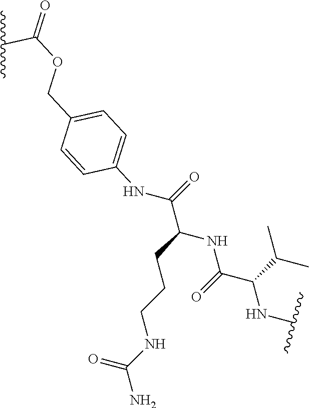 [SMILES: *N[C@H](C(=O)N[C@@H](CCCNC(N)=O)C(=O)Nc1ccc(COC(*)=O)cc1)C(C)C]

wherein the wavy lines indicate attachments to other linker elements.

8. The antibody-drug conjugate of any of embodiments 4 to 7, wherein the spacer element comprises a peptidic flexible oligopeptide, preferably wherein the peptidic flexible oligopeptide consists of G and S, more preferably wherein the peptidic flexible oligopeptide is (GGGGS) 0 with o being 1, 2, 3, 4 or 5.

9. The antibody-drug conjugate of any of embodiments 1 to 8, wherein the antibody drug conjugate has the following structure:
a. A-([oligopeptide linker element-non-cleavable linker element]-T)ₙ and preferably wherein the linker is selected from:

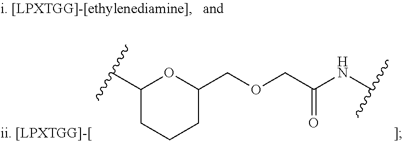 [SMILES: *NC(=O)COCC1CCCC(*)O1]

b. A-([oligopeptide linker element-enzyme cleavable linker element-non-cleavable linker element]-T)ₙ and preferably wherein the linker is selected from:

i.
[LPXTGG]-[vc-PAB]-[N-formyl-N,
N′-dimethylethylenediamine],
and
ii.
[LPXTGG]-[vc-PAB]-[piperazine];

c. A-([spacer element-oligopeptide linker element-non-cleavable linker element]-T)ₙ and preferably wherein the linker is selected from:

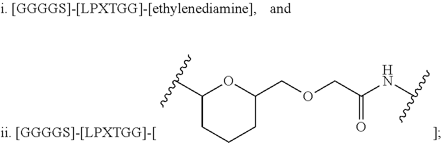 [SMILES: *NC(=O)COCC1CCCC(*)O1]

or
d. A-([spacer element-oligopeptide linker element-enzyme cleavable linker element-non-cleavable element]-T)ₙ and preferably wherein the linker is selected from:

i.
[GGGGS]-[LPXTGG]-[vc-PAB]-[N-formyl-
N,N′-dimethylethylenediamine],
and
ii.
[GGGGS]-[LPXTGG]-[vc-PAB]-[piperazine].

10. The antibody-drug conjugate of embodiment 9, wherein the non-cleavable linker element is ethylenediamine and wherein the oligopeptide linker element is LPXTGG wherein X is Q or E, preferably wherein X is Q.

11. The antibody-drug conjugate of any of embodiments 1 to 10, wherein
a. (L-T) is covalently linked to both light chains of the antibody,
b. (L-T) is covalently linked to both heavy chains of the antibody, or
c. (L-T) is covalently linked to both light chains and both heavy chains of the 5 antibody.

12. The antibody-drug conjugate of any of embodiments 1 to 11, wherein (L-T)
a. is linked to the C-terminus of the antibody light chain or antibody heavy chain, or
b. is linked to an amino acid side chain of the antibody light chain or antibody 10 heavy chain.

13. The antibody-drug conjugate of any of embodiments 1 to 12, wherein the anthracycline derivative has the following formula (I), and is covalently linked to the non-cleavable linker element by the C₁₃ resulting in the loss of the C₁₄ and the hydroxyl group, or is covalently linked to the non-cleavable linker element by the hydroxyl group on C₁₄:

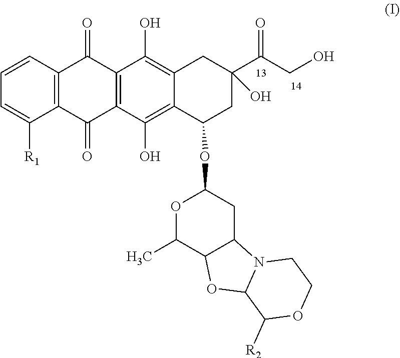

and wherein R₁ is a hydrogen atom, a hydroxy or methoxy group,
and wherein R 2 is a C₁-C₅ alkoxy group.

14. The antibody-drug conjugate of any of embodiments 1 to 13, wherein the anthracycline derivative is a derivative of 3′-deamino-3″,4′-anhydro-[2″(S)-methoxy-3″(R)-oxy-4″-20 morpholinyl]doxorubicin (PNU-159682).

15. The antibody-drug conjugate of any of embodiments 1 to 14, wherein A, the antibody or fragment thereof, comprises:
a. the HCDR1, HCDR2 and HCDR3 sequences of SEQ ID NO: 1, SEQ ID NO: 15 and SEQ ID NO: 3, respectively, and the LCDR1, LCDR2 and LCDR3 sequences of SEQ ID NO: 4, SEQ ID NO: 5 and SEQ ID NO: 6, respectively;
b. the HCDR1, HCDR2 and HCDR3 sequences of SEQ ID NO: 1, SEQ ID NO: 16 and SEQ ID NO: 3, respectively, and the LCDR1, LCDR2 and LCDR3 sequences of SEQ ID NO: 4, SEQ ID NO: 5 and SEQ ID NO: 6, respectively;
c. the HCDR1, HCDR2 and HCDR3 sequences of SEQ ID NO: 1, SEQ ID NO: 16 and SEQ ID NO: 3, respectively, and the LCDR1, LCDR2 and LCDR3 sequences of SEQ ID NO: 17, SEQ ID NO: 14 and SEQ ID NO: 11, respectively;
d. the HCDR1, HCDR2 and HCDR3 sequences of SEQ ID NO: 1, SEQ ID NO: 16 and SEQ ID NO: 3, respectively, and the LCDR1, LCDR2 and LCDR3 sequences of SEQ ID NO: 18, SEQ ID NO: 19 and SEQ ID NO: 11, respectively;
e. the HCDR1, HCDR2 and HCDR3 sequences of SEQ ID NO: 12, SEQ ID NO: 15 and SEQ ID NO: 3, respectively, and the LCDR1, LCDR2 and LCDR3 sequences of SEQ ID NO: 4, SEQ ID NO: 5 and SEQ ID NO: 6, respectively;
f. the HCDR1, HCDR2 and HCDR3 sequences of SEQ ID NO: 1, SEQ ID NO: 20 and SEQ ID NO: 3, respectively, and the LCDR1, LCDR2 and LCDR3 sequences of SEQ ID NO: 4, SEQ ID NO: 5 and SEQ ID NO: 6, respectively;
g. the HCDR1, HCDR2 and HCDR3 sequences of SEQ ID NO: 1, SEQ ID NO: 20 and SEQ ID NO: 3, respectively, and the LCDR1, LCDR2 and LCDR3 sequences of SEQ ID NO: 18, SEQ ID NO: 19 and SEQ ID NO: 11, respectively;
h. the HCDR1, HCDR2 and HCDR3 sequences of SEQ ID NO: 12, SEQ ID NO: 20 and SEQ ID NO: 8, respectively, and the LCDR1, LCDR2 and LCDR3 sequences of SEQ ID NO: 4, SEQ ID NO: 5 and SEQ ID NO: 6, respectively; or
i. the HCDR1, HCDR2 and HCDR3 sequences of SEQ ID NO: 12, SEQ ID NO: 20 and SEQ ID NO: 8, respectively, and the LCDR1, LCDR2 and LCDR3 sequences of SEQ ID NO: 17, SEQ ID NO: 14 and SEQ ID NO: 11, respectively.

16. The antibody-drug conjugate of any of embodiments 1 to 14, wherein A, the antibody or fragment thereof comprises:
a. the HCDR1, HCDR2 and HCDR3 sequences of SEQ ID NO: 1, SEQ ID NO: 2 and SEQ ID NO: 3, respectively, and the LCDR1, LCDR2 and LCDR3 sequences of SEQ ID NO: 4, SEQ ID NO: 5 and SEQ ID NO: 6, respectively;
b. the HCDR1, HCDR2 and HCDR3 sequences of SEQ ID NO: 1, SEQ ID NO: 7 and SEQ ID NO: 8, respectively, and the LCDR1, LCDR2 and LCDR3 sequences of SEQ ID NO: 9, SEQ ID NO: 10 and SEQ ID NO: 11, respectively; or
c. the HCDR1, HCDR2 and HCDR3 sequences of SEQ ID NO: 12, SEQ ID NO: 2 and SEQ ID NO: 3, respectively, and the LCDR1, LCDR2 and LCDR3 sequences of SEQ ID NO: 13, SEQ ID NO: 14 and SEQ ID NO: 11, respectively.

17. The antibody-drug conjugate of any of embodiments 1 to 15, wherein A, the antibody or fragment thereof comprises:
a. a VH sequence of SEQ ID NO: 33 and a VL sequence of SEQ ID NO: 38;
b. a VH sequence of SEQ ID NO: 34 and a VL sequence of SEQ ID NO: 38;
c. a VH sequence of SEQ ID NO: 34 and a VL sequence of SEQ ID NO: 39;
d. a VH sequence of SEQ ID NO: 34 and a VL sequence of SEQ ID NO: 40;
e. a VH sequence of SEQ ID NO: 35 and a VL sequence of SEQ ID NO: 38;
f. a VH sequence of SEQ ID NO: 36 and a VL sequence of SEQ ID NO: 41;
g. a VH sequence of SEQ ID NO: 36 and a VL sequence of SEQ ID NO: 40;
h. a VH sequence of SEQ ID NO: 37 and a VL sequence of SEQ ID NO: 41;
i. a VH sequence of SEQ ID NO: 37 and a VL sequence of SEQ ID NO: 38; or
j. a VH sequence of SEQ ID NO: 37 and a VL sequence of SEQ ID NO: 39.

18. The antibody-drug conjugate of any of embodiments 1 to 14 or 16, wherein A, the antibody or fragment thereof comprises:
a. a VH sequence of SEQ ID NO: 27 and a VL sequence of SEQ ID NO: 28;
b. a VH sequence of SEQ ID NO: 29 and a VL sequence of SEQ ID NO: 30; or
c. a VH sequence of SEQ ID NO: 31 and a VL sequence of SEQ ID NO: 32.

19. The antibody-drug conjugate of any of embodiments 1 to 15 or 17, wherein A, the antibody or fragment thereof, comprises:
a. the heavy chain sequence of SEQ ID NO: 46 and light chain sequence of SEQ ID NO: 51;
b. the heavy chain sequence of SEQ ID NO: 47 and light chain sequence of SEQ ID NO: 51;
c. the heavy chain sequence of SEQ ID NO: 47 and light chain sequence of SEQ ID NO: 52;
d. the heavy chain sequence of SEQ ID NO: 47 and light chain sequence of SEQ ID NO: 53;
e. the heavy chain sequence of SEQ ID NO: 48 and light chain sequence of SEQ ID NO: 51;
f. the heavy chain sequence of SEQ ID NO: 47 and light chain sequence of SEQ ID NO: 54;
g. the heavy chain sequence of SEQ ID NO: 49 and light chain sequence of SEQ ID NO: 53;
h. the heavy chain sequence of SEQ ID NO: 50 and light chain sequence of SEQ ID NO: 54;
i. the heavy chain sequence of SEQ ID NO: 50 and light chain sequence of SEQ ID NO: 51; or
j. the heavy chain sequence of SEQ ID NO: 50 and light chain sequence of, SEQ ID NO: 52
   or versions thereof with an engineered Fc domain.

20. A method of producing an antibody-drug conjugate according to any of embodiments 1 to 19, wherein the method comprises the following steps:
g. providing A, an antibody or fragment thereof with an oligopeptide linker element preferably at its C-terminus, optionally preceded by a spacer element at the antibody light and/or heavy chains,
h. providing one or more toxins T with a non-cleavable linker element, and
i. conjugating the antibody and the toxin resulting in the antibody-drug conjugate.

21. An antibody-drug conjugate consisting of:
the antibody consisting of two heavy chains of the amino acid sequence according to SEQ ID NO: 46, and two light chains of the amino acid sequence according to SEQ ID NO: 51, wherein the antibody binds to CLDN18.2,
the linker [GGGGS]-[LPQTGG]-[ethylenediamine] at the C-terminus of the light chains, and
the anthracycline-based small molecule toxin 3′-deamino-3″,4′-anhydro-[2″(S)-methoxy-3″(R)-oxy-4″-morpholinyl]doxorubicin (PNU-159682), linked covalently to the ethylenediamine of the linker at C₁₃, resulting in the loss of C₁₄ and of the hydroxyl group.

22. An antibody-drug conjugate consisting of:
the antibody consisting of two heavy chains of the amino acid sequence according to SEQ ID NO: 133, and two light chains of the amino acid sequence according to SEQ ID NO: 51, wherein the antibody binds to CLDN18.2,
the linker [GGGGS]-[LPQTGG]-[ethylenediamine] at the C-terminus of the light chains, and
the anthracycline-based small molecule toxin 3′-deamino-3″,4′-anhydro-[2″(S)-methoxy-3″(R)-oxy-4″-morpholinyl]doxorubicin (PNU-159682), linked covalently to the ethylenediamine of the linker at C₁₃, resulting in the loss of C₁₄ and of the hydroxyl group.

23. An antibody-drug conjugate consisting of:
the antibody consisting of two heavy chains of the amino acid sequence according to SEQ ID NO: 134 and two light chains of the amino acid sequence according to SEQ ID NO: 51, wherein the antibody binds to CLDN18.2,
the linker [GGGGS]-[LPQXTGG]-[ethylenediamine] at the C-terminus of the light chains, and
the anthracycline-based small molecule toxin 3′-deamino-3″,4′-anhydro-[2″ (S)-methoxy-3″ (R)-oxy-4″-morpholinyl]doxorubicin (PNU-159682), linked covalently to the ethylenediamine of the linker at C₁₃, resulting in the loss of C₁₄ and of the hydroxyl group.

24. A pharmaceutical composition comprising the antibody-drug conjugate of any of embodiments 1 to 23 and an excipient.

25. The antibody-drug conjugate of any of embodiments 1 to 23 for use in treatment.

26. The antibody-drug conjugate of any of embodiments 1 to 23 for use in the treatment of cancer.

27. The antibody-drug conjugate of embodiment 24, wherein the cancer is selected from pancreatic, gastric, esophageal, ovarian, and lung cancer.

Sequences

| | |
|---|---|
| SEQ ID NO: 1 | DYAMH |
| SEQ ID NO: 2 | WINTYTGKPTYADDFKG |
| SEQ ID NO: 3 | AVFYGYTMDA |
| SEQ ID NO: 4 | RASEDIYSNLA |
| SEQ ID NO: 5 | SVKRLQD |
| SEQ ID NO: 6 | LQGSNFPLT |
| SEQ ID NO: 7 | WINAYTGKPTYADDFKG |
| SEQ ID NO: 8 | AVYYGYTMDA |
| SEQ ID NO: 9 | RTSEDIYSNFA |
| SEQ ID NO: 10 | SVNRLQD |
| SEQ ID NO: 11 | LQGSKFPLT |
| SEQ ID NO: 12 | DYAMY |
| SEQ ID NO: 13 | RTSEDIYSNLA |
| SEQ ID NO: 14 | AIKRLQD |
| SEQ ID NO: 15 | WINTYTGKPTYAQKFQG |
| SEQ ID NO: 16 | WINTYTGKPTYSQKFQG |
| SEQ ID NO: 17 | RTSEDIYSNLA |
| SEQ ID NO: 18 | RTSEDIYSNFA |
| SEQ ID NO: 19 | SVNRLQD |
| SEQ ID NO: 20 | WINAYTGKPTYAQKFQG |
| SEQ ID NO: 21 | DYAMX |
| | X in 5^{th} position is H or Y |
| SEQ ID NO: 22 | WINXYTGKPTYXXXFXG |
| | X in 4^{th} position is T or A; |
| | X in 12^{th} position is A or S; |
| | X in 13^{th} position is D or Q; |
| | X in 14^{th} position is D or K; |
| | X in 16^{th} position is K or Q |
| SEQ ID NO: 23 | AVXYGYTMDA |
| | X in 3^{rd} position is F or Y |
| SEQ ID NO: 24 | RXSEDIYSNXA |
| | X in 2^{nd} position is A or T; |
| | X in 10^{th} position is L or F |
| SEQ | XXXRLQD |
| ID | X in 1^{st} position is S or A; |
| NO: | X in 2^{nd} position is V or I; |
| 25 | X in 3^{rd} position is K or N |
| SEQ | LQGSXFPLT |
| ID | X in 5^{th} position is K or N |
| NO: | |
| 26 | |
| SEQ | cCl1-1 HC variable region |
| ID | QIQLVQSGPELKKPGESVKISCKASGYTFTDYAMHWVKQAPGK |
| NO: | GLKWMGWINTYTGKPTYADDFKGRFVFSLEASASTANLQISNL |
| 27 | KNEDTATYFCARAVFYGYTMDAWGQGTSVTVSS |
| SEQ | cCl1-1 LC variable region |
| ID | DIQMTQSPASLSASLGETISIACRASEDIYSNLAWYQQKSGKSPQ |
| NO: | LLIFSVKRLQDGVPSRFSGSGSGTQYSLKISGMQPEDEGDYFCLQ |
| 28 | GSNFPLTFGSGTKLEIK |
| SEQ | cCl1-2 HC variable region |
| ID | QIQLVQSGPELKKPGESVKISCKTSGYTFTDYAMHWVKQGPGK |
| NO: | GMKWMGWINAYTGKPTYADDFKGRFVLSLEASASTANLQISN |
| 29 | LKNEDTATYFCARAVYYGYTMDAWGQGTSVIVSS |
| SEQ | cCl1-2 LC variable region |
| ID | DIQMTQSPASLSASLGETISIECRTSEDIYSNFAWFQQKSGKSPQL |
| NO: | LIYSVNRLQDGVPSRFSGSGSGTQYSLKISGMQPEDEGDYFCLQ |
| 30 | GSKFPLTFGSGTKLEIK |
| SEQ | cCl1-3 HC variable region |
| ID | QIQLVQSGPELKKPGESVKISCKASGYTFTDYAMYWVKQVPGK |
| NO: | GLRWMGWINTYTGKPTYADDFKGRFVFSLEASASTANLQISNL |
| 31 | KNEDTATYFCARAVFYGYTMDAWGQGTSVTVSS |
| SEQ | cCl1-3 LC variable region |
| ID | DIQMTQSPASLSASLGETISIACRTSEDIYSNLAWYQQKSGKSPQ |
| NO: | LLIFAIKRLQDGVPSRFSGSGSGTQYSLKISGMQPEDEGDYFCLQ |
| 32 | GSKFPLTFGSGTKLEIK |
| SEQ | hCL1a HC variable region |
| ID | QVQLVQSGAEVKKPGASVKVSCKASGYTFTDYAMHWVRQAP |
| NO: | GQRLEWMGWINTYTGKPTYAQKFQGRVTITRDTSASTAYMELS |
| 33 | SLRSEDTAVYYCARAVFYGYTMDAWGQGTLVTVSS |
| SEQ | hCL1b, c and d HC variable region |
| ID | QVQLVQSGAEVKKPGASVKVSCKASGYTFTDYAMHWVRQAP |
| NO: | GQRLEWMGWINTYTGKPTYSQKFQGRVTITRDTSASTAYMELS |
| 34 | SLRSEDTAVYYCARAVFYGYTMDAWGQGTLVTVSS |
| SEQ | hCL1e HC variable region |
| ID | QVQLVQSGAEVKKPGASVKVSCKASGYTFTDYAMYWVRQAP |
| NO: | GQRLEWMGWINTYTGKPTYAQKFQGRVTITRDTSASTAYMELS |
| 35 | SLRSEDTAVYYCARAVFYGYTMDAWGQGTLVTVSS |
| SEQ | hCL1f and g HC variable region |
| ID | QVQLVQSGAEVKKPGASVKVSCKASGYTFTDYAMHWVRQAP |
| NO: | GQRLEWMGWINAYTGKPTYAQKFQGRVTITRDTSASTAYMEL |
| 36 | SSLRSEDTAVYYCARAVFYGYTMDAWGQGTLVTVSS |
| SEQ | hCL1h, i and j HC variable region |
| ID | QVQLVQSGAEVKKPGASVKVSCKASGYTFTDYAMYWVRQAP |
| NO: | GQRLEWMGWINAYTGKPTYAQKFQGRVTITRDTSASTAYMEL |
| 37 | SSLRSEDTAVYYCARAVYYGYTMDAWGQGTLVTVSS |
| SEQ | hCL1a, b, e and i LC variable region |
| ID | DIQMTQSPSSLSASVGDRVTITCRASEDIYSNLAWYQQKPGKAP |
| NO: | KLLIFSVKRLQDGVPSRFSGSGSGTDFTLTISSLQPEDFATYYCLQ |
| 38 | GSNFPLTFGQGTKVEIK |
| SEQ | hCL1c and j LC variable region |
| ID | DIQMTQSPSSLSASVGDRVTITCRTSEDIYSNLAWYQQKPGKAP |
| NO: | KLLIFAIKRLQDGVPSRFSGSGSGTDFTLTISSLQPEDFATYYCLQ |
| 39 | GSKFPLTFGQGTKVEIK |
| SEQ | hCL1d and g LC variable region |
| ID | DIQMTQSPSSLSASVGDRVTITCRTSEDIYSNFAWYQQKPGKAP |
| NO: | KLLIYSVNRLQDGVPSRFSGSGSGTDFTLTISSLQPEDFATYYCL |
| 40 | QGSKFPLTFGQGTKVEIK |
| SEQ | hCL1f and h LC variable region |
| ID | DIQMTQSPSSLSASVGDRVTITCRASEDIYSNLAWYQQKPGKAP |
| NO: | KLLIYSVKRLQDGVPSRFSGSGSGTDFTLTISSLQPEDFATYYCL |
| 41 | QGSNFPLTFGQGTKVEIK |
| SEQ | hCL3a, b and c HC variable region |
| ID | QVQLQESGPGLVKPSETLSLTCAVSGYSVSSNYRWHWIRQPPG |
| NO: | KGLEWIGYINIAGSTNYNPSLKSRVTISVDTSKNQFSLKLSSVTA |
| 42 | ADTAVYYCARNPSITRAMDAWGQGTLVTVSS |
| SEQ | hCL3a LC variable region |
| ID | DIQMTQSPSSLSASVGDRVTITCKSSQNIFKNLEWYQQKPGKAP |
| NO: | KLLIYYTNNLQTGVPSRFSGSGSGTDFTLTISSLQPEDFATYYCY |
| 43 | QYNSGPFTFGQGTKVEIK |
| SEQ | hCL3b LC variable region |
| ID | DIQMTQSPSSLSASVGDRVTITCRSSQNIFKNLEWYQQKPGKAP |
| NO: | KLLIYYTNNLQTGVPSRFSGSGSGTDFTLTISSLQPEDFATYYCY |
| 44 | QYNSGPFTFGQGTKVEIK |
| SEQ | hCL3c LC variable region |
| ID | DIQMTQSPSSLSASVGDRVTITCRSSQNIFKNLEWYQQKPGKAP |
| NO: | KLLIYYTNNLQSGVPSRFSGSGSGTDFTLTISSLQPEDFATYYCY |
| 45 | QYNSGPFTFGQGTKVEIK |
| SEQ | hCL1a HC full |
| ID | QVQLVQSGAEVKKPGASVKVSCKASGYTFTDYAMHWVRQAP |
| NO: | GQRLEWMGWINTYTGKPTYAQKFQGRVTITRDTSASTAYMELS |
| 46 | SLRSEDTAVYYCARAVFYGYTMDAWGQGTLVTVSSASTKGPS |
| | TFPAVLQSSGLYSLSSVVTVPSSSLGTQTYICNVNHKPSNTKVDK |
| | KVEPKSCDKTHTCPPCPAPELLGGPSVFLFPPKPKDTLMISRTPE |
| | VTCVVVDVSHEDPEVKFNWYVDGVEVHNAKTKPREEQYNSTY |
| | RVVSVLTVLHQDWLNGKEYKCKVSNKALPAPIEKTISKAKGQP |
| | REPQVYTLPPSRDELTKNQVSLTCLVKGFYPSDIAVEWESNGQP |
| | ENNYKTTPPVLDSDGSFFLYSKLTVDKSRWQQGNVFSCSVMHE |
| | ALHNHYTQKSLSLSPGX X is K or R |
| SEQ ID NO: 47 | hCL1b, c and d HC full |
| | QVQLVQSGAEVKKPGASVKVSCKASGYTFTDYAMHWVRQAP |
| | GQRLEWMGWINTYTGKPTYSQKFQGRVTITRDTSASTAYMELS |
| | SLRSEDTAVYYCARAVFYGYTMDAWGQGTLVTVSSASTKGPS |
| | VFPLAPSSKSTSGGTAALGCLVKDYFPEPVTVSWNSGALTSGVH |
| | TFPAVLQSSGLYSLSSVVTVPSSSLGTQTYICNVNHKPSNTK VDK |
| | KVEPKSCDKTHTCPPCPAPELLGGPSVFLFPPKPKDTLMISRTPE |
| | VTCVVVDVSHEDPEVKFNWYVDGVEVHNAKTKPREEQYNSTY |
| | RVVSVLTVLHQDWLNGKEYKCKVSNKALPAPIEKTISKAKGQP |
| | REPQVYTLPPSRDELTKNQVSLTCLVKGFYPSDIAVEWESNGQP |
| | ENNYKTTPPVLDSDGSFFLYSKLTVDKSRWQQGNVFSCSVMHE |
| | ALHNHYTQKSLSLSPGX X is K or R |
| SEQ ID NO: 48 | hCL1e HC full |
| | QVQLVQSGAEVKKPGASVKVSCKASGYTFTDYAMYWVRQAP |
| | GQRLEWMGWINTYTGKPTYAQKFQGRVTITRDTSASTAYMELS |
| | SLRSEDTAVYYCARAVFYGYTMDAWGQGTLVTVSSASTKGPS |
| | VFPLAPSSKSTSGGTAALGCLVKDYFPEPVTVSWNSGALTSGVH |
| | TFPAVLQSSGLYSLSSVVTVPSSSLGTQTYICNVNHKPSNTKVDK |
| | KVEPKSCDKTHTCPPCPAPELLGGPSVFLFPPKPKDTLMISRTPE |
| | VTCVVVDVSHEDPEVKFNWYVDGVEVHNAKTKPREEQYNSTY |
| | RVVSVLTVLHQDWLNGKEYKCKVSNKALPAPIEKTISKAKGQP |
| | REPQVYTLPPSRDELTKNQVSLTCLVKGFYPSDIAVEWESNGQP |
| | ENNYKTTPPVLDSDGSFFLYSKLTVDKSRWQQGNVFSCSVMHE |
| | ALHNHYTQKSLSLSPGX X is K or R |
| SEQ ID NO: 49 | hCL1f and g HC full |
| | QVQLVQSGAEVKKPGASVKVSCKASGYTFTDYAMHWVRQAP |
| | GQRLEWMGWINAYTGKPTYAQKFQGRVTITRDTSASTAYMEL |
| | SSLRSEDTAVYYCARAVFYGYTMDAWGQGTLVTVSSASTKGPS |
| | VFPLAPSSKSTSGGTAALGCLVKDYFPEPVTVSWNSGALTSGVH |
| | TFPAVLQSSGLYSLSSVVTVPSSSLGTQTYICNVNHKPSNTKVDK |
| | KVEPKSCDKTHTCPPCPAPELLGGPSVFLFPPKPKDTLMISRTPE |
| | VTCVVVDVSHEDPEVKFNWYVDGVEVHNAKTKPREEQYNSTY |
| | RVVSVLTVLHQDWLNGKEYKCKVSNKALPAPIEKTISKAKGQP |
| | REPQVYTLPPSRDELTKNQVSLTCLVKGFYPSDIAVEWESNGQP |
| | ENNYKTTPPVLDSDGSFFLYSKLTVDKSRWQQGNVFSCSVMHE |
| | ALHNHYTQKSLSLSPGX X is K or R |
| SEQ ID NO: 50 | hCL1h, i and j HC full |
| | QVQLVQSGAEVKKPGASVKVSCKASGYTFTDYAMYWVRQAP |
| | GQRLEWMGWINAYTGKPTYAQKFQGRVTITRDTSASTAYMEL |
| | SSLRSEDTAVYYCARAVYYGYTMDAWGQGTLVTVSSASTKGP |
| | SVFPLAPSSKSTSGGTAALGCLVKDYFPEPVTVSWNSGALTSGV |
| | HTFPAVLQSSGLYSLSSVVTVPSSSLGTQTYICNVNHKPSNTKVD |
| | KKVEPKSCDKTHTCPPCPAPELLGGPSVFLFPPKPKDTLMISRTP |
| | EVTCVVVDVSHEDPEVKFNWYVDGVEVHNAKTKPREEQYNST |
| | YRVVSVLTVLHQDWLNGKEYKCKVSNKALPAPIEKTISKAKGQ |
| | PREPQVYTLPPSRDELTKNQVSLTCLVKGFYPSDIAVEWESNGQ |
| | PENNYKTTPPVLDSDGSFFLYSKLTVDKSRWQQGNVFSCSVMH |
| | EALHNHYTQKSLSLSPGX X is K or R |
| SEQ ID NO: 51 | hCL1a, b, e and i LC full |
| | DIQMTQSPSSLSASVGDRVTITCRASEDIYSNLAWYQQKPGKAP |
| | KLLIFSVKRLQDGVPSRFSGSGSGTDFTLTISSLQPEDFATYYCLQ |
| | GSNFPLTFGQGTKVEIKRTVAAPSVFIFPPSDEQLKSGTASVVCL |
| | LNNFYPREAKVQWKVDNALQSGNSQESVTEQDSKDSTYSLSST |
| | LTLSKADYEKHKVYACEVTHQGLSSPVTKSFNRGEC |
| SEQ ID NO: 52 | hCL1c and j LC full |
| | DIQMTQSPSSLSASVGDRVTITCRTSEDIYSNLAWYQQKPGKAP |
| | KLLIFAIKRLQDGVPSRFSGSGSGTDFTLTISSLQPEDFATYYCLQ |
| | GSKFPLTFGQGTKVEIKRTVAAPSVFIFPPSDEQLKSGTASVVCL |
| | LNNFYPREAKVQWKVDNALQSGNSQESVTEQDSKDSTYSLSST |
| | LTLSKADYEKHKVYACEVTHQGLSSPVTKSFNRGEC |
| SEQ ID NO: 53 | hCL1d and g LC full |
| | DIQMTQSPSSLSASVGDRVTITCRTSEDIYSNFAWYQQKPGKAP |
| | KLLIYSVNRLQDGVPSRFSGSGSGTDFTLTISSLQPEDFATYYCL |
| | QGSKFPLTFGQGTKVEIKRTVAAPSVFIFPPSDEQLKSGTASVVC |
| | LLNNFYPREAKVQWKVDNALQSGNSQESVTEQDSKDSTYSLSS |
| | TLTLSKADYEKHKVYACEVTHQGLSSPVTKSFNRGEC |
| SEQ ID NO: 54 | hCL1f and h LC full |
| | DIQMTQSPSSLSASVGDRVTITCRASEDIYSNLAWYQQKPGKAP |
| | KLLIYSVKRLQDGVPSRFSGSGSGTDFTLTISSLQPEDFATYYCL |
| | QGSNFPLTFGQGTKVEIKRTVAAPSVFIFPPSDEQLKSGTASVVC |
| | LLNNFYPREAKVQWKVDNALQSGNSQESVTEQDSKDSTYSLSS |
| | TLTLSKADYEKHKVYACEVTHQGLSSPVTKSFNRGEC |
| SEQ ID NO: 55 | IMAB362 HC full |
| | QVQLQQPGAELVRPGASVKLSCKASGYTFTSYWINWVKQRPG |
| | QGLEWIGNIYPSDSYTNYNQKFKDKATLTVDKSSSTAYMQLSSP |
| | TSEDSAVYYCTRSWRGNSFDYWGQGTTLTVSSASTKGPSVFPL |
| | APSSKSTSGGTAALGCLVKDYFPEPVTVSWNSGALTSGVHTFPA |
| | VLQSSGLYSLSSVVTVPSSSLGTQTYICNVNHKPSNTKVDKKVE |
| | PKSCDKTHTCPPCPAPELLGGPSVFLFPPKPKDTLMISRTPEVTC |
| | VVVDVSHEDPEVKFNWYVDGVEVHNAKTKPREEQYNSTYRVV |
| | SVLTVLHQDWLNGKEYKCKVSNKALPAPIEKTISKAKGQPREP |
| | QVYTLPPSRDELTKNQVSLTCLVKGFYPSDIAVEWESNGQPENN |
| | YKTTPPVLDSDGSFFLYSKLTVDKSRWQQGNVFSCSVMHEALH |
| | NHYTQKSLSLSPGX X is K or R |
| SEQ ID NO: 56 | IMAB362 LC full |
| | DIVMTQSPSSLTVTAGEKVTMSCKSSQSLLNSGNQKNYLTWYQ |
| | QKPGQPPKLLIYWASTRESGVPDRFTGSGSGTDFTLTISSVQAED |
| | LAVYYCQNDYSYPFTFGSGTKLEIKRTVAAPSVFIFPPSDEQLKS |
| | GTASVVCLLNNFYPREAKVQWKVDNALQSGNSQESVTEQDSK |
| | DSTYSLSSTLTLSKADYEKHKVYACEVTHQGLSSPVTKSFNRGE |
| | C |
| SEQ ID NO: 57 | DQWSTQDLYN |
| SEQ ID NO: 58 | NNPVTAVFNYQ |
| SEQ ID NO: 59 | STQDLYNNPVTAVF |
| SEQ ID NO: 60 | TNFWMSTANMYTG |
| SEQ ID NO: 61 | ALMIVGIVLGAIGLLV |
| SEQ ID NO: 62 | RIGSMEDSAKANMTLTSGIMFIVS |
| SEQ ID NO: 63 | METDTLLLWVLLLWVPGSTGDAAQPARRARRTKLGTELGSTPV |
| | WWNSADGRMDQWSTQDLYNNPVTAVFNYQGLWRSCVRESSG |
| | FTECRGYFTLLGLPAMLQAVRAAIQHSGGRSRRARTKTHLRRG |
| | SE |
| SEQ ID NO: 64 | MDQWSTQDLYNNPVT |
| SEQ ID NO: 65 | LYNNPVTAVFNYQGL |
| SEQ ID NO: 66 | VFNYQGLWRSCVRES |
| SEQ ID NO: 67 | QGLWRSCVRESSGFT |
| SEQ ID NO: 68 | RSCVRESSGFTECRG |
| SEQ ID NO: 69 | TEDEVQSYPSKHDYV |
| SEQ ID NO: 70 | EVQSYPSKHDYV |
| SEQ ID NO: 71 | gactacgcgatgcac |
| SEQ ID NO: 72 | tggatcaacacgtacacggggaagccgacatacgcggacgacttcaagggg |
| SEQ ID NO: 73 | gccgtcttctacggatatacgatggacgcg |
| SEQ ID NO: 74 | cagatccagctcgtccagagcgggccggagctgaagaagccgggggagagcgtgaagatctcgt |
| | gcaaggcgagcggatatacgttcacggactacgcgatgcactgggtcaagcaagcgccggggaaa |
| | gggctgaagtggatggggtggatcaacacgtacacggggaagccgacatacgcggacgacttcaa |
| | ggggcgattcgtgttctcgctggaggcgagcgcgagcacggcgaacctgcaaatctcgaacctgaa |
| | gaacgaggacacggcgacgtacttctgcgcgcgggccgtcttctacggatatacgatggacgcgtg |
| | ggggcagggtaccagcgtgacggtctcgagc |
| SEQ ID NO: 75 | cgggcgagcgaggacatctactcgaacctggcg |
| SEQ ID NO: 76 | tccgtcaagcggctgcaagac |
| SEQ ID NO: 77 | ctgcaagggagcaacttcccgctgacg |
| SEQ ID NO: 78 | gacatccagatgacgcagagcccggcgtcgctgagcgcgagcctgggggagacgatctcgatcgc |
| | gtgccgggcgagcgaggacatctactcgaacctggcgtggtatcaacagaagagcgggaagagcc |
| | cgcagctgctgatcttctccgtcaagcggctgcaagacggcgtcccgagccgattctcggggagcg |
| | ggagcgggacgcagtactcgctgaagatctcggggatgcagccggaggacgagggggactacttc |
| | tgcctgcaagggagcaacttcccgctgacgttcgggtcgggtaccaaactcgagatcaaa |
| SEQ ID NO: 79 | tggatcaacgcgtacacggggaagccgacctacgcggacgacttcaagggg |
| SEQ ID NO: 80 | gccgtctactacggatatacgatggac |
| SEQ ID NO: 81 | cagatccagctcgtccagagcgggccggagctgaagaagccgggggagagcgtgaagatctcgt |
| | gcaagacgagcggatatacgttcacggactacgcgatgcactgggtcaagcaggggccagggaaa |
| | gggatgaagtggatggggtggatcaacgcgtacacggggaagccgacctacgcggacgacttcaa |
| | ggggcgattcgtgctgagcctggaggcgagcgcctcgacggcgaacctgcaaatctcgaacctgaa |
| | gaacgaggacacggcgacgtacttctgcgcgcgggccgtctactacggatatacgatggacgcgtg |
| | ggggcagggtaccagcgtgatcgtctcgagc |
| SEQ ID NO: 82 | cggacgagcgaggacatctactcgaacttcgcg |
| SEQ ID NO: 83 | tcagtcaaccggctgcaagac |
| SEQ ID NO: 84 | ctgcaagggagcaagttcccgctgacg |
| SEQ ID NO: 85 | gacatccagatgacgcagagcccggcgagcctgagcgcgagcctgggggagacgatctcgatcg |
| | agtgccggacgagcgaggacatctactcgaacttcgcgtggttccagcagaagagcgggaagagc |
| | ccgcagctgctgatctactcagtcaaccggctgcaagacggcgtcccgagccgattctcggggagc |
| | gggagcgggacgcagtactcgctgaagatctcggggatgcagccggaggacgagggggactactt |
| | ctgcctgcaagggagcaagttcccgctgacgttcgggagcggtaccaaactcgagatcaaa |
| SEQ ID NO: 86 | gactacgcgatgtac |
| SEQ ID NO: 87 | tggatcaacacgtacacggggaagccgacctacgcggacgacttcaagggg |
| SEQ ID NO: 88 | cagatccagctcgtccagagcgggccggagctgaagaagccgggggagagcgtgaagatctcgt |
| | gcaaggcgagcggatatacgttcacggactacgcgatgtactgggtcaagcaagtgccggggaaa |
| | gggctgcgatggatggggggatcaacacgtacacggggaagccgacctacgcggacgacttcaa |
| | ggggcgattcgtgttctcgctggaggcgagcgcgagcacggcgaacctgcaaatctcgaacctgaa |
| | gaacgaggacacggcgacgtacttctgcgcgcgggccgtcttctacggatatacgatggacgcgtg |
| | ggggcagggtaccagcgtgacggtctcgagc |
| SEQ ID NO: 89 | cggacgagcgaggacatctactcgaacctggcg |
| SEQ ID NO: 90 | gcgatcaagcggctgcaagac |
| SEQ ID NO: 91 | gacatccagatgacgcagagcccggcgagcctgagcgcgagcctgggggagacgatctcgatcg |
| | cgtgccggacgagcgaggacatctactcgaacctggcgtggtatcaacagaagagcgggaagagc |
| | ccgcagctgctgatcttcgcgatcaagcggctgcaagacggcgtcccgagccgattctcggggagc |
| | gggagcgggacgcagtactcgctgaagatctcggggatgcagccggaggacgagggggactactt |
| | ctgcctgcaagggagcaagttcccgctgacgttcgggtcgggtaccaaactcgagatcaaa |
| SEQ ID NO: 92 | tggatcaatacatacacggggaagccgacttatgcgcaaaaattccaagga |
| SEQ ID NO: 93 | gcggtcttctacggatatacgatggatgcc |
| SEQ ID NO: 94 | caggtccaactagtccaaagcggggcggaagtcaagaagcccggagcatccgtcaaagtcagctg |
| | caaggcgagcggatatacatttacggactacgcgatgcactgggtcaggcaagcccctgggcaaag |
| | gctcgaatggatgggatggatcaatacatacacggggaagccgacttatgcgcaaaaattccaagga |
| | agagtcacaattacgcgggatacatccgcatctaccgcctacatggagctaagctcgctgcggagcg |
| | aggatacggcggtctactattgcgcccgagcggtcttctacggatatacgatggatgcctgggggca |
| | gggtaccctggtcacggtctcgagc |
| SEQ ID NO: 95 | agggcctccgaagacatctactccaacctggca |
| SEQ ID NO: 96 | agcgtcaaaagactacaagat |
| SEQ ID NO: 97 | ttgcaaggaagcaatttccccttgact |
| SEQ ID NO: 98 | gacattcaaatgacgcaaagcccatcatcgctgagcgcatcggtcggggatagagtcaccataacat |
| | gcagggcctccgaagacatctactccaacctggcatggtatcaacaaaaaccggggaaggctccga |
| | agctgctgatatttagcgtcaaaagactacaagatggagtaccgagccgattttcgggaagcgggag |
| | cgggacggatttcacgctgaccatatcaagtttgcaaccggaggattttgcgacatactattgcttgcaa |
| | ggaagcaatttccccttgactttcgggcaaggtaccaaggtcgagatcaaa |
| SEQ ID NO: 99 | gattatgcaatgcac |
| SEQ ID NO: 100 | tggattaacacctacacgggcaagcccacatactcccaaaaattccaagga |
| SEQ ID NO: 101 | gctgtattctatggatatacaatggatgcc |
| SEQ ID NO: 102 | caggtccaattagtccaaagcggggcggaagtcaagaagccgggggcgagcgtcaaagtctcatg |
| | caaagcgagcggatacacatttacggattatgcaatgcactgggtcaggcaagcacccggacaaag |
| | gctggaatggatgggatggattaacacctacacgggcaagcccacatactcccaaaaattccaagga |
| | agggtcacgataacgagagacacgagcgcgagcaccggaatggatgggatggattaacacctaca |
| | cgggcaagcccacatactcccaaaaattccaaggaagggtcacgataacgagagacacgagcgcg |
| | agcaccgtaccctggtcaccgtctcgagc |
| SEQ ID NO: 103 | cgaacgagcgaggacatatactcaaaccttgca |
| SEQ ID NO: 104 | gcgataaagaggctgcaagac |
| SEQ ID NO: 105 | ttgcaaggctccaaatttcccctgaca |
| SEQ ID NO: 106 | gacatccaaatgactcaaagcccatcatcgctatcggcatcggtcggggatagagtcacgataacatg |
| | ccgaacgagcgaggacatatactcaaaccttgcatggtatcaacaaaagccggggaaggccccgaa |
| | gctactgatattcgcgataaagaggctgcaagacggagttccatcacgattttcgggatctggctcgg |
| | ggaccgattttacgctgactatatcatcgctgcaaccggaagattttgcaacatactactgcttgcaagg |
| | ctccaaatttcccctgacattcggacaaggtaccaaggtcgagatcaaa |
| SEQ ID NO: 107 | cggacgagcgaggatatttattcgaactttgca |
| SEQ ID NO: 108 | cagtcaatcggctacaagat |
| SEQ ID NO: 109 | gacatccaaatgacgcaatcaccgagctcgctgagcgcatctgtcggggaccgtgtcacaatcacat |
| | gccggacgagcgaggatatttattcgaactttgcatggtatcaacaaaaaccgggcaaggctccgaa |
| | acttttgatttattcagtcaatcggctacaagatggcgtcccgagccgatttagcgggagcggatcggg |
| | aaccgactttacgctgacgatatcatcgctacaaccggaggacttcgcgacttattactgcctacaagg |
| | gagcaaattcccgctgacattcggacaaggtaccaaggtcgagatcaaa |
| SEQ ID NO: 110 | gattacgcaatgtac |
| SEQ ID NO: 111 | tggataaatacctatacgggaaagccaacatacgcccaaaaattccaaggc |
| SEQ ID NO: 112 | gccgtcttttatggatatacgatggacgca |
| SEQ ID NO: 113 | caggtccaactggtccaatcgggggctgaagtcaaaaagccgggggcgagcgtcaaagtcagctg |
| | caaagcatcgggatacacatttacggattacgcaatgtactgggtcaggcaagcacccggccaacga |
| | ctggaatggatgggctggataaatacctatacgggaaagccaacatacgcccaaaaattccaaggcc |
| | gcgtcacaataacgcgggacacgagcgcatcgacggcttatatggaactatcatcgctgcgatcgga |
| | agacacggcggtctattattgcgcacgcgccgtcttttatggatatacgatggacgcatgggggcagg |
| | gtaccctggtcacggtctcgagc |
| SEQ ID NO: 114 | gactacgcaatgcac |
| SEQ ID NO: 115 | tggattaatgcctacacggggaagccgacctacgcacaaaaattccaagga |
| SEQ ID NO: 116 | gccgtcttctatggatatacgatggatgct |
| SEQ ID NO: 117 | caggtccaattggtccaaagcggggcggaggtcaagaagccgggggcgagcgtcaaagtctcatg |
| | caaggcaagcggatatacatttacggactacgcaatgcactgggtccggcaagcccctgggcaacg |
| | gctggaatggatgggatggattaatgcctacacggggaagccgacctacgcacaaaaattccaagg |
| | acgagtcacgattacgcgggatactagcgcgagcaccgcatatatggagctaagctcgctgcgatct |
| | gaggataccgctgtatactactgcgcgagagccgtcttctatggatatacgatggatgcttgggggca |
| | gggtaccctggtcacggtctcgagc |
| SEQ ID NO: 118 | cgagcttcggaggacatctatagcaacttggct |
| SEQ ID NO: 119 | agcgtcaaaaggctccaagac |
| SEQ ID NO: 120 | ctacaaggctctaacttcccattgaca |
| SEQ ID NO: 121 | gatatccaaatgacgcaatcaccatctagcctatcggcctctgtgggggaccgagtcaccatcacatg |
| | ccgagcttcggaggacatctatagcaacttggcttggtatcaacaaaagccggggaaagcaccaaag |
| | ctgctgatatatagcgtcaaaaggctccaagacggagtcccaagccgattctcgggctccggctccg |
| | ggacggattttacgctgacaatttcgagcctgcaaccggaggactttgcaacctactattgcctacaag |
| | gctctaacttcccattgacatttgggcaaggtaccaaggtcgagatcaaa |
| SEQ ID NO: 122 | gactacgctatgtat |
| SEQ ID NO: 123 | tggattaatgcctacaccgggaagccgacttatgcgcaaaaatttcaagga |
| SEQ ID NO: 124 | gcggtctactatggatatacgatggacgca |
| SEQ ID NO: 125 | caggtccaactggttcaatctggagcggaagtcaagaagcccggagcatccgtcaaagtctcgtgca |
| | aggcatctggatacacattcaccgactacgctatgtattgggtccggcaagcccccggacaacggct |
| | ggaatggatgggatggattaatgcctacaccgggaagccgacttatgcgcaaaaatttcaaggaagg |
| | gtcacgattacgcgggacacgagcgcctcaaccgcatacatggagctatcgagcctgcgaagcgag |
| | gacaccgcggtctactactgcgcgcgggcggtctactatggatatacgatggacgcatgggggcag |
| | ggtaccctggtcacggtctcgagc |
| SEQ ID NO: 126 | WINXYTGKPTYXQKFQG |
| | X in 4^{th} position is T or A; |
| | X in 12^{th} position is A or S |
| | [HC CDR2 for hCl1x only, not chimeric clones cCl1-1,2,3] |
| SEQ ID NO: 127 | RTVAAPSVFIFPPSDEQLKSGTASVVCLLNNFYPREAKVQWKVD |
| | NALQSGNSQESVTEQDSKDSTYSLSSTLTLSKADYEKHKVYACE |
| | VTHQGLSSPVTKSFNRGEC [constant light chain-CL domain] |
| SEQ ID NO: 128 | ASTKGPSVFPLAPSSKSTSGGTAALGCLVKDYFPEPVTVSWNSG |
| | ALTSGVHTFPAVLQSSGLYSLSSVVTVPSSSLGTQTYICNVNHKP |
| | SNTKVDKKVEPKSCDKTHTCPPCPAPELLGGPSVFLFPPKPKDTL |
| | MISRTPEVTCVVVDVSHEDPEVKFNWYVDGVEVHNAKTKPREE |
| | QYNSTYRVVSVLTVLHQDWLNGKEYKCKVSNKALPAPIEKTIS |
| | KAKGQPREPQVYTLPPSRDELTKNQVSLTCLVKGFYPSDIAVEW |
| | ESNGQPENNYKTTPPVLDSDGSFFLYSKLTVDKSRWQQGNVFS |
| | CSVMHEALHNHYTQKSLSLSPGX X is K or R [constant heavy |
| | chain-CH1 + Fc domain] |
| SEQ ID NO: 129 | ASTKGPSVFPLAPSSKSTSGGTAALGCLVKDYFPEPVTVSWNSG |
| | ALTSGVHTFPAVLQSSGLYSLSSVVTVPSSSLGTQTYICNVNHKP |
| | SNTKVDKKVEPKSCDKTHTCPPCPAPEAAGGPSVFLFPPKPKDT |
| | LMISRTPEVTCVVVDVSHEDPEVKFNWYVDGVEVHNAKTKPRE |
| | EQYNSTYRVVSVLTVLHQDWLNGKEYKCKVSNKALPAPIEKTI |
| | SKAKGQPREPQVYTLPPSRDELTKNQVSLTCLVKGFYPSDIAVE |
| | WESNGQPENNYKTTPPVLDSDGSFFLYSKLTVDKSRWQQGNVF |
| | SCSVMHEALHNHYTQKSLSLSPGX X is K or R [L234A/L235A |
| | mutation in constant heavy chain-CH1 + Fc domain] |
| SEQ ID NO: 130 | ASTKGPSVFPLAPSSKSTSGGTAALGCLVKDYFPEPVTVSWNSG |
| | ALTSGVHTFPAVLQSSGLYSLSSVVTVPSSSLGTQTYICNVNHKP |
| | SNTKVDKKVEPKSCDKTHTCPPCPAPEAAGGPSVFLFPPKPKDT |
| | LMISRTPEVTCVVVDVSHEDPEVKFNWYVDGVEVHNAKTKPR |
| | EEQYNSTYRVVSVLTVLHQDWLNGKEYKCKVSNKALGAPIEK |
| | TISKAKGQPREPQVYTLPPSRDELTKNQVSLTCLVKGFYPSDIAV |
| | EWESNGQPENNYKTTPPVLDSDGSFFLYSKLTVDKSRWQQGNV |
| | FSCSVMHEALHNHYTQKSLSLSPGX X is K or R |
| | [L236A/L236A/P329G mutation in constant heavy chain-CH1 + Fc |
| | domain] |
| SEQ ID NO: 131 | LPQTGG [sortase tag] |
| SEQ ID NO: 132 | GGGGS-LPQTGG [sortase tag] |
| SEQ ID NO: 133 | hCL1a HC full LALA |
| | QVQLVQSGAEVKKPGASVKVSCKASGYTFTDYAMHWVRQAP |
| | GQRLEWMGWINTYTGKPTYAQKFQGRVTITRDTSASTAYMEL |
| | SSLRSEDTAVYYCARAVFYGYTMDAWGQGTLVTVSSASTKGP |
| | SVFPLAPSSKSTSGGTAALGCLVKDYFPEPVTVSWNSGALTSGV |
| | HTFPAVLQSSGLYSLSSVVTVPSSSLGTQTYICNVNHKPSNTKV |
| | DKKVEPKSCDKTHTCPPCPAPEAAGGPSVFLFPPKPKDTLMISR |
| | TPEVTCVVVDVSHEDPEVKFNWYVDGVEVHNAKTKPREEQYN |
| | STYRVVSVLTVLHQDWLNGKEYKCKVSNKALPAPIEKTISKAK |
| | GQPREPQVYTLPPSRDELTKNQVSLTCLVKGFYPSDIAVEWESN |
| | GQPENNYKTTPPVLDSDGSFFLYSKLTVDKSRWQQGNVFSCSV |
| | MHEALHNHYTQKSLSLSPGX X is K or R |
| SEQ ID NO: 134 | hCL1a HC full LALAPG |
| | QVQLVQSGAEVKKPGASVKVSCKASGYTFTDYAMHWVRQAP |
| | GQRLEWMGWINTYTGKPTYAQKFQGRVTITRDTSASTAYMEL |
| | SSLRSEDTAVYYCARAVFYGYTMDAWGQGTLVTVSSASTKGP |
| | SVFPLAPSSKSTSGGTAALGCLVKDYFPEPVTVSWNSGALTSGV |
| | HTFPAVLQSSGLYSLSSVVTVPSSSLGTQTYICNVNHKPSNTKV |
| | DKKVEPKSCDKTHTCPPCPAPEAAGGPSVFLFPPKPKDTLMISR |
| | TPEVTCVVVDVSHEDPEVKFNWYVDGVEVHNAKTKPREEQYN |
| | STYRVVSVLTVLHQDWLNGKEYKCKVSNKALGAPIEKTISKAK |
| | GQPREPQVYTLPPSRDELTKNQVSLTCLVKGFYPSDIAVEWESN |
| | GQPENNYKTTPPVLDSDGSFFLYSKLTVDKSRWQQGNVFSCSV |
| | MHEALHNHYTQKSLSLSPGX X is K or R |
| SEQ ID NO: 135 | CLDN18.2 |
| | MAVTACQGLGFVVSLIGIAGIIAATCMDQWSTQDLYNNPVTAV |
| | FNYQGLWRSCVRESSGFTECRGYFTLLGLPAMLQAVRALMIVG |
| | IVLGAIGLLVSIFALKCIRIGSMEDSAKANMTLTSGIMFIVSGLCA |
| | IAGVSVFANMLVTNFWMSTANMYTGMGGMVQTVQTRYTFGA |
| | ALFVGWVAGGLTLIGGVMMCIACRGLAPEETNYKAVSYHASG |
| | HSVAYKPGGFKASTGFGSNTKNKKIYDGGARTEDEVQSYPSKH |
| | DYV |
| SEQ ID NO: 136 | LPXTGₘ [sortase tag] |
| | X in 3rd position is any of the 20 natural amino acids, m = 1-21. |
| SEQ ID NO: 137 | LPXAGₘ [sortase tag] |
| | X in 3rd position is any of the 20 natural amino acids, m = 1-21. |
| SEQ ID NO: 138 | LPXSGₘ [sortase tag] |
| | X in 3rd position is any of the 20 natural amino acids, m = 1-21. |
| SEQ ID NO: 139 | LAXTGₘ [sortase tag] |
| | X in 3rd position is any of the 20 natural amino acids, m = 1-21. |
| SEQ ID NO: 140 | LPXTAₘ [sortase tag] |
| | X in 3rd position is any of the 20 natural amino acids, m = 1-21. |
| SEQ ID NO: 141 | NPQTGₘ [sortase tag] |
| SEQ ID NO: 142 | NPQTNₘ [sortase tag] |
| SEQ ID NO: 143 | LPETGG [sortase tag] |
| SEQ ID NO: 144 | (GGGGS)ₒ [oligopeptide], o = 1-5 |
| SEQ ID NO: 145 | LPXTGG [sortase tag] |
| | X in 3rd position is any of the 20 natural amino acids. |
| SEQ ID NO: 146 | GGGGSLPXTGG [sortase tag] |
| | X in 8th position is any of the 20 natural amino acids. |
| SEQ ID NO: 147 | GGGGG [oligopeptide] |
| SEQ ID NO: 148 | LPQTG [sortase tag] |

### REFERENCES

Abbott, W. M., M. M. Damschroder, and D. C. Lowe. 2014. ‘Current approaches to fine mapping of antigen-antibody interactions’, Immunology, 142: 526-35.
Abdiche, Y. N., D. S. Malashock, A. Pinkerton, and J. Pons. 2009. ‘Exploring blocking assays using Octet, ProteOn, and Biacore biosensors’, Anal Biochem, 386: 172-80.
Aguiar, S., J. Dias, A. M. Manuel, R. Russo, P. M. P. Gois, F. A. da Silva, and J. Goncalves. 2018. ‘Chimeric Small Antibody Fragments as Strategy to Deliver Therapeutic Payloads’, Adv Protein Chem Struct Blot, 112: 143-82.
Alegre, M. L., A. M. Collins, V. L. Pulito, R. A. Brosius, W. C. Olson, R. A. Zivin, R. Knowles, J. R. Thistlethwaite, L. K. Jolliffe, and J. A. Bluestone. 1992. ‘Effect of a single amino acid mutation on the activating and immunosuppressive properties of a “humanized” OKT3 monoclonal antibody’, J Immunol, 148: 3461-8.
An, Z., G. Forrest, R. Moore, M. Cukan, P. Haytko, L. Huang, S. Vitelli, J. Z. Zhao, P. Lu, J. Hua, C. R. Gibson, B. R. Harvey, D. Montgomery, D. Zaller, F. Wang, and W. Strohl. 2009. ‘IgG2m4, an engineered antibody isotype with reduced Fc function’, MAbs, 1: 572-9.
Bolt, S., E. Routledge, I. Lloyd, L. Chatenoud, H. Pope, S. D. Gorman, M. Clark, and H. Waldmann. 1993. ‘The generation of a humanized, non-mitogenic CD3 monoclonal antibody which retains in vitro immunosuppressive properties’, Eur J Immunol, 23: 403-11.
Broggini, M. 2008. ‘Nemorubicin’, Top Curr Chem, 283: 191-206.
Chang, Z. L., and Y. Y. Chen. 2017. ‘CARs: Synthetic Immunoreceptors for Cancer Therapy and Beyond’, Trends Mol Med, 23: 430-50.
Chen, X., J. L. Zaro, and W. C. Shen. 2013. ‘Fusion protein linkers: property, design and functionality’, Adv Drug Deliv Rev, 65: 1357-69.
Chu, S. Y., I. Vostiar, S. Karki, G. L. Moore, G. A. Lazar, E. Pong, P. F. Joyce, D. E. Szymkowski, and J. R. Desjarlais. 2008. ‘Inhibition of B cell receptor-mediated activation of primary human B cells by coengagement of CD19 and FcgammaRllb with Fc-engineered antibodies’, Mol Immunol, 45: 3926-33.
“Crosslinking Technical Handbook.” In. 2012. easy molecular bonding—crosslinking technology, edited by Thermo Scientific, 56. USA: Thermo Scientific.
Dall'Acqua, W. F., R. M. Woods, E. S. Ward, S. R. Palaszynski, N. K. Patel, Y. A. Brewah, H. Wu, P. A. Kiener, and S. Langermann. 2002. ‘Increasing the affinity of a human IgG1 for the neonatal Fc receptor: biological consequences’, J Immunol, 169: 5171-80.
Diebolder, C. A., F. J. Beurskens, R. N. de Jong, R. I. Koning, K. Strumane, M. A. Lindorfer, M. Voorhorst, D. Ugurlar, S. Rosati, A. J. Heck, J. G. van de Winkel, I. A. Wilson, A. J. Koster, R. P. Taylor, E. O. Saphire, D. R. Burton, J. Schuurman, P. Gros, and P. W. Parren. 2014. ‘Complement is activated by IgG hexamers assembled at the cell surface’, Science, 343: 1260-3.
Du, Y., A. Walsh, R. Ehrick, W. Xu, K. May, and H. Liu. 2012. ‘Chromatographic analysis of the acidic and basic species of recombinant monoclonal antibodies’, MAbs, 4: 578-85.
Ellerman, D. 2019. ‘Bispecific T-cell engagers: Towards understanding variables influencing the in vitro potency and tumor selectivity and their modulation to enhance their efficacy and safety’, Methods, 154: 102-17.
Gervais, D. 2016. ‘Protein deamidation in biopharmaceutical manufacture: understanding, control and impact’, J Chem Technol Biotechnol, 91: 569-75.
Green, M. R., and J. Sambrook. 2012. Molecular Cloning: A Laboratory Manual (Fourth Edition) (Cold Spring Harbor Laboratory Press).
Hansel, T. T., H. Kropshofer, T. Singer, J. A. Mitchell, and A. J. George. 2010. ‘The safety and side effects of monoclonal antibodies’, Nat Rev Drug Discov, 9: 325-38.
Hashimoto, Y., W. Zhou, K. Hamauchi, K. Shirakura, T. Doi, K. Yagi, T. Sawasaki, Y. Okada, M. Kondoh, and H. Takeda. 2018. ‘Engineered membrane protein antigens successfully induce antibodies against extracellular regions of claudin-5’, Sci Rep, 8: 8383.
Hewitt, K. J., R. Agarwal, and P. J. Morin. 2006. ‘The claudin gene family: expression in normal and neoplastic tissues’, BMC Cancer, 6: 186.
Holte, D., J. P. Lyssikatos, A. M. Valdiosera, Z. Swinney, V. Sisodiya, J. Sandoval, C. Lee, M. A. Aujay, R. B. Tchelepi, 0. M. Hamdy, C. Gu, B. Lin, H. Sarvaiya, M. A. Pysz, A. Laysang, S. Williams, D. Jun Lee, M. K. Holda, J. W. Purcell, and J. Gavrilyuk. 2020. ‘Evaluation of PNU-159682 antibody drug conjugates (ADCs)’, Bioorg Med Chem Lett, 30: 127640.
Idusogie, E. E., P. Y. Wong, L. G. Presta, H. Gazzano-Santoro, K. Totpal, M. Ultsch, and M. G. Mulkerrin. 2001. ‘Engineered antibodies with increased activity to recruit complement’, J Immunol, 166: 2571-5.
Jacobi, A., B. Enenkel, P. Garidel, C. Eckermann, M. Knappenberger, I. Presser, and H. Kaufmann. 2014. ‘Process Development and Manufacturing of Therapeutic Antibodies.’ in S. Duebel and J. M. Reichert (eds.), Handbook of Therapeutic Antibodies, Second Edition (Wiley-VCH Verlag GmbH & Co. KGaA.).
Jain, N., S. W. Smith, S. Ghone, and B. Tomczuk. 2015. ‘Current ADC Linker Chemistry’, Pharm Res, 32: 3526-40.
Jiang, H., Z. Shi, P. Wang, C. Wang, L. Yang, G. Du, H. Zhang, B. Shi, J. Jia, Q. Li, H. Wang, and Z. Li. 2018. ‘Claudin18.2-Specific Chimeric Antigen Receptor Engineered T Cells for the Treatment of Gastric Cancer’, J Natl Cancer Inst.
June, C. H., and M. Sadelain. 2018. ‘Chimeric Antigen Receptor Therapy’, N Engl J Med, 379: 64-73.
Klose, D., M. Woitok, J. Niesen, R. R. Beerli, U. Grawunder, R. Fischer, S. Barth, R. Fendel, and T. Nachreiner. 2017. ‘Generation of an artificial human B cell line test system using Transpo-mAb™ technology to evaluate the therapeutic efficacy of novel antigen-specific fusion proteins’, PLoS One, 12: e0180305.
Lazar, G. A., W. Dang, S. Karki, O. Vafa, J. S. Peng, L. Hyun, C. Chan, H. S. Chung, A. Eivazi, S. C. Yoder, J. Vielmetter, D. F. Carmichael, R. J. Hayes, and B. I. Dahiyat. 2006. ‘Engineered antibody Fc variants with enhanced effector function’, Proc Natl Acad Sci USA, 103: 4005-10.
Leabman, M. K., Y. G. Meng, R. F. Kelley, L. E. DeForge, K. J. Cowan, and S. Iyer. 2013. ‘Effects of altered FcgammaR binding on antibody pharmacokinetics in cynomolgus monkeys’, MAbs, 5: 896-903.
Lo, M., H. S. Kim, R. K. Tong, T. W. Bainbridge, J. M. Vernes, Y. Zhang, Y. L. Lin, S. Chung, M. S. Dennis, Y. J. Zuchero, R. J. Watts, J. A. Couch, Y. G. Meng, J. K. Atwal, R. J. Brerski, C. Spiess, and J. A. Ernst. 2017. ‘Effector-attenuating Substitutions That Maintain Antibody Stability and Reduce Toxicity in Mice’, J Biol Chem, 292: 3900-08.
Lu, X., R. P. Nobrega, H. Lynaugh, T. Jain, K. Barlow, T. Boland, A. Sivasubramanian, M. Vasquez, and Y. Xu. 2019. ‘Deamidation and isomerization liability analysis of 131 clinical-stage antibodies’, MAbs, 11: 45-57.
Martin, A. C. R., and J. Allemn. 2014. ‘Bioinformatics Tools for Analysis of Antibodies.’ in, Handbook of Therapeutic Antibodies, Second Edition (Wiley-VCH Verlag GmbH & Co. KGaA.).
Mimoto, F., T. Igawa, T. Kuramochi, H. Katada, S. Kadono, T. Kamikawa, M. Shida-Kawazoe, and K. Hattori. 2013. ‘Novel asymmetrically engineered antibody Fc variant with superior FcgammaR binding affinity and specificity compared with afucosylated Fc variant’, MAbs, 5: 229-36.
Moldenhauer, G. 2014. ‘Selection Strategies for Monoclonal Antibodies.’ in S. Duebel and J. M. Reichert (eds.), Handbook of Therapeutic Antibodies, Second Edition (Wiley-VCH Verlag GmbH & Co. KGaA.).
Moore, G. L., H. Chen, S. Karki, and G. A. Lazar. 2010. ‘Engineered Fc variant antibodies with enhanced ability to recruit complement and mediate effector functions’, MAbs, 2: 181-9.
Natsume, A., M. In, H. Takamura, T. Nakagawa, Y. Shimizu, K. Kitajima, M. Wakitani, S. Ohta, M. Satoh, K. Shitara, and R. Niwa. 2008. ‘Engineered antibodies of IgG1/IgG3 mixed isotype with enhanced cytotoxic activities’, Cancer Res, 68: 3863-72.
Niimi, T., K. Nagashima, J. M. Ward, P. Minoo, D. B. Zimonjic, N. C. Popescu, and S. Kimura. 2001. ‘claudin-18, a novel downstream target gene for the T/EBP/NKX2.1 homeodomain transcription factor, encodes lung- and stomach-specific isoforms through alternative splicing’, Mol Cell Biol, 21: 7380-90.
Nuijens, T., A. Toplak, M. Schmidt, A. Ricci, and W. Cabri. 2019. ‘Natural Occurring and Engineered Enzymes for Peptide Ligation and Cyclization’, Front Chem, 7: 829.
Ponziani, S., G. Di Vittorio, G. Pitari, A. M. Cimini, M. Ardini, R. Gentile, S. Iacobelli, G. Sala, E. Capone, D. J. Flavell, R. Ippoliti, and F. Giansanti. 2020. ‘Antibody-Drug Conjugates: The New Frontier of Chemotherapy’, Int J Mol Sci, 21.
Quintieri, L., C. Geroni, M. Fantin, R. Battaglia, A. Rosato, W. Speed, P. Zanovello, and M. Floreani. 2005. ‘Formation and antitumor activity of PNU-159682, a major metabolite of nemorubicin in human liver microsomes’, Clin Cancer Res, 11: 1608-17.
Richards, J. O., S. Karki, G. A. Lazar, H. Chen, W. Dang, and J. R. Desjarlais. 2008. ‘Optimization of antibody binding to FcgammaRIIa enhances macrophage phagocytosis of tumor cells’, Mol Cancer Ther, 7: 2517-27.
Rother, R. P., S. A. Rollins, C. F. Mojcik, R. A. Brodsky, and L. Bell. 2007. ‘Discovery and development of the complement inhibitor eculizumab for the treatment of paroxysmal nocturnal hemoglobinuria’, Nat Biotechnol, 25: 1256-64.
Sahin, U., M. Koslowski, K. Dhaene, D. Usener, G. Brandenburg, G. Seitz, C. Huber, and O. Tureci. 2008. ‘Claudin-18 splice variant 2 is a pan-cancer target suitable for therapeutic antibody development’, Clin Cancer Res, 14: 7624-34.
Sahin, U., M. Schuler, H. Richly, S. Bauer, A. Krilova, T. Dechow, M. Jerling, M. Utsch, C. Rohde, K. Dhaene, C. Huber, and O. Tureci. 2018. ‘A phase I dose-escalation study of IMAB362 (Zolbetuximab) in patients with advanced gastric and gastro-oesophageal junction cancer’, Eur J Cancer, 100: 17-26.
Saldanha, J. W. 2014. ‘Humanization Strategies.’ in S. Duebel and J. M. Reichert (eds.), Handbook of Therapeutic Antibodies, Second Edition (Wiley-VCH Verlag GmbH & Co. KGaA.).
Shang, L., B. Daubeuf, M. Triantafilou, R. Olden, F. Depis, A. C. Raby, S. Herren, A. Dos Santos, P. Malinge, I. Dunn-Siegrist, S. Benmkaddem, A. Geinoz, G. Magistrelli, F. Rousseau, V. Buatois, S. Salgado-Pires, W. Reith, R. Monteiro, J. Pugin, O. Leger, W. Ferlin, M. Kosco-Vilbois, K. Triantafilou, and G. Elson. 2014. ‘Selective antibody intervention of Toll-like receptor 4 activation through Fc gamma receptor tethering’, J Biol Chem, 289: 15309-18.
Shields, R. L., A. K. Namenuk, K. Hong, Y. G. Meng, J. Rae, J. Briggs, D. Xie, J. Lai, A. Stadlen, B. Li, J. A. Fox, and L. G. Presta. 2001. ‘High resolution mapping of the binding site on human IgG1 for Fc gamma RI, Fc gamma RII, Fc gamma RIII, and FcRn and design of IgG1 variants with improved binding to the Fc gamma R’, J Biol Chem, 276: 6591-604.
Stavenhagen, J. B., S. Gorlatov, N. Tuaillon, C. T. Rankin, H. Li, S. Burke, L. Huang, S. Vijh, S. Johnson, E. Bonvini, and S. Koenig. 2007. ‘Fc optimization of therapeutic antibodies enhances their ability to kill tumor cells in vitro and controls tumor expansion in vivo via low-affinity activating Fcgamma receptors’, Cancer Res, 67: 8882-90.
Stefan, N., R. Gebleux, L. Waldmeier, T. Hell, M. Escher, F. I. Wolter, U. Grawunder, and R. R. Beerli. 2017. ‘Highly Potent, Anthracycline-based Antibody-Drug Conjugates Generated by Enzymatic, Site-specific Conjugation’, Mol Cancer Ther, 16: 879-92.
Tao, M. H., and S. L. Morrison. 1989. ‘Studies of aglycosylated chimeric mouse-human IgG. Role of carbohydrate in the structure and effector functions mediated by the human IgG constant region’, J Immunol, 143: 2595-601.
Tureci, O., U. Sahin, H. Schulze-Bergkamen, Z. Zvirbule, F. Lordick, D. Koeberle, P. Thuss-Patience, T. Ettrich, D. Arnold, F. Bassermann, S. E. Al-Batran, K. Wiechen, K. Dhaene, D. Maurus, M. Gold, C. Huber, A. Krivoshik, A. Arozullah, J. W. Park, and M. Schuler. 2019. ‘A multicentre, phase 2a study of zolbetuximab as a single agent in patients with recurrent or refractory advanced adenocarcinoma of the stomach or lower oesophagus: the MONO study’, Ann Oncol.
Vafa, O., G. L. Gilliland, R. J. Brerski, B. Strake, T. Wilkinson, E. R. Lacy, B. Scallon, A. Teplyakov, T. J. Malia, and W. R. Strohl. 2014. ‘An engineered Fc variant of an IgG eliminates all immune effector functions via structural perturbations’, Methods, 65: 114-26.
Waldmeier, L., I. Hellmann, C. K. Gutknecht, F. I. Wolter, S. C. Cook, S. T. Reddy, U. Grawunder, and R. R. Beerli. 2016. ‘Transpo-mAb display: Transposition-mediated B cell display and functional screening of full-length IgG antibody libraries’, MAbs, 8: 726-40.
Walker, M. R., J. Lund, K. M. Thompson, and R. Jefferis. 1989. ‘Aglycosylation of human IgG1 and IgG3 monoclonal antibodies can eliminate recognition by human cells expressing Fc gamma RI and/or Fc gamma RII receptors’, Biochem J, 259: 347-53.
Wang, X., M. Mathieu, and R. J. Brerski. 2018. ‘IgG Fc engineering to modulate antibody effector functions’, Protein Cell, 9: 63-73.
Xu, D., M. L. Alegre, S. S. Varga, A. L. Rothermel, A. M. Collins, V. L. Pulito, L. S. Hanna, K. P. Dolan, P. W. Parren, J. A. Bluestone, L. K. Jolliffe, and R. A. Zivin. 2000. ‘In vitro characterization of five humanized OKT3 effector function variant antibodies’, Cell Immunol, 200: 16-26.
Yu, D., and J. R. Turner. 2008. ‘Stimulus-induced reorganization of tight junction structure: the role of membrane traffic’, Biochim Biophys Acta, 1778: 709-16.
Yu, S. F., B. Zheng, M. Go, J. Lau, S. Spencer, H. Raab, R. Soriano, S. Jhunjhunwala, R. Cohen, M. Caruso, P. Polakis, J. Flygare, and A. G. Polson. 2015. ‘A Novel Anti-CD22 Anthracycline-Based Antibody-Drug Conjugate (ADC) That Overcomes Resistance to Auristatin-Based ADCs’, Clin Cancer Res, 21: 3298-306.
Zalevsky, J., A. K. Chamberlain, H. M. Horton, S. Karki, I. W. Leung, T. J. Sproule, G. A. Lazar, D. C. Roopenian, and J. R. Desjarlais. 2010. ‘Enhanced antibody half-life improves in vivo activity’, Nat Biotechnol, 28: 157-9.
CN109762067
U.S. Pat. No. 10,435,471
WO2000/015659
WO2004/047863
WO2005/113587
WO2007/059997
WO2008/145338
WO2010/009124
WO2013/167259
WO2013/174509
WO2014/075788
WO2014/127906
WO2016/102679
WO2016/166122
WO2016/165762
WO2018/006882
WO2019/173420
WO2019/175617
WO2019/219089

1. An antibody-drug conjugate having the general formula A-(L-T)ₙ, wherein
a. A is an antibody or fragment thereof binding to CLDN18.2 comprising the HCDR1, HCDR2 and HCDR3 sequences of SEQ ID NO: 21, SEQ ID NO: 22 and SEQ ID NO: 23 respectively, and the LCDR1, LCDR2 and LCDR3 sequences of SEQ ID NO: 24, SEQ ID NO: 25 and SEQ ID NO: 26 respectively,
b. L is a linker, and
c. Tisa toxin,
wherein the toxin is an anthracycline,
wherein n is an integer between ≥1 and ≤10;
or a pharmaceutically acceptable salt or ester thereof.

2. The antibody-drug conjugate of claim 1, wherein the linker L comprises at least one a non-cleavable linker element, preferably wherein the non-cleavable linker element is selected from the group consisting of
a. ethylenediamine (EDA),
b. N-formyl-N,N′-dimethylethylenediamine,
c. diethylamine (DEA),
d. a piperazine-derived compound of the following formula: 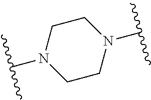 [SMILES: *N1CCN(*)CC1]
wherein the wavy lines indicate attachments to the toxin and another linker element,
e. the compound of the following formula: 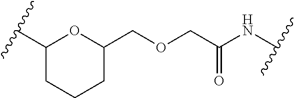 [SMILES: *NC(=O)COCC1CCCC(*)O1]
wherein the wavy lines indicate attachments to the toxin and another linker element,
f. the compound of the following formula: 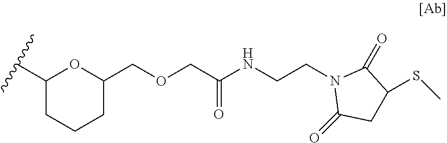 [SMILES: *C1CCCC(COCC(=O)NCCN2C(=O)CC(SC)C2=O)O1]
wherein the wavy lines indicate attachments to the toxin and [Ab] indicates the antibody or fragment thereof,
g. a maleimidocaproyl compound of the following formula: 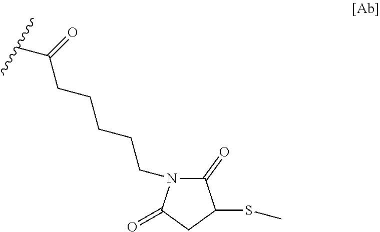 [SMILES: *C(=O)CCCCCN1C(=O)CC(SC)C1=O]
wherein the wavy lines indicate attachments to another linker element and [Ab] indicates the antibody or fragment thereof,
h. the compound of the following formula: 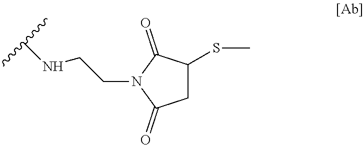 [SMILES: *NCCN1C(=O)CC(SC)C1=O]
wherein the wavy lines indicate attachments to a toxin and [Ab] indicates the antibody or fragment thereof,
and wherein the non-cleavable linker element is conjugated to the toxin by means of an amide bond or an ether bond.

3. The antibody-drug conjugate of claim 2, wherein the linker further comprises an oligopeptide linker element and/or enzyme-cleavable linker element and/or a spacer element.

4. The antibody-drug conjugate of claim 3, wherein one oligopeptide linker element comprises a sortase recognition motif oligopeptide selected from: -LPXTGₘ-, -LPXAGₘ-, -LPXSGₘ-, -LAXTGₘ-, -LPXTGₘ-, -LPXTAₘ-, -NPQTGₘ- or -NPQTNₘ-, with Gₘ being an oligoglycine with m being an integer between ≥1 and ≤21, A m being an oligoalanine with m being an integer between ≥1 and ≤21, Nₘ being an oligoasparagine with m being an integer between ≥1 and ≤21 and X being any conceivable amino acid, preferably the sortase recognition motif oligopeptide being -LPQTGG- or -LPETGG-, preferably wherein the oligopeptide linker element comprises:
a. the sequence SEQ ID NO: 131, or
b. the sequence SEQ ID NO: 132.

5. The antibody-drug conjugate of any of claim 3 or claim 4, wherein the enzyme-cleavable linker element comprises a val-cit-PAB linker according to the compound of the following formula: 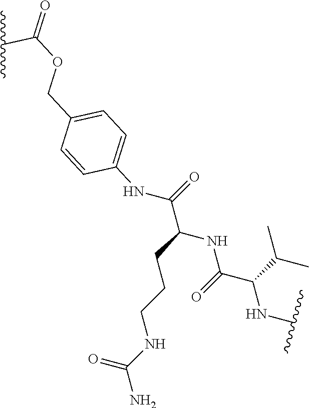 [SMILES: *N[C@H](C(=O)N[C@@H](CCCNC(N)=O)C(=O)Nc1ccc(COC(*)=O)cc1)C(C)C]
wherein the wavy lines indicate attachments to other linker elements.

6. The antibody-drug conjugate of any of claims 3 to 5, wherein the spacer element comprises a peptidic flexible oligopeptide, preferably wherein the peptidic flexible oligopeptide consists of G and S, more preferably wherein the peptidic flexible oligopeptide is (GGGGS)ₒ with o being 1, 2, 3, 4 or 5.

7. The antibody-drug conjugate of any of claims 1 to 6, wherein the antibody drug conjugate has the following structure:
a. A-([oligopeptide linker element-non-cleavable linker element]-T)ₙ and preferably wherein the linker is selected from: 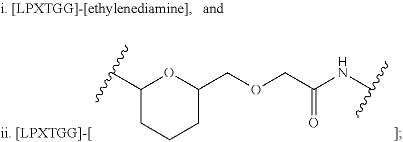 [SMILES: *NC(=O)COCC1CCCC(*)O1]
b. A-([oligopeptide linker element-enzyme cleavable linker element-non-cleavable linker element]-T)ₙ and preferably wherein the linker is selected from:
i.
[LPXTGG]-[vc-PAB]-[N-formyl-N,N′-
dimethylethylenediamine],
and
ii. [LPXTGG]-[vc-PAB]-[piperazine];
c. A-([spacer element-oligopeptide linker element-non-cleavable linker element]-T)ₙ and preferably wherein the linker is selected from: 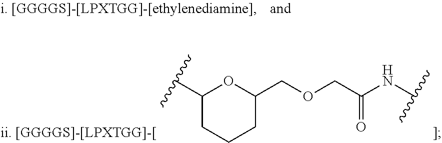 [SMILES: *NC(=O)COCC1CCCC(*)O1]
or
d. A-([spacer element-oligopeptide linker element-enzyme cleavable linker element-non-cleavable element]-T)ₙ and preferably wherein the linker is selected from:
i.
[GGGGS]-[LPXTGG]-[vc-PAB]-[N-formyl-N,N′-
dimethylethylenediamine],
and
ii.
[GGGGS]-[LPXTGG]-[vc-PAB]-[piperazine].

8. The antibody-drug conjugate of claim 7, wherein the non-cleavable linker element is ethylenediamine and wherein the oligopeptide linker element is LPXTGG wherein X is Q or E, preferably wherein X is Q.

9. The antibody-drug conjugate of any of claims 1 to 8, wherein
a. (L-T) is covalently linked to both light chains of the antibody,
b. (L-T) is covalently linked to both heavy chains of the antibody, or
c. (L-T) is covalently linked to both light chains and both heavy chains of the antibody.

10. The antibody-drug conjugate of any of claims 1 to 9, wherein the anthracycline derivative has the following formula (I), and is covalently linked to the non-cleavable linker element by the C₁₃ resulting in the loss of the C₁₄ and the hydroxyl group, or is covalently linked to the non-cleavable linker element by the hydroxyl group on C₁₄: 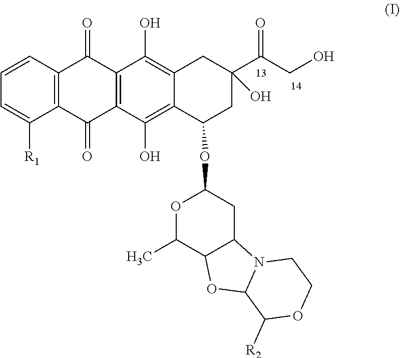
and wherein R₁ is a hydrogen atom, a hydroxy or methoxy group,
and wherein R₂ is a C₁-C₅ alkoxy group, preferably wherein the anthracycline derivative is a derivative of 3′-deamino-3″,4′-anhydro-[2″ (S)-methoxy-3″(R)-oxy-4″-morpholinyl]doxorubicin (PNU-159682).

11. The antibody-drug conjugate of any of claims 1 to 10, wherein A, the antibody or fragment thereof, comprises:
a. the HCDR1, HCDR2 and HCDR3 sequences of SEQ ID NO: 1, SEQ ID NO: 15 and SEQ ID NO: 3, respectively, and the LCDR1, LCDR2 and LCDR3 sequences of SEQ ID NO: 4, SEQ ID NO: 5 and SEQ ID NO: 6, respectively;
b. the HCDR1, HCDR2 and HCDR3 sequences of SEQ ID NO: 1, SEQ ID NO: 16 and SEQ ID NO: 3, respectively, and the LCDR1, LCDR2 and LCDR3 sequences of SEQ ID NO: 4, SEQ ID NO: 5 and SEQ ID NO: 6, respectively;
c. the HCDR1, HCDR2 and HCDR3 sequences of SEQ ID NO: 1, SEQ ID NO: 16 and SEQ ID NO: 3, respectively, and the LCDR1, LCDR2 and LCDR3 sequences of SEQ ID NO: 17, SEQ ID NO: 14 and SEQ ID NO: 11, respectively;
d. the HCDR1, HCDR2 and HCDR3 sequences of SEQ ID NO: 1, SEQ ID NO: 16 and SEQ ID NO: 3, respectively, and the LCDR1, LCDR2 and LCDR3 sequences of SEQ ID NO: 18, SEQ ID NO: 19 and SEQ ID NO: 11, respectively;
e. the HCDR1, HCDR2 and HCDR3 sequences of SEQ ID NO: 12, SEQ ID NO: 15 and SEQ ID NO: 3, respectively, and the LCDR1, LCDR2 and LCDR3 sequences of SEQ ID NO: 4, SEQ ID NO: 5 and SEQ ID NO: 6, respectively;
f. the HCDR1, HCDR2 and HCDR3 sequences of SEQ ID NO: 1, SEQ ID NO: 20 and SEQ ID NO: 3, respectively, and the LCDR1, LCDR2 and LCDR3 sequences of SEQ ID NO: 4, SEQ ID NO: 5 and SEQ ID NO: 6, respectively;
g. the HCDR1, HCDR2 and HCDR3 sequences of SEQ ID NO: 1, SEQ ID NO: 20 and SEQ ID NO: 3, respectively, and the LCDR1, LCDR2 and LCDR3 sequences of SEQ ID NO: 18, SEQ ID NO: 19 and SEQ ID NO: 11, respectively;
h. the HCDR1, HCDR2 and HCDR3 sequences of SEQ ID NO: 12, SEQ ID NO: 20 and SEQ ID NO: 8, respectively, and the LCDR1, LCDR2 and LCDR3 sequences of SEQ ID NO: 4, SEQ ID NO: 5 and SEQ ID NO: 6, respectively; or
i. the HCDR1, HCDR2 and HCDR3 sequences of SEQ ID NO: 12, SEQ ID NO: 20 and SEQ ID NO: 8, respectively, and the LCDR1, LCDR2 and LCDR3 sequences of SEQ ID NO: 17, SEQ ID NO: 14 and SEQ ID NO: 11, respectively.

12. The antibody-drug conjugate of any of claims 1 to 10, wherein A, the antibody or fragment thereof comprises:
a. the HCDR1, HCDR2 and HCDR3 sequences of SEQ ID NO: 1, SEQ ID NO: 2 and SEQ ID NO: 3, respectively, and the LCDR1, LCDR2 and LCDR3 sequences of SEQ ID NO: 4, SEQ ID NO: 5 and SEQ ID NO: 6, respectively;
b. the HCDR1, HCDR2 and HCDR3 sequences of SEQ ID NO: 1, SEQ ID NO: 7 and SEQ ID NO: 8, respectively, and the LCDR1, LCDR2 and LCDR3 sequences 20 of SEQ ID NO: 9, SEQ ID NO: 10 and SEQ ID NO: 11, respectively; or
c. the HCDR1, HCDR2 and HCDR3 sequences of SEQ ID NO: 12, SEQ ID NO: 2 and SEQ ID NO: 3, respectively, and the LCDR1, LCDR2 and LCDR3 sequences of SEQ ID NO: 13, SEQ ID NO: 14 and SEQ ID NO: 11, respectively,
preferably wherein A, the antibody or fragment thereof comprises:
a. a VH sequence of SEQ ID NO: 27 and a VL sequence of SEQ ID NO: 28;
b. a VH sequence of SEQ ID NO: 29 and a VL sequence of SEQ ID NO: 30; or
a VH sequence of SEQ ID NO: 31 and a VL sequence of SEQ ID NO: 32.

13. The antibody-drug conjugate of any of claims 1 to 11, wherein A, the antibody or fragment thereof comprises:
a. a VH sequence of SEQ ID NO: 33 and a VL sequence of SEQ ID NO: 38;
b. a VH sequence of SEQ ID NO: 34 and a VL sequence of SEQ ID NO: 38;
c. a VH sequence of SEQ ID NO: 34 and a VL sequence of SEQ ID NO: 39;
d. a VH sequence of SEQ ID NO: 34 and a VL sequence of SEQ ID NO: 40;
e. a VH sequence of SEQ ID NO: 35 and a VL sequence of SEQ ID NO: 38;
f. a VH sequence of SEQ ID NO: 36 and a VL sequence of SEQ ID NO: 41;
g. a VH sequence of SEQ ID NO: 36 and a VL sequence of SEQ ID NO: 40;
h. a VH sequence of SEQ ID NO: 37 and a VL sequence of SEQ ID NO: 41;
i. a VH sequence of SEQ ID NO: 37 and a VL sequence of SEQ ID NO: 38; or
j. a VH sequence of SEQ ID NO: 37 and a VL sequence of SEQ ID NO: 39, preferably wherein A, the antibody or fragment thereof, comprises:
a. the heavy chain sequence of SEQ ID NO: 46 and light chain sequence of SEQ ID 15 NO: 51;
b. the heavy chain sequence of SEQ ID NO: 47 and light chain sequence of SEQ ID NO: 51;
c. the heavy chain sequence of SEQ ID NO: 47 and light chain sequence of SEQ ID NO: 52;
d. the heavy chain sequence of SEQ ID NO: 47 and light chain sequence of SEQ ID NO: 53;
e. the heavy chain sequence of SEQ ID NO: 48 and light chain sequence of SEQ ID NO: 51;
f. the heavy chain sequence of SEQ ID NO: 47 and light chain sequence of SEQ ID 25 NO: 54;
g. the heavy chain sequence of SEQ ID NO: 49 and light chain sequence of SEQ ID NO: 53;
h. the heavy chain sequence of SEQ ID NO: 50 and light chain sequence of SEQ ID NO: 54;
i. the heavy chain sequence of SEQ ID NO: 50 and light chain sequence of SEQ ID NO: 51; or
j. the heavy chain sequence of SEQ ID NO: 50 and light chain sequence of, SEQ ID NO: 52 or versions thereof with an engineered Fc domain.

14. A method of producing an antibody-drug conjugate according to any of claims 1 to 19, wherein the method comprises the following steps:
a. providing A, an antibody or fragment thereof with an oligopeptide linker element preferably at its C-terminus, optionally preceded by a spacer element at the antibody light and/or heavy chains,
b. providing one or more toxins T with a non-cleavable linker element, and
c. conjugating the antibody and the toxin resulting in the antibody-drug conjugate.

15. An antibody-drug conjugate consisting of:
the antibody consisting of two heavy chains of the amino acid sequence according to SEQ ID NO: 46, and two light chains of the amino acid sequence according to SEQ ID NO: 51, wherein the antibody binds to CLDN18.2,
the linker [GGGGS]-[LPQTGG]-[ethylenediamine] at the C-terminus of the light chains, and
the anthracycline-based small molecule toxin 3′-deamino-3″,4′-anhydro-[2″(S)-methoxy-3″(R)-oxy-4″-morpholinyl]doxorubicin (PNU-159682), linked covalently to the ethylenediamine of the linker at C₁₃, resulting in the loss of C₁₄ and of the hydroxyl group; or
the antibody consisting of two heavy chains of the amino acid sequence according to SEQ ID NO: 133, and two light chains of the amino acid sequence according to SEQ ID NO: 51, wherein the antibody binds to CLDN18.2,
the linker [GGGGS]-[LPQTGG]-[ethylenediamine] at the C-terminus of the light chains, and
the anthracycline-based small molecule toxin 3′-deamino-3″,4′-anhydro-[2″(S)-methoxy-3″(R)-oxy-4″-morpholinyl]doxorubicin (PNU-159682), linked covalently to the ethylenediamine of the linker at C₁₃, resulting in the loss of C₁₄ and of the hydroxyl group; or
the antibody consisting of two heavy chains of the amino acid sequence according to SEQ ID NO: 134 and two light chains of the amino acid sequence according to SEQ ID NO: 51, wherein the antibody binds to CLDN18.2,
the linker [GGGGS]-[LPQXTGG]-[ethylenediamine] at the C-terminus of the light chains, and
the anthracycline-based small molecule toxin 3′-deamino-3″,4′-anhydro-[2″(S)-methoxy-3″(R)-oxy-4″-morpholinyl]doxorubicin (PNU-159682), linked covalently to the ethylenediamine of the linker at C₁₃, resulting in the 15 loss of C₁₄ and of the hydroxyl group.

16. A pharmaceutical composition comprising the antibody-drug conjugate of any of claims 1 to 15 and an excipient.

17. The antibody-drug conjugate of any of claims 1 to 15 for use in treatment.

18. The antibody-drug conjugate of any of claims 1 to 15 for use in the treatment of cancer, preferably wherein the cancer is selected from pancreatic, gastric, esophageal, ovarian, and lung cancer.


SEQUENCE LISTING (148 sequences)

SEQ ID NO: 1 - protein, 5 aa, Artificial Sequence
  cCl1-1, cCl1-2, hCl1a, hCl1b, hCl1c, hCl1d, hCl1f, hCl1g HCDR1
DYAMH

SEQ ID NO: 2 - protein, 17 aa, Artificial Sequence
  cCl1-1, cCl1-3 HCDR2
WINTYTGKPTYADDFKG

SEQ ID NO: 3 - protein, 10 aa, Artificial Sequence
  cCl1-1, cCl1-3 , hCl1a, hCl1b, hCl1c, hCl1d, hCl1e, hCl1f, hCl1g HCDR3
AVFYGYTMDA

SEQ ID NO: 4 - protein, 11 aa, Artificial Sequence
  cCl1-1, hCl1a, hCl1b, hCl1e, hCl1f, hCl1h, hCl1i LCDR1
RASEDIYSNLA

SEQ ID NO: 5 - protein, 7 aa, Artificial Sequence
  cCl1-1, hCl1a, hCl1b, hCl1e, hCl1f, hCl1h, hCl1i LCDR2
SVKRLQD

SEQ ID NO: 6 - protein, 9 aa, Artificial Sequence
  cCl1-1, hCl1a, hCl1b, hCl1e, hCl1f, hCl1h, hCl1i LCDR3
LQGSNFPLT

SEQ ID NO: 7 - protein, 17 aa, Artificial Sequence
  cCl1-2 HCDR2
WINAYTGKPTYADDFKG

SEQ ID NO: 8 - protein, 10 aa, Artificial Sequence
  cCl1-2, hCl1h, hCl1i, hCl1j HCDR3
AVYYGYTMDA

SEQ ID NO: 9 - protein, 11 aa, Artificial Sequence
  cCl1-2 LCDR1
RTSEDIYSNFA

SEQ ID NO: 10 - protein, 7 aa, Artificial Sequence
  cCl1-2 LCDR2
SVNRLQD

SEQ ID NO: 11 - protein, 9 aa, Artificial Sequence
  cCl1-2, cCl1-3, hCl1c, hCl1d, hCl1g, hCl1j LCDR3
LQGSKFPLT

SEQ ID NO: 12 - protein, 5 aa, Artificial Sequence
  cCl1-3, hCl1e, hCl1h, hCl1i, hCl1j HCDR1
DYAMY

SEQ ID NO: 13 - protein, 11 aa, Artificial Sequence
  cCl1-3 LCDR1
RTSEDIYSNLA

SEQ ID NO: 14 - protein, 7 aa, Artificial Sequence
  cCl1-3, hCl1c, hCl1j LCDR2
AIKRLQD

SEQ ID NO: 15 - protein, 17 aa, Artificial Sequence
  hCl1a, hCl1e HCDR2
WINTYTGKPTYAQKFQG

SEQ ID NO: 16 - protein, 17 aa, Artificial Sequence
  hCl1b, hCl1c, hCl1d HCDR2
WINTYTGKPTYSQKFQG

SEQ ID NO: 17 - protein, 11 aa, Artificial Sequence
  hCl1c, hCl1j LCDR1
RTSEDIYSNLA

SEQ ID NO: 18 - protein, 11 aa, Artificial Sequence
  hCl1d, hCl1g LCDR1
RTSEDIYSNFA

SEQ ID NO: 19 - protein, 7 aa, Artificial Sequence
  hCl1d, hCl1g LCDR2
SVNRLQD

SEQ ID NO: 20 - protein, 17 aa, Artificial Sequence
  hCl1f, hCl1g, hCl1h, hCl1i, hCl1j HCDR2
WINAYTGKPTYAQKFQG

SEQ ID NO: 21 - protein, 5 aa, Artificial Sequence
  HCDR1 consensus sequence
  MISC_FEATURE (5)..(5): H or Y
DYAMX

SEQ ID NO: 22 - protein, 17 aa, Artificial Sequence
  HCDR2 consensus sequence
  MISC_FEATURE (4)..(4): T or A
  MISC_FEATURE (12)..(12): A or S
  MISC_FEATURE (13)..(13): D or Q
  MISC_FEATURE (14)..(14): D or K
  MISC_FEATURE (16)..(16): K or Q
WINXYTGKPTYXXXFXG

SEQ ID NO: 23 - protein, 10 aa, Artificial Sequence
  HCDR3 consensus sequence
  MISC_FEATURE (3)..(3): F or Y
AVXYGYTMDA

SEQ ID NO: 24 - protein, 11 aa, Artificial Sequence
  LCDR1 consensus sequence
  MISC_FEATURE (2)..(2): A or T
  MISC_FEATURE (10)..(10): L or F
RXSEDIYSNXA

SEQ ID NO: 25 - protein, 7 aa, Artificial Sequence
  LCDR2 consensus sequence
  MISC_FEATURE (1)..(1): S or A
  MISC_FEATURE (2)..(2): V or I
  MISC_FEATURE (3)..(3): K or N
XXXRLQD

SEQ ID NO: 26 - protein, 9 aa, Artificial Sequence
  LCDR3 consensus sequence
  MISC_FEATURE (5)..(5): K or N
LQGSXFPLT

SEQ ID NO: 27 - protein, 119 aa, Artificial Sequence
  cCl1-1 HC variable region
QIQLVQSGPELKKPGESVKISCKASGYTFTDYAMHWVKQAPGKGLKWMGWINTYTGKPTYADDFKGRFVFSLEASASTANLQISNLKNEDTATYFCARAVFYGYTMDAWGQGTSVTVSS

SEQ ID NO: 28 - protein, 107 aa, Artificial Sequence
  cCl1-1 LC variable region
DIQMTQSPASLSASLGETISIACRASEDIYSNLAWYQQKSGKSPQLLIFSVKRLQDGVPSRFSGSGSGTQYSLKISGMQPEDEGDYFCLQGSNFPLTFGSGTKLEIK

SEQ ID NO: 29 - protein, 119 aa, Artificial Sequence
  cCl1-2 HC variable region
QIQLVQSGPELKKPGESVKISCKTSGYTFTDYAMHWVKQGPGKGMKWMGWINAYTGKPTYADDFKGRFVLSLEASASTANLQISNLKNEDTATYFCARAVYYGYTMDAWGQGTSVIVSS

SEQ ID NO: 30 - protein, 107 aa, Artificial Sequence
  cCl1-2 LC variable region
DIQMTQSPASLSASLGETISIECRTSEDIYSNFAWFQQKSGKSPQLLIYSVNRLQDGVPSRFSGSGSGTQYSLKISGMQPEDEGDYFCLQGSKFPLTFGSGTKLEIK

SEQ ID NO: 31 - protein, 119 aa, Artificial Sequence
  cCl1-3 HC variable region
QIQLVQSGPELKKPGESVKISCKASGYTFTDYAMYWVKQVPGKGLRWMGWINTYTGKPTYADDFKGRFVFSLEASASTANLQISNLKNEDTATYFCARAVFYGYTMDAWGQGTSVTVSS

SEQ ID NO: 32 - protein, 107 aa, Artificial Sequence
  cCl1-3 LC variable region
DIQMTQSPASLSASLGETISIACRTSEDIYSNLAWYQQKSGKSPQLLIFAIKRLQDGVPSRFSGSGSGTQYSLKISGMQPEDEGDYFCLQGSKFPLTFGSGTKLEIK

SEQ ID NO: 33 - protein, 119 aa, Artificial Sequence
  hCl1a HC variable region
QVQLVQSGAEVKKPGASVKVSCKASGYTFTDYAMHWVRQAPGQRLEWMGWINTYTGKPTYAQKFQGRVTITRDTSASTAYMELSSLRSEDTAVYYCARAVFYGYTMDAWGQGTLVTVSS

SEQ ID NO: 34 - protein, 119 aa, Artificial Sequence
  hCl1b, hCl1c and hCl1d HC variable region
QVQLVQSGAEVKKPGASVKVSCKASGYTFTDYAMHWVRQAPGQRLEWMGWINTYTGKPTYSQKFQGRVTITRDTSASTAYMELSSLRSEDTAVYYCARAVFYGYTMDAWGQGTLVTVSS

SEQ ID NO: 35 - protein, 119 aa, Artificial Sequence
  hCl1e HC variable region
QVQLVQSGAEVKKPGASVKVSCKASGYTFTDYAMYWVRQAPGQRLEWMGWINTYTGKPTYAQKFQGRVTITRDTSASTAYMELSSLRSEDTAVYYCARAVFYGYTMDAWGQGTLVTVSS

SEQ ID NO: 36 - protein, 119 aa, Artificial Sequence
  hCl1f and hCl1g HC variable region
QVQLVQSGAEVKKPGASVKVSCKASGYTFTDYAMHWVRQAPGQRLEWMGWINAYTGKPTYAQKFQGRVTITRDTSASTAYMELSSLRSEDTAVYYCARAVFYGYTMDAWGQGTLVTVSS

SEQ ID NO: 37 - protein, 119 aa, Artificial Sequence
  hCl1h, hCl1i and hCl1j HC variable region
QVQLVQSGAEVKKPGASVKVSCKASGYTFTDYAMYWVRQAPGQRLEWMGWINAYTGKPTYAQKFQGRVTITRDTSASTAYMELSSLRSEDTAVYYCARAVYYGYTMDAWGQGTLVTVSS

SEQ ID NO: 38 - protein, 107 aa, Artificial Sequence
  hCl1a, hCl1b, hCl1e and hCl1i LC variable region
DIQMTQSPSSLSASVGDRVTITCRASEDIYSNLAWYQQKPGKAPKLLIFSVKRLQDGVPSRFSGSGSGTDFTLTISSLQPEDFATYYCLQGSNFPLTFGQGTKVEIK

SEQ ID NO: 39 - protein, 107 aa, Artificial Sequence
  hCl1c and hCl1j LC variable region
DIQMTQSPSSLSASVGDRVTITCRTSEDIYSNLAWYQQKPGKAPKLLIFAIKRLQDGVPSRFSGSGSGTDFTLTISSLQPEDFATYYCLQGSKFPLTFGQGTKVEIK

SEQ ID NO: 40 - protein, 107 aa, Artificial Sequence
  hCl1d and hCl1g LC variable region
DIQMTQSPSSLSASVGDRVTITCRTSEDIYSNFAWYQQKPGKAPKLLIYSVNRLQDGVPSRFSGSGSGTDFTLTISSLQPEDFATYYCLQGSKFPLTFGQGTKVEIK

SEQ ID NO: 41 - protein, 107 aa, Artificial Sequence
  hCl1f and hCl1h LC variable region
DIQMTQSPSSLSASVGDRVTITCRASEDIYSNLAWYQQKPGKAPKLLIYSVKRLQDGVPSRFSGSGSGTDFTLTISSLQPEDFATYYCLQGSNFPLTFGQGTKVEIK

SEQ ID NO: 42 - protein, 119 aa, Artificial Sequence
  hCl3a, hCl1b and hCl1c HC variable region
QVQLQESGPGLVKPSETLSLTCAVSGYSVSSNYRWHWIRQPPGKGLEWIGYINIAGSTNYNPSLKSRVTISVDTSKNQFSLKLSSVTAADTAVYYCARNPSITRAMDAWGQGTLVTVSS

SEQ ID NO: 43 - protein, 107 aa, Artificial Sequence
  hCl3a LC variable region
DIQMTQSPSSLSASVGDRVTITCKSSQNIFKNLEWYQQKPGKAPKLLIYYTNNLQTGVPSRFSGSGSGTDFTLTISSLQPEDFATYYCYQYNSGPFTFGQGTKVEIK

SEQ ID NO: 44 - protein, 107 aa, Artificial Sequence
  hCl3b LC variable region
DIQMTQSPSSLSASVGDRVTITCRSSQNIFKNLEWYQQKPGKAPKLLIYYTNNLQTGVPSRFSGSGSGTDFTLTISSLQPEDFATYYCYQYNSGPFTFGQGTKVEIK

SEQ ID NO: 45 - protein, 107 aa, Artificial Sequence
  hCl3c LC variable region
DIQMTQSPSSLSASVGDRVTITCRSSQNIFKNLEWYQQKPGKAPKLLIYYTNNLQSGVPSRFSGSGSGTDFTLTISSLQPEDFATYYCYQYNSGPFTFGQGTKVEIK

SEQ ID NO: 46 - protein, 449 aa, Artificial Sequence
  hCl1a HC full
  MISC_FEATURE (449)..(449): X is Lys or Arg
QVQLVQSGAEVKKPGASVKVSCKASGYTFTDYAMHWVRQAPGQRLEWMGWINTYTGKPTYAQKFQGRVTITRDTSASTAYMELSSLRSEDTAVYYCARAVFYGYTMDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGX

SEQ ID NO: 47 - protein, 449 aa, Artificial Sequence
  hCl1b, hCl1c and hCl1d HC full
  MISC_FEATURE (449)..(449): X is Lys or Arg
QVQLVQSGAEVKKPGASVKVSCKASGYTFTDYAMHWVRQAPGQRLEWMGWINTYTGKPTYSQKFQGRVTITRDTSASTAYMELSSLRSEDTAVYYCARAVFYGYTMDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGX

SEQ ID NO: 48 - protein, 449 aa, Artificial Sequence
  hCl1e HC full
  MISC_FEATURE (449)..(449): X is Lys or Arg
QVQLVQSGAEVKKPGASVKVSCKASGYTFTDYAMYWVRQAPGQRLEWMGWINTYTGKPTYAQKFQGRVTITRDTSASTAYMELSSLRSEDTAVYYCARAVFYGYTMDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGX

SEQ ID NO: 49 - protein, 449 aa, Artificial Sequence
  hCl1f and hCl1g HC full
  MISC_FEATURE (449)..(449): X is Lys or Arg
QVQLVQSGAEVKKPGASVKVSCKASGYTFTDYAMHWVRQAPGQRLEWMGWINAYTGKPTYAQKFQGRVTITRDTSASTAYMELSSLRSEDTAVYYCARAVFYGYTMDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGX

SEQ ID NO: 50 - protein, 449 aa, Artificial Sequence
  hCl1h, hCl1i and hCl1j HC full
  MISC_FEATURE (449)..(449): X is Lys or Arg
QVQLVQSGAEVKKPGASVKVSCKASGYTFTDYAMYWVRQAPGQRLEWMGWINAYTGKPTYAQKFQGRVTITRDTSASTAYMELSSLRSEDTAVYYCARAVYYGYTMDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGX

SEQ ID NO: 51 - protein, 214 aa, Artificial Sequence
  hCl1a, hCl1b, hCl1e and hCl1i LC full
DIQMTQSPSSLSASVGDRVTITCRASEDIYSNLAWYQQKPGKAPKLLIFSVKRLQDGVPSRFSGSGSGTDFTLTISSLQPEDFATYYCLQGSNFPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 214 aa, Artificial Sequence
  hCl1c and hCl1j LC full
DIQMTQSPSSLSASVGDRVTITCRTSEDIYSNLAWYQQKPGKAPKLLIFAIKRLQDGVPSRFSGSGSGTDFTLTISSLQPEDFATYYCLQGSKFPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - protein, 214 aa, Artificial Sequence
  hCl1d and hCl1g LC full
DIQMTQSPSSLSASVGDRVTITCRTSEDIYSNFAWYQQKPGKAPKLLIYSVNRLQDGVPSRFSGSGSGTDFTLTISSLQPEDFATYYCLQGSKFPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 54 - protein, 214 aa, Artificial Sequence
  hCl1f and hCl1h LC full
DIQMTQSPSSLSASVGDRVTITCRASEDIYSNLAWYQQKPGKAPKLLIYSVKRLQDGVPSRFSGSGSGTDFTLTISSLQPEDFATYYCLQGSNFPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 448 aa, Artificial Sequence
  IMAB362 HC full
  MISC_FEATURE (448)..(448): X is Lys or Arg
QVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCTRSWRGNSFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGX

SEQ ID NO: 56 - protein, 220 aa, Artificial Sequence
  IMAB362 LC full
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - protein, 10 aa, Artificial Sequence
  N-terminal extracellular of CLDN18.2, independent of glycosylation
DQWSTQDLYN

SEQ ID NO: 58 - protein, 11 aa, Artificial Sequence
  N-terminal extracellular of CLDN18.2, mainly unglycosylated
NNPVTAVFNYQ

SEQ ID NO: 59 - protein, 14 aa, Artificial Sequence
  N-terminal extracellular domain of CLDN18.2, unglycosylated
STQDLYNNPVTAVF

SEQ ID NO: 60 - protein, 13 aa, Artificial Sequence
  pan-CLDN18 peptide in the C-terminal extracellular domain common to both CLDN18.1 and CLDN18.2 isoforms
TNFWMSTANMYTG

SEQ ID NO: 61 - protein, 16 aa, Artificial Sequence
  Specific epitope of CLDN18.2 as disclosed by WO2005/113587
ALMIVGIVLGAIGLLV

SEQ ID NO: 62 - protein, 24 aa, Artificial Sequence
  Specific epitope of CLDN18.2 as disclosed by WO2005/113587
RIGSMEDSAKANMTLTSGIMFIVS

SEQ ID NO: 63 - protein, 129 aa, Artificial Sequence
  First extracellular domain of CLDN18.2 with N- and C-terminal extensions
METDTLLLWVLLLWVPGSTGDAAQPARRARRTKLGTELGSTPVWWNSADGRMDQWSTQDLYNNPVTAVFNYQGLWRSCVRESSGFTECRGYFTLLGLPAMLQAVRAAIQHSGGRSRRARTKTHLRRGSE

SEQ ID NO: 64 - protein, 15 aa, Artificial Sequence
  Overlapping peptide within the first extracellular domain as disclosed by WO2008/145338
MDQWSTQDLYNNPVT

SEQ ID NO: 65 - protein, 15 aa, Artificial Sequence
  Overlapping peptide within the first extracellular domain as disclosed by WO2008/145338
LYNNPVTAVFNYQGL

SEQ ID NO: 66 - protein, 15 aa, Artificial Sequence
  Overlapping peptide within the first extracellular domain as disclosed by WO2008/145338
VFNYQGLWRSCVRES

SEQ ID NO: 67 - protein, 15 aa, Artificial Sequence
  Overlapping peptide within the first extracellular domain as disclosed by WO2008/145338
QGLWRSCVRESSGFT

SEQ ID NO: 68 - protein, 15 aa, Artificial Sequence
  Overlapping peptide within the first extracellular domain as disclosed by WO2008/145338
RSCVRESSGFTECRG

SEQ ID NO: 69 - protein, 15 aa, Artificial Sequence
  C-terminal epitope of CLDN18.2 as disclosed by WO2013/167259
TEDEVQSYPSKHDYV

SEQ ID NO: 70 - protein, 12 aa, Artificial Sequence
  C-terminal epitope of CLDN18.2 as disclosed by WO2013/167259
EVQSYPSKHDYV

SEQ ID NO: 71 - DNA, 15 bp, Artificial Sequence
  cCl1-1, cCl1-2, hCl1a HCDR1
gactacgcgatgcac

SEQ ID NO: 72 - DNA, 51 bp, Artificial Sequence
  cCl1-1 HCDR2
tggatcaacacgtacacggggaagccgacatacgcggacgacttcaagggg

SEQ ID NO: 73 - DNA, 30 bp, Artificial Sequence
  cCl1-1, cCl1-3 HCDR3
gccgtcttctacggatatacgatggacgcg

SEQ ID NO: 74 - DNA, 357 bp, Artificial Sequence
  cCl1-1 VH
cagatccagctcgtccagagcgggccggagctgaagaagccgggggagagcgtgaagatctcgtgcaaggcgagcggatatacgttcacggactacgcgatgcactgggtcaagcaagcgccggggaaagggctgaagtggatggggtggatcaacacgtacacggggaagccgacatacgcggacgacttcaaggggcgattcgtgttctcgctggaggcgagcgcgagcacggcgaacctgcaaatctcgaacctgaagaacgaggacacggcgacgtacttctgcgcgcgggccgtcttctacggatatacgatggacgcgtgggggcagggtaccagcgtgacggtctcgagc

SEQ ID NO: 75 - DNA, 33 bp, Artificial Sequence
  cCl1-1 LCDR1
cgggcgagcgaggacatctactcgaacctggcg

SEQ ID NO: 76 - DNA, 21 bp, Artificial Sequence
  cCl1-1 LCDR2
tccgtcaagcggctgcaagac

SEQ ID NO: 77 - DNA, 27 bp, Artificial Sequence
  cCl1-1 LCDR3
ctgcaagggagcaacttcccgctgacg

SEQ ID NO: 78 - DNA, 321 bp, Artificial Sequence
  cCl1-1 VL
gacatccagatgacgcagagcccggcgtcgctgagcgcgagcctgggggagacgatctcgatcgcgtgccgggcgagcgaggacatctactcgaacctggcgtggtatcaacagaagagcgggaagagcccgcagctgctgatcttctccgtcaagcggctgcaagacggcgtcccgagccgattctcggggagcgggagcgggacgcagtactcgctgaagatctcggggatgcagccggaggacgagggggactacttctgcctgcaagggagcaacttcccgctgacgttcgggtcgggtaccaaactcgagatcaaa

SEQ ID NO: 79 - DNA, 51 bp, Artificial Sequence
  cCl1-2 HCDR2
tggatcaacgcgtacacggggaagccgacctacgcggacgacttcaagggg

SEQ ID NO: 80 - DNA, 27 bp, Artificial Sequence
  cCl1-2 HCDR3
gccgtctactacggatatacgatggac

SEQ ID NO: 81 - DNA, 357 bp, Artificial Sequence
  cCl1-2 VH
cagatccagctcgtccagagcgggccggagctgaagaagccgggggagagcgtgaagatctcgtgcaagacgagcggatatacgttcacggactacgcgatgcactgggtcaagcaggggccagggaaagggatgaagtggatggggtggatcaacgcgtacacggggaagccgacctacgcggacgacttcaaggggcgattcgtgctgagcctggaggcgagcgcctcgacggcgaacctgcaaatctcgaacctgaagaacgaggacacggcgacgtacttctgcgcgcgggccgtctactacggatatacgatggacgcgtgggggcagggtaccagcgtgatcgtctcgagc

SEQ ID NO: 82 - DNA, 33 bp, Artificial Sequence
  cCl1-2 LCDR1
cggacgagcgaggacatctactcgaacttcgcg

SEQ ID NO: 83 - DNA, 21 bp, Artificial Sequence
  cCl1-2 LCDR2
tcagtcaaccggctgcaagac

SEQ ID NO: 84 - DNA, 27 bp, Artificial Sequence
  cCl1-2, cCl1-3, hCl1d, hCl1g LCDR3
ctgcaagggagcaagttcccgctgacg

SEQ ID NO: 85 - DNA, 321 bp, Artificial Sequence
  cCl1-1 VL
gacatccagatgacgcagagcccggcgagcctgagcgcgagcctgggggagacgatctcgatcgagtgccggacgagcgaggacatctactcgaacttcgcgtggttccagcagaagagcgggaagagcccgcagctgctgatctactcagtcaaccggctgcaagacggcgtcccgagccgattctcggggagcgggagcgggacgcagtactcgctgaagatctcggggatgcagccggaggacgagggggactacttctgcctgcaagggagcaagttcccgctgacgttcgggagcggtaccaaactcgagatcaaa

SEQ ID NO: 86 - DNA, 15 bp, Artificial Sequence
  cCl1-3 HCDR1
gactacgcgatgtac

SEQ ID NO: 87 - DNA, 51 bp, Artificial Sequence
  cCl1-3 HCDR2
tggatcaacacgtacacggggaagccgacctacgcggacgacttcaagggg

SEQ ID NO: 88 - DNA, 357 bp, Artificial Sequence
  cCl1-3 VH
cagatccagctcgtccagagcgggccggagctgaagaagccgggggagagcgtgaagatctcgtgcaaggcgagcggatatacgttcacggactacgcgatgtactgggtcaagcaagtgccggggaaagggctgcgatggatggggtggatcaacacgtacacggggaagccgacctacgcggacgacttcaaggggcgattcgtgttctcgctggaggcgagcgcgagcacggcgaacctgcaaatctcgaacctgaagaacgaggacacggcgacgtacttctgcgcgcgggccgtcttctacggatatacgatggacgcgtgggggcagggtaccagcgtgacggtctcgagc

SEQ ID NO: 89 - DNA, 33 bp, Artificial Sequence
  cCl1-3 LCDR1
cggacgagcgaggacatctactcgaacctggcg

SEQ ID NO: 90 - DNA, 21 bp, Artificial Sequence
  cCl1-3 LCDR2
gcgatcaagcggctgcaagac

SEQ ID NO: 91 - DNA, 321 bp, Artificial Sequence
  cCl1-3 VL
gacatccagatgacgcagagcccggcgagcctgagcgcgagcctgggggagacgatctcgatcgcgtgccggacgagcgaggacatctactcgaacctggcgtggtatcaacagaagagcgggaagagcccgcagctgctgatcttcgcgatcaagcggctgcaagacggcgtcccgagccgattctcggggagcgggagcgggacgcagtactcgctgaagatctcggggatgcagccggaggacgagggggactacttctgcctgcaagggagcaagttcccgctgacgttcgggtcgggtaccaaactcgagatcaaa

SEQ ID NO: 92 - DNA, 51 bp, Artificial Sequence
  hCl1a HCDR2
tggatcaatacatacacggggaagccgacttatgcgcaaaaattccaagga

SEQ ID NO: 93 - DNA, 30 bp, Artificial Sequence
  hCl1a HCDR3
gcggtcttctacggatatacgatggatgcc

SEQ ID NO: 94 - DNA, 357 bp, Artificial Sequence
  cCl1a VH
caggtccaactagtccaaagcggggcggaagtcaagaagcccggagcatccgtcaaagtcagctgcaaggcgagcggatatacatttacggactacgcgatgcactgggtcaggcaagcccctgggcaaaggctcgaatggatgggatggatcaatacatacacggggaagccgacttatgcgcaaaaattccaaggaagagtcacaattacgcgggatacatccgcatctaccgcctacatggagctaagctcgctgcggagcgaggatacggcggtctactattgcgcccgagcggtcttctacggatatacgatggatgcctgggggcagggtaccctggtcacggtctcgagc

SEQ ID NO: 95 - DNA, 33 bp, Artificial Sequence
  hCl1a, hCl1b, hCl1e, hCl1i LCDR1
agggcctccgaagacatctactccaacctggca

SEQ ID NO: 96 - DNA, 21 bp, Artificial Sequence
  hCl1a, hCl1b, hCl1e, hCl1i LCDR2
agcgtcaaaagactacaagat

SEQ ID NO: 97 - DNA, 27 bp, Artificial Sequence
  hCl1a, hCl1b, hCl1e, hCl1i LCDR3
ttgcaaggaagcaatttccccttgact

SEQ ID NO: 98 - DNA, 321 bp, Artificial Sequence
  cCl1a, hCl1b, hCl1e, hCl1i VL
gacattcaaatgacgcaaagcccatcatcgctgagcgcatcggtcggggatagagtcaccataacatgcagggcctccgaagacatctactccaacctggcatggtatcaacaaaaaccggggaaggctccgaagctgctgatatttagcgtcaaaagactacaagatggagtaccgagccgattttcgggaagcgggagcgggacggatttcacgctgaccatatcaagtttgcaaccggaggattttgcgacatactattgcttgcaaggaagcaatttccccttgactttcgggcaaggtaccaaggtcgagatcaaa

SEQ ID NO: 99 - DNA, 15 bp, Artificial Sequence
  hCl1b, hCl1c, hCl1d HCDR1
gattatgcaatgcac

SEQ ID NO: 100 - DNA, 51 bp, Artificial Sequence
  hCl1b, hCl1c, hCl1d HCDR2
tggattaacacctacacgggcaagcccacatactcccaaaaattccaagga

SEQ ID NO: 101 - DNA, 30 bp, Artificial Sequence
  hCl1b, hCl1c, hCl1d HCDR3
gctgtattctatggatatacaatggatgcc

SEQ ID NO: 102 - DNA, 357 bp, Artificial Sequence
  hCl1b, hCl1c, hCl1d VH
caggtccaattagtccaaagcggggcggaagtcaagaagccgggggcgagcgtcaaagtctcatgcaaagcgagcggatacacatttacggattatgcaatgcactgggtcaggcaagcacccggacaaaggctggaatggatgggatggattaacacctacacgggcaagcccacatactcccaaaaattccaaggaagggtcacgataacgagagacacgagcgcgagcaccggaatggatgggatggattaacacctacacgggcaagcccacatactcccaaaaattccaaggaagggtcacgataacgagagacacgagcgcgagcaccgtaccctggtcaccgtctcgagc

SEQ ID NO: 103 - DNA, 33 bp, Artificial Sequence
  hCl1c, hCl1j LCDR1
cgaacgagcgaggacatatactcaaaccttgca

SEQ ID NO: 104 - DNA, 21 bp, Artificial Sequence
  hCl1c, hCl1j LCDR2
gcgataaagaggctgcaagac

SEQ ID NO: 105 - DNA, 27 bp, Artificial Sequence
  hCl1c, hCl1j LCDR3
ttgcaaggctccaaatttcccctgaca

SEQ ID NO: 106 - DNA, 321 bp, Artificial Sequence
  hCl1c, hCl1j VL
gacatccaaatgactcaaagcccatcatcgctatcggcatcggtcggggatagagtcacgataacatgccgaacgagcgaggacatatactcaaaccttgcatggtatcaacaaaagccggggaaggccccgaagctactgatattcgcgataaagaggctgcaagacggagttccatcacgattttcgggatctggctcggggaccgattttacgctgactatatcatcgctgcaaccggaagattttgcaacatactactgcttgcaaggctccaaatttcccctgacattcggacaaggtaccaaggtcgagatcaaa

SEQ ID NO: 107 - DNA, 33 bp, Artificial Sequence
  hCl1d, hCl1g LCDR1
cggacgagcgaggatatttattcgaactttgca

SEQ ID NO: 108 - DNA, 20 bp, Artificial Sequence
  hCl1d, hCl1g LCDR2
cagtcaatcggctacaagat

SEQ ID NO: 109 - DNA, 321 bp, Artificial Sequence
  hCl1d, hCl1g VL
gacatccaaatgacgcaatcaccgagctcgctgagcgcatctgtcggggaccgtgtcacaatcacatgccggacgagcgaggatatttattcgaactttgcatggtatcaacaaaaaccgggcaaggctccgaaacttttgatttattcagtcaatcggctacaagatggcgtcccgagccgatttagcgggagcggatcgggaaccgactttacgctgacgatatcatcgctacaaccggaggacttcgcgacttattactgcctacaagggagcaaattcccgctgacattcggacaaggtaccaaggtcgagatcaaa

SEQ ID NO: 110 - DNA, 15 bp, Artificial Sequence
  hCl1e HCDR1
gattacgcaatgtac

SEQ ID NO: 111 - DNA, 51 bp, Artificial Sequence
  hCl1e HCDR2
tggataaatacctatacgggaaagccaacatacgcccaaaaattccaaggc

SEQ ID NO: 112 - DNA, 30 bp, Artificial Sequence
  hCl1e HCDR3
gccgtcttttatggatatacgatggacgca

SEQ ID NO: 113 - DNA, 357 bp, Artificial Sequence
  hCl1e VH
caggtccaactggtccaatcgggggctgaagtcaaaaagccgggggcgagcgtcaaagtcagctgcaaagcatcgggatacacatttacggattacgcaatgtactgggtcaggcaagcacccggccaacgactggaatggatgggctggataaatacctatacgggaaagccaacatacgcccaaaaattccaaggccgcgtcacaataacgcgggacacgagcgcatcgacggcttatatggaactatcatcgctgcgatcggaagacacggcggtctattattgcgcacgcgccgtcttttatggatatacgatggacgcatgggggcagggtaccctggtcacggtctcgagc

SEQ ID NO: 114 - DNA, 15 bp, Artificial Sequence
  hCl1f, hCl1g HCDR1
gactacgcaatgcac

SEQ ID NO: 115 - DNA, 51 bp, Artificial Sequence
  hCl1f, hCl1g HCDR2
tggattaatgcctacacggggaagccgacctacgcacaaaaattccaagga

SEQ ID NO: 116 - DNA, 30 bp, Artificial Sequence
  hCl1f, hCl1g HCDR3
gccgtcttctatggatatacgatggatgct

SEQ ID NO: 117 - DNA, 357 bp, Artificial Sequence
  hCl1f, hCl1g VH
caggtccaattggtccaaagcggggcggaggtcaagaagccgggggcgagcgtcaaagtctcatgcaaggcaagcggatatacatttacggactacgcaatgcactgggtccggcaagcccctgggcaacggctggaatggatgggatggattaatgcctacacggggaagccgacctacgcacaaaaattccaaggacgagtcacgattacgcgggatactagcgcgagcaccgcatatatggagctaagctcgctgcgatctgaggataccgctgtatactactgcgcgagagccgtcttctatggatatacgatggatgcttgggggcagggtaccctggtcacggtctcgagc

SEQ ID NO: 118 - DNA, 33 bp, Artificial Sequence
  hCl1f, hCl1h LCDR1
cgagcttcggaggacatctatagcaacttggct

SEQ ID NO: 119 - DNA, 21 bp, Artificial Sequence
  hCl1f, hCl1h LCDR2
agcgtcaaaaggctccaagac

SEQ ID NO: 120 - DNA, 27 bp, Artificial Sequence
  hCl1f, hCl1h LCDR3
ctacaaggctctaacttcccattgaca

SEQ ID NO: 121 - DNA, 321 bp, Artificial Sequence
  hCl1f, hCl1h VL
gatatccaaatgacgcaatcaccatctagcctatcggcctctgtgggggaccgagtcaccatcacatgccgagcttcggaggacatctatagcaacttggcttggtatcaacaaaagccggggaaagcaccaaagctgctgatatatagcgtcaaaaggctccaagacggagtcccaagccgattctcgggctccggctccgggacggattttacgctgacaatttcgagcctgcaaccggaggactttgcaacctactattgcctacaaggctctaacttcccattgacatttgggcaaggtaccaaggtcgagatcaaa

SEQ ID NO: 122 - DNA, 15 bp, Artificial Sequence
  hCl1h, hCl1i, hCl1j HCDR1
gactacgctatgtat

SEQ ID NO: 123 - DNA, 51 bp, Artificial Sequence
  hCl1h, hCl1i, hCl1j HCDR2
tggattaatgcctacaccgggaagccgacttatgcgcaaaaatttcaagga

SEQ ID NO: 124 - DNA, 30 bp, Artificial Sequence
  hCl1h, hCl1i, hCl1j HCDR3
gcggtctactatggatatacgatggacgca

SEQ ID NO: 125 - DNA, 357 bp, Artificial Sequence
  hCl1h, hCl1i, hCl1j VH
caggtccaactggttcaatctggagcggaagtcaagaagcccggagcatccgtcaaagtctcgtgcaaggcatctggatacacattcaccgactacgctatgtattgggtccggcaagcccccggacaacggctggaatggatgggatggattaatgcctacaccgggaagccgacttatgcgcaaaaatttcaaggaagggtcacgattacgcgggacacgagcgcctcaaccgcatacatggagctatcgagcctgcgaagcgaggacaccgcggtctactactgcgcgcgggcggtctactatggatatacgatggacgcatgggggcagggtaccctggtcacggtctcgagc

SEQ ID NO: 126 - protein, 17 aa, Artificial Sequence
  HC CDR2 for hCl1x only, not chimeric clones cCl1-1,2,3
  MISC_FEATURE (4)..(4): T or A
  MISC_FEATURE (12)..(12): A or S
WINXYTGKPTYXQKFQG

SEQ ID NO: 127 - protein, 107 aa, Artificial Sequence [sequence omitted]

SEQ ID NO: 128 - protein, 330 aa, Artificial Sequence
  constant heavy chain - CH1 + Fc domain
  MISC_FEATURE (330)..(330): X is Lys or Arg
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGX

SEQ ID NO: 129 - protein, 330 aa, Artificial Sequence
  L234A/L235A mutation in constant heavy chain - CH1 + Fc domain
  MISC_FEATURE (330)..(330): X is Lys or Arg
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGX

SEQ ID NO: 130 - protein, 330 aa, Artificial Sequence
  L236A/L236A/P329G mutation in constant heavy chain - CH1 + Fc domain
  MISC_FEATURE (330)..(330): X is Lys or Arg
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGX

SEQ ID NO: 131 - protein, 6 aa, Artificial Sequence
  Sortase tag
LPQTGG

SEQ ID NO: 132 - protein, 11 aa, Artificial Sequence
  Sortase tag
GGGGSLPQTGG

SEQ ID NO: 133 - protein, 449 aa, Artificial Sequence
  hCL1a HC full LALA
  MISC_FEATURE (449)..(449): X is Lys or Arg
QVQLVQSGAEVKKPGASVKVSCKASGYTFTDYAMHWVRQAPGQRLEWMGWINTYTGKPTYAQKFQGRVTITRDTSASTAYMELSSLRSEDTAVYYCARAVFYGYTMDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGX

SEQ ID NO: 134 - protein, 449 aa, Artificial Sequence
  hCL1a HC full LALAPG
  MISC_FEATURE (449)..(449): X is Lys or Arg
QVQLVQSGAEVKKPGASVKVSCKASGYTFTDYAMHWVRQAPGQRLEWMGWINTYTGKPTYAQKFQGRVTITRDTSASTAYMELSSLRSEDTAVYYCARAVFYGYTMDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGX

SEQ ID NO: 135 - protein, 261 aa, Homo sapiens
MAVTACQGLGFVVSLIGIAGIIAATCMDQWSTQDLYNNPVTAVFNYQGLWRSCVRESSGFTECRGYFTLLGLPAMLQAVRALMIVGIVLGAIGLLVSIFALKCIRIGSMEDSAKANMTLTSGIMFIVSGLCAIAGVSVFANMLVTNFWMSTANMYTGMGGMVQTVQTRYTFGAALFVGWVAGGLTLIGGVMMCIACRGLAPEETNYKAVSYHASGHSVAYKPGGFKASTGFGSNTKNKKIYDGGARTEDEVQSYPSKHDYV

SEQ ID NO: 136 - protein, 5 aa, Artificial Sequence
  Sortase tag
  MISC_FEATURE (3)..(3): X is any of the 20 natural amino acids
  REPEAT (5)..(5): oligoglycine may comprise 1-21 repeats
LPXTG

SEQ ID NO: 137 - protein, 5 aa, Artificial Sequence
  Sortase tag
  MISC_FEATURE (3)..(3): X is any of the 20 natural amino acids
  REPEAT (5)..(5): oligoglycine may comprise 1-21 repeats
LPXAG

SEQ ID NO: 138 - protein, 5 aa, Artificial Sequence
  Sortase tag
  MISC_FEATURE (3)..(3): X is any of the 20 natural amino acids
  REPEAT (5)..(5): oligoglycine may comprise 1-21 repeats
LPXSG

SEQ ID NO: 139 - protein, 5 aa, Artificial Sequence
  Sortase tag
  MISC_FEATURE (3)..(3): X is any of the 20 natural amino acids
  REPEAT (5)..(5): oligoglycine may comprise 1-21 repeats
LAXTG

SEQ ID NO: 140 - protein, 5 aa, Artificial Sequence
  Sortase tag
  MISC_FEATURE (3)..(3): X is any of the 20 natural amino acids
  REPEAT (5)..(5): oligoalanine may comprise 1-21 repeats
LPXTA

SEQ ID NO: 141 - protein, 5 aa, Artificial Sequence
  Sortase tag
  REPEAT (5)..(5): oligoglycine may comprise 1-21 repeats
NPQTG

SEQ ID NO: 142 - protein, 5 aa, Artificial Sequence
  Sortase tag
  REPEAT (5)..(5): oligoasparagine may comprise 1-21 repeats
NPQTN

SEQ ID NO: 143 - protein, 6 aa, Artificial Sequence
  Sortase tag
LPETGG

SEQ ID NO: 144 - protein, 5 aa, Artificial Sequence
  Oligopeptide
  REPEAT (1)..(5): oligopeptide may comprise 1-5 repeats
GGGGS

SEQ ID NO: 145 - protein, 6 aa, Artificial Sequence
  Sortase tag
  MISC_FEATURE (3)..(3): X is any of the 20 natural amino acids
LPXTGG

SEQ ID NO: 146 - protein, 11 aa, Artificial Sequence
  Sortase tag
  MISC_FEATURE (8)..(8): X is any of the 20 natural amino acids
GGGGSLPXTGG

SEQ ID NO: 147 - protein, 5 aa, Artificial Sequence
  Oligopeptide
GGGGG

SEQ ID NO: 148 - protein, 5 aa, Artificial Sequence
  Sortase tag
LPQTG